(19) United States
(12) Patent Application Publication
(10) Pub. No.: US 2023/0063116 A1
(43) Pub. Date: Mar. 2, 2023

(54) VEGF MINI-TRAPS AND METHOD OF USE THEREOF

(71) Applicant: REGENERON PHARMACEUTICALS, INC., Tarrytown, NY (US)

(72) Inventors: Joel MARTIN, Tarrytown, NY (US); Samuel DAVIS, Tarrytown, NY (US); Shawn LAWRENCE, Nyack, NY (US); Amy JOHNSON, Briarcliff Manor, NY (US); Meghan CASEY, Ringwood, NJ (US); Jaimie MASTROGIACOMO, White Plains, NY (US); Shunhai WANG, Ossining, NY (US); Ning LI, New Canaan, CT (US); Andrew TUSTIAN, Milwood, NY (US); Ankit VARTAK, Elmsford, NY (US); Matthew FRANKLIN, Great Neck, NY (US)

(73) Assignee: REGENERON PHARMACEUTICALS, INC., Tarrytown, NY (US)

(21) Appl. No.: 17/782,508

(22) PCT Filed: Dec. 4, 2020
(86) PCT No.: PCT/US20/63238

§ 371 Date: Jun. 3, 2022

Related U.S. Application Data
(60) Provisional application No. 62/944,635, filed on Dec. 6, 2019.

(51) Int. Cl. C07K 16/28; A61K 9/00; C07K 14/52; C07K 14/71; C07K 1/16

(52) U.S. Cl. CPC C07K 16/2863; A61K 9/0048; C07K 14/52; C07K 14/71; C07K 1/16; A61K 38/00

(57) ABSTRACT

The present invention provides VEGF mini-trap molecules and method of treating or preventing angiogenic disorders such as angiogenic eye disorders and cancer.

### CROSS-REFERENCE TO RELATED APPLICATIONS

This application claims the benefit of U.S. provisional patent application No. 62/944,635; filed Dec. 6, 2019 which is herein incorporated by reference in its entirety.

### SEQUENCE LISTING

The sequence listing of the present application is submitted electronically as an ASCII formatted sequence listing with a file name “250298_000141_seqlist.TXT”, creation date of Dec. 2, 2020, and a size of 115,306 bytes. This sequence listing submitted is part of the specification and is herein incorporated by reference in its entirety.

### FIELD OF THE INVENTION

The present invention provides VEGF mini-trap molecules, pharmaceutical compositions thereof as well as methods of use thereof, e.g., for treating angiogenic eye disorders and cancer.

### BACKGROUND OF THE INVENTION

Several eye disorders are associated with pathological angiogenesis. For example, the development of age-related macular degeneration (AMD) is associated with a process called choroidal neovascularization (CNV). Leakage from the CNV causes macular edema and collection of fluid beneath the macula resulting in vision loss. Diabetic macular edema (DME) is another eye disorder with an angiogenic component. DME is the most prevalent cause of moderate vision loss in patients with diabetes and is a common complication of diabetic retinopathy, a disease affecting the blood vessels of the retina. Clinically significant DME occurs when fluid leaks into the center of the macula, the light-sensitive part of the retina responsible for sharp, direct vision. Fluid in the macula can cause severe vision loss or blindness. Yet another eye disorder associated with abnormal angiogenesis is central retinal vein occlusion (CRVO). CRVO is caused by obstruction of the central retinal vein that leads to a back-up of blood and fluid in the retina. The retina can also become ischemic, resulting in the growth of new, inappropriate blood vessels that can cause further vision loss and more serious complications. Release of vascular endothelial growth factor (VEGF) contributes to increased vascular permeability in the eye and inappropriate new vessel growth. Thus, inhibiting the angiogenic-promoting properties of VEGF is an effective strategy for treating angiogenic eye disorders.

Various VEGF inhibitors, such as the VEGF trap Eylea (aflibercept), have been approved to treat such eye disorders. The treatment protocols for delivering VEGF traps involve intravitreal injection. Such protocols are painful and inconvenient to the patient, psychologically and physically traumatic and involve the potential for adverse effects such as infection with each treatment event. Though aflibercept has proven to be highly effective in the treatment of various angiogenic eye disorders, dosing occurs as frequently as once a month. Therapeutic VEGF trap treatments that exhibit comparable efficacy and may be dosed less frequently are of great interest. Dosing with greater molar amounts of VEGF mini-trap, relative to aflibercept, would necessitate fewer dosing events while still benefiting from the high therapeutic efficacy of aflibercept.

### SUMMARY OF THE INVENTION

The present invention provides an isolated VEGF mini-trap (e.g., REGN7483^{F}) (which may be, for example, a monomer, homodimer or homomultimer) comprising the following domain structure: (R1D2)-(R2D3)-(MC), wherein one or more histidines of said VEGF mini-trap are oxidized to 2-oxo-histidine, and/or one or more tryptophans are dioxidated (e.g., to N-formylkynurenine) or oxidized to hydroxytryptophan or di-hydroxytrypophan or tri-hydroxyl tryptophan, and/or one or more asparagines thereof are glycosylated, or, ((R1D2)-(R2D3)-(R2D4))ₐ-(MC)_{b}, ((R1D2)-(R2D3))_{c}-linker-((R1D2)-(R2D3))_{d}, or ((R1D2)-(R2D3)-(R2D4))ₑ-linker-((R1D2)-(R2D3)-(R2D4))_{f}, wherein, R1D2 is the VEGFR1 Ig domain 2; R2D3 is the VEGFR2 Ig domain 3; R2D4 is the VEGFR2 Ig domain 4; MC is a multimerizing component consisting of the amino acid sequence: DKTHTCPPC (SEQ ID NO: 22), DKTHTCPPCPPC (SEQ ID NO: 23), DKTHTCPPCPPCPPC (SEQ ID NO: 24), DKTHTC (PPC)ₕ (SEQ ID NO: 25), wherein his 1, 2, 3, 4, or 5, DKTHTCPPCPAPELLG (SEQ ID NO: 6), DKTHTCPLCPAPELLG (SEQ ID NO: 7), DKTHTC (SEQ ID NO: 8) or DKTHTCPLCPAP (SEQ ID NO: 9) and linker is a peptide comprising about 5, 6, 7, 8, 9, 10, 11, 12, 13, 14, 15 or 16 amino acids; and, independently, a=1, 2, 3, 4, 5, 6, 7, 8, 9, 10, 11, 12, 13, 14 or 15; b=1, 2, 3, 4, 5, 6, 7, 8, 9, 10, 11, 12, 13, 14 or 15; c=1, 2, 3, 4, 5, 6, 7, 8, 9, 10, 11, 12, 13, 14 or 15; d=1, 2, 3, 4, 5, 6, 7, 8, 9, 10, 11, 12, 13, 14 or 15; e=1, 2, 3, 4, 5, 6, 7, 8, 9, 10, 11, 12, 13, 14 or 15; and f=1, 2, 3, 4, 5, 6, 7, 8, 9, 10, 11, 12, 13, 14 or 15; or a composition thereof, e.g., a aqueous composition. In an embodiment of the invention, the concentration of mini-trap (e.g., REGN7483^{F}) is about 90 mg/ml. For example, in an embodiment of the invention, the VEGF mini-trap includes or consists of an amino acid sequence set forth in a member selected from the group consisting of that set forth in SEQ ID NO: 10, 11, 12, 13, 26, 27, 28, 29, 30, 32 or 33. In an embodiment of the invention, the mini-trap comprises the domain structure: (i) ((R1D2)-(R2D3))ₐ-linker-((R1D2)-(R2D3))_{b}, or (ii) ((R1D2)-(R2D3)-(R2D4))_{c}-linker-((R1D2)-(R2D3)-(R2D4))_{d}, and has a secondary structure wherein: (i) said R1D2 domains coordinate; (ii) said R2D3 domains coordinate; and/or (iii) said R2D4 domains coordinate, to form a VEGF (e.g., VEGF-a) binding domain. In an embodiment of the invention, the linker is (Gly₄Ser)ₙ, e.g., wherein n is 1, 2, 3, 4, 5, 6, 7, 8, 9, 10, 11, 12, 13, 14 or 15. In an embodiment of the invention, the VEGF mini-trap or composition thereof includes one or more histidines that are oxidized to 2-oxo-histidine, and/or one or more tryptophans that are dioxidated, and/or one or more asparagines that are glycosylated. In an embodiment of the invention, a composition (e.g., an aqueous composition) includes that VEGF mini-trap wherein between 0.1% and 2% of histidines in the VEGF mini-trap are 2-oxo-histidine. In an embodiment of the invention, a composition includes said VEGF mini-trap such that oligopeptide products of digestion of the VEGF mini-trap (e.g., with S. pyogenes IdeS or a sequence variant thereof), which comprises one or more carboxymethylated cysteines and 2-oxo-histidines, with Lys-C and trypsin proteases are: EIGLLTC*EATVNGH*LYK (amino acids 73-89 of SEQ ID NO: 12) which comprises about 0.006-0.013% 2-oxo-histidines, QTNTIIDVVLSPSH*GIELSVGEK (amino acids 97-119 of SEQ ID NO: 12) which comprises about 0.019-0.028% 2-oxo-histidines, ELNVGIDFNWEYPSSKH*QHK (amino acids 128-148 of SEQ ID NO: 12) which comprises about 0.049-0.085% 2-oxo-histidines, DKTH*TC*PPC*PAPELLG (amino acids 206-221 of SEQ ID NO: 12) which comprises about 0.057-0.092% 2-oxo-histidines, TNYLTH*R (amino acids 90-96 of SEQ ID NO: 12) which comprises about 0.010-0.022% 2-oxo-histidines, and/or IIWDSR (amino acids 56-61 of SEQ ID NO: 12) which comprises about 0.198-0.298% 2-oxo-histidines, wherein H* is a histidine that may be oxidized to 2-oxo-histidine and wherein C* is a cysteine which may be carboxymethylated, optionally wherein the one or more tryptophans of said oligopeptides are dioxidated;
or
EIGLLTC*EATVNGH*LYK (amino acids 73-89 of SEQ ID NO: 12) which comprises about 0.0095 or 0.01% 2-oxo-histidines, QTNTIIDVVLSPSH*GIELSVGEK (amino acids 97-119 of SEQ ID NO: 12) which comprises about 0.0235 or 0.24% 2-oxo-histidines, TELNVGIDFNWEYPSSKH*QHK (amino acids 128-148 of SEQ ID NO: 12) which comprises about 0.067 or 0.07% 2-oxo-histidines, DKTH*TC*PPC*PAPELLG (amino acids 206-221 of SEQ ID NO: 12) which comprises about 0.0745 or 0.075% 2-oxo-histidines, TNYLTH*R (amino acids 90-96 of SEQ ID NO: 12) which comprises about 0.016 or 0.02% 2-oxo-histidines, and/or IIWDSR (amino acids 56-61 of SEQ ID NO: 12) which comprises about 0.248 or 0.25% 2-oxo-histidines, wherein H* is a histidine that may be oxidized to 2-oxo-histidine and wherein C* is a cysteine which may be carboxymethylated, optionally wherein the one or more tryptophans of said oligopeptides are dioxidated. In an embodiment of the invention, the 2-oxo-histidine is characterized by the chemical formula:

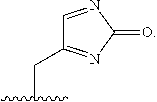 [SMILES: *CC1=NC(=O)N=C1]

The present invention includes a composition (e.g., an aqueous composition) including the VEGF mini-trap (e.g., REGN7850, REGN7851, REGN7483^{F} or REGN7483^{R}) wherein the composition characterized by a color:
(i) which is no more brown-yellow than European Color Standard BY2;
(ii) which is no more brown-yellow than European Color Standard BY3;
(iii) which is no more brown-yellow than European Color Standard BY4;
(iv) which is no more brown-yellow than European Color Standard BY5;
(v) which is no more brown-yellow than European Color Standard BY6;
(vi) which is no more brown-yellow than European Color Standard BY7;
(vi) which is between European Color Standard BY2 and BY3;
(vii) which is between European Color Standard BY2 and BY4;
(vii) wherein, in the CIEL*a*b* color space, L is about 70-99, a is about −2-0 and b is about 20 or less;
(viii) wherein, in the CIEL*a*b* color space, L is about 70-99, a is about −2-0 and b is about 10-31, about 10, about 14, about 12, about 14, about 15, about 18, about 21, about 27 or about 31;
(ix) wherein, in the CIEL*a*b* color space, L*, a* and b* are about those set forth in any of the rows in Table 9-3 herein or the BY value is about that set forth in Table 9-3, optionally wherein the concentrations are also approximately as set forth in the Table;
(x) wherein, in the CIEL*a*b* color space, L*, a* and b* are about those set forth in any of the rows in Table 17-1 herein, optionally wherein the concentrations are also approximately as set forth in the Table; optionally wherein the concentration of VEGF mini-trap is about 70-200 mg/ml (e.g., 70, 80, 90, 100, 110, 120, 130, 140, 150, 160, 170, 180, 190 or 200 mg/ml), or optionally, wherein the concentration of VEGF mini-trap is about 70-200 mg/ml (e.g., 70, 80, 90, 100, 110, 120, 130, 140, 150, 160, 170, 180, 190 or 200 mg/ml), but is characterized by said color when diluted to about 10 or 11 or 10-11 mg/ml. In an embodiment of the invention, a composition includes a mini-trap of the present invention wherein the color of the composition is characterized by the following formula: 0.046+(0.066×concentration of mini-trap (mg/ml))=b* or 0.05+(0.07×concentration of mini-trap (mg/ml))=b* or b*=(0.11×concentration of mini-trap (mg/ml)−0.56), wherein L*=about 97-99 and a=about −0.085-0.06 (e.g., about 0).

The present invention also includes a composition (e.g., an aqueous composition) including VEGF mini-trap (e.g., REGN7850, REGN7851, REGN7483^{F} or REGN7483^{R}) that is the product of a process comprising subjecting the mini-trap to anion exchange chromatography (e.g., in a loading buffer at a pH of about 8.3-8.6 and/or a conductivity of about 2 mS/cm) wherein the mini-trap is collected in the flow-through chromatographic fraction. For example, in an embodiment of the invention, the method comprises: (i) expressing aflibercept or said VEGF mini-trap in a host cell (e.g., Chinese hamster ovary cell) in a chemically-defined liquid medium wherein said aflibercept or VEGF mini-trap is secreted from the host cell into the medium; and (ii) if aflibercept is expressed, further comprising proteolytic cleavage of the aflibercept to produce peptides comprising the Fc domain, or a fragment thereof, and said VEGF mini-trap, and removal of the Fc domain or fragment thereof from the VEGF mini-trap; (iii) applying the VEGF mini-trap to an anion-exchange chromatography resin (e.g., having the functional group of a quaternary amine; —O—CH₂CHOHCH₂OCH₂CHOHCH₂N⁺(CH₃)₃; —N⁺(CH₃)₃, or a quaternized polyethyleneimine); and (iv) retaining said VEGF mini-trap polypeptide in the chromatographic flow-through thereof. In an embodiment of the invention, if said aflibercept is expressed, the process further comprises, prior to said proteolytic cleavage, protein-A purification of the aflibercept. In an embodiment of the invention, the proteolytic cleavage is performed by incubating the aflibercept with Streptococcus pyogenes IdeS protease or a variant thereof comprising one or more point mutations. In an embodiment of the VEGF mini-trap is applied to the anion-exchange chromatography resin which has been equilibrated in an aqueous buffer comprising: a buffer at a pH of about 8.4 or 7.7 and a conductivity of about 2.0 mS/cm, e.g., 50 mM Tris pH 8.4±0.1 and having a conductivity of 2.0 mS/cm, or 50 mM Tris, 60 mM NaCl, pH 7.7±0.1. In an embodiment of the invention, the VEGF mini-trap is applied to the anion-exchange resin when it is in an aqueous buffer at a pH of about 8.4 or 7.7 and a conductivity of about 2.0 mS/cm, e.g., 50 mM Tris pH 8.4±0.1 and having a conductivity of 2.0 mS/cm, or 50 mM Tris, 60 mM NaCl, pH 7.7±0.1. The resin may be washed with said aqueous buffer after the composition is applied to it and this wash may be retained. In an embodiment of the invention, the aflibercept Fc domain or fragment thereof is chromatographically removed from the VEGF mini-trap composition, following proteolytic cleavage, by applying the composition comprising Fc domain or fragment and VEGF mini-trap to a protein-A chromatography resin and retaining the VEGF mini-trap in the flow-through fraction. In an embodiment of the invention, the process further comprises adjustment to a more acidic pH (e.g., about 5.5, 5.6, 5.7, 5.8, 5.9, 6.0, 6.1, 6.2), filtration, depth filtration, ultrafiltration, diafiltration, viral inactivation, cation-exchange chromatography, protein-A chromatographic purification and/or hydrophobic interaction chromatographic purification (e.g., with a phenyl, octyl, or butyl functional group and/or run in bind-and-elute mode or flow-through mode), e.g., Phenyl sepharose FF, Capto Phenyl (GE Healthcare, Uppsala, Sweden), Phenyl 650-M (Tosoh Bioscience, Tokyo, Japan) or Sartobind Phenyl (Sartorius corporation, New York, USA). In an embodiment of the invention, the cysteine (e.g., cysteine HCl H₂O) concentration in the initial (day 0) chemically-defined liquid medium is about 1.5 mM and additional cysteine feeds are added to the culture medium at 1.3 mM, 1.7 mM or 2.1 mM (per volume or culture medium) every two days (e.g., days 2, 4, 6 and 8); the chemically-defined liquid medium comprises EDTA and/or citric acid, iron, copper, zinc and nickel; and/or the chemically-defined liquid medium comprises hypotaurine, taurine, glycine, thioctic acid and/or vitamin C.

In an embodiment of the invention, the VEGF mini-trap of the present invention (e.g., REGN7850, REGN7851, REGN7483^{F} or REGN7483^{R}; for example, which has been expressed in CDM (e.g., in CHO cells) and purified as set forth herein by AEX flow-through chromatography), is characterized as follows: one or more asparagines of the VEGF mini-trap are N-glycosylated; one or more serines or threonines of the VEGF mini-trap are O-glycosylated; one or more asparagines of the VEGF mini-trap are deamidated; one or more Aspartate-Glycine motifs of the VEGF mini-trap are converted to iso-aspartate-glycine and/or Asn-Gly; one or more methionines of the VEGF mini-trap are oxidized; one or more tryptophans of the VEGF mini-trap are converted to N-formylkynurenin; one or more arginines of the VEGF mini-trap are converted to Arg 3-deoxyglucosone; the C-terminal Glycine (or other C-terminal residue) of the VEGF mini-trap is not present; there are one or more non-glycosylated potential glycosites in the VEGF mini-trap; the VEGF mini-trap comprises about 40% to about 50% total fucosylated glycans; the VEGF mini-trap comprises about 30% to about 55% total sialylated glycans; the VEGF mini-trap comprises about 6% to about 15% mannose-5; the VEGF mini-trap comprises about 60% to about 79% galactosylated glycans; the VEGF mini-trap is xylosylated; the VEGF mini-trap is glycated at a lysine; the VEGF mini-trap comprises a cystine with a free-thiol group; the VEGF mini-trap comprises a trisulfide bridge; the VEGF mini-trap comprises an intrachain disulfide bridge; the VEGF mini-trap comprises disulfide bridges in parallel orientation; and/or the VEGF mini-trap comprises a lysine or arginine which is carboxymethylated; and/or as follows: wherein one or more asparagines of the VEGF mini-trap comprises: G0-GlcNAc glycosylation; G1-GlcNAc glycosylation; G1S-GlcNAc glycosylation; G0 glycosylation; G1 glycosylation; G1S glycosylation; G2 glycosylation; G2S glycosylation; G2S2 glycosylation; G0F glycosylation; G2F2S glycosylation; G2F2S2 glycosylation; G1F glycosylation; G1FS glycosylation; G2F glycosylation; G2FS glycosylation; G2FS2 glycosylation; G3FS glycosylation; G3FS3 glycosylation; G0-2GlcNAc glycosylation; Man4 glycosylation; Man4_A1G1 glycosylation; Man4_A1G1S1 glycosylation; Man5 glycosylation; Man5_A1G1 glycosylation; Man5_A1G1S1 glycosylation; Man6 glycosylation; Man6_G0+Phosphate glycosylation; Man6+Phosphate glycosylation; and/or Man7 glycosylation, e.g., which comprises Man5 glycosylation at about 30-36% (e.g., about 30, 31, 32, 32-35, 33, 34, 35 or 36%) of asparagine 123 residues and/or at about 25-30% (e.g., about 25, 26, 27, 27-30, 28, 29 or 30%) of asparagine 196 residues; about 6-8% (e.g., about 6, 7, 8%) of asparagine 36 glycosylated with Man6-phosphate; and/or about 3-4% (e.g., about 3 or 4 or 4.5%) of asparagine 123 glycosylated with Man7. In an embodiment of the invention, a mini-trap of the present invention (e.g., REGN7483^{F}, for example, which has been expressed in CDM (e.g., in CHO cells) and purified as set forth herein by AEX flow-through chromatography) has about 38% of asparagine 123 residues with high mannose glycosylation and/or about 29% of asparagine 196 residues with high mannose glycosylation.

The present invention provides a pharmaceutical formulation comprising the VEGF mini-trap (e.g., REGN7850, REGN7851, REGN7483^{F} or REGN7483^{R}) or composition (e.g., an aqueous composition) as set forth herein and a pharmaceutically acceptable carrier. Injection devices (e.g., pre-filled syringe (PFS), e.g., a sterile PFS) comprising the VEGF mini-trap polypeptide, composition or pharmaceutical formulation are also part of the present invention.

In an embodiment of the invention, a VEGF mini-trap (e.g., REGN7850, REGN7851, REGN7483^{F} or REGN7483^{R}), composition (e.g., an aqueous composition) or pharmaceutical formulation as set forth herein is in association with a further therapeutic agent.

The present invention also provides a polynucleotide, e.g., DNA, that encodes the polypeptide of a VEGF mini-trap (e.g., REGN7850, REGN7851, REGN7483^{F} or REGN7483^{R}) which is set forth herein. The present invention also provides a vector comprising the polynucleotide as well as a host cell (e.g., Chinese hamster ovary (CHO) cell) comprising the VEGF mini-trap, polynucleotide and/or vector.

The present invention also includes a method for making a VEGF mini-trap (e.g., REGN7850, REGN7851, REGN7483^{F} or REGN7483^{R}) as set forth herein comprising introducing a polynucleotide encoding a polypeptide of the mini-trap into a host cell (e.g., CHO cell), culturing the host cell in a medium under conditions wherein the polypeptide is expressed and, optionally, isolating the polypeptide from the host cell and/or medium. A VEGF mini-trap or composition (e.g., an aqueous composition) thereof which is the product of such a method is also part of the present invention.

The present invention also includes a method for making a VEGF mini-trap as set forth herein (e.g., REGN7850, REGN7851, REGN7483^{F} or REGN7483^{R}) comprising or consisting essentially of proteolyzing a VEGF Trap (e.g., aflibercept or conbercept) with an enzyme that cleaves an immunoglobulin Fc polypeptide after the following sequence: DKTHTCPPCPAPELLG (SEQ ID NO: 20), e.g., S. pyogenes IdeS or Streptococcus equi subspecies zooepidemicus IdeZ. A VEGF mini-trap or composition thereof which is the product of such a method is also part of the present invention.

The present invention also includes a method for administering a VEGF mini-trap as set forth herein (e.g., REGN7850, REGN7851, REGN7483^{F} or REGN7483^{R}) or composition thereof (e.g., an aqueous composition) or a pharmaceutical formulation thereof to a subject (e.g., a human) comprising introducing the VEGF mini-trap, composition or formulation, and optionally a further therapeutic agent, into the body of the subject, e.g., by intraocular injection, e.g., by intravitreal injection (e.g., about 100 microliters or less, e.g., about 70 microliters).

The present invention also includes a method for treating an angiogenic eye disorder (e.g., age-related macular degeneration (wet), age-related macular degeneration (dry), macular edema, macular edema following retinal vein occlusion, retinal vein occlusion (RVO), central retinal vein occlusion (CRVO), branch retinal vein occlusion (BRVO), diabetic macular edema (DME), choroidal neovascularization (CNV), iris neovascularization, neovascular glaucoma, post-surgical fibrosis in glaucoma, proliferative vitreoretinopathy (PVR), optic disc neovascularization, corneal neovascularization, retinal neovascularization, vitreal neovascularization, pannus, pterygium, vascular retinopathy, diabetic retinopathy, wherein the subject also has diabetic macular edema; and/or diabetic retinopathy) in a subject (e.g., a human) in need thereof, the method comprising intraocularly (e.g., intravitreally) injecting a therapeutically effective amount (e.g., 0.5 mg, 2 mg, 4 mg, 6 mg, 8 mg or 10 mg) of the VEGF mini-trap (e.g., REGN7850, REGN7851, REGN7483^{F} or REGN7483^{R}) or composition (e.g., an aqueous composition) or pharmaceutical formulation thereof (e.g., about 100 microliters or less, e.g., about 70 microliters), and optionally a further therapeutic agent, into an eye of the subject.

### BRIEF DESCRIPTION OF THE FIGURES

FIG. 1. Description of a VEGF mini-trap molecule which is the product of proteolysis of aflibercept with Streptococcus pyogenes IdeS (FabRICATOR)(REGN7483^{F}). The homodimeric molecule is depicted with the Ig hinge domain fragments binding each polypeptide together. The VEGFR1 domain, the VEGFR2 domain and the hinge domain fragment (MC) is indicated. The point in aflibercept where IdeS cleavage occurs is indicated with an “//”. The cleaved off Fc fragment from aflibercept is also indicated.

FIG. 2. Description of a single chain VEGF mini-trap depicting domain coordination. The VEGFR1, VEGFR2 and linker domains are indicated. The linker shown is (G₄S)₆ (REGN7080). The present invention includes single chain VEGF mini-traps with a (G₄S)₃; (G₄S)₉ or (G₄S)₁₂ linker.

FIG. 3(A-C). HEK293/D9/Flt-IL18Rα/Flt-IL18Rβ clone V3H9 cells were treated with increasing concentrations of VEGF₁₁₀, VEGF₁₂₁, or VEGF₁₆₅ (Panels A-C, respectively, black open squares), resulting in an increase in relative luminescence units (RLU), which reflects activation of the chimeric VEGF receptor. In the presence of 20 pM VEGF₁₁₀, VEGF₁₂₁, or VEGF₁₆₅, neutralization was observed with serial dilutions of REGN3 (black closed circles), REGN6824 (black closed squares), and REGN7080 (closed triangles).

FIG. 4(A-B). HEK293/D9/Flt-IL18Rα/Flt-IL18Rβ clone V3H9 cells were treated with increasing concentrations of VEGF₁₂₁ or VEGF₁₁₀ (Panels A-B, respectively, open squares), resulting in an increase in relative luminescence units (RLU), which reflects activation of the chimeric VEGF receptor. In the presence of 20 pM VEGF₁₂₁ or VEGF₁₁₀, neutralization was observed with serial dilutions of REGN3 (VEGF-Trap; closed circles), REGN7991 (black closed squares), and REGN7992 (open triangles).

FIG. 5(A-F). HEK293/D9/Flt-IL18Rα/Flt-IL18Rβ clone V3H9 cells were treated with increasing concentrations of VEGF₁₁₀ (A-B), VEGF₁₂₁ (C-D), or VEGF₁₆₅ (E-F), resulting in an increase in relative luminescence units (RLU), which reflects activation of the chimeric VEGF receptor. In the presence of 20 pM VEGF₁₁₀, 40 pM VEGF₁₂₁, or 40 pM VEGF₁₆₅, neutralization was determined with serial dilutions of REGN3 (VEGF-Trap; small closed black squares); REGN7483^{F} (large closed black squares or open grey squares (separate lots)); REGN7483^{R} (small black closed triangles); REGN112 (open triangles); REGN7850 (closed grey circles); REGN7851 (open circles) or VEGF control (open black squares).

FIG. 6. REGN6824:REGN110 complexes were analyzed by size exclusion chromatography coupled to multi angle light scattering (SEC-MALS). Relative UV absorbance at 280 nm (right Y-axis) as a function of retention time (X-axis) is shown for each sample and the measured molar mass of resolved peaks are indicated (left Y-axis). Peak 1 indicate the complex, peak 2 represents REGN6824 alone and Peak 3 represents REGN110 alone.

FIG. 7. REGN7080:REGN110 complexes were analyzed by Size exclusion chromatography coupled to multi angle light scattering (SEC-MALS). Relative UV absorbance at 280 nm (right Y-axis) as a function of retention time (X-axis) is shown for each sample and the measured molar mass of resolved peaks are indicated (left Y-axis). Peak 1 indicates the complex, peak 2 represents REGN7080 alone and Peak 3 represents REGN110 alone.

FIG. 8. REGN7483^{F}:REGN110 complexes were analyzed by Size exclusion chromatography coupled to multi angle light scattering (SEC-MALS). Relative UV absorbance at 280 nm (right Y-axis) as a function of retention time (X-axis) is shown for each sample and the measured molar mass of resolved peaks are indicated (left Y-axis). Peak 1 indicates the complex, Peak 1a is consistent with a mixture of REGN7483^{F} alone and the REGN110:REGN7483^{F} complex, peak 2 represents REGN7483^{F} alone and Peak 3 represents REGN110 alone.

FIG. 9. Surface area of abnormal neovascularization observed in OIR (oxygen induced retinopathy) model mice following intravitreal administration of control hFc, VEGF Trap (aflibercept), single chain mini-trap, REGN7080, or dimer mini-Trap, REGN7483^{F} are shown.

FIG. 10(A-B). Surface area of abnormal neovascularization observed in OIR (oxygen induced retinopathy) model mice following systemic (ip) administration of dimeric mini-trap, REGN7483^{F} (3 mg/kg, 30 mg/kg or 100 mg/kg; or 3 mg/kg control hFc) are shown (A). A historic study of the surface area (normalized against hFc control protein) in OIR mice systemically (ip) administered 2.5 mg/kg, 6.25 mg/kg, 25 mg/kg or 50 mg/kg aflibercept (VEGF Trap) (B) is also shown.

FIG. 11. Reducing and non-reducing SDS-PAGE gel of REGN112 (R112), REGN7850 (R7850), and REGN7851 (R7851) molecules (M=molecular weight marker). Dimer and monomer are indicated.

FIG. 12. Graphic summary of VEGF trap and mini-trap constructs.

FIG. 13. Graphic representation of the CIEL*a*b* color space.

FIG. 14(A-D). Post-translational modifications observed on CDM-expressed and non-CDM-expressed aflibercept (Eylea). The table in (A) shows site-specific asparagine-linked glycosylation observed on REGN7483^{F} and aflibercept (Eylea). The degree of shading of each box correlates with the degree of the indicated glycosylation at the indicated residue. % High Manose was calculated by summing up Man4, Man5, Man6 and Man7. The table in (B) shows other post-translational modifications including non-glycosylation at the N-linked glycosites observed on REGN7483^{F} and aflibercept (Eylea). The Table in (C) shows site-specific asparagine-linked glycosylation observed on REGN7483^{F} (mini-trap production 10), REGN7483^{R}, REGN7711 and aflibercept (Eylea). These tables only show the glycoforms with level >1% in any sample. (D) shows the structure of additional glycans.

FIG. 15. Baseline vascular permeability (leak/disc areas) across groups (aflibercept (500 μg and 2 mg dose); REGN7483^{R} (Minitrap R, 250.5 μg dose); REGN7483^{F}(Minitrap F, 254.4 μg and 1.4 mg dose) and placebo)).

FIG. 16. Vascular permeability inhibition over time (as a percentage of baseline) at equimolar doses of aflibercept (500 μg), REGN7483^{R} (MiniTrap Recombinant, 250.5 μg), REGN7483^{F} (MiniTrap Fabricator, 254.4 μg) and placebo.

FIG. 17. Vascular permeability inhibition over time (as a percentage of baseline) at high doses of aflibercept (2 mg) or REGN7483^{F} (MiniTrap Fabricator, 1.4 mg); or placebo.

FIG. 18. Intraocular pressure over time in rabbits in each treatment group (aflibercept (500 μg and 2 mg dose); REGN7483^{R} (MiniTrap Recombinant, 250.5 μg dose); REGN7483^{F} (MiniTrap Fabricator, 254.4 μg and 1.4 mg dose) and placebo)).

FIG. 19. Percent pathological vascular regression in each group (aflibercept (500 μg and 2 mg dose); REGN7483^{F} (MiniTrap F, 254.4 μg and 1.4 mg dose) and placebo)).

FIG. 20. Baseline vascular permeability in aflibercept (500 μg), REGN7483^{F}(Minitrap, 213 μg) or placebo groups.

FIG. 21. Percentage vascular permeability inhibition over time in aflibercept (500 μg), REGN7483^{F} (MiniTrap (F), 213 μg) or placebo groups.

FIG. 22. Color analysis of BY color standards in CIEL*a*b* color space.

FIG. 23. Evaluation of the percentage of 2-oxo-histidines (and tryptophan dioxidation) in commercial aflibercept and in oligopeptides from protease digested mini-trap production 10 which has been purified by AEX chromatography and oligopeptides from protease digested mini-trap production 10 which has been stripped from AEX chromatography.

FIG. 24(A-B). Effect of incubation of various components with aflibercept in fresh CDM on the generation of color (CIEL*a*b* predicted b*-value) (A); and actual by predicted b*-value plot. B-vitamin group is thiamine, niacinamide, pantothenic acid, biotin and pyridoxine.

FIG. 25. Effect of metal content and cysteine reduction on color (CIEL*a*b* predicted b*-value).

FIG. 26(A-B). Effect on the predicted b*-value of various anti-oxidants spiked into spent CDM containing aflibercept drug substance; graph (A) and tabular summary (B).

FIG. 27. Effect of REGN7483 concentration (mini-trap production 23) on b*-value.

FIG. 28. Results of an experiment performed to compare the acidic species present in different mini-trap productions and in fractions obtained on performing a strong cation exchange (CEX) chromatography.

FIG. 29. Strong cation-exchange chromatograms performed according to an exemplary embodiment for the mini-trap production 23 (prior to any purification procedure, ≤BY3) was subjected to CEX and for enriched variants of desialylated Mini-Trap (dsMT1) using a dual salt-pH gradient.

FIG. 30. Imaged capillary isoelectric focusing (iciEF) electropherograms performed according to an exemplary embodiment for the VEGF mini-trap production 23 (prior to any purification procedure, ≤BY3) was subjected to CEX and for enriched variants of desialylated VEGF mini-trap (dsMT1).

FIG. 31(A-C). (A) full-view of the chart of absorbance versus time (minutes) for VEGF Mini-Trap obtained by IdeS (FabRICATOR) cleavage of aflibercept produced using the commercial process (non-CDM) (MT4) and mini-trap production 10 (M1) at 350 nm; (B) full-view of the chart of absorbance versus time (16-30 minutes) for MT4 and MT1 at 350 nm; (C) full-view of the chart of absorbance versus time (30-75 minutes) for MT4 and MT1 at 350 nm.

FIG. 32(A-B). Natural log plots of decay curves of (A) VEGF Trap REGN3 and (B) VEGF mini-trap REGN7483^{F} in vitreous of New Zealand White rabbits (Rabbits 428, 429, 430, 434, 435 and 436). OD=oculus dexter; OS=oculus sinister.

FIG. 33(A-C). Natural log plots of decay curves of (A) VEGF Trap REGN3, (B) VEGF mini-trap REGN7850 and (C) VEGF mini-trap REGN7851 in vitreous of New Zealand White rabbits (Rabbits 472, 473, 475, 476, 477, 431, 432 and 433). OD=oculus dexter; OS=oculus sinister.

FIG. 34. 2-way ANOVA showed no significant IOP change before and after 20 minutes post-IVT injection between VEGF Trap REGN3 and VEGF mini-trap REGN7483^{F}groups.

### DETAILED DESCRIPTION OF THE INVENTION

The present invention provides VEGF mini-trap molecules (e.g., REGN7483^{F}) and compositions thereof which have several advantageous properties and are the result of efforts to overcome significant technical hurdles. Expression of mini-traps in chemically defined media (CDM) resulted in significant brown-yellow color. While expression in CDM is the preferred modern method for protein expression (e.g., CDM offers greater reproducibility/consistency over hydrolysate-based media), the addition of a colored material to the eye, a visual organ, could have negative effects on vision. Through analyses and development of optimized purification processes and host cell growth conditions, a possible cause of the color (2-oxo-histidine modification) was identified and its presence in the final purified product has been significantly reduced. In addition, evidence suggests that the mini-traps of the present invention have a shorter systemic half-life than that of aflibercept (Eylea) which could avoid certain adverse events associated with intravitreal administration. The cause of this effect is not clear, but it may be due to a higher mannose content on mini-traps than on aflibercept.

Thus, the present invention encompasses fusion polypeptides capable of binding vascular endothelial cell growth factor (VEGF) as well as therapeutic methods of use thereof.

A “variant” of a polypeptide (e.g., of a VEGFR Ig domain) refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence (e.g., any of SEQ ID NOs: 1-5 or 10-13); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

Variants of a polypeptide (e.g., of a VEGFR Ig domain) may also refer to a polypeptide comprising a referenced amino acid sequence except for one or more (e.g., 1, 2, 3, 4, 5, 6, 7, 8, 9 or 10) mutations such as, for example, missense mutations (e.g., conservative substitutions), non-sense mutations, deletions, or insertions relative to any of SEQ ID NOs: 1-5, 10-13, 26-30, 32, 33 or 36.

The present invention includes VEGF mini-traps comprising polypeptides which are variants of those whose amino acid sequences are specifically set forth herein.

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. 0. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

The sequences and domain structures of VEGF, VEGFR1, VEGFR2 and VEGFR3 are known. In an embodiment of the invention, the VEGF amino acid sequence is set forth under Genbank accession no. AH001553, the VEGFR1 amino acid sequence is set forth under Uniprot accession no. P17948; the VEGFR2 amino acid sequence is set forth under Uniprot accession no. P35968; and/or the VEGFR3 amino acid sequence is set forth under Uniprot accession no. P35916. Holash et al., VEGF-Trap: a VEGF blocker with potent antitumor effects, Proc Natl Acad Sci USA. 2002 Aug. 20; 99(17):11393-8.

### VEGF Mini-Traps

The present invention provides VEGF mini-traps capable of binding vascular endothelial growth factor (VEGF) which are therapeutically useful for treating or preventing conditions and diseases which are treatable or preventable by inhibition of VEGF (e.g., VEGF₁₁₀, VEGF₁₂₁ or VEGF₁₆₅) such as angiogenic eye disorders and cancer—the term “VEGF”, in the context of “VEGF mini-trap” and the like indicates that the mini-trap binds VEGF and has said uses. A summary of the VEGF mini-traps of the present invention are set forth in FIG. 11.

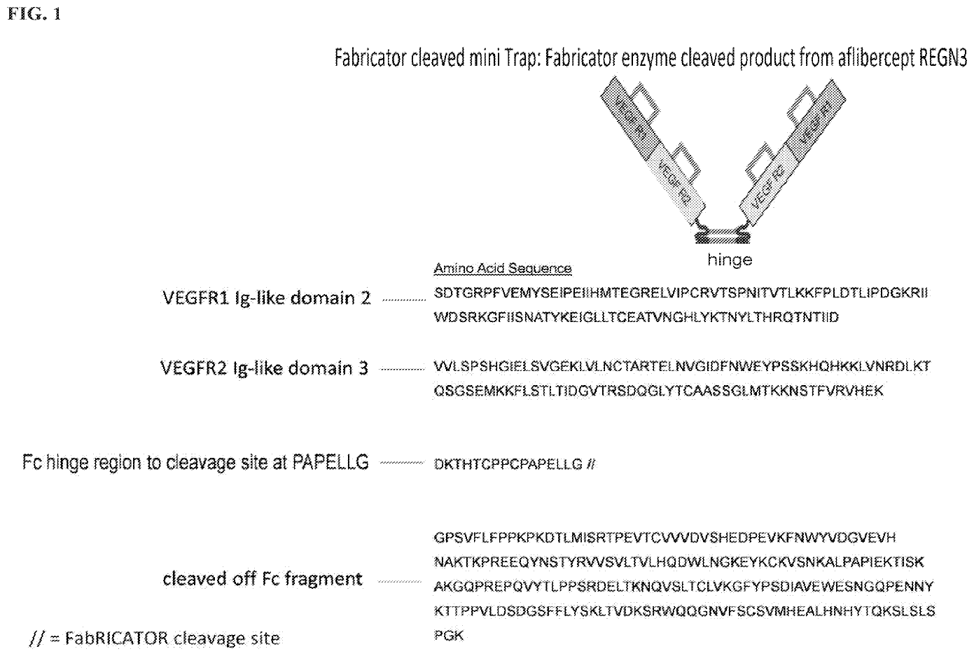

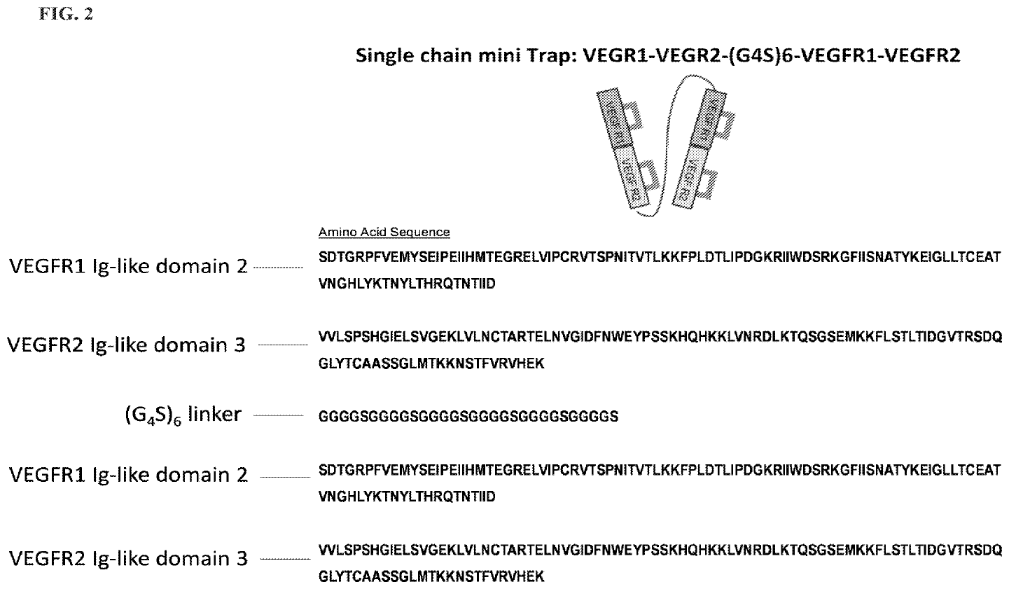

A VEGF mini-trap is a molecule or complex of molecules that binds to VEGF having one or more sets of VEGF receptor Ig-like domains (or variants thereof) (e.g., VEGFR1 Ig domain 2 and/or VEGFR2 Ig domain 3 and/or 4) and a truncated or absent multimerizing component (MC), e.g., wherein the MC is a truncated immunoglobulin Fc. Said truncation may be the result of proteolytic digestion of a VEGF trap (e.g., aflibercept or conbercept) or direct expression of the resulting polypeptide chains with the shortened MC sequence. See the molecular structure depicted in FIG. 1. FIG. 1 is a description of a VEGF mini-trap molecule which is the product of proteolysis of aflibercept with Streptococcus pyogenes IdeS. The homodimeric molecule is depicted with the Ig hinge domain fragments connected by two parallel disulfide bonds. The VEGFR1 domain, the VEGFR2 domain and the hinge domain fragment (MC) is indicated. The point in aflibercept where IdeS cleavage occurs is indicated with an “//”. The cleaved off Fc fragment from aflibercept is also indicated. A single such chimeric polypeptide, which is not dimerized, may also be a VEGF mini-trap if it has VEGF binding activity. The term “VEGF mini-trap” includes a single polypeptide comprising a first set of one or more VEGF receptor Ig domains (or variants thereof), lacking an MC, but fused with a linker (e.g., a peptide linker) to one or more further sets of one or more VEGF receptor Ig domains (or variants thereof). The VEGF binding domains in a VEGF mini-trap of the present invention may be identical or different from another. See WO2005/00895.

For example, in an embodiment of the invention, the untruncated immunoglobulin Fc domain comprises the amino acid sequence or amino acids 1-226 thereof:

DKTHTCPX₁CPAPELLGGPSVFLFPPKPKDTLMISRTPEVTCV
VVDVSHEDPEVKFNWYVDGVEVHNAKTKPREEQYNSTYRVVSV
LTVLHQDWLNGKEYKCKVSNKALPAPIEKTISKAKGQPREPQV
YTLPPSRDELTKNQVSLTCLVKGFYPSDIAVEWESNGQPENNY
KX₂TPPVLDSDGSFFLYSKLTVDKSRWQQGNVFSCSVMHEALH
NHYTQKSLSLSPGK
(SEQ ID NO: 21; wherein X₁ is L or P
and X₂ is A or T)

Inhibition of VEGF includes, for example, antagonism of VEGF binding to VEGF receptor, e.g., by competition with VEGF receptor for VEGF (e.g., VEGF₁₁₀, VEGF₁₂₁ and/or VEGF₁₆₅) binding. Such inhibition may result in inhibition of VEGF-mediated activation of VEGFR, e.g., inhibition of luciferase expression in a cell line (e.g., HEK293) expressing chimeric VEGF Receptor (e.g., a homodimer thereof) having VEGFR extracellular domains fused to IL18Rα and/or IL18Rβ intracellular domains on the cell surface and also having an NFkB-luciferase-IRES-eGFP reporter gene, e.g., the cell line HEK293/D9/Flt-IL18Rα/Flt-IL18Rβ as set forth herein.

The VEGF receptor Ig domain components of the VEGF mini-traps of the present invention can include:
(i) one or more of the immunoglobulin-like (Ig) domain 2 of VEGFR1 (Flt1) (R1D2),
(ii) one or more of the Ig domain 3 of VEGFR2 (Flk1 or KDR) (Flk1D3) (R2D3),
(iii) one or more of the Ig domain 4 of VEGFR2 (Flk1 or KDR) (Flk1D4) (R2D4) and/or
(iv) one or more of the Ig domain 3 of VEGFR3 (Flt4) (FltD3 or R3D3).

Immunoglobulin-like domains of VEGF receptors may be referred to herein as VEGFR “Ig” domains. VEGFR Ig domains which are referenced herein, e.g., R1D2 (which may be referred to herein as VEGFR1(d2)), R2D3 (which may be referred to herein as VEGFR2(d3)), R2D4 (which may be referred to herein as VEGFR2(d4)) and R3D3 (which may be referred to herein as VEGFR3(d3)), are intended to encompass not only the complete wild-type Ig domain, but also variants thereof which substantially retain the functional characteristics of the wild-type domain, e.g., retain the ability to form a functioning VEGF binding domain when incorporated into a VEGF mini-trap. It will be readily apparent to one of skill in the art that numerous variants of the above Ig domains, which will retain substantially the same functional characteristics as the wild-type domain, can be obtained.

The present invention provides a VEGF mini-trap polypeptide comprising the following domain structure: p
((R1D2)-(R2D3))ₐ-linker-((R1D2)-(R2D3))_{b},
((R1D2)-(R2D3)-(R2D4))_{c}-linker-((R1D2)-(R2D3)-(R2D4))_{d},
((R1D2)-(R2D3))ₑ-(MC)_{g}, or
((R1D2)-(R2D3)-(R2D4))_{f}-(MC)_{g},
   wherein,
R1D2 is the VEGF receptor 1 (VEGFR1) Ig domain 2 (D2);
R2D3 is the VEGFR2 Ig domain 3;
R2D4 is the VEGFR2 Ig domain 4;
MC is a multimerizing component (e.g., an IgG1),
linker is a peptide comprising about 5, 6, 7, 8, 9, 10, 11, 12, 13, 14, 15 or 16 amino acids, for example, (GGGS)_{g};
   and,
independently,
a=1, 2, 3, 4, 5, 6, 7, 8, 9, 10, 11, 12, 13, 14 or 15;
b=1, 2, 3, 4, 5, 6, 7, 8, 9, 10, 11, 12, 13, 14 or 15;
c=1, 2, 3, 4, 5, 6, 7, 8, 9, 10, 11, 12, 13, 14 or 15;
d=1, 2, 3, 4, 5, 6, 7, 8, 9, 10, 11, 12, 13, 14 or 15;
e=1, 2, 3, 4, 5, 6, 7, 8, 9, 10, 11, 12, 13, 14 or 15;
f=1, 2, 3, 4, 5, 6, 7, 8, 9, 10, 11, 12, 13, 14 or 15; and
g=1, 2, 3, 4, 5, 6, 7, 8, 9, 10, 11, 12, 13, 14 or 15.

In an embodiment of the invention, R1D2 comprises the amino acid sequence: SDTGRPFVEMYSEIPEIIHMTEGRELVIPCRVTSPNITVTLKKFPLDTLIPDGKRIIWDSRKGFIISNATYKEIGLL TCEATVNGHLYKTNYLTHRQTNTIID

(SEQ ID NO: 1). In an embodiment of the invention, the R1D2 lacks the N-terminal SDT.

In an embodiment of the invention, R1D2 comprises the amino acid sequence:

(SEQ ID NO: 2)
PFVEMYSEIPEIIHMTEGRELVIPCRVTSPNITVTLKKFPLDT
LIPDGKRIIWDSRKGFIISNATYKEIGLLTCEATVNGHLYKTN
YLTHRQT.

In an embodiment of the invention, R2D3 comprises the amino acid sequence:

(SEQ ID NO: 3)
VVLSPSHGIELSVGEKLVLNCTARTELNVGIDFNWEYPSSKHQ
HKKLVNRDLKTQSGSEMKKFLSTLTIDGVTRSDQGLYTCAASS
GLMTKKNSTFVRVHEK.

In an embodiment of the invention, R2D4 comprises the amino acid sequence:

(SEQ ID NO: 4)
PFVAFGSGMESLVEATVGERVRIPAKYLGYPPPEIKWYKNGIP
LESNHTIKAGHVLTIMEVSERDTGNYTVILTNPISKEKQSHVV
SLVVYVPPGPG.

In an embodiment of the invention, R2D4 comprises the amino acid sequence:

(SEQ ID NO: 5)
FVAFGSGMESLVEATVGERVRIPAKYLGYPPPEIKWYKNGIPL
ESNHTIKAGHVLTIMEVSERDTGNYTVILTNPIKSEKQSHVVS
LVVYVP.

In an embodiment of the invention, a multimerizing component (MC) for use in a VEGF mini-tap is a peptide, for example, a truncated Fc immunoglobulin (e.g., IgG1) which is capable of binding to another multimerizing component. In an embodiment of the invention, an MC is a truncated Fc immunoglobulin that includes the immunoglobulin hinge region or a fragment thereof. For example, in an embodiment of the invention, an MC is a peptide comprising one or more (e.g., 1, 2, 3, 4, 5 or 6) cysteines that are able to form one or more cysteine bridges with cysteines in another MC, e.g., DKTHTCPPC (SEQ ID NO: 22),

(SEQ ID NO: 23)
DKTHTCPPCPPC,
(SEQ ID NO: 24)
DKTHTCPPCPPCPPC,
(SEQ ID NO: 25)
DKTHTC(PPC)ₕ,
wherein h is 1,2, 3, 4, or 5,
(SEQ ID NO: 6)
DKTHTCPPCPAPELLG,
(SEQ ID NO: 7)
DKTHTCPLCPAPELLG,
(SEQ ID NO: 8)
DKTHTC
or
(SEQ ID NO: 9)
DKTHTCPLCPAP.

The present invention also provides a VEGF mini-trap polypeptide comprising the following domain structure:
(i) ((R1D2)-(R2D3))ₐ-(MC)_{b}, or
(ii) ((R1D2)-(R2D3)-(R2D4))_{c}-(MC)_{d},
   which may be homodimerized with a second of said polypeptides e.g., by binding between the MCs of each polypeptide,
wherein
(i) said R1D2 domains coordinate;
(ii) said R2D3 domains coordinate; and/or
(iii) said R2D4 domains coordinate,
   to form a dimeric VEGF binding domain.

In an embodiment of the invention, the VEGF mini-trap polypeptide comprises or consists of the amino acid sequence:

SDTGRPFVEMYSEIPEIIHMTEGRELVIPCRVTSPN₃₆ITVTLKKFPLDTLIPDGKRIIWDSRKGFIISN₆₈ATYKEIG
LLTCEATVNGHLYKTNYLTHRQTNTIIDVVLSPSHGIELSVGEKLVLN₁₂₃CTARTELNVGIDFNWEYPSSKHQHKKLV
NRDLKTQSGSEMKKFLSTLTIDGVTRSDQGLYTCAASSGLMTKKN₁₉₆STFVRVHEKDKTHTCPPCPAPELLG
(SEQ ID NO: 12; MC underscored; REGN7483 (REGN7483^{F}/REGN7483^{R}));
GRPFVEMYSEIPEIIHMTEGRELVIPCRVTSPNITVTLKKFPLDTLIPDGKRIIWDSRKGFIISNATYKEIGLLTCE
ATVNGHLYKTNYLTHRQTNTIIDVVLSPSHGIELSVGEKLVLNCTARTELNVGIDFNWEYPSSKHQHKKLVNRDLKT
QSGSEMKKFLSTLTIDGVTRSDQGLYTCAASSGLMTKKNSTFVRVHENLSVAFGSGMESLVEATVGERVRIPAKYLG
YPPPEIKWYKNGIPLESNHTIKAGHVLTIMEVSERDTGNYTVILTNPISKEKQSHVVSLVVYVPPGPGDKTHTCPLC
PAPELLG
(SEQ ID NO: 13; MC underscored);
SDTGRPFVEMYSEIPEIIHMTEGRELVIPCRVTSPN₃₆ITVTLKKFPLDTLIPDGKRIIWDSRKGFIISN₆₈ATYKEIG
LLTCEATVNGHLYKTNYLTHRQTNTIIDVVLSPSHGIELSVGEKLVLN₁₂₃CTARTELNVGIDFNWEYPSSKHQHKKLV
NRDLKTQSGSEMKKFLSTLTIDGVTRSDQGLYTCAASSGLMTKKN₁₉₆STFVRVHEKDKTHTCPPC
(SEQ ID NO: 26; MC underscored (REGN112));
SDTGRPFVEMYSEIPEIIHMTEGRELVIPCRVTSPN₃₆ITVTLKKFPLDTLIPDGKRIIWDSRKGFIISN₆₈ATYKEIG
LLTCEATVNGHLYKTNYLTHRQTNTIIDVVLSPSHGIELSVGEKLVLN₁₂₃CTARTELNVGIDFNWEYPSSKHQHKKLV
NRDLKTQSGSEMKKFLSTLTIDGVTRSDQGLYTCAASSGLMTKKN₁₉₆STFVRVHEKDKTHTCPPCPPC
(SEQ ID NO: 27; MC underscored; REGN7850);
SDTGRPFVEMYSEIPEIIHMTEGRELVIPCRVTSPN₃₆lTVTLKKFPLDTLIPDGKRIIWDSRKGFIISN₆₈ATYKEIG
LLTCEATVNGHLYKTNYLTHRQTNTIIDVVLSPSHGIELSVGEKLVLN₁₂₃CTARTELNVGIDFNWEYPSSKHQHKKLV
NRDLKTQSGSEMKKFLSTLTIDGVTRSDQGLYTCAASSGLMTKKN₁₉₆STFVRVHEKDKTHTCPPCPPCPPC
(SEQ ID NO: 28; MC underscored; REGN7851);
or
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKDKTHTC-(PPC)ₓ

(SEQ ID NO: 29; MC underscored; wherein x is 1, 2, 3, 4 or 5). As discussed, such polypeptides may be multimerized (e.g., dimerized (e.g., homodimerized)) wherein binding between the polypeptides is mediated via the multimerizing components. Such multimers and single polypeptides are part of the present invention.

In an embodiment of the invention, in REGN7483^{F or R}, REGN7850 or REGN7851, N36, N68, N123 and/or N196 are N-glycosylated. In an embodiment of the invention, in REGN7483^{F or R}, REGN7850 or REGN7851, there are intrachain disulfide bridges between (i) C30 and C79 and/or (ii) C124 and C185.

In an embodiment of the invention, in the hinge region of REGN7483^{F or R}, REGN7850 or REGN7851, THTCPPCPAPELLG (amino acids 208-221 of SEQ ID NO: 12), interchain disulfide bridges are parallel (between each C211 and between each C214) or crossed (between C211 and C214). In an embodiment of the invention, the majority of disulfide bridges are parallel.

In an embodiment of the invention, in REGN7483^{F or R}, REGN7850 or REGN7851, the C-terminal glycine is missing.

In an embodiment of the invention, the VEGFR1 Ig-like domain 2 of the monomeric VEGF mini-traps of the present invention, have N-linked glycosylation at N36 and/or N68; and/or an intrachain disulfide bridge between C30 and C79; and/or, the VEGFR2 Ig-like domain 3 of the monomeric VEGF mini-traps of the present invention, have N-linked glycosylation at N123 and/or N196; and/or an intrachain disulfide bridge between C124 and C185.

In an embodiment of the invention, the VEGF mini-trap comprises the structure:
(R1D2)₁-(R2D3)₁-(G45)₃-(R1D2)₁-(R2D3)₁,
(R1D2)₁-(R2D3)₁-(G45)₆-(R1D2)₁-(R2D3)₁,
(R1D2)₁-(R2D3)₁-(G₄S)₉-(R1D2)₁-(R2D3)₁, or
(R1D2)₁-(R2D3)₁-(G₄S)₁₂-(R1D2)₁-(R2D3)₁.

### G₄S is -Gly-Gly-Gly-Gly-Ser-

In an embodiment of the invention, the VEGF mini-trap comprises the amino acid sequence:

(i)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKGGGGSGGGGSGGGGSGGGGSGGGGSG
GGGSSDTGRPFVEMYSEIPEIIHMTEGRELVIPCRVTSPNITVTLKKFPLDTLIPDGKRIIWDSRKGFIISNATYKE
IGLLTCEATVNGHLYKTNYLTHRQTNTIIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFVRVHEK (SEQ ID NO: 10; linker
underscored (REGN7080));
(iii)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKGGGGSGGGGSGGGGSSDTGRPFVEMY
SEIPEIIHMTEGRELVIPCRVTSPNITVTLKKFPLDTLIPDGKRIIWDSRKGFIISNATYKEIGLLTCEATVNGHLY
KTNYLTHRQTNTIIDVVLSPSHGIELSVGEKLVLNCTARTELNVGIDFNWEYPSSKHQHKKLVNRDLKTQSGSEMKK
FLSTLTIDGVTRSDQGLYTCAASSGLMTKKNSTFVRVHEK (SEQ ID NO: 11; linker underscored
(REGN6824));
(iv)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKGGGGSGGGGSGGGGSGGGGSGGGGSG
GGGSGGGGSGGGGSGGGGSSDTGRPFVEMYSEIPEIIHMTEGRELVIPCRVTSPNITVTLKKFPLDTLIPDGKRIIW
DSRKGFIISNATYKEIGLLTCEATVNGHLYKTNYLTHRQTNTIIDVVLSPSHGIELSVGEKLVLNCTARTELNVGID
FNWEYPSSKHQHKKLVNRDLKTQSGSEMKKFLSTLTIDGVTRSDQGLYTCAASSGLMTKKNSTFVRVHEK (SEQ ID
NO: 32; linker underscored (REGN7991))
(v)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKGGGGSGGGGSGGGGSGGGGSGGGGSG
GGGSGGGGSGGGGSGGGGSGGGGSGGGGSGGGGSSDTGRPFVEMYSEIPEIIHMTEGRELVIPCRVTSPNITVTLKK
FPLDTLIPDGKRIIWDSRKGFIISNATYKEIGLLTCEATVNGHLYKTNYLTHRQTNTIIDVVLSPSHGIELSVGEKL
VLNCTARTELNVGIDFNWEYPSSKHQHKKLVNRDLKTQSGSEMKKFLSTLTIDGVTRSDQGLYTCAASSGLMTKKNS
TFVRVHEK (SEQ ID NO: 33; linker underscored (REGN7992));
(vi)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEK-(GGGGS)x-
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEK;
(SEQ ID NO: 30; wherein x is 1, 2, 3, 4, 5, 6, 7, 8, 9,
10, 11, 12, 13, 14 or 15);
or;
(vii)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKGGGGSGGGGSGGGGSGGGGSGGGGSG
GGGSSDTGRPFVEMYSEIPEIIHMTEGRELVIPCRVTSPNITVTLKKFPLDTLIPDGKRIIWDSRKGFIISNATYKE
IGLLTCEATVNGHLYKTNYLTHRQTNTIIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFVRVHEK (SEQ ID NO: 36;
REGN7711).

As discussed herein, these polypeptides may comprise a secondary structure wherein like VEGFR Ig domains associate to form an intra-chain VEGF binding domain (see e.g., FIG. 2). In an embodiment of the invention, two or more of such polypeptides multimerize (e.g., dimerize (e.g., homodimerize)) wherein the VEGFR Ig domains of each chain associate with like Ig domains of another chain to form an inter-chain VEGF binding domain.

In a certain embodiment of the invention, a VEGF mini-trap of the present invention lacks any significant modification of the amino acid residues of a VEGF mini-trap polypeptide (e.g., directed chemical modification such as PEGylation or iodoacetamidation, for example at the N- and/or C-terminus).

In an embodiment of the invention, the polypeptide comprises a secondary structure wherein like VEGFR Ig domains in a single chimeric polypeptide (e.g., ((R1D2)-(R2D3))ₐ-linker-((R1D2)-(R2D3))_{b}, or ((R1D2)-(R2D3)-(R2D4))_{c}-linker-((R1D2)-(R2D3)-(R2D4))_{d} or in separate chimeric polypeptides (e.g., homodimers) coordinate to form a VEGF binding domain. For example, wherein
(i) said R1D2 domains coordinate;
(ii) said R2D3 domains coordinate; and/or
(iii) said R2D4 domains coordinate,
   to form a VEGF binding domain. FIG. 2 is a description of a single chain VEGF mini-trap depicting such domain coordination. The VEGFR1, VEGFR2 and linker domains are indicated. The linker shown is (G₄S)₆. The present invention includes single chain VEGF mini-traps with a (G₄S)₃; (G₄S)₉ or (G₄S)₁₂ linker.

In addition, the present invention also provides a complex comprising a VEGF mini-trap as discussed herein complexed with a VEGF polypeptide or a fragment thereof or fusion thereof. In an embodiment of the invention, the VEGF (e.g., VEGF₁₆₅) is homodimerized and/or the VEGF mini-trap is homodimerized in a 2:2 complex (2 VEGFs:2 mini-traps). Complexes can include homodimerized VEGF molecules bound to homodimerized VEGF mini-trap polypeptides. In an embodiment of the invention, the complex is in vitro (e.g., is immobilized to a solid substrate) or is in the body of a subject. The present invention also includes a composition of complexes of a VEGF dimer (e.g., VEGF₁₆₅) complexed with a VEGF mini-trap, e.g., REGN6824, REGN7080 or REGN7483^{F}, at a molar ratio as set forth in Table 3-3 herein.

### IdeS and Variants Thereof

The present invention includes VEGF mini-traps and compositions thereof that have been produced by proteolytic digestion of aflibercept with Streptococcus pyogenes IdeS (FabRICATOR) and variants thereof. FabRICATOR is commercially available from Genovis, Inc.; Cambridge, Mass.; Lund, Sweden.

In one embodiment, the IdeS polypeptide comprises an amino acid sequence with at least 70% sequence identity over a full length of the isolated an amino acid sequence as set forth in the group consisting of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51 and SEQ ID NO: 52. In one aspect, the isolated an amino acid sequence has at least about 80% sequence identity over a full length of the isolated an amino acid sequence. In another aspect, the isolated an amino acid sequence has at least about 90% sequence identity over a full length of the isolated an amino acid sequence. In another aspect, the isolated an amino acid sequence has about 100% sequence identity over a full length of the isolated an amino acid sequence. In one aspect, the polypeptide can be capable of cleaving a target protein into fragments. In a particular aspect, the target protein is an IgG. In another particular aspect, the target protein is a fusion protein. In yet another particular aspect, the fragments can comprise a Fab fragment and/or a Fc fragment.

In one embodiment, the IdeS amino acid sequence comprises a parental amino acid sequence defined by SEQ ID NO: 37 but having an asparagine residue at position 87, 130, 182 and/or 274 mutated to an amino acid other than asparagine. In one aspect, the mutation can confer an increased chemical stability at alkaline pH-values compared to the parental amino acid sequence. In another aspect, the mutation can confer an increase in chemical stability by 50% at alkaline pH-values compared to the parental amino acid sequence. In one aspect, the amino acid can be selected from aspartic acid, leucine, and arginine. In a particular aspect, the asparagine residue at position 87 is mutated to aspartic acid residue. In another particular aspect, the asparagine residue at position 130 is mutated to arginine residue. In a yet another particular aspect, the asparagine residue at position 182 is mutated to a leucine residue. In a yet another particular aspect, the asparagine residue at position 274 is mutated to aspartic acid residue. In a yet another particular aspect, the asparagine residue at position 87 and 130 are mutated. In a yet another particular aspect, the asparagine residue at position 87 and 182 are mutated. In a yet another particular aspect, the asparagine residue at position 87 and 274 are mutated. In a yet another particular aspect, the asparagine residue at position 130 and 182 are mutated. In a yet another particular aspect, the asparagine residue at position 130 and 274 are mutated. In a yet another particular aspect, the asparagine residue at position 182 and 274 are mutated. In a yet another particular aspect, the asparagine residue at position 87, 130 and 182 are mutated. In a yet another particular aspect, the asparagine residue at position 87, 182 and 274 are mutated. In a yet another particular aspect, the asparagine residue at position 130, 182 and 274 are mutated. In a yet another particular aspect, the asparagine residue at position 87, 130, 182 and 274 are mutated.

Aflibercept can be cleaved by IdeS that has been immobilized to a solid support, e.g., a chromatography bead. For example, a sample including aflibercept in a buffered aqueous solution (in a cleavage buffer) can be applied to the immobilized IdeS, e.g., in a chromatography column. The column can be incubated, e.g., for 30 minutes, e.g., at about 18° C. The column can then be washed with the cleavage buffer. After cleavage, the digestion and wash solutions can be applied to a protein A column to capture cleaved Fc by-product wherein mini-trap product is retained in the flow-through fraction. In an embodiment of the invention, the cleavage buffer and/or the protein-A column equilibration and wash solutions are at pH 7, e.g., 40 mM Tris, 54 mM Acetate pH 7.0±0.1.

SEQ ID NO: 37
MRKRCYSTSAAVLAAVTLFVLSVDRGVIADSFSANQEIRYSEVTPYHVTSVWTKGVTPPANFTQGEDVFHAPYVANQ
GWYDITKTFNGKDDLLCGAATAGNMLHWWFDQNKDQIKRYLEEHPEKQKINFNGEQMFDVKEAIDTKNHQLDSKLFE
YFKEKAFPYLSTKHLGVFPDHVIDMFINGYRLSLTNHGPTPVKEGSKDPRGGIFDAVFTRGDQSKLLTSRHDFKEKN
LKEISDLIKKELTEGKALGLSHTYANVRINHVINLWGADFDSNGNLKAIYVTDSDSNASIGMKKYFVGVNSAGKVAI
SAKEIKEDNIGAQVLGLFTLSTGQDSWNQTN
SEQ ID NO: 38
MRKRCYSTSAAVLAAVTLFVLSVDRGVIADSFSANQEIRYSEVTPYHVTSVWTKGVTPPANFTQGEDVFHAPYVANQ
GWYDITKTFDGKDDLLCGAATAGNMLHWWFDQNKDQIKRYLEEHPEKQKINFNGEQMFDVKEAIDTKNHQLDSKLFE
YFKEKAFPYLSTKHLGVFPDHVIDMFINGYRLSLTNHGPTPVKEGSKDPRGGIFDAVFTRGDQSKLLTSRHDFKEKN
LKEISDLIKKELTEGKALGLSHTYANVRINHVINLWGADFDSNGNLKAIYVTDSDSNASIGMKKYFVGVNSAGKVAI
SAKEIKEDNIGAQVLGLFTLSTGQDSWNQTN
SEQ ID NO: 39
MRKRCYSTSAAVLAAVTLFVLSVDRGVIADSFSANQEIRYSEVTPYHVTSVWTKGVTPPANFTQGEDVFHAPYVANQ
GWYDITKTFNGKDDLLCGAATAGNMLHWWFDQNKDQIKRYLEEHPEKQKINFRGEQMFDVKEAIDTKNHQLDSKLFE
YFKEKAFPYLSTKHLGVFPDHVIDMFINGYRLSLTNHGPTPVKEGSKDPRGGIFDAVFTRGDQSKLLTSRHDFKEKN
LKEISDLIKKELTEGKALGLSHTYANVRINHVINLWGADFDSNGNLKAIYVTDSDSNASIGMKKYFVGVNSAGKVAI
SAKEIKEDNIGAQVLGLFTLSTGQDSWNQTN
SEQ ID NO: 40
MRKRCYSTSAAVLAAVTLFVLSVDRGVIADSFSANQEIRYSEVTPYHVTSVWTKGVTPPANFTQGEDVFHAPYVANQ
GWYDITKTFNGKDDLLCGAATAGNMLHWWFDQNKDQIKRYLEEHPEKQKINFNGEQMFDVKEAIDTKNHQLDSKLFE
YFKEKAFPYLSTKHLGVFPDHVIDMFILGYRLSLTNHGPTPVKEGSKDPRGGIFDAVFTRGDQSKLLTSRHDFKEKN
LKEISDLIKKELTEGKALGLSHTYANVRINHVINLWGADFDSNGNLKAIYVTDSDSNASIGMKKYFVGVNSAGKVAI
SAKEIKEDNIGAQVLGLFTLSTGQDSWNQTN
SEQ ID NO: 41
MRKRCYSTSAAVLAAVTLFVLSVDRGVIADSFSANQEIRYSEVTPYHVTSVWTKGVTPPANFTQGEDVFHAPYVANQ
GWYDITKTFNGKDDLLCGAATAGNMLHWWFDQNKDQIKRYLEEHPEKQKINFNGEQMFDVKEAIDTKNHQLDSKLFE
YFKEKAFPYLSTKHLGVFPDHVIDMFINGYRLSLTNHGPTPVKEGSKDPRGGIFDAVFTRGDQSKLLTSRHDFKEKN
LKEISDLIKKELTEGKALGLSHTYANVRINHVINLWGADFDSDGNLKAIYVTDSDSNASIGMKKYFVGVNSAGKVAI
SAKEIKEDNIGAQVLGLFTLSTGQDSWNQTN
SEQ ID NO: 42
MRKRCYSTSAAVLAAVTLFVLSVDRGVIADSFSANQEIRYSEVTPYHVTSVWTKGVTPPANFTQGEDVFHAPYVANQ
GWYDITKTFDGKDDLLCGAATAGNMLHWWFDQNKDQIKRYLEEHPEKQKINFRGEQMFDVKEAIDTKNHQLDSKLFE
YFKEKAFPYLSTKHLGVFPDHVIDMFINGYRLSLTNHGPTPVKEGSKDPRGGIFDAVFTRGDQSKLLTSRHDFKEKN
LKEISDLIKKELTEGKALGLSHTYANVRINHVINLWGADFDSNGNLKAIYVTDSDSNASIGMKKYFVGVNSAGKVAI
SAKEIKEDNIGAQVLGLFTLSTGQDSWNQTN
SEQ ID NO: 43
MRKRCYSTSAAVLAAVTLFVLSVDRGVIADSFSANQEIRYSEVTPYHVTSVWTKGVTPPANFTQGEDVFHAPYVANQ
GWYDITKTFDGKDDLLCGAATAGNMLHWWFDQNKDQIKRYLEEHPEKQKINFNGEQMFDVKEAIDTKNHQLDSKLFE
YFKEKAFPYLSTKHLGVFPDHVIDMFILGYRLSLTNHGPTPVKEGSKDPRGGIFDAVFTRGDQSKLLTSRHDFKEKN
LKEISDLIKKELTEGKALGLSHTYANVRINHVINLWGADFDSNGNLKAIYVTDSDSNASIGMKKYFVGVNSAGKVAI
SAKEIKEDNIGAQVLGLFTLSTGQDSWNQTN
SEQ ID NO: 44
MRKRCYSTSAAVLAAVTLFVLSVDRGVIADSFSANQEIRYSEVTPYHVTSVWTKGVTPPANFTQGEDVFHAPYVANQ
GWYDITKTFDGKDDLLCGAATAGNMLHWWFDQNKDQIKRYLEEHPEKQKINFNGEQMFDVKEAIDTKNHQLDSKLFE
YFKEKAFPYLSTKHLGVFPDHVIDMFINGYRLSLTNHGPTPVKEGSKDPRGGIFDAVFTRGDQSKLLTSRHDFKEKN
LKEISDLIKKELTEGKALGLSHTYANVRINHVINLWGADFDSDGNLKAIYVTDSDSNASIGMKKYFVGVNSAGKVAI
SAKEIKEDNIGAQVLGLFTLSTGQDSWNQTN
SEQ ID NO: 45
MRKRCYSTSAAVLAAVTLFVLSVDRGVIADSFSANQEIRYSEVTPYHVTSVWTKGVTPPANFTQGEDVFHAPYVANQ
GWYDITKTFNGKDDLLCGAATAGNMLHWWFDQNKDQIKRYLEEHPEKQKINFRGEQMFDVKEAIDTKNHQLDSKLFE
YFKEKAFPYLSTKHLGVFPDHVIDMFILGYRLSLTNHGPTPVKEGSKDPRGGIFDAVFTRGDQSKLLTSRHDFKEKN
LKEISDLIKKELTEGKALGLSHTYANVRINHVINLWGADFDSNGNLKAIYVTDSDSNASIGMKKYFVGVNSAGKVAI
SAKEIKEDNIGAQVLGLFTLSTGQDSWNQTN
SEQ ID NO: 46
MRKRCYSTSAAVLAAVTLFVLSVDRGVIADSFSANQEIRYSEVTPYHVTSVWTKGVTPPANFTQGEDVFHAPYVANQ
GWYDITKTFNGKDDLLCGAATAGNMLHWWFDQNKDQIKRYLEEHPEKQKINFRGEQMFDVKEAIDTKNHQLDSKLFE
YFKEKAFPYLSTKHLGVFPDHVIDMFINGYRLSLTNHGPTPVKEGSKDPRGGIFDAVFTRGDQSKLLTSRHDFKEKN
LKEISDLIKKELTEGKALGLSHTYANVRINHVINLWGADFDSDGNLKAIYVTDSDSNASIGMKKYFVGVNSAGKVAI
SAKEIKEDNIGAQVLGLFTLSTGQDSWNQTN
SEQ ID NO: 47
MRKRCYSTSAAVLAAVTLFVLSVDRGVIADSFSANQEIRYSEVTPYHVTSVWTKGVTPPANFTQGEDVFHAPYVANQ
GWYDITKTFNGKDDLLCGAATAGNMLHWWFDQNKDQIKRYLEEHPEKQKINFNGEQMFDVKEAIDTKNHQLDSKLFE
YFKEKAFPYLSTKHLGVFPDHVIDMFILGYRLSLTNHGPTPVKEGSKDPRGGIFDAVFTRGDQSKLLTSRHDFKEKN
LKEISDLIKKELTEGKALGLSHTYANVRINHVINLWGADFDSDGNLKAIYVTDSDSNASIGMKKYFVGVNSAGKVAI
SAKEIKEDNIGAQVLGLFTLSTGQDSWNQTN
SEQ ID NO: 48
MRKRCYSTSAAVLAAVTLFVLSVDRGVIADSFSANQEIRYSEVTPYHVTSVWTKGVTPPANFTQGEDVFHAPYVANQ
GWYDITKTFDGKDDLLCGAATAGNMLHWWFDQNKDQIKRYLEEHPEKQKINFRGEQMFDVKEAIDTKNHQLDSKLFE
YFKEKAFPYLSTKHLGVFPDHVIDMFILGYRLSLTNHGPTPVKEGSKDPRGGIFDAVFTRGDQSKLLTSRHDFKEKN
LKEISDLIKKELTEGKALGLSHTYANVRINHVINLWGADFDSNGNLKAIYVTDSDSNASIGMKKYFVGVNSAGKVAI
SAKEIKEDNIGAQVLGLFTLSTGQDSWNQTN
SEQ ID NO: 49
MRKRCYSTSAAVLAAVTLFVLSVDRGVIADSFSANQEIRYSEVTPYHVTSVWTKGVTPPANFTQGEDVFHAPYVANQ
GWYDITKTFDGKDDLLCGAATAGNMLHWWFDQNKDQIKRYLEEHPEKQKINFRGEQMFDVKEAIDTKNHQLDSKLFE
YFKEKAFPYLSTKHLGVFPDHVIDMFINGYRLSLTNHGPTPVKEGSKDPRGGIFDAVFTRGDQSKLLTSRHDFKEKN
LKEISDLIKKELTEGKALGLSHTYANVRINHVINLWGADFDSDGNLKAIYVTDSDSNASIGMKKYFVGVNSAGKVAI
SAKEIKEDNIGAQVLGLFTLSTGQDSWNQTN
SEQ ID NO: 50
MRKRCYSTSAAVLAAVTLFVLSVDRGVIADSFSANQEIRYSEVTPYHVTSVWTKGVTPPANFTQGEDVFHAPYVANQ
GWYDITKTFDGKDDLLCGAATAGNMLHWWFDQNKDQIKRYLEEHPEKQKINFNGEQMFDVKEAIDTKNHQLDSKLFE
YFKEKAFPYLSTKHLGVFPDHVIDMFILGYRLSLTNHGPTPVKEGSKDPRGGIFDAVFTRGDQSKLLTSRHDFKEKN
LKEISDLIKKELTEGKALGLSHTYANVRINHVINLWGADFDSDGNLKAIYVTDSDSNASIGMKKYFVGVNSAGKVAI
SAKEIKEDNIGAQVLGLFTLSTGQDSWNQTN
SEQ ID NO: 51
MRKRCYSTSAAVLAAVTLFVLSVDRGVIADSFSANQEIRYSEVTPYHVTSVWTKGVTPPANFTQGEDVFHAPYVANQ
GWYDITKTFNGKDDLLCGAATAGNMLHWWFDQNKDQIKRYLEEHPEKQKINFRGEQMFDVKEAIDTKNHQLDSKLFE
YFKEKAFPYLSTKHLGVFPDHVIDMFILGYRLSLTNHGPTPVKEGSKDPRGGIFDAVFTRGDQSKLLTSRHDFKEKN
LKEISDLIKKELTEGKALGLSHTYANVRINHVINLWGADFDSDGNLKAIYVTDSDSNASIGMKKYFVGVNSAGKVAI
SAKEIKEDNIGAQVLGLFTLSTGQDSWNQTN
SEQ ID NO: 52
MRKRCYSTSAAVLAAVTLFVLSVDRGVIADSFSANQEIRYSEVTPYHVTSVWTKGVTPPANFTQGEDVFHAPYVANQ
GWYDITKTFDGKDDLLCGAATAGNMLHWWFDQNKDQIKRYLEEHPEKQKINFRGEQMFDVKEAIDTKNHQLDSKLFE
YFKEKAFPYLSTKHLGVFPDHVIDMFILGYRLSLTNHGPTPVKEGSKDPRGGIFDAVFTRGDQSKLLTSRHDFKEKN
LKEISDLIKKELTEGKALGLSHTYANVRINHVINLWGADFDSDGNLKAIYVTDSDSNASIGMKKYFVGVNSAGKVAI
SAKEIKEDNIGAQVLGLFTLSTGQDSWNQTN

### Protein Purification

Compositions including proteins of interest (e.g., VEGF mini-traps (e.g., REGN7850, REGN7851, REGN7483^{F} or REGN7483^{R}) produced by a method including a combination of different purification techniques, including, but not limited to, affinity, ion exchange, mixed mode, and hydrophobic interaction chromatography singularly or in combination are envisaged to be within the scope of the present invention. In an embodiment, the method includes purifying aflibercept which is enzymatically cleaved to generate REGN7483^{F}. These chromatographic steps separate mixtures of proteins of a sample matrix on the basis of their charge, degree of hydrophobicity, or size, or any combination thereof, depending on the particular form of separation. Several different chromatography resins are available for each of the techniques alluded to herein, allowing accurate tailoring of the purification scheme to the particular protein involved. Each separation method results in the protein traversing at different rates through a column, to achieve a physical separation that increases as they pass further through the column or adhere selectively to the separation medium. The proteins are then either (i) differentially eluted using an appropriate elution buffers and/or (ii) collected from flow-through fractions obtained from the column used, optionally, from washing the column with an appropriate equilibration buffer. In some cases, the protein of interest is separated from impurities (protein variants) when the impurities preferentially adhere to the column and the protein of interest less so, i.e., the protein of interest does not adsorb to the solid phase of a particular column and thus flows through the column. In some cases, the impurities are separated from the protein of interest when they fail to adsorb to the column and thus flow through the column.

The purification process may begin at the separation step after the recombinant protein has been produced using upstream production methods described herein and/or by alternative production methods conventional in the art. Once a clarified solution or mixture comprising the protein of interest, e.g., a VEGF mini-trap (e.g., REGN7850, REGN7851, REGN7483), has been obtained, separation of the protein of interest from process-related impurities (such as the other proteins produced by the cell (like HCPs), as well as product-related substances, such acidic or basic variants) is performed. In certain non-limiting embodiments, such separation is performed using CEX, AEX, and/or MM (mixed mode) chromatography. In certain embodiments, a combination of one or more different purification techniques, including affinity, ion exchange, mixed-mode, and/or hydrophobic interaction chromatography can be employed. Such additional purification steps separate mixtures of components within a sample matrix on the basis of their, e.g., charge, degree of hydrophobicity, and/or size. Numerous chromatography resins are commercially available for each of the chromatography techniques mentioned herein allowing accurate tailoring of the purification scheme to a particular protein involved. Each of the separation methods allow proteins to either traverse at different rates through a column achieving a physical separation that increases as they pass further through the column, or to adsorb selectively to a separation resin (or medium). The proteins are then differentially eluted using an appropriate buffer. In some cases, the protein of interest is separated from components of a sample matrix when these other components specifically adsorb to a column's resin and the protein of interest does not, while in other cases the protein of interest will adsorb to the column's resin, while the other components are extruded from the column during a wash cycle.

### Primary Recovery and Virus Inactivation

In certain embodiments, the initial steps of the purification methods disclosed herein involve the clarification and primary recovery of VEGF mini-trap (e.g., REGN7850, REGN7851, REGN7483) from a sample matrix. In certain embodiments, the primary recovery will include one or more centrifugation steps to separate the protein of interest, e.g., VEGF mini-trap (e.g., REGN7850, REGN7851, REGN7483), from the host cell and attendant cell debris. Centrifugation of the sample can be performed at, for example, but not by way of limitation, 7,000×g to approximately 12,750×g. In the context of large-scale purification, such centrifugation can occur on-line with a flow rate set to achieve, for example, a turbidity level of 150 NTU in the resulting supernatant. Such supernatant can then be collected for further purification, or in-line filtered through one or more depth filters for further clarification of the sample.

In certain exemplary embodiments, the primary recovery may include the use of one or more depth filtration steps to clarify the sample matrix and, thereby, aid in purifying the proteins of interest in the present invention (e.g., REGN7850, REGN7851, REGN7483). In other embodiments, the primary recovery may include the use of one or more depth filtration steps post centrifugation to further clarify the sample matrix. Non-limiting examples of depth filters that can be used in the context of the instant invention include the Millistak+ X0HC, F0HC, D0HC, A1HC, B1HC depth filters (EMD Millipore), 3M™ model 30/60ZA, 60/90 ZA, VR05, VR07, delipid depth filters (3M Corp.). A 0.2 μ [symbol] η filter such as Sartorius's 0.45/0.2 μ [symbol] η Sartopore™ bi-layer or Millipore's Express SHR or SHC filter cartridges typically follows the depth filters. Other filters well known to the skilled artisan can also be used.

In certain embodiments, the primary recovery process can also be a point to reduce or inactivate viruses that can be present in a sample matrix. For example, any one or more of a variety of methods of viral reduction/inactivation can be used during the primary recovery phase of purification including heat inactivation (pasteurization), pH inactivation, buffer/detergent treatment, UV and γ-ray irradiation and the addition of certain chemical inactivating agents such as β-propiolactone or e.g., copper phenanthroline as described in U.S. Pat. No. 4,534,972. In certain exemplary embodiments of the present invention, the sample matrix is exposed to detergent viral inactivation during the primary recovery phase. In other embodiments, the sample matrix may be exposed to low pH inactivation during the primary recovery phase.

In those embodiments where viral reduction/inactivation is employed, the sample mixture can be adjusted, as needed, for further purification steps. For example, following low pH viral inactivation, the pH of the sample mixture is typically adjusted to a more neutral pH, e.g., from about 4.5 to about 8.5, prior to continuing the purification process. Additionally, the mixture may be diluted with water for injection (WFI) to obtain a desired conductivity

VEGF mini-traps and compositions comprising VEGF mini-trap which is a product of a purification process including primary recovery, filtration and/or viral inactivation, e.g., under conditions as discussed herein, are part of the present invention. VEGF mini-traps and compositions comprising VEGF mini-traps which are a product of a purification process including primary recovery, filtration and/or viral inactivation of VEGF trap, such as aflibercept, which is later cleaved with an IdeS protease to generate the VEGF mini-trap, e.g., under conditions as discussed herein, are part of the present invention

### Affinity Chromatography

In certain exemplary embodiments, it may be advantageous to subject a sample matrix to affinity chromatography for purification of a protein of interest. In certain embodiments, the chromatographic material is capable of selectively or specifically binding to the protein of interest exploiting a particular moiety of the protein. Non-limiting examples of such chromatographic material include: Protein A & Protein G. Also, chromatographic material comprising, for example, a protein or portion thereof capable of binding to the protein of interest. In an embodiment of the invention, aflibercept, which may be enzymatically cleaved with IdeS is purified by protein A or protein G chromatography. In an embodiment of the invention, Fc fragment removed from aflibercept by IdeS cleavage is removed from a sample including mini-trap by protein A or protein G chromatography.

In particular embodiments, affinity chromatography may involve subjecting a sample matrix to a column comprising a suitable Protein A resin. In certain aspects, Protein A resin is useful for affinity purification and isolation of a variety of VEGF mini-trap isotypes by interacting specifically with the Fc portion of a contaminant molecule should it possess that region (wherein mini-trap lacking affinity to Protein-A is in the flow-through fraction). Protein A is a bacterial cell wall protein that binds to mammalian IgGs primarily through their Fc regions. In its native state, Protein A has five IgG binding domains as well as other domains of unknown function. In specific embodiments, the affinity chromatography step involves subjecting the primary recovery sample to a column comprising an anti-protein of interest antibody.

There are several commercial sources for Protein A resin. One suitable resin is MabSelect™ from GE Healthcare. Suitable resins include, but not limited to, MabSelect SuRe™, MabSelect SuRe LX, MabSelect, MabSelect Xtra, rProtein A Sepharose from GE Healthcare, ProSep HC, ProSep Ultra, and ProSep Ultra Plus from EMD Millipore, MapCapture from Life Technologies. A non-limiting example of a suitable column packed with MabSelect™ is an about 1.0 cm diameter×about 21.6 cm long column (17 mL bed volume). This size column can be used for small scale purifications and can be compared with other columns used for scale ups. For example, a 20 cm×21 cm column whose bed volume is about 6.6 L can be used for larger purifications. A suitable column may comprise a resin such as MabSelect™ SuRe or an analogous resin.

An affinity column can be equilibrated with a suitable buffer prior to sample loading. Following the loading of the column, the column can be washed one or multiple times using a suitable buffer. Once loaded, the column can then be eluted using an appropriate elution buffer. For example, glycine-HCL, acetic acid, or citric acid can be used as an elution buffer. The eluate can be monitored using techniques well known to those skilled in the art such as a UV detector. The eluate fractions of interest can be collected and then prepared for further processing.

In one aspect, the eluate may be subjected to viral inactivation, e.g., either by detergent or low pH. A suitable detergent concentration or pH (and time) can be selected to obtain a desired viral inactivation result. After viral inactivation, the eluate is usually pH and/or conductivity adjusted for subsequent purification steps.

The eluate may be subjected to filtration through a depth filter to remove turbidity and/or various impurities from the protein of interest prior to additional chromatographic polishing steps. Examples of depth filters include, but are not limited to, Millistak+ XOHC, FOHC, DOHC, AIHC, X0SP, and BIHC Pod filters (EMD Millipore), or Zeta Plus 30ZA/60ZA, 60ZA/90ZA, delipid, VR07, and VR05 filters (3M). The Emphaze AEX Hybrid Purifier multimechanism filter may also be used to clarify the eluate. The eluate pool may need to be conditioned to proper pH and conductivity to obtain desired impurity removal and product recovery from the depth filtration step. The invention is not limited to capture of the protein of interest using chromatography.

Other affinity purification resins including a capture moiety which is capable of binding to VEGF mini-trap, such as VEGF, VEGF₁₆₅, an anti-VEGFR antibody or antigen-binding fragment thereof, anti-VEGFR1 antibody or antigen-binding fragment thereof or an anti-VEGFR2 antibody or antigen-binding fragment thereof.

VEGF mini-traps and compositions comprising VEGF mini-trap which is a product of a purification process including affinity purification (e.g., as performed in flow-through mode), e.g., under conditions as discussed herein, are part of the present invention. VEGF mini-traps and compositions comprising VEGF mini-traps which are a product of a purification process including affinity purification (e.g., as performed in bind-and-elute mode) of VEGF trap, such as aflibercept, which is later cleaved with an IdeS protease to generate the VEGF mini-trap, e.g., under conditions as discussed herein, are part of the present invention

In an embodiment of the invention, affinity columns are washed with phosphate buffered saline (PBS), e.g., Dulbecco's Phosphate-Buffered Saline.

### Anion Exchange Chromatography

In certain embodiments, a mini-trap is produced by subjecting a sample matrix to at least one anion exchange separation step. In an aspect, the anion exchange step will occur after the above-described affinity chromatography, e.g., Protein-A affinity. In certain other embodiments, the anion exchange step will occur before the above-described affinity chromatography, e.g., Protein-A affinity. In certain other embodiments, the anion exchange step will occur both before and after the above-described affinity chromatography, e.g., Protein-A affinity.

The use of an anionic exchange material versus a cationic exchange material, such as those cation exchange materials discussed in detail herein, is based on the local charges of the protein of interest under suitable conditions. Anion exchange chromatography can be used in combination with other chromatographic procedures.

In performing a separation, the initial protein composition (sample matrix) can be contacted with an anion exchange material by using any of a variety of techniques, e.g., using a batch purification technique or a chromatographic technique.

For example, in the context of batch purification, anion exchange material is prepared in, or equilibrated to, the desired starting buffer. Upon preparation, or equilibration, a slurry of the anion exchange material is obtained. The protein of interest, e.g., VEGF mini-trap, solution is contacted with the slurry to allow for protein adsorption to the anion exchange material. The solution comprising the acidic species that do not bind to the AEX material is separated from the slurry, e.g., by allowing the slurry to settle and removing the supernatant. The slurry can be subjected to one or more washing steps and/or elution steps.

In the context of chromatographic separation, a chromatographic column is used to house chromatographic support material (resin or solid phase). A sample matrix comprising a protein of interest is loaded onto a particular chromatographic column for separation. The column can then be subjected to one or more wash steps using a suitable buffer. Components of a sample matrix that have not adsorbed onto the resin will likely flow through the column. Components that have adsorbed to the resin can be differentially eluted using an appropriate buffer.

In certain embodiments, a wash step can be performed in the context of AEX chromatography using conditions similar to the load conditions or alternatively by decreasing the pH and/or increasing the ionic strength/conductivity of the wash in a step wise or linear gradient manner. In certain exemplary embodiments, the aqueous salt solution used in both the loading and wash buffer has a pH that at or near the isoelectric point (p1) of the protein of interest. In certain exemplary embodiments the pH is about 0 to 2 units higher or lower than the pl of the protein of interest. In certain exemplary embodiments, it will be in the range of 0 to 0.5 units higher or lower. In certain exemplary embodiments, it will be at the pl of the protein of interest.

In an embodiment of the invention, an AEX chromatography column is washed with (i) a pH 8.40 and 2.00 mS/cm wash buffer, (ii) a pH 8.00 and 2.50 mS/cm wash buffer or (iii) a pH 7.80 and 4.00 mS/cm wash buffer; after a sample containing a VEGF mini-trap (e.g., REGN7483, REGN7850 or REGN7851) is applied and the VEGF mini-trap is retained in the AEX flow-through fraction. Wash buffers are retained after passage through the column. In an embodiment of the invention, the wash buffer contains Tris (e.g., 50 mM) and, optionally, NaCl. In an embodiment of the invention, the AEX column is pre-equilibrated with NaCl (e.g., 2M NaCl). In an embodiment of the invention, the AEX column is equilibrated with wash buffer.

In certain non-limiting embodiments, the anionic agent is selected from the group consisting of acetate, chloride, formate, and combinations thereof. In certain non-limiting embodiments, the cationic agent is selected from the group consisting of Tris, arginine, sodium, and combinations thereof. In one embodiment, the buffer solution is a Tris/formate buffer. In another exemplary embodiment, the buffer is selected from the group consisting of pyridine, piperazine, L-histidine, Bis-tris, Bis-Tris propane, imidazole, N-ethylmorpholine, TEA (triethanolamine), Tris, morpholine, N-methyldiethanolamine, AMPD (2-amino-2-methyl-I,3-propanediol), diethanolamine, ethanolamine, AMP (2-amino-2-methyl-I-propaol), piperazine, 1,3-diaminopropane and piperidine.

A packed anion-exchange chromatography column, anion-exchange membrane device, anion-exchange monolithic device, or depth filter media can be operated either in bind-elute mode, flow-through mode, or a hybrid mode wherein the product exhibits binding to the chromatographic material and yet can be washed from the column using a buffer that is the same or substantially similar to the loading buffer. In the bind-elute mode, the column or the membrane device is first conditioned with a buffer with appropriate ionic strength and pH under conditions where certain proteins will be immobilized on the resin-based matrix. For example, during the feed load, the protein of interest will be adsorbed to the resin due to electrostatic attraction. After washing the column or the membrane device with the equilibration buffer or another buffer with different pH and/or conductivity, the product recovery is achieved by increasing the ionic strength (i.e., conductivity) of the elution buffer to compete with the solute for the charged sites of the anion exchange matrix. Changing the pH and thereby altering the charge of the solute is another way to achieve elution of the solute. The change in conductivity or pH may be gradual (gradient elution) or stepwise (step elution). In the flow-through mode, the column or the membrane device is operated at selected pH and conductivity such that the protein of interest does not bind to the resin or the membrane while the acidic species will either be retained on the column or will have a distinct elution profile as compared to the protein of interest. In the context of this hybrid strategy, acidic species will bind to the chromatographic material (or flow through) in a manner distinct from the protein of interest, e.g., while the protein of interest and certain aggregates and/or fragments of the protein of interest may bind the chromatographic material, washes that preferentially remove the protein of interest can be applied. The column is then regenerated before next use.

Non-limiting examples of anionic exchange resins include diethylaminoethyl (DEAE), quaternary aminoethyl (QAE) and quaternary amine (Q) groups. Additional Non-limiting examples include: Poros 50PI and Poros 50HQ, which are a rigid polymeric bead with a backbone consisting of cross-linked poly[styrene-divinylbenzene]; Capto Q Impres and Capto DEAE, which are a high flow agarose bead; Toyopearl QAE-550, Toyopearl DEAE-650, and Toyopearl GigaCap Q-650, which are a polymeric base bead; Fractogel® EMD TMAE Hicap, which is a synthetic polymeric resin with a tentacle ion exchanger; Sartobind STIC® PA nano, which is a salt-tolerant chromatographic membrane with a primary amine ligand; Sartobind Q nano; which is a strong anion exchange chromatographic membrane; CUNO BioCap; which is a zeta-plus depth filter media constructed from inorganic filter aids, refined cellulose, and an ion exchange resin; and XOHC, which is a depth-filter media constructed from inorganic filter aid, cellulose, and mixed cellulose esters.

In certain embodiments, the protein load of the mixture comprising a protein of interest is adjusted to a total protein load to the column of between about 50 and 500 g/L, or between about 75 and 350 g/L, or between about 200 and 300 g/L. In certain exemplary embodiments, the protein concentration of the load protein mixture is adjusted to a protein concentration of the material loaded to the column of about 0.5 and 50 g/L, between about 1 and 20 g/L, or between 3 and 10 g/L. In certain exemplary embodiments, the protein concentration of the load protein mixture is adjusted to a protein centration of the material to the column of about 37 g/L.

In certain exemplary embodiments, additives such as polyethylene glycol (PEG), detergents, amino acids, sugars, chaotropic agents can be added to enhance the performance of the separation, so as to achieve better recovery or product quality.

The methods of the instant invention can be used to selectively remove, significantly reduce, or essentially remove at least 10% of protein variants in the flow through while enriching for the same in the elution fraction or strip in the case of ion exchange, thereby producing protein compositions that have reduced protein variants or are essentially free of protein variants.

In certain embodiments, the protein variants can include modifications of one or more residues as follows: one or more asparagines are deamidated; one or more aspartic acids are converted aspartate-glycine and/or Asn-Gly; one or more methionines are oxidized; one or more tryptophans are converted to N-formylkynurenin; one or more tryptophans are mono-hydroxyl tryptophan; one or more tryptophans are di-hydroxyl tryptophan; one or more tryptophans are tri-hydroxyl tryptophan; one or more arginines are converted to Arg 3-deoxyglucosone; the C-terminal glycine is not present; and/or there are one or more non-glycosylated glycosites.

In certain exemplary embodiments, the protein variants of aflibercept or VEGF mini-trap can include one or more of (i) oxidized histidines, e.g., from the histidine residues selected from His86, His110, His145, His209, His95, His19 and/or His203; (ii) oxidized tryptophan residues, e.g., selected from tryptophan residues at Trp58 and/or Trp138; (iii) oxidized tyrosine residues, e.g., at Tyr64; (iv) oxidized phenylalanine residues, e.g., selected from Phe44 and/or Phe166 and/or (v) oxidized methionine residues, e.g., selected from Met10, Met 20, Met163 and/or Met192. Such oxidized histidines have been correlated with an undesirable brown-yellow color.

VEGF mini-traps and compositions comprising VEGF mini-trap which is a product of a purification process including AEX chromatography (e.g., as performed in flow-through mode), e.g., under conditions as discussed herein, are part of the present invention. VEGF mini-traps and compositions comprising VEGF mini-traps which are a product of a purification process including AEX chromatography of VEGF trap, such as aflibercept, which is later cleaved with an IdeS protease to generate the VEGF mini-trap, e.g., under conditions as discussed herein, are part of the present invention.

### Cation Exchange Chromatography

The compositions of the present invention can be produced by subjecting the composition, e.g., a primary recovery sample, to at least one cation exchange (CEX) separation step. In certain exemplary embodiments, the CEX step will occur either before or after the above-described AEX. Further, a CEX step can occur throughout the purification procedure.

The use of a cationic exchange material versus an anionic exchange material, such as those anionic exchange materials discussed herein, is based on the local charges of the protein of interest in a given solution. Therefore, it is within the scope of this invention to employ a cationic exchange step prior to the use of an anionic exchange step, or an anionic exchange step prior to the use of a cationic exchange step. Furthermore, it is within the scope of this invention to employ only a cationic exchange step, only an anionic exchange step, or any serial combination of the two (including serial combinations of one or both ion exchange steps with the other chromatographic separation technologies described herein).

In performing the separation, the initial protein mixture can be contacted with a cation exchange material by using any of a variety of techniques, e.g., using a batch purification technique or a chromatographic technique, as described above in connection with Protein A or AEX.

In certain exemplary embodiments, the aqueous salt solution used as both the loading and wash buffer has a pH that is lower than the isoelectric point (pI) of the protein of interest. In certain exemplary embodiments, the pH is about 0 to 5 units lower than the pl of the protein. In certain exemplary embodiments, it is in the range of 1 to 2 units lower. In certain exemplary embodiments, it is in the range of 1 to 1.5 units lower.

In certain exemplary embodiments, the concentration of the anionic agent in aqueous salt solution is increased or decreased to achieve a pH of between about 3.5 and 10.5, or between about 4 and 10, or between about 4.5 and 9.5, or between about 5 and 9, or between about 5.5 and 8.5, or between about 6 and 8, or between about 6.5 and 7.5. In certain exemplary embodiments, the concentration of anionic agent is increased or decreased in the aqueous salt solution to achieve a pH of 5, or 5.5, or 6, or 6.5, or 6.8, or 7.5. Buffer systems suitable for use in the CEX methods include, but are not limited to, Tris formate, Tris acetate, ammonium sulfate, sodium chloride, and sodium sulfate.

In certain exemplary embodiments, the conductivity and pH of the aqueous salt solution is adjusted by increasing or decreasing the concentration of a cationic agent. In certain exemplary embodiments, the cationic agent is maintained at a concentration ranging from about 20 mM to 500 mM, about 50 mM to 350 mM, about 100 to 300 mM, or about 100 mM to 200 mM. In certain non-limiting embodiments, the cationic agent is selected from the group consisting of sodium, Tris, tromethalmine, ammonium, arginine, and combinations thereof. In certain non-limiting embodiments, the anionic agent is selected from the group consisting of formate, acetate, citrate, chloride anion, sulphate, phosphate and combinations thereof.

A packed cation-exchange chromatography column or a cation-exchange membrane device can be operated either in bind-elute mode, flow-through mode, or a hybrid mode wherein the product exhibits binding to the chromatographic material yet can be washed from the column using a buffer that is the same or substantially similar to the loading buffer. The details of these modes are outlined above.

Cationic substituents include carboxymethyl (CM), sulfoethyl (SE), sulfopropyl (SP), phosphate (P) and sulfonate (S). Additional cationic materials include but are not limited to: Capto SP ImpRes, which is a high flow agarose bead; CM Hyper D grade F; which is a ceramic bead coated and permeated with a functionalized hydrogel, 250-400 ionic groups μeq/mL; Eshmuno S, which is a hydrophilic polyvinyl ether base matrix with 50-100 μeq/mL ionic capacity; Nuvia C Prime, which is a hydrophobic cation exchange media composed of a macroporous highly crosslinked hydrophilic polymer matrix 55-75 με/ [symbol] ; Nuvia S, which has a UNOsphere base matrix with 90-150 με/ [symbol] . ionic groups; Poros HS; which is a rigid polymetic bead with a backbone consisting of cross-linked poly[styrene-divinylbenzene]; Poros XS; which is a rigid polymetic bead with a backbone consisting of cross-linked poly[styrene divinyl-benzene]; Toyo Pearl Giga Cap CM 650M, which is a polymeric base bead with 0.225 meq/mL ionic capacity; Toyo Pearl Giga Cap S 650M which is a polymeric base bead; Toyo Pearl MX TRP, which is a polymeric base bead. It is noted that CEX chromatography can be used with MM resins, described herein.

In certain exemplary embodiments, the protein load of the mixture comprising protein of interest (e.g., VEGF mini-trap) is adjusted to a total protein load to the column of between about 5 and 150 g/L, or between about 10 and 100 g/L, between about 20 and 80 g/L, between about 30 and 50 g/L, or between about 40 and 50 g/L. In certain exemplary embodiments, the protein concentration of the load protein mixture is adjusted to a protein concentration of the material loaded to the column of about 0.5 and 50 g/L, or between about 1 and 20 g/L.

In certain exemplary embodiments, additives such as polyethylene glycol, detergents, amino acids, sugars, chaotropic agents can be added to enhance the performance of the separation so as to achieve better recovery or product quality.

In certain embodiments, the methods of the instant invention can be used to selectively remove, significantly reduce, or essentially remove all of the variants in a sample matrix where the protein of interest will essentially be in the flow through of a CEX step while the oxo-variants will be substantially captured by the column media.

In an embodiment of the invention, CEX is loaded with a sample containing VEGF mini-trap in a loading buffer at pH5.0, e.g., 20 mM acetate, pH 5.0. In an embodiment of the invention, the column is also washed with the loading buffer. A wash may be performed with a pH 7.0 wash buffer, e.g., 10 mM phosphate, pH7.0. Elution of VEGF mini-trap from the CEX column can be performed with (NH₄)₂SO₄, e.g., at pH 8.5, e.g., 50 mM Tris, 62.5 mM (NH₄)₂SO₄, pH 8.5.

VEGF mini-traps and compositions comprising VEGF mini-trap which is a product of a purification process including CEX chromatography, e.g., under conditions as discussed herein, are part of the present invention. VEGF mini-traps and compositions comprising VEGF mini-traps which are a product of a purification process including CEX chromatography of VEGF trap, such as aflibercept, which is later cleaved with an IdeS protease to generate the VEGF mini-trap, e.g., under conditions as discussed herein, are part of the present invention.

### Mixed Mode Chromatography

Mixed mode (“MM”) chromatography may also be used to prepare the compositions of the invention. MM chromatography, also referred to herein as “multimodal chromatography”, is a chromatographic strategy that utilizes a support comprising a ligand that is capable of providing at least two different interactions with a substance to be bound. In certain exemplary embodiments, one of these sites provides an attractive type of charge-charge interaction between the ligand and the substance of interest and the other site provides for electron acceptor-donor interaction and/or hydrophobic and/or hydrophilic interactions. Electron donor-acceptor interactions include interactions such as hydrogen-bonding, π-π, cation-π, charge transfer, dipole-dipole, induced dipole etc.

In certain embodiments, the resin employed for a mixed mode separation is Capto Adhere. Capto Adhere is a strong anion exchanger with multimodal functionality. Its base matrix is a highly cross-linked agarose with a ligand (N-benzyl-N-methyl ethanol amine) that exhibits different functionalities for interaction, such as ionic interaction, hydrogen bonding and hydrophobic interaction. In certain aspects, the resin employed for a mixed mode separation is selected from PPA-HyperCel and HEA-HyperCel. The base matrices of PPA-HyperCel and HEA-HyperCel are high porosity cross-linked cellulose. Their ligands are phenylpropylamine and hexylamine, respectively. Phenylpropylamine and hexylamine offer different selectivity and hydrophobicity options for protein separations. Additional mixed mode chromatographic supports include, but are not limited to, Nuvia C Prime, Toyo Pearl MX Trp 650M, and Eshmuno® HCX. In certain aspects, the mixed mode chromatography resin is comprised of ligands coupled to an organic or inorganic support, sometimes denoted a base matrix, directly or via a spacer. The support may be in the form of particles, such as essentially spherical particles, a monolith, filter, membrane, surface, capillaries, and the like. In certain aspects, the support is prepared from a native polymer, such as cross-linked carbohydrate material, such as agarose, agar, cellulose, dextran, chitosan, konjac, carrageenan, gellan, alginate, and the like. To obtain high adsorption capacities, the support can be porous, and ligands are then coupled to the external surfaces as well as to the pore surfaces. Such native polymer supports can be prepared according to standard methods, such as inverse suspension gelation (S Hjerten: Biochim Biophys Acta 79(2), 393-398 (1964)). Alternatively, the support can be prepared from a synthetic polymer, such as cross-linked synthetic polymers, e.g. styrene or styrene derivatives, divinylbenzene, acrylamides, acrylate esters, methacrylate esters, vinyl esters, vinyl amides, and the like. Such synthetic polymers can be produced according to standard methods, see e.g. “Styrene based polymer supports developed by suspension polymerization” (R Arshady: Chimica e L'Industria 70(9), 70-75 (1988)). Porous native or synthetic polymer supports are also available from commercial sources, such as GE Healthcare, Uppsala, Sweden.

In certain embodiments, the protein load of the mixture comprising the protein of interest is adjusted to a total protein load to the column of between about 25 and 750 g/L, or between about 75 and 500 g/L, or between about 100 and 300 g/L. In certain exemplary embodiments, the protein concentration of the load protein mixture is adjusted to a protein concentration of the material loaded to the column of about 1 and 50 g/L, or between about 9 and 25 g/L.

In certain embodiments, additives such as polyethylene glycol, detergents, amino acids, sugars, chaotropic agents can be added to enhance the performance of the separation, so as to achieve better recovery or product quality.

The methods of the instant invention can be used to selectively remove, significantly reduce, or essentially remove all of PTMs, such as 2-oxo-histdine comprising proteins, in the flow through fractions while enriching for the same in the stripped fractions.

The methods for producing the composition of the invention can also be implemented in a continuous chromatography mode. In this mode, at least two columns are employed (referred to as a “first” column and a “second” column). In certain exemplary embodiments, this continuous chromatography mode can be performed such that the eluted fractions and/or stripped fractions can containing the higher level of PTMs, such as 2-oxo-histdine comprising proteins, can then be loaded subsequently or concurrently onto the second column (with or without dilution), such that the operation of the two columns are not in tandem, reducing complexity of the operation.

In one embodiment, the media choice for the continuous modes can be one of many chromatographic resins with pendant hydrophobic and anion exchange functional groups, monolithic media, membrane adsorbent media or depth filtration media.

VEGF mini-traps and compositions comprising VEGF mini-trap which is a product of a purification process including MM chromatography, e.g., under conditions as discussed herein, are part of the present invention. VEGF mini-traps and compositions comprising VEGF mini-traps which are a product of a purification process including MM chromatography of VEGF trap, such as aflibercept, which is later cleaved with an IdeS protease to generate the VEGF mini-trap, e.g., under conditions as discussed herein, are part of the present invention.

### Hydrophobic Interaction Chromatography

The compositions of the invention may also be prepared using a hydrophobic interaction chromatography (HIC).

In performing the separation, the sample mixture is contacted with a HIC material, e.g., using a batch purification technique or using a column or membrane chromatography. Prior to HIC purification, it may be desirable to adjust the concentration of the salt buffer to achieve desired protein binding to the resin or the membrane.

Whereas ion exchange chromatography relies on the local charge of the protein of interest for selective separation, hydrophobic interaction chromatography exploits the hydrophobic properties of proteins to achieve selective separation. Hydrophobic groups on the protein interact with hydrophobic groups of the resin or the membrane. The more hydrophobic a protein, the stronger it will interact with the column or the membrane under suitable conditions. Thus, HIC can be used to remove process-related impurities (e.g., HCPs) as well as product-related substances (e.g., aggregates and fragments) under suitable conditions.

Like ion exchange chromatography, a HIC column or a HIC membrane device can also be operated in product an elution mode, a flow-through, or a hybrid mode wherein the product exhibits binding to the chromatographic material, yet can be washed from the column using a buffer that is the same or substantially similar to the loading buffer (the details of these modes are outlined herein in connection with AEX purification). As hydrophobic interactions are strongest at high ionic strength, this form of separation is conveniently performed following salt elution step, such as those that are typically used in connection with ion exchange chromatography. Alternatively, salts can be added into a low salt level feed stream before this step. Adsorption of the VEGF mini-trap to a HIC column is favored by high salt concentrations, but the actual concentrations can vary over a wide range depending on the nature of the protein of interest, salt type and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba²⁺; Ca²⁺; Mg²⁺; Li⁺; Cs⁺; Na⁺; K⁺; Rb⁺; NH₄⁺, while anions may be ranked in terms of increasing chaotropic effect as P0₄³⁻; S0₄²⁻; CH₃C0₃⁻; Cl⁻; Br⁻; N0₃⁻; ClO₄⁻; I⁻; SCN⁻.

In general, Na⁺, K⁺ or NH₄⁺ sulfates effectively promote ligand-protein interaction using HIC. Salts may be formulated that influence the strength of the interaction as given by the following relationship: (NH₄)₂S0₄>Na₂S0₄>NaCl>NH₄Cl>NaBr>NaSCN. In general, salt concentrations of between about 0.75 M and about 2 M ammonium sulfate or between about 1 and 4 M NaCl are useful.

HIC media normally comprise a base matrix (e.g., cross-linked agarose or synthetic copolymer material) to which hydrophobic ligands (e.g., alkyl or aryl groups) are coupled. A suitable HIC media comprises an agarose resin or a membrane functionalized with phenyl groups (e.g., a Phenyl Sepharose™ from GE Healthcare or a Phenyl Membrane from Sartorius). Many HIC resins are available commercially. Examples include, but are not limited to, Capto Phenyl, Phenyl Sepharose™ 6 Fast Flow with low or high substitution, Phenyl Sepharose™ High Performance, Octyl Sepharose™ High Performance (GE Healthcare); Fractogel™ EMD Propyl or Fractogel™ EMD Phenyl (E. Merck, Germany); Macro-Prep™ Methyl or Macro-Prep™ t-Butyl columns (Bio-Rad, California), WP HI-Propyl (C3)™ (J. T. Baker, New Jersey); and Toyopearl™ ether, phenyl or butyl (TosoHaas, Pa.).

VEGF mini-traps and compositions comprising VEGF mini-trap which is a product of a purification process including HIC chromatography, e.g., under conditions as discussed herein, are part of the present invention. VEGF mini-traps and compositions comprising VEGF mini-traps which are a product of a purification process including HIC chromatography of VEGF trap, such as aflibercept, which is later cleaved with an IdeS protease to generate the VEGF mini-trap, e.g., under conditions as discussed herein, are part of the present invention.

### Viral Filtration

Viral filtration is a dedicated viral reduction step in a purification process. This step is usually performed post chromatographic polishing steps. Viral reduction can be achieved via the use of suitable filters including, but not limited to, Planova 20N™ 50 N or BioEx from Asahi Kasei Pharma, Viresolve™ filters from EMD Millipore, ViroSart CPV from Sartorius, or Ultipor DV20 or DV50™ filter from Pall Corporation. It will be apparent to one of ordinary skill in the art to select a suitable filter to obtain desired filtration performance.

VEGF mini-traps and compositions comprising VEGF mini-trap which is a product of a purification process including viral filtration, e.g., under conditions as discussed herein, are part of the present invention. VEGF mini-traps and compositions comprising VEGF mini-traps which are a product of a purification process including viral filtration of VEGF trap, such as aflibercept, which is later cleaved with an IdeS protease to generate the VEGF mini-trap, e.g., under conditions as discussed herein, are part of the present invention.

### Ultrafiltration/Diafiltration

Certain embodiments of the present invention employ ultrafiltration and diafiltration to further concentrate and formulate a protein of interest, e.g., a mini-trap. Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). One filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size of smaller than 0.1 μm. By employing filters having such small pore size, the volume of the sample can be reduced through permeation of the sample buffer through the filter membrane pores while proteins, such as VEGF mini-traps, are retained above the membrane surface.

One of ordinary skill in the art can select an appropriate membrane filter device for the UF/DF operation. Examples of membrane cassettes suitable for the present invention include, but not limited to, Pellicon 2 or Pellicon 3 cassettes with 10 kD, 30kD or 50 kD membranes from EMD Millipore, Kvick 10 kD, 30 kD or 50 kD membrane cassettes from GE Healthcare, and Centramate or Centrasette 10 kD, 30 kD or 50 kD cassettes from Pall Corporation.

VEGF mini-traps and compositions comprising VEGF mini-trap which is a product of a purification process including UF and/or DF, e.g., under conditions as discussed herein, are part of the present invention. VEGF mini-traps and compositions comprising VEGF mini-traps which are a product of a purification process including UF and/or DF of VEGF trap, such as aflibercept, which is later cleaved with an IdeS protease to generate the VEGF mini-trap, e.g., under conditions as discussed herein, are part of the present invention.

### Exemplary Purification Schemes

In certain exemplary embodiments, primary recovery can proceed by sequentially employing pH reduction, centrifugation, and filtration to remove cells and cell debris (including HCPs) from the production bioreactor harvest. In certain embodiments, the present invention is directed to subjecting a sample mixture from the primary recovery to one or more AEX, CEX, and/or MM purification steps. Certain aspects of the present invention will include further purification steps. Examples of additional purification procedures which can be performed prior to, during, or following the ion exchange chromatography method include ethanol precipitation, isoelectric focusing, size-exclusion chromatography, reverse phase HPLC, chromatography on silica, chromatography on heparin Sepharose™, further anion exchange chromatography and/or further cation exchange chromatography, chromatofocusing, SDS-PAGE, ammonium sulfate precipitation, hydroxyapatite chromatography, gel electrophoresis, dialysis, and affinity chromatography (e.g., using protein G or A, an antibody, a specific substrate, ligand or antigen as the capture reagent). In certain aspects, the column temperature can be independently varied to improve the separation efficiency and/or yield of any particular purification step.

In certain embodiments the unbound flow-through and wash fractions can be further fractionated and a combination of fractions providing a target product purity can be pooled.

In certain exemplary embodiments, the loading & washing steps can be controlled by in-line, at-line or off-line measurement of the product related impurity/substance levels, either in the column effluent, or the collected pool or both, so as to achieve the target product quality and/or yield. In certain embodiments, the loading concentration can be dynamically controlled by in-line or batch or continuous dilutions with buffers or other solutions to achieve the partitioning necessary to improve the separation efficiency and/or yield.

Examples of such purification procedures are as follows. The present invention includes VEGF mini-traps which are the product of a process including the steps of any of such purification processes.
(1) A method of manufacturing aflibercept can comprise
(a) expressing aflibercept in a CDM;
(b) capturing aflibercept using a first chromatography support which can include an affinity capture chromatography; and
(c) contacting at least a portion of aflibercept of step (b) to a second chromatography support which can include an anion-exchange chromatography.
   Step (c) can further comprise collecting flow-through fraction(s) of the mixture containing the aflibercept that does not bind to the second chromatographic support. Optionally, step (c) can comprise stripping the second chromatographic support and collecting stripped fractions. The steps can be carried out by routine methodology in conjunction with methodology mentioned herein.

Other additional exemplary embodiment can include (d) contacting at least a portion of said aflibercept of step (c) to a third chromatography support. In one aspect of such an embodiment, the manufacture can include (e) contacting at least a portion of said aflibercept of step (d) to a fourth chromatography support. In one aspect of this embodiment, the manufacturing can optionally comprise subjecting said aflibercept of step (c) to a pH less than 5.5. In one aspect of this embodiment, the method of can optionally comprise clarifying a solution having fusion binding molecule before said capture step (a). In one aspect of this embodiment, the method can optionally comprise eluting said fusion binding molecule of step (a). In yet another aspect of this embodiment, the method of manufacturing aflibercept can optionally comprise collecting flow-through fraction(s) of step (c). In yet another aspect of this embodiment, the method of manufacturing aflibercept can optionally comprise eluting said aflibercept of step (d). In yet another aspect of this embodiment, the method of manufacturing aflibercept can optionally comprise eluting said aflibercept of step (e). In one aspect of this embodiment, the first chromatographic support and/or the second chromatographic support and/or the third chromatographic support and/or the fourth chromatographic support can be same or distinct and can comprise affinity chromatography media, ion-exchange chromatography media, or hydrophobic interaction chromatography media. In a specific aspect of this embodiment, the ion-exchange chromatography media can be an anion-exchange chromatography media. In another specific aspect of this embodiment, the ion-exchange chromatography media can be a cation-exchange chromatography media. In one aspect of this embodiment, the method of manufacturing aflibercept can optionally comprise filtering said aflibercept of any of the steps using virus filtration. In one aspect of this embodiment, the manufacturing can optionally comprise filtering said aflibercept of any of the steps using ultrafiltration and/or diafiltration procedure (UF/DF).

The present invention includes VEGF mini-traps which are the product of a process including the step of cleaving such aflibercept with an IdeS protease.

(2) A method of manufacturing a VEGF mini-trap can comprise
(a) expressing aflibercept in a CDM;
(b) capturing aflibercept using a first chromatography support which can include an affinity capture chromatography;
(c) cleaving the aflibercept (e.g., with an IdeS protease) thereby forming a mixture containing a VEGF mini-trap and Fc fragment from the aflibercept;
(d) contacting said mixture to a second chromatographic support which can be affinity capture chromatography; and
(e) contacting at least a portion of said VEGF mini-trap of step (c) to a third chromatography support which can include an anion-exchange chromatography.
   Step (d) can optionally also comprise collecting flow-through fraction(s) of the mixture containing the VEGF mini-trap that does not bind to the second chromatographic support of step. Step (e) can further comprise collecting flow-through fraction(s) of the mixture containing the VEGF mini-trap that does not bind to the third chromatographic support. Optionally, step (d) can comprise stripping the third chromatographic support and collecting stripped fractions. The steps can be carried out by routine methodology in conjunction with methodology mentioned herein.

Other additional exemplary embodiments can include (f) contacting at least a portion of said VEGF mini-trap of step (e) to a fourth chromatography support. In one aspect of such an embodiment, the manufacture can include (g) contacting at least a portion of said VEGF mini-trap of step (f) to a fifth chromatography support. In one aspect of this embodiment, the manufacturing can optionally comprise subjecting said VEGF mini-trap of step (d) to a pH less than 5.5. In one aspect of this embodiment, the method of manufacturing a VEGF mini-trap can optionally comprise clarifying a solution having fusion binding molecule before said capture step (a). In one aspect of this embodiment, the method of manufacturing a VEGF mini-trap can optionally comprise eluting said fusion binding molecule of step (a). In yet another aspect of this embodiment, the method of manufacturing a VEGF mini-trap can optionally comprise collecting flow-through fraction(s) of step (e). In yet another aspect of this embodiment, the method of manufacturing a VEGF mini-trap can optionally comprise eluting said VEGF mini-trap of step (f). In yet another aspect of this embodiment, the method of manufacturing a VEGF mini-trap can optionally comprise eluting said VEGF mini-trap of step (g). In one aspect of this embodiment, the first chromatographic support and/or the second chromatographic support and/or the third chromatographic support and/or the fourth chromatographic support and/or the fifth chromatographic support can be same or distinct and can comprise affinity chromatography media, ion-exchange chromatography media, or hydrophobic interaction chromatography media. In a specific aspect of this embodiment, the ion-exchange chromatography media can be an anion-exchange chromatography media. In another specific aspect of this embodiment, the ion-exchange chromatography media can be a cation-exchange chromatography media. In one aspect of this embodiment, the method of manufacturing a VEGF mini-trap can optionally comprise filtering said VEGF mini-trap of any of the steps using virus filtration. In one aspect of this embodiment, the manufacturing can optionally comprise filtering said VEGF mini-trap of any of the steps using ultrafiltration and/or diafiltration procedure (UF/DF).

The present invention includes VEGF mini-traps which are the product of such a process.

(3) A method of manufacturing a aflibercept can comprise:
(a) expressing aflibercept in a CDM;
(b) capturing aflibercept using a first chromatography support which can include a cation exchange chromatography; and
(c) contacting at least a portion of aflibercept of step (b) to a second chromatography support which can include an anion-exchange chromatography.
   Step (c) can further comprise collecting flow-through fraction(s) of the mixture containing the aflibercept that does not bind to the second chromatographic support. Optionally, step (c) can comprise stripping the second chromatographic support and collecting stripped fractions. The steps can be carried out by routine methodology in conjunction with methodology mentioned herein.

Other additional exemplary embodiment can include (d) contacting at least a portion of said aflibercept of step (c) to a third chromatography support. In one aspect of such an embodiment, the manufacture can include (e) contacting at least a portion of said aflibercept of step (d) to a fourth chromatography support. In one aspect of this embodiment, the manufacturing can optionally comprise subjecting said aflibercept of step (c) to a pH less than 5.5. In one aspect of this embodiment, the method of can optionally comprise clarifying a solution having fusion binding molecule before said capture step (a). In one aspect of this embodiment, the method can optionally comprise eluting said fusion binding molecule of step (a). In yet another aspect of this embodiment, the method of manufacturing Aflibercept can optionally comprise collecting flow-through fraction(s) of step (c). In yet another aspect of this embodiment, the method of manufacturing aflibercept can optionally comprise eluting said aflibercept of step (d). In yet another aspect of this embodiment, the method of manufacturing aflibercept can optionally comprise eluting said aflibercept of step (e). In one aspect of this embodiment, the first chromatographic support and/or the second chromatographic support and/or the third chromatographic support and/or the fourth chromatographic support can be same or distinct and can comprise affinity chromatography media, ion-exchange chromatography media, or hydrophobic interaction chromatography media. In a specific aspect of this embodiment, the ion-exchange chromatography media can be an anion-exchange chromatography media. In another specific aspect of this embodiment, the ion-exchange chromatography media can be a cation-exchange chromatography media. In one aspect of this embodiment, the method of manufacturing aflibercept can optionally comprise filtering said aflibercept of any of the steps using virus filtration. In one aspect of this embodiment, the manufacturing can optionally comprise filtering said aflibercept of any of the steps using ultrafiltration and/or diafiltration procedure (UF/DF).

The present invention includes VEGF mini-traps which are the product of a process including the step of cleaving such aflibercept with an IdeS protease.
(4) A method of manufacturing a VEGF mini-trap can comprise:
(a) expressing aflibercept in a CDM;
(b) capturing aflibercept using a first chromatography support which can include an cation exchange chromatography;
(c) cleaving the aflibercept (e.g., with an IdeS protease) thereby forming a mixture containing a VEGF mini-trap and Fc fragment from the aflibercept;
(d) contacting said mixture to a second chromatographic support which can be affinity capture chromatography; and
(e) contacting at least a portion of said VEGF mini-trap of step (c) to a third chromatography support which can include an anion-exchange chromatography.
   Step (d) can optionally also comprise collecting flow-through fraction(s) of the mixture containing the VEGF mini-trap that does not bind to the second chromatographic support of step. Step (e) can further comprise collecting flow-through fraction(s) of the mixture containing the VEGF mini-trap that does not bind to the third chromatographic support. Optionally, step (d) can comprise stripping the third chromatographic support and collecting stripped fractions. The steps can be carried out by routine methodology in conjunction with methodology mentioned herein.

Other additional exemplary embodiment can include (f) contacting at least a portion of said VEGF mini-trap of step (e) to a fourth chromatography support. In one aspect of such an embodiment, the manufacture can include (g) contacting at least a portion of said VEGF mini-trap of step (f) to a fifth chromatography support. In one aspect of this embodiment, the manufacturing can optionally comprise subjecting said VEGF mini-trap of step (d) to a pH less than 5.5. In one aspect of this embodiment, the method of manufacturing a VEGF mini-trap can optionally comprise clarifying a solution having fusion binding molecule before said capture step (a). In one aspect of this embodiment, the method of manufacturing a VEGF mini-trap can optionally comprise eluting said fusion binding molecule of step (a). In yet another aspect of this embodiment, the method of manufacturing a VEGF mini-trap can optionally comprise collecting flow-through fraction(s) of step (e). In yet another aspect of this embodiment, the method of manufacturing a VEGF mini-trap can optionally comprise eluting said VEGF mini-trap of step (f). In yet another aspect of this embodiment, the method of manufacturing a VEGF mini-trap can optionally comprise eluting said VEGF mini-trap of step (g). In one aspect of this embodiment, the first chromatographic support and/or the second chromatographic support and/or the third chromatographic support and/or the fourth chromatographic support and/or the fifth chromatographic support can be same or distinct and can comprise affinity chromatography media, ion-exchange chromatography media, or hydrophobic interaction chromatography media. In a specific aspect of this embodiment, the ion-exchange chromatography media can be an anion-exchange chromatography media. In another specific aspect of this embodiment, the ion-exchange chromatography media can be a cation-exchange chromatography media. In one aspect of this embodiment, the method of manufacturing a VEGF mini-trap can optionally comprise filtering said VEGF mini-trap of any of the steps using virus filtration. In one aspect of this embodiment, the manufacturing can optionally comprise filtering said VEGF mini-trap of any of the steps using ultrafiltration and/or diafiltration procedure (UF/DF).

The present invention includes VEGF mini-traps which are the product of such a process.

### Mini-Trap Post-Translational Modifications

VEGF mini-traps of the present invention and compositions thereof may be characterized by various post-translational modifications.

Oxidized Species

2-oxo-histidine is a result of histidine oxidation and can function as a marker for protein oxidation. 2-oxo-histidine has been correlated with the presence of a brown-yellow color in VEGF mini-trap (e.g., REGN7483^{F}, REGN7483^{R}, REGN7850 or REGN7851) preparations that have been expressed from cells in chemically-defined media (CDM). Chemically-defined cell growth media offers several significant advantages to biopharmaceutical manufacturing including the reduction of lot-to-lot variability and greater safety, for example, from infectious agents. In order to realize a benefit from these advantages with respect to mini-trap for ophthalmic injection, however, a reduction in the brown-yellow color of the mini-trap compositions expressed in CDM is necessary. Reduction of the 2-oxo-histidine content of mini-trap is a means by which the color may be reduced to a level which is acceptable for intravitreal injection. The present invention presents, in part, methods by which to reduce 2-oxo-histidine and, thus, brown-yellow color as well as compositions which are the result of such methods.

Brown-yellow color is particularly undesirable in any biological product which will be injected into the eye (e.g., VEGF mini-trap). Only very minimal 2-oxo-histidine has been observed in commercially available VEGF Trap molecules (e.g., Eylea) expressed in non-chemically-defined media, such as media containing hydrolysates (e.g., soy hydrolysates). Since eyes are visual organs, the introduction of a colored liquid into the vitreous could have a negative effect on vision. Vision is particularly sensitive to any obstruction of the inner eye. For example, clear microdroplets of silicone oil, sloughed off of the syringe wall and injected into the vitreous, have been reported to disturb vision in the form of floaters. Yu et al., Am J Ophthalmol Case Rep. 2018 June; 10: 142-144.

A chemically-defined medium (CDM) or synthetic medium are terms commonly used in the art and refer to a medium in which the chemical composition is known. A CDM does not include hydrolysate such as, for example, soy hydrolysate. A suitable CDM includes Dulbecco's Modified Eagle's (DME) medium, Ham's Nutrient Mixture, EX-CELL medium, IS CHO-CD medium, and other CDMs known to those skilled in the art whose uses are contemplated to be within the scope of the present invention.

Two chemical versions of 2-oxo-histidine (2-oxo-his) can be produced,

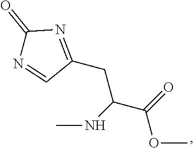

having a 13.98 Da increase in molecular weight relative to histidine (13.98 Da version); or

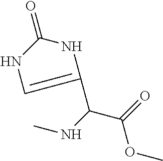

having a 15.99 Da increase in molecular weight relative to histidine (15.99 Da version); wherein the 13.98 Da version of 2-oxo-histidine is the predominant moiety observed in mini-trap expressed in CDM. The content of the 13.98 Da version of 2-oxo-histidine in a peptide can be evaluated spectrophotometrically since this moiety has an enhanced absorbance of light at 350 nM wavelength whereas the 15.99 Da version does not have such an enhanced absorbance. Formation of the 13.98 Da version of 2-oxo-histidine in mini-trap may be catalyzed by light whereas formation of the 15.99 Da version may be catalyzed by metal such as copper (Cu²⁺). The brown-yellow color in CDM-expressed mini-trap has not been correlated with the presence of the 15.99 Da version of 2-oxo-histidine.

Other oxidized species of amino acid which may lead to the brown-yellow color include oxidized tryptophan, methionine, phenylalanine and/or tyrosine. Methods described herein may also be used to reduce the presence of such oxidized amino acids in the VEGF mini-traps discussed herein. Composition comprising such VEGF mini-traps also form part of the present invention.

Oxidation of tryptophan can give a complex mixture of products. The primary products can be N-formylkynurenine and kynurenine along with mono-oxidation, di-oxidation and/or tri-oxidation products. Peptides bearing oxidized Trp modifications generally exhibit mass increases of 4, 16, 32 and 48 Da, corresponding to the formation of kynurenine (KYN), hydroxytryptophan (Wₒₓ₁), and N-formylkynurenine/dihydroxytryptophan (NFK/Wₒₓ₂, referred to also as “doubly oxidized Trp”), trihydroxytryptophan (Wₒₓ₃, referred to also as “triply oxidized Trp”), and their combinations, such as hydroxykynurenine (KYNₒₓ₁, +20 Da). Oxidation to hydroxytryptophan (Wₒₓ₁) (Mass spectrometric identification of oxidative modifications of tryptophan residues in proteins: chemical artifact or post-translational modification? J Am Soc Mass Spectrom. 2010 July; 21(7): 1114-1117). Tryptophan oxidation, but not methionine and histidine oxidation have been found to produce a color change in protein products (Characterization of the Degradation Products of a Color-Changed Monoclonal Antibody: Tryptophan-Derived Chromophores. dx.doi.org/10.1021/ac404218t|Anal. Chem. 2014, 86, 6850-6857). Similar to tryptophan, oxidation of tyrosine primarily yields 3,4-dihydroxyphenylalanine (DOPA) and dityrosine (Li, S, C Schoneich, and R T. Borchardt. 1995. Chemical Instability of Protein Pharmaceuticals: Mechanisms of Oxidation and Strategies for Stabilization. Biotechnol. Bioeng. 48:490-500).

The present invention includes mini-traps (e.g., REGN7483^{F}) comprising one or more tryptophan residues that have been oxidized (e.g., as discussed herein) and compositions thereof, e.g., wherein no more than about 0.1-10% (e.g., about 0.1, 0.2, 0.25, 0.3, 0.4, 0.5, 1, 2, 3, 4, 5, 6, 7, 8, 9, or 10%) of tryptophan residues in the composition are oxidized.

The present invention includes mini-trap molecules described herein (e.g., REGN7483^{F} or REGN7483^{R}) wherein one or more (e.g., 1, 2, 3, 4, 5, 6, 7 or 8) histidines are oxidized to 2-oxo-his (e.g., selected from H19, H86, H95, H110, H145, H147, H203 and/or H203) as well as compositions thereof (e.g., aqueous compositions).

The present invention also compositions (e.g., aqueous compositions) that comprise VEGF mini-traps of the present invention (e.g., REGN7483^{F}, REGN7483^{R}, REGN7850 or REGN7851) (e.g., that were expressed in CDM, e.g., in a host cell such as a CHO cell) wherein no more than about 1% or 2%, no more than about 0.1% or about 0.1-1%, 0.2-1%, 0.3-1%, 0.4-1%, 0.5-1%, 0.6-1%, 0.7-1%, 0.8-1% or 0.9-1% of histidines in the composition are 2-oxo-histidine. In such compositions, the mini-trap polypeptides are a heterogeneous population of peptides each having a varying number of 2-oxo-histidine residues and un-oxidized histidine residues. Thus, the percentage of 2-oxo-histidine in a composition refers to the 2-oxo-histidines among all of the mini-trap molecules÷total histidines in the mini-trap molecules (oxidized+un-oxidized)×100. In an embodiment of the invention, the composition is characterized by a brown-yellow color profile as described herein (e.g., no darker than BY3, 4, 5, 6 or 7; or clear).

One method to quantitate the level of 2-oxo-histidines in a composition is to digest the VEGF mini-trap (e.g., REGN7483^{F}, REGN7483^{R}, REGN7850 or REGN7851) (e.g., that was expressed in CDM) with a protease (e.g., Lys-C and/or trypsin) and analyze the quantity of 2-oxo-histidines in the resulting peptides, for example, by mass spectrometry (ms). In an embodiment of the invention, before digestion of the mini-trap polypeptides, cysteines sulfhydryl groups are blocked by reaction with iodoacetamide (IDAM); resulting in a residue represented by the following chemical structure:

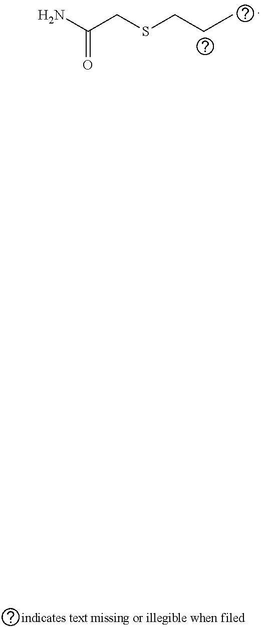

Such modification protects free thiols from reforming disulfide bridges and prevents disulfide bond scrambling. The present invention includes compositions (e.g., aqueous compositions) including VEGF mini-traps (e.g., REGN7483^{F}) comprising polypeptides which, when modified with IDAM and digested with protease (e.g., Lys-C and trypsin) and analyzed by mass spectrometry, comprise the following peptides:
EIGLLTC*EATVNGH*LYK (amino acids 73-89 of SEQ ID NO: 12) which comprises about 0.0095% 2-oxo-histidines,
QTNTIIDVVLSPSH*GIELSVGEK (amino acids 97-119 of SEQ ID NO: 12) which comprises about 0.0235% 2-oxo-histidines,
TELNVGIDFNWEYPSSKH*QHK (amino acids 128-148 of SEQ ID NO: 12) which comprises about 0.067% 2-oxo-histidines,
DKTH*TC*PPC*PAPELLG (amino acids 206-221 of SEQ ID NO: 12) which comprises about 0.0745% 2-oxo-histidines, and/or
TNYLTH*R (amino acids 90-96 of SEQ ID NO: 12) which comprises about 0.016% 2-oxo-histidines, and/or
optionally, IIW*DSR (amino acids 56-61 of SEQ ID NO: 12) which comprises about 0.248% dioxidated tryptophans,
   wherein H* is 2-oxo-histidine, W* is dioxidated tryptophan, and wherein C* is carboxymethylated cysteine;
or
   EIGLLTC*EATVNGH*LYK (amino acids 73-89 of SEQ ID NO: 12) which comprises about 0.006-0.013% 2-oxo-histidines,
   QTNTIIDVVLSPSH*GIELSVGEK (amino acids 97-119 of SEQ ID NO: 12) which comprises about 0.019-0.028% 2-oxo-histidines,
   TELNVGIDFNWEYPSSKH*QHK (amino acids 128-148 of SEQ ID NO: 12) which comprises about 0.049-0.085% 2-oxo-histidines,
   DKTH*TC*PPC*PAPELLG (amino acids 206-221 of SEQ ID NO: 12) which comprises about 0.057-0.092% 2-oxo-histidines, and/or
   TNYLTH*R (amino acids 90-96 of SEQ ID NO: 12) which comprises about 0.010-0.022% 2-oxo-histidines, and/or
   Optionally, IIW*DSR (amino acids 56-61 of SEQ ID NO: 12) which comprises about 0.198-0.298% dioxidated tryptophans,
   wherein H* is 2-oxo-histidine, W* is dioxidated tryptophan and wherein C* is carboxymethylated cysteine. In an embodiment of the invention, the peptides are deglycosylated e.g., with PNGase F.

Brown-Yellow Color

Brown-yellow color of polypeptide compositions set forth herein may be described in relation to the European Color Standards. See European Pharmacopoeia. Chapter 2.2.2. Degree of coloration of liquids. 8th ed. EP Color is used typically in the pharmaceutical industry to assign a color rating to liquid samples indicative, for example, of product quality. The European Pharmacopoeia Color is a visual liquid color scale used in the pharmaceutical industry. EP 2.2.2. Degree of Coloration of Liquids 2 outlines the preparation of 37 separate “Reference Solutions” that belong to the following five color families: greenish-yellow (GY), yellow (Y), brownish-yellow (BY), brown (B), and red (R). Of the 7 brown-yellow standards (BY standards), BY1 is the darkest standard and BY7 is the least dark. Matching a given sample to that of a BY color standard is routinely done in the art. The composition of European brown-yellow color standards are described in Table A, below.

TABLE A
Composition of European Brown-Yellow Color Standards

Volumes in millilitres

| Reference | Standard | Hydrochloric acid |
|---|---|---|
| solution | solution BY | (10 g/l HCl) |
| BY₁ | 100.0 | 0.0 |
| BY₂ | 75.0 | 25.0 |
| BY₃ | 50.0 | 50.0 |
| BY₄ | 25.0 | 75.0 |
| BY₅ | 12.5 | 87.5 |
| BY₆ | 5.0 | 95.0 |
| BY₇ | 2.5 | 97.5 |

Brownish-Yellow Standard Solution (BY): 10.8 g/L FeCl₃•6H₂O, 6.0 g/L CoCl₂•6H₂O and 2.5 g/L CuSO₄•5H₂O

The test for color of liquids is carried out by comparing a test solution with a standard color solution. The composition of the standard color solution is selected depending on the hue and intensity of the color of the test solution. Typically, comparison is carried out in flat-bottomed tubes of colorless, transparent, neutral glass that are matched as closely as possible in internal diameter and in all other respects (e.g., tubes of about 12, 15, 16 or 25 mm diameter). For example, a comparison can be between 2 or 10 mL of the test solution and standard color solution. The depth of liquids, for example, can be about 15, 25, 40 or 50 mm. The color assigned to the test solution should not be more intense than that of the standard color. Color comparisons are typically carried out in diffused light (e.g., daylight) against a white background. Colors can be compared down the vertical axis or horizontal axis of the tubes. In an embodiment of the invention, color of a composition comprising a VEGF mini-trap (e.g., REGN7483^{F}) is performed as described above.

The color of the BY standards can also be expressed under the CIEL*a*b* color space (“CIELAB” or “CIELab” color space). See Table B. In the CIE L*a*b* coordinate system, L* represents the degree of lightness of a color on a scale of 0-100, with 0 being the darkest and 100 the lightest, a* represents the redness or greenness of a color (positive values of a* represent red, whereas negative values of a* represent green), and b* represents the yellowness or blueness of a sample, with positive values of b* representing yellow and negative values of b* representing blue. Color difference from a standard, or from an initial sample in an evaluation, can be represented by a change in the individual color components ΔL*, Δa*, and Δb*. The composite change, or difference in color, can be calculated as a simple Euclidian distance in space using the formula: dE*=\sqrt{(ΔL*)²+(Δa*)²+(Δb*)²}. CIEL*a*b* color coordinates can be generated, for example, using the Hunter Labs UltrascanPro (Hunter Associates Laboratory, Reston, Va.) or on the BYK Gardner LCS IV (BYK-Gardner, Columbia, Md.). For the Hunter Labs UltraScan Pro, the Didymium Filter Test can be executed for wavelength calibration. The instrument can be standardized in TTRAN with the 0.780-inch port insert and DIW before use; thus, establishing the top (L*=100) and bottom (L*=0) of the photometric scale using a light trap and black card. See Pack et al., Modernization of Physical Appearance and Solution Color Tests Using Quantitative Tristimulus Colorimetry: Advantages, Harmonization, and Validation Strategies, J. Pharmaceutical Sci. 104: 3299-3313 (2015).

TABLE B
Characterization of European Brown-Yellow Color
Standards in the CIEL*a*b* Color Space

| Std. | L*\hat{ } | a*\hat{ } | b*\hat{ } | L*^{~} | a*^{~} | b*^{~} |
|---|---|---|---|---|---|---|
| BY1 | 93.95 | −2.76 | 28.55 | 92.84 | −3.16 | 31.15 |
| BY2 | 94.76 | −2.96 | 22.69 | 94.25 | −3.77 | 26.28 |
| BY3 | 96.47 | −2.84 | 16.41 | 95.92 | −3.44 | 18.52 |
| BY4 | 97.17 | −1.94 | 9.07 | 97.67 | −2.63 | 10.70 |
| BY5 | 98.91 | −1.19 | 4.73 | 98.75 | −1.61 | 5.77 |
| BY6 | 99.47 | −0.59 | 2.09 | 99.47 | −0.71 | 2.38 |
| BY7 | 99.37 | −0.31 | 1.13 | 99.71 | −0.37 | 1.17 |

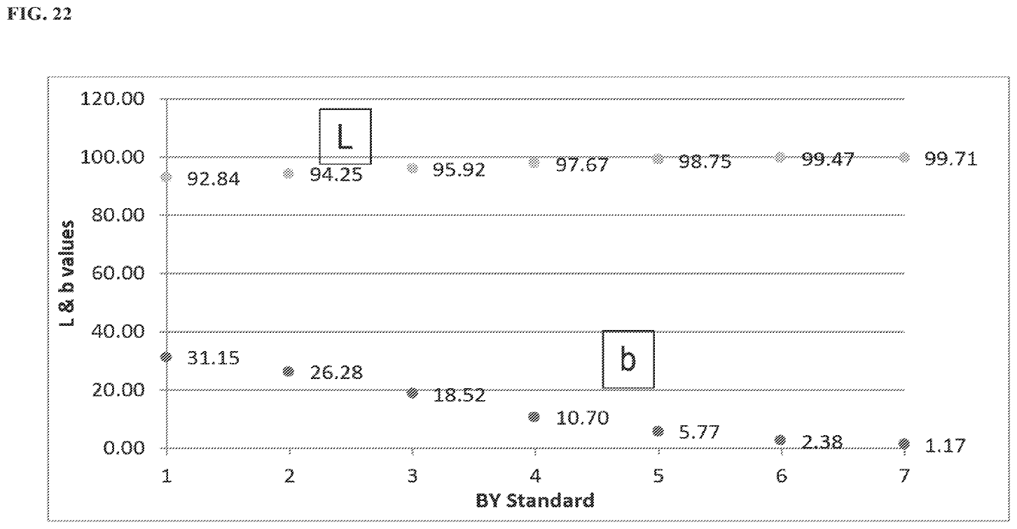

\hat{ }Reported by Pack et al.
^{~}Measured experimentally herein-the L* and b* values, for each BY color standard, in the CIEL*a*b* color space are set forth in FIG. 22.

The present invention provides compositions (e.g., aqueous compositions) comprising a VEGF mini-trap of the present invention (e.g., REGN7483^{F}, REGN7483^{R}, REGN7850 or REGN7851) (e.g., that was expressed in CDM, e.g., in a host cell such as a CHO cell) characterized by having a brown-yellow color which approximates that of BY2, BY3, BY4, BY5, BY6, BY7; or is no darker than BY2, no darker than BY3, no darker than BY4, no darker than BY5, no darker than BY6, no darker than BY7; or is between that of BY2 and BY3, between that of BY2 and BY4, between that of BY3 and BY4; between that of BY3 and BY5; between that of BY4 and BY5; between that of BY4 and BY6; between that of BY5 and BY6; between that of BY5 and BY7; or between that of BY6 and BY7.

The present invention also provides compositions (e.g., aqueous compositions) comprising a VEGF mini-trap of the present invention (e.g., REGN7483^{F}, REGN7483^{R}, REGN7850 or REGN7851) (e.g., that was expressed in CDM, e.g., in a host cell such as a CHO cell) characterized by a color in the CIEL*a*b* color space as follows:
L*=about 88.61, a*=about 0.53, b*=about 31.17; for example, wherein the mini-trap concentration is about 169 mg/ml;
L*=about 89, a*=about 0.5, b*=about 31; for example, wherein the mini-trap concentration is about 170 mg/ml;
L*=about 95.01, a*=about −1.68, b*=about 18.16; for example, wherein the mini-trap concentration is about 161 mg/ml;
L*=about 95, a*=about −1.5, b*=about 18; for example, wherein the mini-trap concentration is about 160 mg/ml;
L*=about 96.1, a*=about −1.05, b*=about 14.34; for example, wherein the mini-trap concentration is about 158 mg/ml;
L*=about 96, a*=about −1, b*=about 14 for example, wherein the mini-trap concentration is about 160 mg/ml;
L*=about 97.18, a*=about −0.93, b*=about 10.31; for example, wherein the mini-trap concentration is about 106 mg/ml;
L*=about 97, a*=about −1, b*=about 10 for example, wherein the mini-trap concentration is about 110 mg/ml;
L*=about 96.06, a*=about −1.02, b*=about 14.48; for example, wherein the mini-trap concentration is about 154 mg/ml;
L*=about 96, a*=about −1, b*=about 14.5; for example, wherein the mini-trap concentration is about 150 mg/ml;
L*=about 96.96, a*=about −0.85, b*=about 14.89; for example, wherein the mini-trap concentration is about 159 mg/ml;
L*=about 97, a*=about −1, b*=about 15; for example, wherein the mini-trap concentration is about 160 mg/ml;
L*=about 97.76, a*=about −1.02, b*=about 12.16; for example, wherein the mini-trap concentration is about 128 mg/ml;
L*=about 98, a*=about −1, b*=about 12; for example, wherein the mini-trap concentration is about 130 mg/ml;
L*=about 95.06, a*=about −1.07, b*=about 20.87; for example, wherein the mini-trap concentration is about 205 mg/ml;
L*=about 95, a*=about −1, b*=about 21; for example, wherein the mini-trap concentration is about 205 mg/ml;
L*=about 96.93, a*=about −1.55, b*=about 14.02; for example, wherein the mini-trap concentration is about 158 mg/ml;
L*=about 97, a*=about −1.5, b*=about 14 for example, wherein the mini-trap concentration is about 160 mg/ml;
L*=about 97.36, a*=about −0.39, b*=about 10.64; for example, wherein the mini-trap concentration is about 150 mg/ml;
L*=about 97, a*=about −0.5, b*=about 11; for example, wherein the mini-trap concentration is about 150 mg/ml;
L*=about 99.16, a*=about −0.35, b*=about 3.41; for example, wherein the mini-trap concentration is about 144 mg/ml;
L*=about 99, a*=about −0.5, b*=about 3; for example, wherein the mini-trap concentration is about 145 mg/ml;
L*=about 99.33, a*=about −0.19, b*=about 2.39 for example, wherein the mini-trap concentration is about 79.3 mg/ml;
L*=about 99, a*=about 0, b*=about 2.4 for example, wherein the mini-trap concentration is about 79 mg/ml;
L*=about 97.37, a*=about −1.12, b*=about 9.58; for example, wherein the mini-trap concentration is about 80 mg/ml;
L*=about 97, a*=about −1, b*=about 9.6; for example, wherein the mini-trap concentration is about 80 mg/ml;
L*=about 97.1, a*=about −0.85, b*=about 9.97 for example, wherein the mini-trap concentration is about 154 mg/ml;
L*=about 97, a*=about −1, b*=about 10; for example, wherein the mini-trap concentration is about 150 mg/ml;
L*=about 98.04, a*=about −0.67, b*=about 6.75; for example, wherein the mini-trap concentration is about 100 mg/ml;
L*=about 98, a*=about −1, b*=about 6.8; for example, wherein the mini-trap concentration is about 100 mg/ml;
L*=about 98.5, a*=about −0.51, b*=about 5.03; for example, wherein the mini-trap concentration is about 75 mg/ml;
L*=about 99, a*=about −0.5, b*=about 5; for example, wherein the mini-trap concentration is about 75 mg/ml;
L*=about 98.94, a*=about −0.36, b*=about 3.58; for example, wherein the mini-trap concentration is about 50 mg/ml;
L*=about 99, a*=about −0.5, b*=about 3.6; for example, wherein the mini-trap concentration is about 50 mg/ml;
L*=about 99.47, a*=about −0.13, b*=about 1.65; for example, wherein the mini-trap concentration is about 25 mg/ml;
L*=about 99.5, a*=about 0, b*=about 1.7; for example, wherein the mini-trap concentration is about 25 mg/ml;
L*=about 99.77, a*=about −0.02, b*=about 0.66; for example, wherein the mini-trap concentration is about 10 mg/ml;
L*=about 100, a*=about 0, b*=about 0.7; for example, wherein the mini-trap concentration is about 10 mg/ml;
L*=about 99.9, a*=about 0.01, b*=about 0.36; for example, wherein the mini-trap concentration is about 5 mg/ml;
L*=about 100, a*=about 0, b*=about 0.4; for example, wherein the mini-trap concentration is about 5 mg/ml;
L*=about 99.95, a*=about 0.06, b*=about 0.08; for example, wherein the mini-trap concentration is about 3 mg/ml;
L*=about 100, a*=about 0.1, b*=about 0.1; for example, wherein the mini-trap concentration is about 3 mg/ml;
L*=about 98.89, a*=about 0.01, b*=about 1.05; for example, wherein the mini-trap concentration is about 10 mg/ml;
L*=about 99, a*=about 0, b*=about 1.1; for example, wherein the mini-trap concentration is about 10 mg/ml;
L*=about 98.3, a*=about −0.03, b*=about 0.96; for example, wherein the mini-trap concentration is about 10 mg/ml;
L*=about 98, a*=about 0, b*=about 1; for example, wherein the mini-trap concentration is about 10 mg/ml;
L*=about 99.07, a*=about −0.07, b*=about 1.33; for example, wherein the mini-trap concentration is about 10 mg/ml;
L*=about 99, a*=about 0, b*=about 1.3; for example, wherein the mini-trap concentration is about 10 mg/ml;
L*=about 99.42, a*=about −0.04, b*=about 1.35; for example, wherein the mini-trap concentration is about 10 mg/ml;
L*=about 99, a*=about 0, b*=about 1.4; for example, wherein the mini-trap concentration is about 10 mg/ml;
L*=about 99.19, a*=about −0.09, b*=about 1.55; for example, wherein the mini-trap concentration is about 10 mg/ml;
L*=about 99, a*=about 0, b*=about 1.6; for example, wherein the mini-trap concentration is about 10 mg/ml;
L*=about 94-100, a*=−3-1 or −3-0 and b*=no more than about 23;
L*=about 94-100, a*=−3-1 or −3-0 and b*=no more than about 22;
L*=about 94-100, a*=−3-1 or −3-0 and b*=no more than about 21;
L*=about 94-100, a*=−3-1 or −3-0 and b*=no more than about 20;
L*=about 94-100, a*=−3-1 or −3-0 and b*=no more than about 19;
L*=about 94-100, a*=−3-1 or −3-0 and b*=no more than about 18;
L*=about 94-100, a*=−3-1 or −3-0 and b*=no more than about 17;
L*=about 94-100, a*=−3-1 or −3-0 and b*=no more than about 16;
L*=about 94-100, a*=−3-1 or −3-0 and b*=no more than about 15;
L*=about 94-100, a*=−3-1 or −3-0 and b*=no more than about 14;
L*=about 94-100, a*=−3-1 or −3-0 and b*=no more than about 13;
L*=about 94-100, a*=−3-1 or −3-0 and b*=no more than about 12;
L*=about 94-100, a*=−3-1 or −3-0 and b*=no more than about 11;
L*=about 94-100, a*=−3-1 or −3-0 and b*=no more than about 10;
L*=about 94-100, a*=−3-1 or −3-0 and b*=no more than about 9;
L*=about 94-100, a*=−3-1 or −3-0 and b*=no more than about 8;
L*=about 94-100, a*=−3-1 or −3-0 and b*=no more than about 7;
L*=about 94-100, a*=−3-1 or −3-0 and b*=no more than about 6;
L*=about 94-100, a*=−3-1 or −3-0 and b*=no more than about 5;
L*=about 94-100, a*=−3-1 or −3-0 and b*=no more than about 4;
L*=about 94-100, a*=−3-1 or −3-0 and b*=no more than about 3;
L*=about 94-100, a*=−3-1 or −3-0 and b*=no more than about 2;
L*=about 94-100, a*=−3-1 or −3-0 and b*=no more than about 1;
L*=about 94-100, a*=−3-1 or −3-0 and b*=about 23;
L*=about 94-100, a*=−3-1 or −3-0 and b*=about 22;
L*=about 94-100, a*=−3-1 or −3-0 and b*=about 21;
L*=about 94-100, a*=−3-1 or −3-0 and b*=about 20;
L*=about 94-100, a*=−3-1 or −3-0 and b*=about 19;
L*=about 94-100, a*=−3-1 or −3-0 and b*=about 18;
L*=about 94-100, a*=−3-1 or −3-0 and b*=about 17;
L*=about 94-100, a*=−3-1 or −3-0 and b*=about 16;
L*=about 94-100, a*=−3-1 or −3-0 and b*=about 15;
L*=about 94-100, a*=−3-1 or −3-0 and b*=about 14;
L*=about 94-100, a*=−3-1 or −3-0 and b*=about 13;
L*=about 94-100, a*=−3-1 or −3-0 and b*=about 12;
L*=about 94-100, a*=−3-1 or −3-0 and b*=about 11;
L*=about 94-100, a*=−3-1 or −3-0 and b*=about 10;
L*=about 94-100, a*=−3-1 or −3-0 and b*=about 9;
L*=about 94-100, a*=−3-1 or −3-0 and b*=about 8;
L*=about 94-100, a*=−3-1 or −3-0 and b*=about 7;
L*=about 94-100, a*=−3-1 or −3-0 and b*=about 6;
L*=about 94-100, a*=−3-1 or −3-0 and b*=about 5;
L*=about 94-100, a*=−3-1 or −3-0 and b*=about 4;
L*=about 94-100, a*=−3-1 or −3-0 and b*=about 3;
L*=about 94-100, a*=−3-1 or −3-0 and b*=about 2;
L*=about 94-100, a*=−3-1 or −3-0 and b*=about 1;
L*=about 94-100, a*=−3-1 or −3-0 and b*=about 3-5;
L*=about 94-100, a*=−3-1 or −3-0 and b*=about 4-6;
L*=about 94-100, a*=−3-1 or −3-0 and b*=about 5-7;
L*=about 94-100, a*=−3-1 or −3-0 and b*=about 6-8;
L*=about 94-100, a*=−3-1 or −3-0 and b*=about 7-9;
L*=about 94-100, a*=−3-1 or −3-0 and b*=about 8-10;
L*=about 94-100, a*=−3-1 or −3-0 and b*=about 9-11;
L*=about 94-100, a*=−3-1 or −3-0 and b*=about 10-12;
L*=about 94-100, a*=−3-1 or −3-0 and b*=about 11-13;
L*=about 94-100, a*=−3-1 or −3-0 and b*=about 14-16;
L*=about 94-100, a*=−3-1 or −3-0 and b*=about 15-17;
L*=about 94-100, a*=−3-1 or −3-0 and b*=about 16-18;
L*=about 94-100, a*=−3-1 or −3-0 and b*=about 17-19;
L*=about 94-100, a*=−3-1 or −3-0 and b*=about 18-20;
L*=about 94-100, a*=−3-1 or −3-0 and b*=about 19-21;
L*=about 94-100, a*=−3-1 or −3-0 and b*=about 20-22;
L*=about 94-100, a*=−3-1 or −3-0 and b*=about 21-23;
L*=about 94-100, a*=−3-1 or −3-0 and b*=about 17-23;
L*=about 94-100, a*=−3-1 or −3-0 and b*=about 10-23;
L*=about 94-100, a*=−3-1 or −3-0 and b*=about 5-23;
L*=about 94-100, a*=−3-1 or −3-0 and b*=about 3-23;
L*=about 94-100, a*=−3-1 or −3-0 and b*=about 1-23;
L*=about 94-100, a*=−3-1 or −3-0 and b*=about 10-17;
L*=about 94-100, a*=−3-1 or −3-0 and b*=about 5-17;
L*=70-99, a*=−2-0 and b*=20 or less; and/or
L*=70-99, a*=−2-0 and b*=10-31, about 10, about 14, about 12, about 14, about 15, about 18, about 21, about 27 or about 31. In an embodiment of the invention, a composition comprising a VEGF mini-trap having such a color profile as described above has a concentration of mini-trap of about 70 mg/ml or more, 75-200 mg/ml; or 70-205 mg/ml; 10 mg/ml; 11 mg/ml; 12 mg/ml; 13 mg/ml; 14 mg/ml; 15 mg/ml; 16 mg/ml; 17 mg/ml; 18 mg/ml; 19 mg/ml; 20 mg/ml; 21 mg/ml; 22 mg/ml; 23 mg/ml; 24 mg/ml; 25 mg/ml; 26 mg/ml; 27 mg/ml; 28 mg/ml; 29 mg/ml; 30 mg/ml; 31 mg/ml; 32 mg/ml; 33 mg/ml; 34 mg/ml; 35 mg/ml; 36 mg/ml; 37 mg/ml; 38 mg/ml; 39 mg/ml; 40 mg/ml; 41 mg/ml; 42 mg/ml; 43 mg/ml; 44 mg/ml; 45 mg/ml; 46 mg/ml; 47 mg/ml; 48 mg/ml; 49 mg/ml; 50 mg/ml; 51 mg/ml; 52 mg/ml; 53 mg/ml; 54 mg/ml; 55 mg/ml; 56 mg/ml; 57 mg/ml; 58 mg/ml; 59 mg/ml; 60 mg/ml; 61 mg/ml; 62 mg/ml; 63 mg/ml; 64 mg/ml; 65 mg/ml; 66 mg/ml; 67 mg/ml; 68 mg/ml; 69 mg/ml; 70 mg/ml; 71 mg/ml; 72 mg/ml; 73 mg/ml; 74 mg/ml; 75 mg/ml; 76 mg/ml; 77 mg/ml; 78 mg/ml; 79 mg/ml; 80 mg/ml; 81 mg/ml; 82 mg/ml; 83 mg/ml; 84 mg/ml; 85 mg/ml; 86 mg/ml; 87 mg/ml; 88 mg/ml; 89 mg/ml; 90 mg/ml; 91 mg/ml; 92 mg/ml; 93 mg/ml; 94 mg/ml; 95 mg/ml; 96 mg/ml; 97 mg/ml; 98 mg/ml; 99 mg/ml; 100 mg/ml; 101 mg/ml; 102 mg/ml; 103 mg/ml; 104 mg/ml; 105 mg/ml; 106 mg/ml; 107 mg/ml; 108 mg/ml; 109 mg/ml; 110 mg/ml; 111 mg/ml; 112 mg/ml; 113 mg/ml; 114 mg/ml; 115 mg/ml; 116 mg/ml; 117 mg/ml; 118 mg/ml; 119 mg/ml; 120 mg/ml; 121 mg/ml; 122 mg/ml; 123 mg/ml; 124 mg/ml; 125 mg/ml; 126 mg/ml; 127 mg/ml; 128 mg/ml; 129 mg/ml; 130 mg/ml; 131 mg/ml; 132 mg/ml; 133 mg/ml; 134 mg/ml; 135 mg/ml; 136 mg/ml; 137 mg/ml; 138 mg/ml; 139 mg/ml; 140 mg/ml; 141 mg/ml; 142 mg/ml; 143 mg/ml; 144 mg/ml; 145 mg/ml; 146 mg/ml; 147 mg/ml; 148 mg/ml; 149 mg/ml; 150 mg/ml; 151 mg/ml; 152 mg/ml; 153 mg/ml; 154 mg/ml; 155 mg/ml; 156 mg/ml; 157 mg/ml; 158 mg/ml; 159 mg/ml; 160 mg/ml; 161 mg/ml; 162 mg/ml; 163 mg/ml; 164 mg/ml; 165 mg/ml; 166 mg/ml; 167 mg/ml; 168 mg/ml; 169 mg/ml; 170 mg/ml; 171 mg/ml; 172 mg/ml; 173 mg/ml; 174 mg/ml; 175 mg/ml; 176 mg/ml; 177 mg/ml; 178 mg/ml; 179 mg/ml; 180 mg/ml; 181 mg/ml; 182 mg/ml; 183 mg/ml; 184 mg/ml; 185 mg/ml; 186 mg/ml; 187 mg/ml; 188 mg/ml; 189 mg/ml; 190 mg/ml; 191 mg/ml; 192 mg/ml; 193 mg/ml; 194 mg/ml; 195 mg/ml; 196 mg/ml; 197 mg/ml; 198 mg/ml; 199 mg/ml; 200 mg/ml; 201 mg/ml; 202 mg/ml; 203 mg/ml; 204 mg/ml; or 205 mg/ml.

Alternatively, in an embodiment of the invention, a composition has a VEGF mini-trap concentration of about 70 or more, about 75, about 90, about 106, about 128, about 147, about 154, 158, about 159, about 161, about 169, about 200, about 205, about 75-200 or about 70-205 g/l, but has such a color profile as described above when diluted, for example, to about 10 mg/ml; 11 mg/ml; 12 mg/ml; 13 mg/ml; 14 mg/ml; 15 mg/ml; 16 mg/ml; 17 mg/ml; 18 mg/ml; 19 mg/ml; 20 mg/ml; 21 mg/ml; 22 mg/ml; 23 mg/ml; 24 mg/ml; 25 mg/ml; 26 mg/ml; 27 mg/ml; 28 mg/ml; 29 mg/ml; 30 mg/ml; 31 mg/ml; 32 mg/ml; 33 mg/ml; 34 mg/ml; 35 mg/ml; 36 mg/ml; 37 mg/ml; 38 mg/ml; 39 mg/ml; 40 mg/ml; 41 mg/ml; 42 mg/ml; 43 mg/ml; 44 mg/ml; 45 mg/ml; 46 mg/ml; 47 mg/ml; 48 mg/ml; 49 mg/ml; 50 mg/ml; 51 mg/ml; 52 mg/ml; 53 mg/ml; 54 mg/ml; 55 mg/ml; 56 mg/ml; 57 mg/ml; 58 mg/ml; 59 mg/ml; 60 mg/ml; 61 mg/ml; 62 mg/ml; 63 mg/ml; 64 mg/ml; 65 mg/ml; 66 mg/ml; 67 mg/ml; 68 mg/ml; 69 mg/ml; 70 mg/ml; 71 mg/ml; 72 mg/ml; 73 mg/ml; 74 mg/ml; 75 mg/ml; 76 mg/ml; 77 mg/ml; 78 mg/ml; 79 mg/ml; 80 mg/ml; 81 mg/ml; 82 mg/ml; 83 mg/ml; 84 mg/ml; 85 mg/ml; 86 mg/ml; 87 mg/ml; 88 mg/ml; 89 mg/ml; 90 mg/ml; 91 mg/ml; 92 mg/ml; 93 mg/ml; 94 mg/ml; 95 mg/ml; 96 mg/ml; 97 mg/ml; 98 mg/ml; 99 mg/ml; 100 mg/ml; 101 mg/ml; 102 mg/ml; 103 mg/ml; 104 mg/ml; 105 mg/ml; 106 mg/ml; 107 mg/ml; 108 mg/ml; 109 mg/ml; 110 mg/ml; 111 mg/ml; 112 mg/ml; 113 mg/ml; 114 mg/ml; 115 mg/ml; 116 mg/ml; 117 mg/ml; 118 mg/ml; 119 mg/ml; 120 mg/ml; 121 mg/ml; 122 mg/ml; 123 mg/ml; 124 mg/ml; 125 mg/ml; 126 mg/ml; 127 mg/ml; 128 mg/ml; 129 mg/ml; 130 mg/ml; 131 mg/ml; 132 mg/ml; 133 mg/ml; 134 mg/ml; 135 mg/ml; 136 mg/ml; 137 mg/ml; 138 mg/ml; 139 mg/ml; 140 mg/ml; 141 mg/ml; 142 mg/ml; 143 mg/ml; 144 mg/ml; 145 mg/ml; 146 mg/ml; 147 mg/ml; 148 mg/ml; 149 mg/ml; 150 mg/ml; 151 mg/ml; 152 mg/ml; 153 mg/ml; 154 mg/ml; 155 mg/ml; 156 mg/ml; 157 mg/ml; 158 mg/ml; 159 mg/ml; 160 mg/ml; 161 mg/ml; 162 mg/ml; 163 mg/ml; 164 mg/ml; 165 mg/ml; 166 mg/ml; 167 mg/ml; 168 mg/ml; 169 mg/ml; 170 mg/ml; 171 mg/ml; 172 mg/ml; 173 mg/ml; 174 mg/ml; 175 mg/ml; 176 mg/ml; 177 mg/ml; 178 mg/ml; 179 mg/ml; or 180 mg/ml.

In an embodiment of the invention, a composition comprising a VEGF mini-trap (e.g., REGN7483^{F}) that has been expressed in a host cell (e.g., a CHO cell), for example, in CDM, includes no more than about 50 parts per million (ppm) host cell protein.

The color of compositions may, in an embodiment of the invention, be correlated with VEGF mini-trap (e.g., that was expressed in CDM) concentration in a composition (e.g., an aqueous composition) wherein the correlation is expressed by the following equation:

0.046+(0.066×concentration of mini-tap (mg/ml))=b*,

e.g., wherein L*=about 97-99 and a=about −0.085-0.06. In an embodiment of the invention, the equation is:

b*=(0.11×concentration of mini-trap (mg/mi)−0.56).

In an embodiment of the invention, the concentration of the VEGF mini-trap in a composition or pharmaceutical formulation of the present invention is about 90, 100, 110 or 120 mg/ml (or any of the concentrations described above) and is characterized by a color, in the CIEL*a*b* color space, according to such an equation.

The color of a composition including the VEGF mini-trap (e.g., that was expressed in CDM) is also correlated with pH and conductivity under which the AEX chromatographic purification (flow-through mode) is performed. In an embodiment of the invention, the composition is the product of a process including AEX chromatographic purification under a pH which is about 8.0 or more or 8.4 or more and the conductivity is about 2.0 mS/cm or lower or 4 mS/cm or lower. Thus, in an embodiment of the invention, the AEX chromatography condition is a pH of higher than about 8.1 or 8.4 (e.g., about 8.1-8.4) and/or a conductivity lower than about 6.5 (e.g., about 2.0, 4.0 or 2-4 mS/cm). In an embodiment of the invention, the composition is the flow-through fraction from the AEX column and has said pH (e.g., 8.4) and conductivity (e.g., 2.0 mS/cm). In an embodiment of the invention, composition is the product of a process which includes the AEX chromatographic purification and further comprises the adjustment of the composition to a lower pH, e.g., to about 6.0 (e.g., 5.5, 5.6, 5.7, 5.8, 5.9, 6.0, 6.1, 6.2).

Thus, the present invention includes compositions (e.g., aqueous compositions) comprising VEGF mini-traps (e.g., REGN7483^{F}, REGN7483^{R}, REGN7850 or REGN7851) which have been expressed in a chemically-defined medium wherein about 0.1%-1% of all histidines in the composition are modified to 2-oxo-histidine and wherein the color of the composition is as discussed herein, e.g., no darker than, for example, the European Brown-Yellow Color Standard BY2, BY3 or BY4 and/or having a color characterized in the CIEL*a*b* color space as L*=94-100, a*=-3-0 and b*=about 3-6; e.g., having a concentration of about 90, 100, 110 or 120 mg/ml; or any of the concentrations discussed above.

Acidic and Basic Species

Protein variants can include both acidic species and basic species. Acidic species are variants that elute earlier than the main peak from CEX or later than the main peak from AEX, while basic species are the variants that elute later than the main peak from CEX or earlier than the main peak from AEX.

The terms “acidic species,” “AS,” “acidic region,” and “AR,” refer to the variants of a protein which are characterized by an overall acidic charge. For example, in recombinant protein preparations, such acidic species can be detected by various methods, such as ion exchange, for example, WCX-10 HPLC (a weak cation exchange chromatography), or IEF (isoelectric focusing). Acidic species of a VEGF mini-trap may include variants, structure variants, and/or fragmentation variants. Exemplary variants can include, but are not limited to, deamidation variants, afucosylation variants, oxidation variants, methylglyoxal (MGO) variants, glycation variants, and citric acid variants. Exemplary structure variants include, but are not limited to, glycosylation variants and acetonation variants. Exemplary fragmentation variants include any modified protein species from the target molecule due to dissociation of peptide chain, enzymatic and/or chemical modifications, including, but not limited to, Fc and Fab fragments, fragments missing a Fab, fragments missing a heavy chain variable domain, C-terminal truncation variants, variants with excision of N-terminal Asp in the light chain, and variants having N-terminal truncation of the light chain. Other acidic species variants include variants containing unpaired disulfides, host cell proteins, and host nucleic acids, chromatographic materials, and media components. Commonly, acidic species elute earlier than the main peak during CEX or later than the main peak during AEX analysis.

In an embodiment of the invention, a protein composition can comprise more than one type of acidic species variant. For example, but not by way of limitation, the total acidic species can be divided based on chromatographic retention time of the peaks appearing. Another example in which the total acidic species can be divided can be based on the type of variant—variants, structure variants, or fragmentation variant.

The term “acidic species” or “AS” does not refer to process-related impurities. The term “process-related impurity,” as used herein, refers to impurities that are present in a composition comprising a protein, but are not derived from the protein itself. Process-related impurities include, but are not limited to, host cell proteins (HCPs), host cell nucleic acids, chromatographic materials, and media components.

In some exemplary embodiments of the invention, a composition of the present invention can comprise a VEGF mini-trap and acidic species of the VEGF mini-trap, wherein the amount of the acidic species in the composition compared to the VEGF mini-trap can be at most about 15%, 14%, 13%, 12%, 11%, 10%, 9%, 8%, 7%, 6%, 5%, 4.5%, 4%, 3.5%, 3%, 2.5%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1%, or 0.0% and ranges within one or more of the preceding.

In an exemplary embodiment of the invention, the composition can comprise VEGF mini-trap and acidic species of the VEGF mini-trap, wherein the amount of the acidic species in the composition compared to the VEGF mini-trap can be about 0% to about 15% e.g., about 0% to about 15%, about 0.05% to about 15%, about 0.1% to about 15%, about 0.2% to about 15%, about 0.3% to about 15%, about 0.4% to about 15%, about 0.5% to about 15%, about 0.6% to about 15%, about 0.7% to about 15%, about 0.8% to about 15%, about 0.9% to about 15%, about 1% to about 15%, about 1.5% to about 15%, about 2% to about 15%, about 3% to about 15%, about 4% to about 15%, about 5% to about 15%, about 6% to about 15%, about 7% to about 15%, about 8% to about 15%, about 9% to about 15%, about 10% to about 15%, about 0% to about 10%, about 0.05% to about 10%, about 0.1% to about 10%, about 0.2% to about 10%, about 0.3% to about 10%, about 0.4% to about 10%, about 0.5% to about 10%, about 0.6% to about 10%, about 0.7% to about 10%, about 0.8% to about 10%, about 0.9% to about 10%, about 1% to about 10%, about 1.5% to about 10%, about 2% to about 10%, about 3% to about 10%, about 4% to about 10%, about 5% to about 10%, about 6% to about 10%, about 7% to about 10%, about 8% to about 10%, about 9% to about 10%, about 0% to about 7.5%, about 0.05% to about 7.5%, about 0.1% to about 7.5%, about 0.2% to about 7.5%, about 0.3% to about 7.5%, about 0.4% to about 7.5%, about 0.5% to about 7.5%, about 0.6% to about 7.5%, about 0.7% to about 7.5%, about 0.8% to about 7.5%, about 0.9% to about 7.5%, about 1% to about 7.5%, about 1.5% to about 7.5%, about 2% to about 7.5%, about 3% to about 7.5%, about 4% to about 7.5%, about 5% to about 7.5%, about 6% to about 7.5%, about 7% to about 7.5%, about 0% to about 5%, about 0.05% to about 5%, about 0.1% to about 5%, about 0.2% to about 5%, about 0.3% to about 5%, about 0.4% to about 5%, about 0.5% to about 5%, about 0.6% to about 5%, about 0.7% to about 5%, about 0.8% to about 5%, about 0.9% to about 5%, about 1% to about 5%, about 1.5% to about 5%, about 2% to about 5%, about 3% to about 5%, about 4% to about 5% and ranges within one or more of the preceding.

All peaks eluting prior to the protein of interest can be summed as the acidic region, and all peaks eluting after the protein of interest can be summed as the basic region. In some exemplary embodiments, the acidic species can be eluted as two or more acidic regions and can be numbered AR1, AR2, AR3 and so on based on a certain retention time of the peaks and on the ion exchange column used.

In one exemplary embodiment of the invention, a composition can comprise a VEGF mini-trap and acidic species of the VEGF mini-trap, wherein AR1 compared to region of the VEGF mini-trap is 15%, 14%, 13%, 12%, 11%, 10%, 9%, 8%, 7%, 6%, 5%, 4.5%, 4%, 3.5%, 3%, 2.5%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1%, or 0.0%, and ranges within one or more of the preceding. In another exemplary embodiment, the composition can comprise VEGF mini-trap and acidic species of the VEGF mini-trap, wherein AR1 compared to region of the anti-VEGF protein is about 0.0% to about 10%, about 0.0% to about 5%, about 0.0% to about 4%, about 0.0% to about 3%, about 0.0% to about 2%, about 3% to about 5%, about 5% to about 8%, or about 8% to about 10%, or about 10% to about 15%, and ranges within one or more of the preceding.

In one exemplary embodiment of the invention, the composition can comprise VEGF mini-trap and acidic species of the VEGF mini-trap, wherein AR2 compared to region of the anti-VEGF protein is 15%, 14%, 13%, 12%, 11%, 10%, 9%, 8%, 7%, 6%, 5%, 4.5%, 4%, 3.5%, 3%, 2.5%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1%, or 0.0%, and ranges within one or more of the preceding. In another exemplary embodiment, the composition can comprise VEGF mini-trap and acidic species of the VEGF mini-trap, wherein AR2 compared to region of the VEGF mini-trap is about 0.0% to about 10%, about 0.0% to about 5%, about 0.0% to about 4%, about 0.0% to about 3%, about 0.0% to about 2%, about 3% to about 5%, about 5% to about 8%, or about 8% to about 10%, or about 10% to about 15%, and ranges within one or more of the preceding.

Among the chemical degradation pathways responsible for acidic or basic species, the two most commonly observed covalent modifications in proteins and peptides are deamination and oxidation. Methionine, cysteine, histidine, tryptophan, and tyrosine are of the amino acids that are most susceptible to oxidation: Met and Cys because of their sulfur atoms and His, Trp, and Tyr because of their aromatic rings.

The terms “basic species,” “basic region,” and “BR,” refer to the variants of a protein which are characterized by an overall basic charge. For example, in recombinant protein preparations, such basic species can be detected by various methods, such as ion exchange, for example, WCX-10 HPLC (a weak cation exchange chromatography), or IEF (isoelectric focusing). Exemplary variants can include, but are not limited to, lysine Variants, isomerization of aspartic acid, succinimide formation at Asparagine, Methionine oxidation, amidation, incomplete disulfide bond formation, mutation from Serine to Arginine, aglycosylation, fragmentation and aggregation. Commonly, basic species elute later than the main peak during CEX or earlier than the main peak during AEX analysis. (Chromatographic analysis of the acidic and basic species of recombinant monoclonal antibodies. MAbs. 2012 Sep 1; 4(5): 578-585).

In certain exemplary embodiments of the invention, a protein composition can comprise more than one type of basic species variant. For example, but not by way of limitation, the total basic species can be divided based on chromatographic retention time of the peaks appearing. Another example in which the total basic species can be divided can be based on the type of variant—variants, structure variants, or fragmentation variant.

As illustrated for acidic species, the term “basic species” does not include process-related impurities and the basic species may be the result of product preparation (referred to herein as “preparation-derived basic species”), or the result of storage (referred to herein as “storage-derived basic species”).

In some exemplary embodiments of the invention, the composition can comprise VEGF mini-trap and basic species of the VEGF mini-trap, wherein the amount of the basic species in the composition compared to the VEGF mini-trap can be at most about 15%, 14%, 13%, 12%, 11%, 10%, 9%, 8%, 7%, 6%, 5%, 4.5%, 4%, 3.5%, 3%, 2.5%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1%, or 0.0% and ranges within one or more of the preceding.

In other exemplary embodiment of the invention, the composition can comprise VEGF mini-trap and basic species of the VEGF mini-trap, wherein the amount of the basic species in the composition compared to the VEGF mini-trap can be about 0% to about 15% e.g., about 0% to about 15%, about 0.05% to about 15%, about 0.1% to about 15%, about 0.2% to about 15%, about 0.3% to about 15%, about 0.4% to about 15%, about 0.5% to about 15%, about 0.6% to about 15%, about 0.7% to about 15%, about 0.8% to about 15%, about 0.9% to about 15%, about 1% to about 15%, about 1.5% to about 15%, about 2% to about 15%, about 3% to about 15%, about 4% to about 15%, about 5% to about 15%, about 6% to about 15%, about 7% to about 15%, about 8% to about 15%, about 9% to about 15%, about 10% to about 15%, about 0% to about 10%, about 0.05% to about 10%, about 0.1% to about 10%, about 0.2% to about 10%, about 0.3% to about 10%, about 0.4% to about 10%, about 0.5% to about 10%, about 0.6% to about 10%, about 0.7% to about 10%, about 0.8% to about 10%, about 0.9% to about 10%, about 1% to about 10%, about 1.5% to about 10%, about 2% to about 10%, about 3% to about 10%, about 4% to about 10%, about 5% to about 10%, about 6% to about 10%, about 7% to about 10%, about 8% to about 10%, about 9% to about 10%, about 0% to about 7.5%, about 0.05% to about 7.5%, about 0.1% to about 7.5%, about 0.2% to about 7.5%, about 0.3% to about 7.5%, about 0.4% to about 7.5%, about 0.5% to about 7.5%, about 0.6% to about 7.5%, about 0.7% to about 7.5%, about 0.8% to about 7.5%, about 0.9% to about 7.5%, about 1% to about 7.5%, about 1.5% to about 7.5%, about 2% to about 7.5%, about 3% to about 7.5%, about 4% to about 7.5%, about 5% to about 7.5%, about 6% to about 7.5%, about 7% to about 7.5%, about 0% to about 5%, about 0.05% to about 5%, about 0.1% to about 5%, about 0.2% to about 5%, about 0.3% to about 5%, about 0.4% to about 5%, about 0.5% to about 5%, about 0.6% to about 5%, about 0.7% to about 5%, about 0.8% to about 5%, about 0.9% to about 5%, about 1% to about 5%, about 1.5% to about 5%, about 2% to about 5%, about 3% to about 5%, about 4% to about 5% and ranges within one or more of the preceding.

In some exemplary embodiments of the invention, the basic species can be eluted as two or more basic regions and can be numbered BR1, BR2, BR3 and so on based on a certain retention time of the peaks and ion exchange used.

In one exemplary embodiment of the invention, the composition can comprise VEGF mini-trap and basic species of the VEGF mini-trap, wherein BR1 compared to region of the VEGF mini-trap is 15%, 14%, 13%, 12%, 11%, 10%, 9%, 8%, 7%, 6%, 5%, 4.5%, 4%, 3.5%, 3%, 2.5%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1%, or 0.0%, and ranges within one or more of the preceding. In another exemplary embodiment, the composition can comprise VEGF mini-trap and acidic species of the VEGF mini-trap, wherein BR1 compared to region of the anti-VEGF protein is about 0.0% to about 10%, about 0.0% to about 5%, about 0.0% to about 4%, about 0.0% to about 3%, about 0.0% to about 2%, about 3% to about 5%, about 5% to about 8%, or about 8% to about 10%, or about 10% to about 15%, and ranges within one or more of the preceding.

In one exemplary embodiment of the invention, the composition can comprise VEGF mini-trap and basic species of the VEGF mini-trap, wherein BR2 compared to region of the VEGF mini-trap is 15%, 14%, 13%, 12%, 11%, 10%, 9%, 8%, 7%, 6%, 5%, 4.5%, 4%, 3.5%, 3%, 2.5%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1%, or 0.0%, and ranges within one or more of the preceding. In another exemplary embodiment, the composition can comprise an VEGF mini-trap and acidic species of the VEGF mini-trap, wherein BR2 compared to region of the anti-VEGF protein is about 0.0% to about 10%, about 0.0% to about 5%, about 0.0% to about 4%, about 0.0% to about 3%, about 0.0% to about 2%, about 3% to about 5%, about 5% to about 8%, or about 8% to about 10%, or about 10% to about 15%, and ranges within one or more of the preceding.

The levels of protein variants and/or acidic species in the chromatographic samples produced using the techniques described herein may be analyzed as described in the Examples section. In certain embodiments, a clEF method is employed using an iCE3 analyzer (ProteinSimple) with a fluorocarbon coated capillary cartridge (100 μm×5 cm). The ampholyte solution consisted of a mixture of 0.35% methyl cellulose (MC), 4% Pharmalyte 3-10 carrier ampholytes, 4% Pharmalyte 5-8 carrier ampholytes, 10 mM L-Arginine HCl, 24% formamide, and pl markers 5.12 and 9.77 in purified water. The anolyte was 80 mM phosphoric acid, and the catholyte was 100 mM sodium hydroxide, both in 0.10% methylcellulose. Samples were diluted in purified water to 10 mg/mL. Samples were mixed with the ampholyte solution and then focused by introducing a potential of 1500 V for one minute, followed by a potential of 3000 V for 7 minutes. An image of the focused variants was obtained by passing 280 nm ultraviolet light through the capillary and into the lens of a charge coupled device digital camera. This image was then analyzed to determine the distribution of the various charge variants.

Anion-Exchange (AEX) Chromatography

In an embodiment of the invention, the VEGF mini-trap (e.g., in the AEX equilibration buffer) is loaded on the AEX resin which has been equilibrated with a buffer, for example, at pH 8.4 (e.g., in Tris buffer such as 50 mM Tris), e.g., 50 mM Tris pH 8.4, 2.0 mS/cm (millisiemens per centimeter) and the flow-through fraction is collected. In an embodiment of the invention, equilibration buffer is Tris hydrochloride at a pH of about 8.3 to about 8.6. For example, the flow-through can be collected along with the wash fraction from the column. A wash of the column can be performed, for example, with one or more column volumes (CV) of equilibration buffer (e.g., 2 CVs). In an embodiment of the invention, prior to AEX chromatography, aflibercept is cleaved with IdeS protease (e.g., from Streptococcus pyogenes, e.g., FabRICATOR) and protein-A chromatography is used to remove the cleaved Fc fragment from the mini-trap product. As discussed, the mini-trap is then purified by AEX chromatography (flow-through mode).

Thus, the present invention provides a composition comprising a VEGF mini-trap of the present invention (e.g., REGN7483^{F}) which is produced by a method comprising the steps of:
(i) expressing aflibercept in host cell (e.g., Chinese hamster ovary (CHO) cells) which is grown in a CDM (e.g., wherein the aflibercept is secreted from the host cell into the CDM);
(ii) removal of the aflibercept from the medium and/or host cells;
(iii) optionally, purifying the aflibercept by protein-A chromatography;
(iii) proteolytic digestion of the aflibercept with S. pyogenes IdeS protease (e.g., FabRICATOR) or a variant thereof to generate mini-trap and Fc fragment; optionally, the Fc is removed from the composition by protein-A chromatography wherein the Fc binds to the protein-A resin;
(iv) application of the mini-trap to an AEX chromatographic resin (e.g., a column containing the resin), e.g., at a rate of about 50-500 g/L resin; and
(v) retention of the mini-trap in the flow-through fraction of the resin; and
(vi) optionally, further purification of the mini-trap, e.g., by hydrophobic interaction chromatography (HIC).

In an embodiment of the invention, the AEX resin is Q-sepharose Fast Flow or comprises the active group: —O—CH₂CHOHCH₂OCH₂CHOHCH₂N⁺(CH₃)₃ or —N⁺(CH₃)₃ or a quaternary amine. In an embodiment of the invention, the resin is POROS 50 HQ or comprises a quaternary polyethyleneimine active group.

In an embodiment of the invention, the conditions for the AEX chromatographic purification of VEGF mini-trap, in flow-through mode, is as follows:
(1) The AEX column is POROS 50 HQ (or an AEX resin with a quaternized polyethyleneimine functional group) which is equilibrated with a buffer at pH 8.30-8.50 having a conductivity of 1.90-2.10 mS/cm;
(2) The AEX column is Q Sepharose FF (or an AEX resin with a —O—CH₂CHOHCH₂OCH₂CHOHCH₂N⁺(CH₃)₃ or —N⁺(CH₃)₃ or a quaternary amine functional group) which is equilibrated with a buffer at pH 7.90-8.10 having a conductivity of 2.40-2.60 mS/cm;
(3) The AEX column is POROS 50 HQ (or an AEX resin with a quaternized polyethyleneimine functional group) which is equilibrated with a buffer at pH 7.90-8.10 having a conductivity of 2.40-2.60 mS/cm,
(4) The AEX column is Q Sepharose FF (or an AEX resin with a —O—CH₂CHOHCH₂OCH₂CHOHCH₂N⁺(CH₃)₃ or —N⁺(CH₃)₃ or a quaternary amine functional group) which is equilibrated with a buffer at pH 7.70-7.90 having a conductivity of 3.90-4.10 mS/cm,
(5) The AEX column is POROS 50 HQ (or an AEX resin with a quaternized polyethyleneimine functional group) which is equilibrated with a buffer at pH 7.70-7.90 having a conductivity of 3.90-4.10 mS/cm;
(6) The AEX column is Q Sepharose FF (or an AEX resin with a —O—CH₂CHOHCH₂OCH₂CHOHCH₂N⁺(CH₃)₃ or —N⁺(CH₃)₃ or a quaternary amine functional group) which is equilibrated with a buffer at pH 7.70±0.1 having a conductivity of 9.0±0.1 mS/cm; or
(7) The AEX column is POROS 50 HQ (or an AEX resin with a quaternized polyethyleneimine functional group) which is equilibrated with a buffer at pH 8.4±0.1 having a conductivity of 2.0±0.1 mS/cm.
   In an embodiment of the invention, the pH 8.30-8.50 buffer comprises: 50 mM Tris pH 8.4 and 2.0 mS/cm, the pH 7.90-8.10 buffer comprises: 50 mM Tris, 10 mM Acetate pH 8.0 and 2.5 mS/cm, the pH 7.70-7.90 buffer comprises: 50 mM Tris, 10 mM Acetate, 10 mM NaCl pH 7.8 and 4.0 mS/cm, the pH7.7 0+0.1 buffer comprises 50 mM Tris, 60 mM NaCl, pH7.7±0.1; and/or the at pH 8.4±0.1 buffer comprises 50 mM Tris pH 8.4±0.1.

In an embodiment of the invention, aflibercept, which is to be proteolytically cleaved, e.g., by S. pyogenes IdeS or a variant thereof, to generate VEGF mini-trap, is harvested from host cells and/or host cell chemically-defined growth media and, then cleaved prior to any AEX chromatographic purification.

In an embodiment of the invention, the AEX chromatography column is loaded at a rate of 40 grams of protein per liter of resin.

In an embodiment of the invention, prior to and/or after AEX chromatography, the VEGF mini-trap is purified by additional chromatography (e.g., mixed-mode chromatography, cation-exchange chromatography, protein-A chromatography and/or hydrophobic interaction chromatography (in flow-through mode or bind-and-elute mode)) and/or filtration steps (e.g., depth filtration, viral filtration, diafiltration and/or ultrafiltration).

Ion-exchange chromatography resins have charged functional groups bound to resin beads which attract biomolecules of the opposite charge or surface exposed patches of the opposite charge. Cation exchange resins are negatively charged, and anion exchange resins are positively charged. Ion-exchange resins are also categorized as “weak” or “strong” exchangers. These terms refer to the extent that the ionization state of the functional groups varies with pH. A “weak” exchanger is ionized over only a limited pH range, while a “strong” exchanger shows no variation in ion exchange capacity with changes in pH. Weak exchange resins can take up or lose protons with changes in buffer pH, and that added variation in charge offers an additional dimension of selectivity for binding and elution. Strong exchangers do not vary and remain fully charged over a broad pH range, which can make optimization of separation simpler than with weak exchangers. For example, strong anion exchange resins include, for example, Q Sepharose FF or Capto Q which have the functional group of a quaternary amine, e.g., —N⁺—(CH₃)₃; or POROS 50 HQ which has a quaternary polyethyleneimine functional group. In an embodiment of the invention, the AEX purification is performed with a strong or a weak anion exchanger, e.g., having a —N⁺—(CH₃)₃; or quaternary polyethyleneimine functional group.

The present invention also provides a method for making a VEGF mini-trap of the present invention (e.g., REGN7483^{F}, REGN7483^{R}, REGN7850 or REGN7851) comprising the steps of:
(i) culturing a host cell including a polynucleotide encoding the mini-trap or aflibercept under conditions such that the mini-trap or aflibercept is expressed and, optionally, secreted from the host cell into the growth medium (the host cell may be grown in CDM); and
(ii) removing the mini-trap or aflibercept from the host cells and/or medium;
(iii) optionally, purifying the aflibercept, if expressed, by protein-A chromatography; and
(iii) if aflibercept is expressed, proteolytically digesting the aflibercept with IdeS protease (e.g., FabRICATOR) or a variant thereof to generate mini-trap and Fc fragment; optionally, the Fc is removed from the composition by protein-A chromatography wherein the Fc binds to the protein-A resin. Mini-traps which are the product of such a method, and compositions thereof (e.g., aqueous compositions) are also part of the present invention.

Light Exposure

The brown-yellow color that characterizes VEGF mini-trap (e.g., REGN7483^{F}, REGN7483^{R}, REGN7850 or REGN7851) expressed in CDM can be reduced by anion exchange (AEX) chromatographic purification. Mini-trap, for example, which has been expressed by a host cell (e.g., Chinese hamster ovary (CHO) cell) in a chemically-defined liquid growth medium can be removed from the growth medium (after the host cells have been removed), by application to an AEX resin (e.g., a strong AEX resin) and retention of the material in the flow-through fraction. In addition, exposure of the mini-trap to light has been found to increase the appearance of the brown-yellow color. Thus, minimization of light exposure can reduce the appearance of the color. The present invention includes placing the VEGF mini-trap in a tinted vessel for storage (e.g., a brown vial). In an embodiment of the invention, the purification process (e.g., comprising AEX (flow-through) chromatography) and/or expression in CDM and/or storage is done while preventing light exposure of any greater than about 0.24, 0.6, 0.96, 1.2 or 2.4 million lux*hr white light exposure; and/or any greater than about 40, 100, 160, 200 or 400 W*h/m² ultra-violet A (UVA) light exposure.

Cell Culture Conditions

Other means by which to reduce brown-yellow color in compositions that include VEGF mini-traps expressed in CDM include the modulation of the concentration of various components of the culture medium. The presence of cysteine, particularly when in the presence of iron and zinc, has been shown to correlate with the brown-yellow color. For example, reducing cysteine concentration in CDM and culture feeds has been shown to reduce brown-yellow color. One method for reducing color or reducing cysteine concentration is to replace cysteine with cystine or cysteine sulfate and/or by reduction of the metal iron and/or zinc and/or nickel and/or copper and/or chelate content in CDM. For example, in an embodiment of the invention, cysteine concentration in the CDM in which a host cell is initially grown (on day 0) is about 1.3-1.6 (e.g., 1.3, 1.4, 1.5 or 1.6) millimoles per liter and additional cysteine feeds are added throughout the culture growth, e.g., a feed of 1.1-1.4 (e.g., 1.1, 1.2, 1.3 or 1.4) millimoles per liter of culture, 1.6-1.9 (e.g., 1.6, 1.7, 1.8 or 1.9) millimoles per liter of culture or 2.0-2.3 (2.0, 2.1, 2.2 or 2.3) millimoles per liter of culture, e.g., which is added every two days, e.g., on days 2, 4, 6 and 8. In an embodiment of the invention, iron (Fe), zinc (Zn), copper (Cu) and nickel (Ni) are included in the initial culture medium along with a chelating agent such as ethylenediaminetetraacetic acid (EDTA) and/or citric acid. In an embodiment of the invention, the chelator is EDTA present at a concentration of about 38-190 (e.g., 80, 85, 90, 95, 100, 105, 110, 115, 120, 130, 140, 150, 160, 170, 180 or 190) micromolar and citrate present at a concentration of about 22-110 (e.g., 22, 30, 40, 50, 60, 70, 80, 90, 100 or 110) micromolar; the Fe is present at a concentration of about 34-125 (e.g., 34, 40, 50, 60, 70, 75, 80, 90, 100, 120 or 125) micromolar; the Zn is present at a concentration of about 3-10 (e.g., 3, 4, 5, 6, 6.5, 7, 8, 8.5 9 or 10) micromolar; the Cu is present at a concentration of about 0.05-0.4 (e.g., 0.05, 0.06, 0.07, 0.08, 0.1, 0.15, 0.16, 0.17, 0.18, 0.19, 0.2, 0.3 or 0.4) millimolar; and the Ni is present at a concentration of about 0.25-2.0 (e.g., 0.25, 0.5, 0.6, 0.64, 0.65, 0.70, 0.75, 1, 1.5 or 2.0) millimolar. In an embodiment of the invention, the ratio of Fe:Zn:Cu:EDTA:citrate:Ni is about 441:38:1:500:294:4.

The inclusion of antioxidants in CDM in which VEGF mini-trap is expressed has also been shown to cause a reduction of brown-yellow color. For example, in an embodiment of the invention an antioxidant is hypotaurine, taurine, glycine, a combination of hypotaurine, taurine and glycine; a combination of hypotaurine, taurine, glycine and glutathione; thioctic acid and/or vitamin C. Other antioxidants that may be introduced include choline, hydrocortisone and vitamin E. In an embodiment of the invention, the initial culture medium has taurine at a concentration of about 10 mM of culture; hypotaurine at a concentration of about 10 mM of culture; glycine at a concentration of about 10 mM of culture; thioctic acid at a concentration of about 0.0024 mM of culture; and/or Vitamin C (ascorbic acid) at a concentration of about 0.028 mM of culture. Optionally, the initial culture medium has glutathione at a concentration of about 2 mM of culture; choline chloride at a concentration of about 1.43 mM of culture; hydrocortisone at a concentration of about 0.0014 mM of culture; and/or vitamin E (a-tocopherol) at a concentration of about 0.009 mM of culture.

With respect to the concentration of culture medium components, the term “cumulative” refers to a total amount or a total concentration of a particular component or components added over the course of the cell culture to form the CDM, including components added at the beginning of the culture (CDM at day 0) and subsequently added components (“chemically defined feeds”). Medium components are metabolized during culture so that cultures with the same cumulative amounts of given components will have different absolute levels if those components are added at different times (e.g., all present initially vs. some added by feeds).

In some embodiments of the invention, a modified CDM is used to produce a VEGF mini-trap of the present invention or a composition thereof (e.g., aqueous composition). Mini-traps produced by host cells cultured in modified CDMs and compositions comprising such mini-traps (e.g., having any of the color characteristics discussed herein) form part of the present invention. A modified CDM can be obtained by decreasing or increasing cumulative concentrations of amino acids in a CDM. Non-limiting examples of such amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine (or salts thereof). The increase or decrease in the cumulative amount of these amino acids in the modified CDM can be of about 1%, 5%, 10%, 15%, 20%, 25%, 30%, 35%, 40%, 45%, 50%, 55%, 60%, 65%, 70%, 75%, 80%, 85%, 90%, 95%, 100% as compared to the CDM, and ranges within one or more of the preceding. Alternatively, the increase or decrease in the cumulative amount of the one or more amino acids in the modified CDM can be about 5 to about 20%, about 10 to about 30%, about 30% to about 40%, about 30% to about 50%, about 40% to about 60%, about 60% to about 70%, about 70% to about 80%, about 80% to about 90%, or about 90% to about 100% as compared to the unmodified CDM, and ranges within one or more of the preceding.

In some embodiments, a modified CDM can be obtained by decreasing the cumulative concentration of cysteine in a CDM. The decrease in the amount of the cysteine in the CDM to form the modified CDM can be about 1%, 5%, 10%, 15%, 20%, 25%, 30%, 35%, 40%, 45%, 50%, 55%, 60%, 65%, 70%, 75%, 80%, 85%, 90%, 95%, 100% as compared to the unmodified CDM, and ranges within one or more of the preceding. Alternatively, the decrease in the cumulative amount of the cysteine in the modified CDM can be about 5 to about 20%, about 10-about 30%, about 30% to about 40%, about 30% to about 50%, about 40% to about 60%, about 60% to about 70%, about 70% to about 80%, about 80% to about 90%, or about 90% to about 100% as compared to the CDM, and ranges within one or more of the preceding. In one aspect, the amount of cumulative cysteine in modified CDM is less than a about 1 mM, about 2 mM, about 3 mM, about 4 mM, about 5 mM, about 6 mM, about 7 mM, about 8 mM, about 9 mM or about 10 mM.

In some embodiments, the modified CDM can be obtained by replacing at least a certain % of cumulative cysteine in a CDM with cystine. The replacement can be about 1%, 5%, 10%, 15%, 20%, 25%, 30%, 35%, 40%, 45%, 50%, 55%, 60%, 65%, 70%, 75%, 80%, 85%, 90%, 95%, 100% as compared to the CDM, and ranges within one or more of the preceding. Alternatively, the replacement can be about 5 to about 20%, about 10% to about 30%, about 30% to about 40%, about 30% to about 50%, about 40% to about 60%, about 60% to about 70%, about 70% to about 80%, about 80% to about 90%, or about 90% to about 100% as compared to the unmodified CDM, and ranges within one or more of the preceding.

In some embodiments, the modified CDM can be obtained by replacing at least a certain % of cumulative cysteine in a CDM with cysteine sulfate. The replacement can be about 1%, 5%, 10%, 15%, 20%, 25%, 30%, 35%, 40%, 45%, 50%, 55%, 60%, 65%, 70%, 75%, 80%, 85%, 90%, 95%, 100% as compared to the CDM, and ranges within one or more of the preceding. Alternatively, the replacement can be about 5 to about 20%, about 10-about 30%, about 30% to about 40%, about 30% to about 50%, about 40% to about 60%, about 60% to about 70%, about 70% to about 80%, about 80% to about 90%, or about 90% to about 100% as compared to the unmodified CDM, and ranges within one or more of the preceding.

In one embodiment, a VEGF mini-trap is produced by a method that comprises culturing a host cell in a CDM under suitable conditions, wherein the suitable conditions are obtained by lowering the cumulative concentration of iron in the CDM to less than or equal to about 50 μM. In one embodiment, a VEGF mini-trap is produced by a method that comprises culturing a host cell in a CDM under suitable conditions, wherein the suitable conditions are obtained by lowering the cumulative concentration of copper in the CDM to less than or equal to about 0.1 μM. In one embodiment, a VEGF mini-trap is produced by a method that comprises culturing a host cell in a CDM under suitable conditions, wherein the suitable conditions are obtained by lowering the cumulative concentration of zinc in the CDM to less than or equal to about 5 μM. Compositions comprising such mini-traps (e.g., aqueous compositions) are part of the present invention, e.g., wherein such compositions have color characteristics as set forth herein.

In some embodiments, the modified CDM can be obtained by decreasing or increasing cumulative concentration of metals in a CDM. Non-limiting examples of metals include iron, copper, manganese, molybdenum, zinc, nickel, calcium, potassium and sodium. The increase or decrease in the amount of the one or more metals in the modified CDM can be of about 1%, 5%, 10%, 15%, 20%, 25%, 30%, 35%, 40%, 45%, 50%, 55%, 60%, 65%, 70%, 75%, 80%, 85%, 90%, 95%, 100% as compared to the CDM, and ranges within one or more of the preceding. Alternatively, the increase or decrease in the cumulative amount of the one or more metals in the modified CDM can be about 5 to about 20%, about 10 to about 30%, about 30% to about 40%, about 30% to about 50%, about 40% to about 60%, about 60% to about 70%, about 70% to about 80%, about 80% to about 90%, or about 90% to about 100% as compared to the unmodified CDM, and ranges within one or more of the preceding.

In some embodiments, the modified CDM comprises one or more anti-oxidants. Non-limiting examples of anti-oxidants can include taurine, hypotaurine, glycine, thioctic acid, glutathione, choline chloride, hydrocortisone, Vitamin C, Vitamin E and combinations thereof. In some embodiments, the modified CDM comprises about 0.01 mM to about 20 mM of taurine, i.e., 0.01 mM to about 1 mM, about 0.01 mM to about 5 mM, about 0.01 mM to about 10 mM, 0.1 mM to about 1 mM, about 0.1 mM to about 5 mM, about 0.1 mM to about 10 mM, about 1 mM to about 5 mM, about 1 mM to about 10 mM, and ranges within one or more of the preceding. In some embodiments, the modified CDM comprises about 0.01 mM to about 20 mM of hypotaurine, i.e., 0.01 mM to about 1 mM, about 0.01 mM to about 5 mM, about 0.01 mM to about 10 mM, 0.1 mM to about 1 mM, about 0.1 mM to about 5 mM, about 0.1 mM to about 10 mM, about 1 mM to about 5 mM, about 1 mM to about 10 mM, and ranges within one or more of the preceding. In some embodiments, the modified CDM comprises about 0.01 mM to about 20 mM of glycine, 0.01 mM to about 1 mM, about 0.01 mM to about 5 mM, about 0.01 mM to about 10 mM, 0.1 mM to about 1 mM, about 0.1 mM to about 5 mM, about 0.1 mM to about 10 mM, about 1 mM to about 5 mM, about 1 mM to about 10 mM, and ranges within one or more of the preceding. In some embodiments, the modified CDM comprises about 0.01 nM to about 5 nM of thioctic acid, i.e., about 0.01 nM to about 0.1 nM, about 0.1 nM to about 1 nM, about 1 nM to about 2.5 nM, about 1 nM to about 3 nM, about 1 nM to about 5 nM, and ranges within one or more of the preceding. In some embodiments, the modified CDM comprises about 0.01 mM to about 5 mM of glutathione, i.e., 0.01 mM to about 1 mM, 0.1 mM to about 1 mM, about 0.1 mM to about 5 mM, about 1 mM to about 5 mM, and ranges within one or more of the preceding. In some embodiments, the modified CDM comprises about 0.01 mM to about 5 mM of choline chloride i.e., 0.01 mM to about 1 mM, 0.1 mM to about 1 mM, about 0.1 mM to about 5 mM, about 1 mM to about 5 mM, and ranges within one or more of the preceding. In some embodiments, the modified CDM comprises about 0.01 nM to about 5 nM of hydrocortisone, i.e., about 0.01 nM to about 0.1 nM, about 0.1 nM to about 1 nM, about 1 nM to about 2.5 nM, about 1 nM to about 3 nM, about 1 nM to about 5 nM, and ranges within one or more of the preceding. In some embodiments, the modified CDM comprises about 1 mM to about 50 mM of vitamin C, i.e., about 1 mM to about 5 mM, about 5 mM to about 20 mM, about 10 mM to about 30 mM, about 5 mM to about 30 mM, about 20 mM to about 50 mM, about 25 mM to about 50 mM, and ranges within one or more of the preceding. In some embodiments, the modified CDM comprises about 1 mM to about 50 mM of vitamin E, i.e., about 1 mM to about 5 mM, about 5 mM to about 20 mM, about 10 mM to about 30 mM, about 5 mM to about 30 mM, about 20 mM to about 50 mM, about 25 mM to about 50 mM, and ranges within one or more of the preceding.

Glycosylation

VEGF mini-traps (e.g., REGN7483, REGN7850 or REGN7851) produced by methods for modulating glycosylation thereof as well as compositions thereof (e.g., aqueous compositions), for example, having a color characteristic as discussed herein form part of the present invention. Glycosylation can be varied by varying cumulative concentrations of certain components in the CDMs in which host cells expressing the mini-traps are grown. Based on the cumulative amount of components added to the CDM, the total % fucosylation, total % galactosylation, total % sialylation and mannose-5 can be varied.

In an embodiment of the invention, VEGF mini-trap is desialylated.

In some exemplary embodiments, the method of modulating glycosylation of the VEGF mini-traps can comprise supplementing the CDM with uridine. The VEGF mini-traps can have about 40% to about 50% total fucosylated glycans, about 30% to about 55% total sialylated glycans, about 6% to about 15% mannose-5, and about 60% to about 79% galactosylated glycans.

In some exemplary embodiments, the method of modulating glycosylation of the VEGF mini-traps can comprise supplementing the CDM with manganese. The CDM as discussed here is devoid of manganese before supplementation. The VEGF mini-traps can have about 40% to about 50% total fucosylated glycans, about 30% to about 55% total sialylated glycans, about 6% to about 15% mannose-5, and about 60% to about 79% galactosylated glycans.

In some exemplary embodiments, the method of modulating glycosylation of the VEGF mini-traps can comprise supplementing the CDM with galactose. The CDM as discussed here is devoid of galactose before supplementation. The VEGF mini-traps can have about 40% to about 50% total fucosylated glycans, about 30% to about 55% total sialylated glycans, about 6% to about 15% mannose-5, and about 60% to about 79% galactosylated glycans.

In some exemplary embodiments, the method of modulating glycosylation of the VEGF mini-traps can comprise supplementing the CDM with dexamethasone. The CDM as discussed here is devoid of dexamethasone before supplementation. The VEGF mini-traps can have about 40% to about 50% total fucosylated glycans, about 30% to about 55% total sialylated glycans, about 6% to about 15% mannose-5, and about 60% to about 79% galactosylated glycans.

In some exemplary embodiments, the method of modulating glycosylation of the VEGF mini-traps can comprise supplementing the CDM with one or more of uridine, manganese, galactose and dexamethasone. The CDM as discussed here is devoid of one or more of uridine, manganese, galactose and dexamethasone before supplementation. The anti-VEGF protein can have about 40% to about 50% total fucosylated glycans, about 30% to about 55% total sialylated glycans, about 6% to about 15% mannose-5, and about 60% to about 79% galactosylated glycans.

In an embodiment of the invention, in VEGF mini-traps (e.g., REGN7483, REGN7850 or REGN7851) in a composition of the present invention,
less than about 0.1% are tri-xylosylated
about 1.5% are di-xylosylated;
about 15% are mono-xylosylated;
about 0.9% or less than about 1% are modified with xylose-galactose; and/or
about 0.7% or less than about 1% are modified with xylose-galactose-sialic acid.

In an embodiment of the invention,
about 8% of the Arginine 5 residues;
less than about 0.1% of the Arginine 153 residues; and/or
less than about 0.1% of the Arginine 96 residues;
   in VEGF mini-traps (e.g., REGN7483, REGN7850 or REGN7851) in a composition of the present invention are modified with 3-deoxyglucosone.

In an embodiment of the invention,
about 0.1% of the Arginine 5 residues;
about 1.0 or 1.1% of the Lysine 62 residues;
about 0.4% or less of the Lysine 68 residues;
about 0.6% or less of the Lysine 149 residues; and/or
less than about 0.1% of the Lysine 185 residues;
   in VEGF mini-traps (e.g., REGN7483, REGN7850 or REGN7851) in a composition of the present invention are glycated.

In an embodiment of the invention, in a composition comprising a VEGF mini-trap (e.g., REGN7483, REGN7850 or REGN7851),
about 98% or more of the asparagine 36 residues, e.g., corresponding to (R) VTSPNITVTLK (underscored) (amino acids 31-42 of SEQ ID NO: 12);
about 51, 52, 53, 54 or 55% of the asparagine 68 residues, e.g., corresponding to (K) GFIISNATYK (underscored) (amino acids 62-72 of SEQ ID NO: 12);
about 99% or more of the asparagine 123 residues, e.g., corresponding to (K) LVLNCTAR (underscored) (amino acids 119-127 of SEQ ID NO: 12); and/or
about 44, 50, 60, 70, 80, 90, 98 or 99% of the asparagine 196 residues, e.g., corresponding to (K)NSTFVR (amino acids 195-201 of SEQ ID NO: 12); are N-glycosylated.

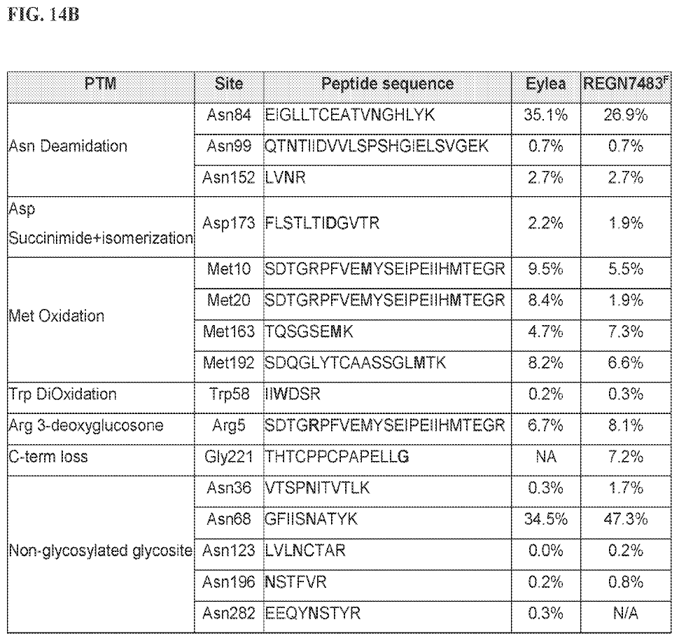

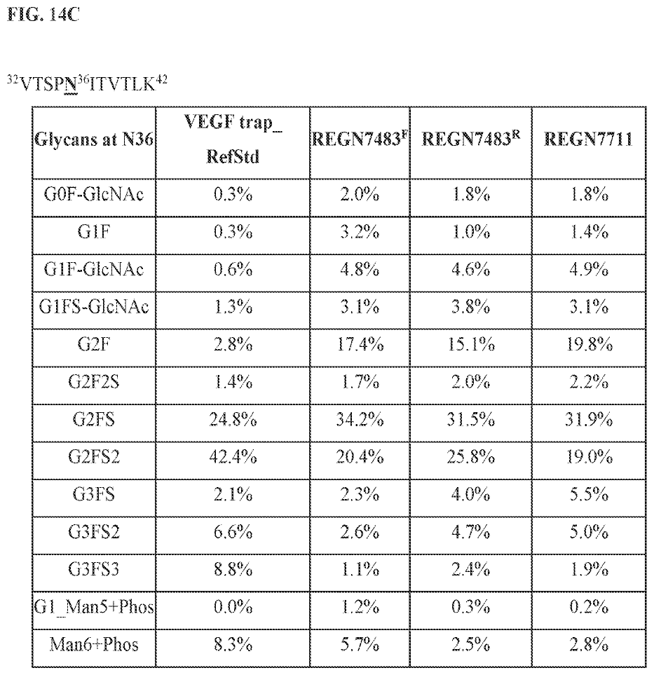

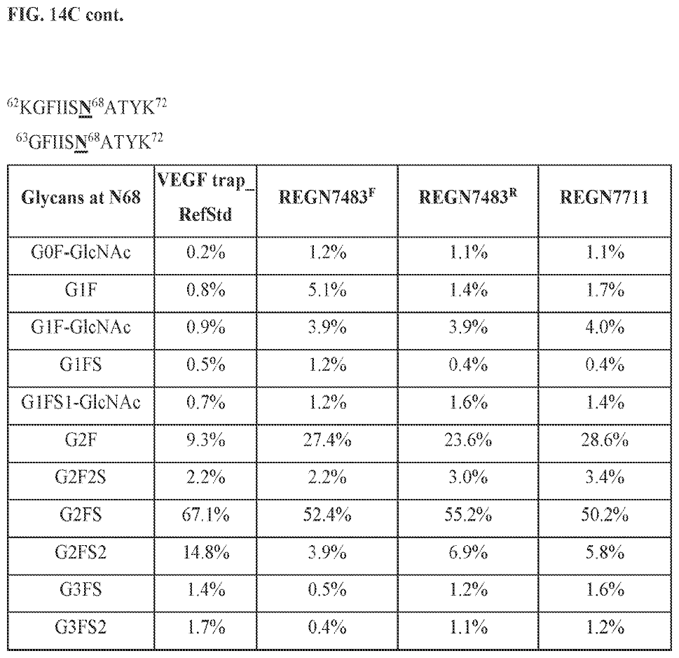

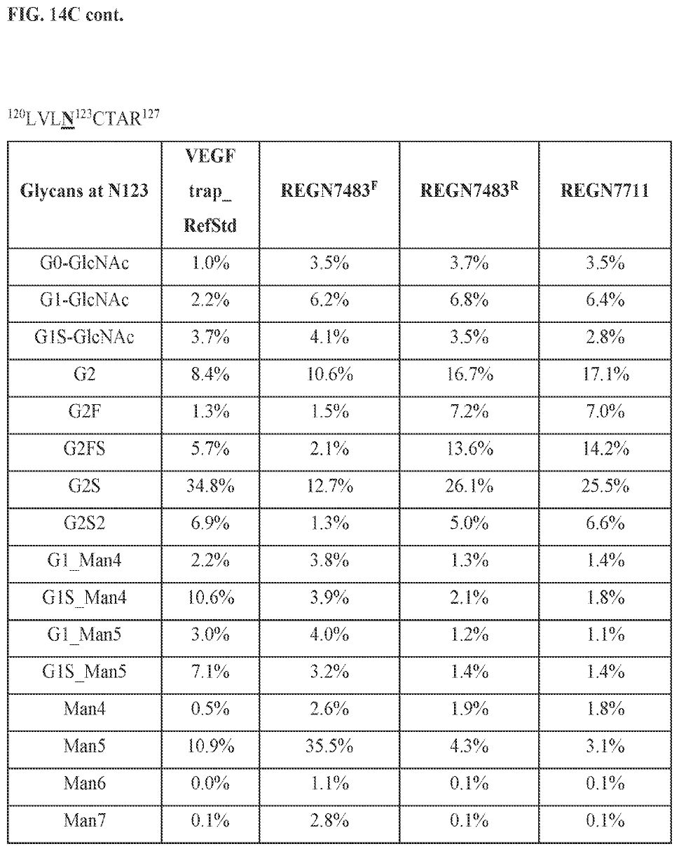

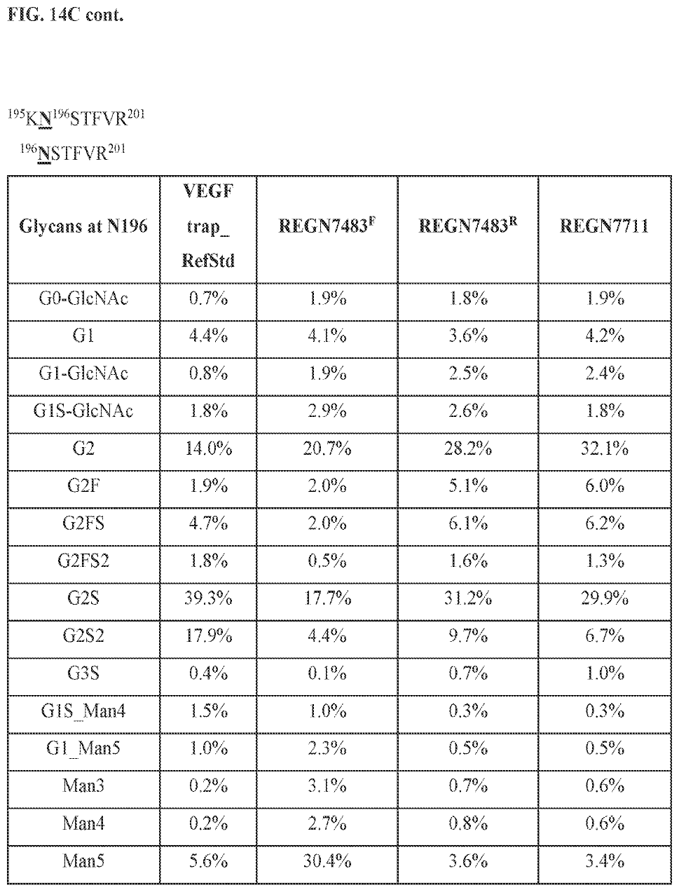

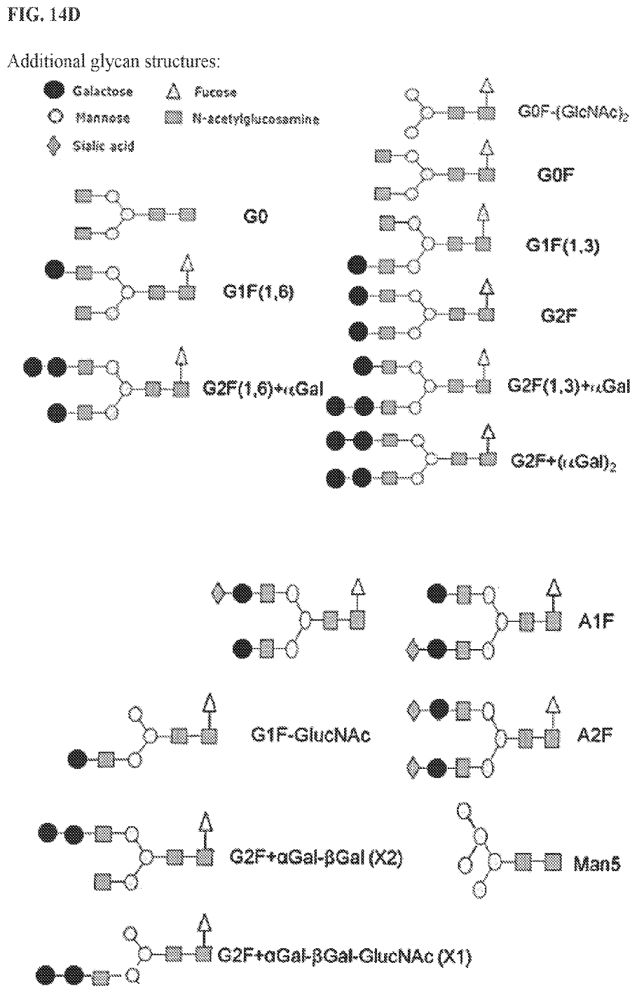

Glycosylation has been shown to have a great impact on the safety and function of biotherapeutics. Obtaining mini-traps with a favorable glycosylation profile would be highly beneficial to their successful use for treating angiogenic eye disorders. VEGF antagonists, in general, have been shown to have common adverse vascular effects attributable directly or indirectly to their anti-VEGF effects, including hypertension, renal vascular injury, often manifested by proteinuria and thrombotic microangiopathy, and congestive heart failure. Thus, any means by which to reduce the systemic exposure of a subject receiving intravitreally injected mini-trap would be beneficial. Intravitreally injected VEGF antagonists are thought to leak, in small amounts, into systemic circulation wherein they can have such adverse effects. See e.g., Avery RL et al., Comparison of Systemic Pharmacokinetics Post Anti-VEGF Intravitreal Injections of Ranibizumab, Bevacizumab and Aflibercept (abstract). Presented at the 2013 Annual Meeting of the American Society of Retina Specialists (ASRS); Toronto, 25 Aug. 2013; Avery et al., Intravitreal bevacizumab (Avastin) in the treatment of proliferative diabetic retinopathy. Ophthalmology 2006;113:1695-705; Matsuyama et al., Plasma levels of vascular endothelial growth factor and pigment epithelium-derived factor before and after intravitreal injection of bevacizumab. Br J Ophthalmol 2010;94:1215-18; and Carneiro et al., Vascular endothelial growth factor plasma levels before and after treatment of neovascular age-related macular degeneration with bevacizumab or ranibizumab. Acta Ophthalmol 2012;90:e25-30. In vivo studies presented herein suggest that mini-trap has a shorter half-life than aflibercept when administered systemically (see Example 6). One reason for this effect may be the glycosylation profile of mini-trap. REGN7483^{F}, which has been produced in chemically-defined media, is known to have a particularly high level of high mannose glycans on N123 and N196-a greater level than observed on aflibercept. As discussed further below, in Table C and in FIG. 14(A and C), about 30-40% of the N123s and N196s in the composition tested were highly mannosylated. Aflibercept had about 6-13% of these residues were observed to be highly mannosylated in aflibercept. High mannose glycans, in antibodies, have been shown to lead to rapid systemic clearance and shorter half-life. See Goetze et al., High-mannose glycans on the Fc region of therapeutic IgG antibodies increase serum clearance in humans, Glycobiology 21(7): 949-959 (2011). This may be due to binding by the mannose receptor which removes high mannose containing pathogens from the blood. A similar mechanism may be causing the rapid systemic clearance of mini-trap.

In one aspect of the invention, the glycosylation profile of a composition of VEGF mini-trap is as follows: about 40% to about 50% total fucosylated glycans, about 30% to about 55% total sialylated glycans, about 6% to about 15% mannose-5, and about 60% to about 79% galactosylated glycans. In an aspect of the invention, the mini-trap has Man5 glycosylation at about 32.4% of Asparagine 123 residues and/or about 27.1% of Asparagine 196 residues.

For example, a composition of the present invention includes a VEGF mini-trap of the present invention (e.g., REGN7483, REGN7850 or REGN7851), for example, which has been expressed in CHO cells and in CDM and purified by AEX flow-through chromatography as set forth herein, comprises:
Man5 glycosylation at about 30-35% of asparagine 123 residues;
Man5 glycosylation at about 25-30% of asparagine 196 residues;
Man6-phosphate glycosylation at about 6-8% of asparagine 36 residues;
Man7 glycosylation at about 3-4% of asparagine 123 residues;
High mannose glycosylation at about 38% of asparagine 123 residues; and/or
High mannose glycosylation at about 29% of asparagine 196 residues.

The present invention also includes a VEGF mini-trap (e.g., REGN7483^{F} or REGN7483^{R}) which comprises Man5 glycosylation at Asn123; Man5 glycosylation at Asn196; Man6-phosphate glycosylation at Asn36; and/or Man7 glycosylation at Asn123.

A VEGF mini-trap of the present invention, in an embodiment of the invention, may include one of more of the glycosylations listed below. Compositions (e.g., aqueous compositions) comprising mini-traps of the present invention including mini-trap molecules having such glycosylations, e.g., at the indicated percentage frequencies are also part of the present invention.
G0-GlcNAc glycosylation at Asn36 (e.g., at about 0%), Asn68 (e.g., about 0%), Asn123 (e.g., about 1.00%) and/or Asn196 (e.g., about 1.40%);
G1-GlcNAc glycosylation at Asn36 (e.g., at about 0%), Asn68 (e.g., about 0%), Asn123 (e.g., about 4.80%) and/or Asn196 (e.g., about 2.70%);
G1S-GlcNAc glycosylation at Asn36 (e.g., at about 0%), Asn68 (e.g., about 0%), Asn123 (e.g., about 4.10%) and/or Asn196 (e.g., about 2.20%);
G0 glycosylation at Asn36 (e.g., at about 0%), Asn68 (e.g., about 0%), Asn123 (e.g., about 0%) and/or Asn196 (e.g., about 0%);
G1 glycosylation at Asn36 (e.g., at about 0%), Asn68 (e.g., about 0%), Asn123 (e.g., about 0%) and/or Asn196 (e.g., about 6.10%);
G1S glycosylation at Asn36 (e.g., at about 0%), Asn68 (e.g., about 0%), Asn123 (e.g., about 0%) and/or Asn196 (e.g., about 1.90%);
G2 glycosylation at Asn36 (e.g., at about 0%), Asn68 (e.g., about 0%), Asn123 (e.g., about 11.50%) and/or Asn196 (e.g., about 18.10%);
G2S glycosylation at Asn36 (e.g., at about 0%), Asn68 (e.g., about 0%), Asn123 (e.g., about 14.50%) and/or Asn196 (e.g., about 18.40%);
G2S2 glycosylation at Asn36 (e.g., at about 0%), Asn68 (e.g., about 0%), Asn123 (e.g., about 1.50%) and/or Asn196 (e.g., about 3.70%);
G0F glycosylation at Asn36 (e.g., at about 0%), Asn68 (e.g., about 0%), Asn123 (e.g., about 0%) and/or Asn196 (e.g., about 0%);
G2F2S glycosylation at Asn36 (e.g., at about 2.00%), Asn68 (e.g., about 2.00%), Asn123 (e.g., about 0%) and/or Asn196 (e.g., about 0%);
G2F2S2 glycosylation at Asn36 (e.g., at about 1.60%), Asn68 (e.g., about 0.50%), Asn123 (e.g., about 0%) and/or Asn196 (e.g., about 0%);
G1F glycosylation at Asn36 (e.g., at about 5.60%), Asn68 (e.g., about 6.10%), Asn123 (e.g., about 0%) and/or Asn196 (e.g., about 0%);
G1FS glycosylation at Asn36 (e.g., at about 0%), Asn68 (e.g., about 3.80%), Asn123 (e.g., about 0%) and/or Asn196 (e.g., about 0%);
G2F glycosylation at Asn36 (e.g., at about 20.20%), Asn68 (e.g., about 28.00%), Asn123 (e.g., about 1.80%) and/or Asn196 (e.g., about 2.10%);
G2FS glycosylation at Asn36 (e.g., at about 35.20%), Asn68 (e.g., about 48.90%), Asn123 (e.g., about 2.80%) and/or Asn196 (e.g., about 2.20%);
G2FS2 glycosylation at Asn36 (e.g., at about 22.40%), Asn68 (e.g., about 9.10%), Asn123 (e.g., about 0.30%) and/or Asn196 (e.g., about 0.60%);
G3FS glycosylation at Asn36 (e.g., at about 3.40%), Asn68 (e.g., about 1.60%), Asn123 (e.g., about 0%) and/or Asn196 (e.g., about 0%);
G3FS3 glycosylation at Asn36 (e.g., at about 1.70%), Asn68 (e.g., about 0%), Asn123 (e.g., about 0%) and/or Asn196 (e.g., about 0%);
G0-2GlcNAc glycosylation at Asn36 (e.g., at about 0%), Asn68 (e.g., about 0%), Asn123 (e.g., about 3.40%) and/or Asn196 (e.g., about 2.60%);
Man4 glycosylation at Asn36 (e.g., at about 0%), Asn68 (e.g., about 0%), Asn123 (e.g., about 0.50%) and/or Asn196 (e.g., about 1.60%);
Man4_A1G1 glycosylation at Asn36 (e.g., at about 0%), Asn68 (e.g., about 0%), Asn123 (e.g., about 3.60%) and/or Asn196 (e.g., about 2.10%);
Man4_A1G1S1 glycosylation at Asn36 (e.g., at about 0%), Asn68 (e.g., about 0%), Asn123 (e.g., about 4.60%) and/or Asn196 (e.g., about 3.00%);
Man5 glycosylation at Asn36 (e.g., at about 0%), Asn68 (e.g., about 0%), Asn123 (e.g., about 32.40%) and/or Asn196 (e.g., about 27.10%);
Man5_A1G1 glycosylation at Asn36 (e.g., at about 0%), Asn68 (e.g., about 0%), Asn123 (e.g., about 4.80%) and/or Asn196 (e.g., about 2.80%);
Man5_A1G1S1 glycosylation at Asn36 (e.g., at about 0%), Asn68 (e.g., about 0%), Asn123 (e.g., about 3.30%) and/or Asn196 (e.g., about 1.50%);
Man6 glycosylation at Asn36 (e.g., at about 0%), Asn68 (e.g., about 0%), Asn123 (e.g., about 1.30%) and/or Asn196 (e.g., about 0%);

Man6_G0+Phosphate glycosylation at Asn36 (e.g., at about 1.70%), Asn68 (e.g., about 0%), Asn123 (e.g., about 0%) and/or Asn196 (e.g., about 0%);
Man6+Phosphate glycosylation at Asn36 (e.g., at about 6.20%), Asn68 (e.g., about 0%), Asn123 (e.g., about 0%) and/or Asn196 (e.g., about 0%);
Man7 glycosylation at Asn36 (e.g., at about 0%), Asn68 (e.g., about 0%), Asn123 (e.g., about 3.60%) and/or Asn196 (e.g., about 0%);
Any of the glycosylations (e.g., at about the indicated levels) at indicated asparagines in FIG. 14(A or C) whether for REGN7483^{F}, REGN7483^{R}, REGN7711 or another VEGF mini-trap set forth herein that comprises a VTSPNITVTLK; KGFIISNATYK; GFIISNATYK; LVLNCTAR; KNSTFVR; or NSTFVR motif and/or a N36, N68, N123 or N196 residue;
Any of the glycosylations (e.g., at about the indicated levels) at indicated asparagines in Table C (a or b) herein whether for REGN7483^{F} or another VEGF mini-trap set forth herein that comprises an N36, N68, N123 or N196 residue; and/or Any of the glycosylations (e.g., at about the indicated levels) Table D herein whether for REGN7483^{F} or another VEGF mini-trap set forth herein

A composition (e.g., aqueous composition) including VEGF mini-traps of the present invention may include one of more of the glycosylations indicated in Table C, e.g., at the percentage frequencies which are shown (e.g., all of the glycosylations at the indicated percentages, e.g.,±10% of the indicated percentage number). A VEGF mini-trap of the present invention, in an embodiment of the invention, may include one of more of the glycosylations listed below, e.g., at one or more of the residues indicated.

TABLE C*
(a) Glycosyl Post-translational Modifications

| | REGN7483^{F} | | | |
|---|---|---|---|---|
| Glycan Annotation | N36 | N68 | N123 | N196 |
| G0-GlcNAc | x | x | 1.0% | 1.4% |
| G1-GlcNAc | x | x | 4.8% | 2.7% |
| G1S-GlcNAc | x | x | 4.1% | 2.2% |
| G0 | x | x | x | x |
| G1 | x | x | x | 6.1% |
| G1S | x | x | x | 1.9% |
| G2 | x | x | 11.5% | 18.1% |
| G2S | x | x | 14.5% | 18.4% |
| G2S2 | x | x | 1.5% | 3.7% |
| G0F | x | x | x | x |
| G2F2S | 2.0% | 2.0% | x | x |
| G2F2S2 | 1.6% | 0.5% | x | x |
| G1F | 5.6% | 6.1% | x | x |
| G1FS | x | 3.8% | x | x |
| G2F | 20.2% | 28.0% | 1.8% | 2.1% |
| G2FS | 35.2% | 48.9% | 2.8% | 2.2% |
| G2FS2 | 22.4% | 9.1% | 0.3% | 0.6% |
| G3FS | 3.4% | 1.6% | x | x |
| G3FS3 | 1.7% | x | x | x |
| G0-2GlcNAc | x | x | 3.4% | 2.6% |
| Man4 | x | x | 0.5% | 1.6% |
| Man4_A1G1 | x | x | 3.6% | 2.1% |
| Man4_A1G1S1 | x | x | 4.6% | 3.0% |
| Man5 | x | x | 32.4% | 27.1% |
| Man5_A1G1 | x | x | 4.8% | 2.8% |
| Man5_A1G1S1 | x | x | 3.3% | 1.5% |
| Man6 | x | x | 1.3% | x |
| Man6_G0 + Phosphate | 1.7% | x | x | x |
| Man6 + Phosphate | 6.2% | x | x | x |
| Man7 | x | x | 3.6% | x |
| % Fucosylation | 90.6% | 99.5% | 4.9% | 4.8% |
| % Galactosylation | 89.4% | 95.0% | 45.0% | 56.2% |
| % Sialyation | 46.0% | 37.7% | 16.4% | 18.8% |
| % High Mannose | 0.0% | 0.0% | 37.9% | 28.7% |
| % Total Glycosylation | 99.7% | 65.5% | 100.0% | 99.8% |
| Occupancy | | | | |

(b) Glycans observed on N36, N68, N123 and N196 in
REGN7483^{R} and REGN7483^{F} (second experiment)

| Glycans at N36 | REGN7483^{F} | REGN7483^{R} |
|---|---|---|
| G0F-GlcNAc | 2.0% | 1.8% |
| G1F | 3.2% | 1.0% |
| G1F-GlcNAc | 4.8% | 4.6% |
| G1FS-GlcNAc | 3.1% | 3.8% |
| G2F | 17.4% | 15.1% |
| G2F2S | 1.7% | 2.0% |
| G2FS | 34.2% | 31.5% |
| G2FS2 | 20.4% | 25.8% |
| G3FS | 2.3% | 4.0% |
| G3FS2 | 2.6% | 4.7% |
| G3FS3 | 1.1% | 2.4% |
| G1_Man5 + Phos | 1.2% | 0.3% |
| Man6 + Phos | 5.7% | 2.5% |

| Glycans at N68 | REGN7483^{F} | REGN7483^{R} |
|---|---|---|
| G0F-GlcNAc | 1.2% | 1.1% |
| G1F | 5.1% | 1.4% |
| G1F-GlcNAc | 3.9% | 3.9% |
| G1FS | 1.2% | 0.4% |
| G1FS1-GlcNAc | 1.2% | 1.6% |
| G2F | 27.4% | 23.6% |
| G2F2S | 2.2% | 3.0% |
| G2FS | 52.4% | 55.2% |
| G2FS2 | 3.9% | 6.9% |
| G3FS | 0.5% | 1.2% |
| G3FS2 | 0.4% | 1.1% |
| Glycans at N123 | REGN7483^{F} | REGN7483^{R} |
| G0-GlcNAc | 3.5% | 3.7% |
| G1-GlcNAc | 6.2% | 6.8% |
| G1S-GlcNAc | 4.1% | 3.5% |
| G2 | 10.6% | 16.7% |
| G2F | 1.5% | 7.2% |
| G2FS | 2.1% | 13.6% |
| G2S | 12.7% | 26.1% |
| G2S2 | 1.3% | 5.0% |
| G1_Man4 | 3.8% | 1.3% |
| G1S_Man4 | 3.9% | 2.1% |
| G1_Man5 | 4.0% | 1.2% |
| G1S_Man5 | 3.2% | 1.4% |
| Man4 | 2.6% | 1.9% |
| Man5 | 35.5% | 4.3% |
| Man6 | 1.1% | 0.1% |
| Man7 | 2.8% | 0.1% |
| Glycans at N196 | REGN7483^{F} | REGN7483^{R} |
| G0-GlcNAc | 1.9% | 1.8% |
| G1 | 4.1% | 3.6% |
| G1-GlcNAc | 1.9% | 2.5% |
| G1S-GlcNAc | 2.9% | 2.6% |
| G2 | 20.7% | 28.2% |
| G2F | 2.0% | 5.1% |
| G2FS | 2.0% | 6.1% |
| G2FS2 | 0.5% | 1.6% |
| G2S | 17.7% | 31.2% |
| G2S2 | 4.4% | 9.7% |
| G3S | 0.1% | 0.7% |
| G1S_Man4 | 1.0% | 0.3% |
| G1_Man5 | 2.3% | 0.5% |
| Man3 | 3.1% | 0.7% |
| Man4 | 2.7% | 0.8% |
| Man5 | 30.4% | 3.6% |

*The structures of the glycan residues (G0-GlcNAc; G1-GlcNAc; G1S-GlcNAc; G0; G1; G1S; G2; G2S; G2S2; G0F; G2F2S; G2F2S2; G1F; G1FS; G2F; G2FS; G2FS2; G3FS; G3FS3; G0-2GlcNAc; Man4; Man4_A1G1; Man4_A1G1S1; Man5; Man5_A1G1; Man5_A1G1S1; Man6; Man6_G0 + Phosphate; Man6 + Phosphate and Man7) are standardized-see Varki et al., Symbol nomenclature for glycan
Representation, Proteomics 9: 5398-5399 (2009); Harvey et al., Proposal for a standard system for drawing structural diagrams of N- and O-linked carbohydrates and related compounds. Proteomics 2009, 9, 3796-3801; Kornfeld et al., The synthesis of complex-type oligosaccharides II characterization of the processing intermediates in the synthesis of the complex oligosaccharide units of the vesicular stomatitis virus G protein. J Biol Chem. 1978, 253, 7771-7778; Varki et al. (Eds.), Essentials of Glycobiology, 1st Edn., Cold Spring Harbor Laboratory Press, Plainview, NY 1999; Varki et al. (Eds.), Essentials of Glycobiology, 2nd Edn., Cold Spring Harbor Laboratory Press, Plainview, NY 2009; and Dwek, Glycobiology: Moving into the mainstream. Cell 2009, 137, 1175-1176. REGN7483^{R} and aflibercept used to make REGN7483^{F} expressed in CDM.

The present invention includes compositions comprising VEGF mini-traps (e.g., REGN7483^{F}) comprising any one or more of the percent glycosylations indicated below in Table D.

TABLE D
Percent Glycosylation in REGN7483^{F} Samples

| | REGN7483^{F} | REGN7483^{F} | REGN7483^{F} |
|---|---|---|---|
| % Fucosylation | 42.9% | 57.8% | 57.2% |
| % Galactosylation | 71.6% | 92.9% | 93.7% |
| % Sialylation | 33.1% | 47.6% | 44.8% |
| % High Mannose | 17.6% | 2.6% | 2.3% |
| % Bisecting | 1.9% | 0.4% | 0.4% |

In some exemplary embodiments of the invention, the VEGF mini-traps can have a decreased level of fucosylated glycans by about 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40%, 45%, 50%, 55%, 60%, 65%, 70%, 75%, 80%, 85%, 90%, 95% or 99%. Ranges within one or more of the preceding values, e.g., 1-10%, 1-15%, 1-20%, 1-25%, 1-30%, 1-35%, 1-40%, 1-41%, 1-42%, 1-43%, 1-44%, 1-45%, 1-46%, 1-47%, 1-48%, 1-49%, 1-50%, 2-10%, 2-15%, 2-20%, 2-25%, 2-30%, 2-35%, 2-40%, 2-41%, 2-42%, 2-43%, 2-44%, 2-45%, 2-46%, 2-47%, 2-48%, 2-49%, 2-50%, 3-10%, 3-15%, 3-20%, 3-25%, 3-30%, 3-35%, 3-40%, 3-41%, 3-42%, 3-43%, 3-44%, 3-45%, 3-46%, 3-47%, 3-48%, 3-49%, 3-50%, 4-10%, 4-15%, 4-20%, 4-25%, 4-30%, 4-35%, 4-40%, 4-41%, 4-42%, 4-43%, 4-44%, 4-45%, 4-46%, 4-47%, 4-48%, 4-49%, 4-50% or 1-99% compared to level of fucosylated glycans in a VEGF mini-trap produced using a non-CDM.

In some exemplary embodiments of the invention, a VEGF mini-trap can have a decreased level of sialylated glycans by about 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40%, 45%, 50%, 55%, 60%, 65%, 70%, 75%, 80%, 85%, 90%, 95% or 99%. Ranges within one or more of the preceding values, e.g., 1-10%, 1-15%, 1-20%, 1-25%, 1-30%, 1-35%, 1-40%, 1-41%, 1-42%, 1-43%, 1-44%, 1-45%, 1-46%, 1-47%, 1-48%, 1-49%, 1-50%, 2-10%, 2-15%, 2-20%, 2-25%, 2-30%, 2-35%, 2-40%, 2-41%, 2-42%, 2-43%, 2-44%, 2-45%, 2-46%, 2-47%, 2-48%, 2-49%, 2-50%, 3-10%, 3-15%, 3-20%, 3-25%, 3-30%, 3-35%, 3-40%, 3-41%, 3-42%, 3-43%, 3-44%, 3-45%, 3-46%, 3-47%, 3-48%, 3-49%, 3-50%, 4-10%, 4-15%, 4-20%, 4-25%, 4-30%, 4-35%, 4-40%, 4-41%, 4-42%, 4-43%, 4-44%, 4-45%, 4-46%, 4-47%, 4-48%, 4-49%, 4-50% or 1-99% compared to level of sialylated glycans in a VEGF mini-trap produced using a non-CDM.

In some exemplary embodiments of the invention, a VEGF mini-trap can have an decreased level of galactosylated glycans by about 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40%, 45%, 50%, 55%, 60%, 65%, 70%, 75%, 80%, 85%, 90%, 95% or 99%. Ranges within one or more of the preceding values, e.g., 1-10%, 1-15%, 1-20%, 1-25%, 1-30%, 1-35%, 1-40%, 1-41%, 1-42%, 1-43%, 1-44%, 1-45%, 1-46%, 1-47%, 1-48%, 1-49%, 1-50%, 2-10%, 2-15%, 2-20%, 2-25%, 2-30%, 2-35%, 2-40%, 2-41%, 2-42%, 2-43%, 2-44%, 2-45%, 2-46%, 2-47%, 2-48%, 2-49%, 2-50%, 3-10%, 3-15%, 3-20%, 3-25%, 3-30%, 3-35%, 3-40%, 3-41%, 3-42%, 3-43%, 3-44%, 3-45%, 3-46%, 3-47%, 3-48%, 3-49%, 3-50%, 4-10%, 4-15%, 4-20%, 4-25%, 4-30%, 4-35%, 4-40%, 4-41%, 4-42%, 4-43%, 4-44%, 4-45%, 4-46%, 4-47%, 4-48%, 4-49%, 4-50% or 1-99% compared to level of galactosylated glycans in VEGF mini-trap produced using a non-CDM.

In some exemplary embodiments of the invention, a VEGF mini-trap can have an increased level of mannosylated glycans by about 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40%, 45%, 50%, 55%, 60%, 65%, 70%, 75%, 80%, 85%, 90%, 95% or 99%. Ranges within one or more of the preceding values, e.g., 1-10%, 1-15%, 1-20%, 1-25%, 1-30%, 1-35%, 1-40%, 1-41%, 1-42%, 1-43%, 1-44%, 1-45%, 1-46%, 1-47%, 1-48%, 1-49%, 1-50%, 2-10%, 2-15%, 2-20%, 2-25%, 2-30%, 2-35%, 2-40%, 2-41%, 2-42%, 2-43%, 2-44%, 2-45%, 2-46%, 2-47%, 2-48%, 2-49%, 2-50%, 3-10%, 3-15%, 3-20%, 3-25%, 3-30%, 3-35%, 3-40%, 3-41%, 3-42%, 3-43%, 3-44%, 3-45%, 3-46%, 3-47%, 3-48%, 3-49%, 3-50%, 4-10%, 4-15%, 4-20%, 4-25%, 4-30%, 4-35%, 4-40%, 4-41%, 4-42%, 4-43%, 4-44%, 4-45%, 4-46%, 4-47%, 4-48%, 4-49%, 4-50% or 1-99% compared to level of mannosylated glycans in VEGF mini-trap produced using a non-CDM.

Other Post-Translational Modifications (PTMs)

In an embodiment of the invention, a composition includes VEGF mini-traps of the present invention (e.g., REGN7483, REGN7850 or REGN7851) having other PTMs such as free thiols, trisulfide bonds, deamidations, methionine oxidations and C-terminal amino acid loss.

In an embodiment of the invention, about 0% of the cysteines in the hinge region of VEGF mini-traps of the present invention (e.g., REGN7483, REGN7850 or REGN7851) in a composition and/or about 0.3% or less of the cysteines in the VEGFR1 and/or VEGFR2 domains of VEGF mini-traps of the present invention (e.g., REGN7483, REGN7850 or REGN7851) in a composition are free thiols; for example; with regard to the cysteine(s) in the VEGFR1 (corresponding to ELVIPCR, underscored), in the VEGFR2 (corresponding to LVLNCTAR, underscored (amino acids 120-127 of SEQ ID NO: 12)) and/or in the hinge region (corresponding to THTCPPCPAPELLG (amino acids 208-221 of SEQ ID NO: 12) or THTCPPCPPC (amino acids 208-217 of SEQ ID NO: 28), underscored).

In an embodiment of the invention, about 4% or less of the cysteines in the hinge region of VEGF mini-traps (e.g., REGN7483, REGN7850 or REGN7851) in a composition of the present invention and/or about 0.1% or less of the cysteines in the VEGFR1 and/or VEGFR2 domains of VEGF mini-traps (e.g., REGN7483, REGN7850 or REGN7851) in a composition of the present invention are in a trisulfide bridge; for example; with regard to the cysteine(s) in the VEGFR1 (corresponding to ELVIPCR (amino acids 25-31 of SEQ ID NO: 12) & EIGLLTCEATVNGHLYK (amino acids 73-89 of SEQ ID NO: 12), underscored), in the VEGFR2 (corresponding to LVLNCTAR (amino acids 120-127 of SEQ ID NO: 12) & SDQGLYTCAASSGLMTK (K) (amino acids 178-195 of SEQ ID NO: 12), underscored) and/or in the hinge region (corresponding to THTCPPCPAPELLG & THTCPPCPAPELL (G) (amino acids 208-221 of SEQ ID NO: 12) or THTCPPCPPC & THTCPPCPPC (amino acids 208-217 of SEQ ID NO: 28), underscored).

In an embodiment of the invention, less than about 0.1% of the cysteines in VEGF mini-traps (e.g., REGN7483, REGN7850 or REGN7851) in a composition of the present invention are in an intrachain disulfide and/or trisulfide bond.

In an embodiment of the invention, greater than about 99% (e.g., about 99.8%) of the disulfide bridges in VEGF mini-traps (e.g., REGN7483, REGN7850 or REGN7851) in a composition of the present invention are in parallel form.

In an embodiment of the invention, about 3% of the asparagine 84 residues, e.g., the asparagine corresponding to EIGLLTCEATVNGHLYK (amino acids 73-89 of SEQ ID NO: 12) (underscored), in VEGF mini-traps (e.g., REGN7483, REGN7850 or REGN7851) in a composition of the present invention, are deamidated to form succinimide. In an embodiment of the invention, about 18, 19, 20, 21 or 22% of said asparagines are deamidated to form aspartate/isoaspartate.

In an embodiment of the invention, less than about 5% of the asparagine 99 residues e.g., the asparagine corresponding to QTNTIIDVVLSPSHGIELSVGEK (amino acids 97-119 of SEQ ID NO: 12) (underscored) in VEGF mini-traps (e.g., REGN7483, REGN7850 or REGN7851) in a composition of the present invention are deamidated to form succinimide. In an embodiment of the invention, less than about 1% of said asparagines are deamidated to form aspartate/isoaspartate.

In an embodiment of the invention, about 2% or less of the methionine 10 residues e.g., the methionine corresponding to SDTGRPFVEMYSEIPEIIHMTEGR (amino acids 1-24 of SEQ ID NO: 12) (underscored) in VEGF mini-traps (e.g., REGN7483, REGN7850 or REGN7851) in a composition of the present invention are oxidized.

In an embodiment of the invention, about 3% or less of the methionine 20 residues e.g., the methionine corresponding to SDTGRPFVEMYSEIPEIIHMTEGR (amino acids 1-24 of SEQ ID NO: 12) (underscored) in VEGF mini-traps (e.g., REGN7483, REGN7850 or REGN7851) in a composition of the present invention are oxidized.

In an embodiment of the invention, about 2% or less of the methionine 163 residues e.g., the methionine corresponding to TQSGSEMK (amino acids 157-164 of SEQ ID NO: 12) (underscored) in VEGF mini-traps (e.g., REGN7483, REGN7850 or REGN7851) in a composition of the present invention are oxidized.

In an embodiment of the invention, about 4.3% or less of the methionine 192 residues e.g., the methionine corresponding to SDQGLYTCAASSGLMTK (amino acids 178-194 of SEQ ID NO: 12) (underscored) in VEGF mini-traps (e.g., REGN7483, REGN7850 or REGN7851) in a composition of the present invention are oxidized.

In an embodiment of the invention, about 0.1%, 0.5%, 1%, 1.5% or 2% of the C-terminal glycines in VEGF mini-traps (e.g., REGN7483, REGN7850 or REGN7851) in a composition of the present invention are lost/missing.

In an embodiment of the invention,
about 1.5% or less of the Arginine 5 residues;
less than about 0.1% of the Lysine 62 residues; and/or
less than about 0.1% of the Lysine 185 residues;
   in VEGF mini-traps (e.g., REGN7483, REGN7850 or REGN7851) in a composition of the present invention are carboxymethylated.

VEGF mini-traps and compositions comprising VEGF mini-traps having any one or more of the following characteristics also form part of the present invention:
Asparagine deamidation, for example, at Asn84 (e.g., about 27%), Asn99 (e.g., about 0.5-1.0%) and/or Asn152 (e.g., about 2.5-3.0%).
Asp Succinimide+isomerization, for example, at Asp173 (e.g., about 2%). For example, wherein Aspartate-Glycine converts to an L-succinamidyl intermediate

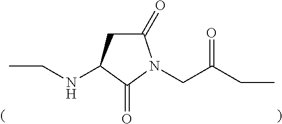

and isomerizes to iso-aspartate-glycine and/or Asn-glycine. See e.g., Stephenson & Clarke, Succinimide Formation from Aspartyl and Asparaginyl Peptides as a Model for the Spontaneous Degradation of Proteins, J. Biol. Chem. 264(11): 6164-6170 (1989).
Methionine oxidation, for example, at Met10 (e.g., about 5-6%), Met20 (e.g., about 2%), Met163 (e.g., about 7%) and/or Met192 (e.g., about 6-7%), for example, at methionine sulfoxide and/or methionine sulfone.
Trp Dioxidation, for example, of Trp58 (e.g., about 0.3%), for example, to form N-formylkynurenin.
Arg 3-deoxyglucosone formation, for example, at Arg5 (e.g., about 8.1%).
C-terminal Glycine loss (e.g., about 7.2%).
Non-glycosylated N-linked glycosites, for example, at Asn36 (e.g., about 1.7%), Asn68 (e.g., about 47.3%), Asn123 (e.g., about 0.2%) and/or Asn196 (e.g., about 0.8%).

### Polynucleotides and Methods of Making

An isolated polynucleotide encoding any VEGF mini-trap polypeptide set forth herein forms part of the present invention as does a vector comprising the polynucleotide and/or a host cell (e.g., Chinese hamster ovary (CHO) cell) comprising the polynucleotide, vector, VEGF mini-trap and/or a polypeptide set forth herein. Such host cells also form part of the present invention.

A polynucleotide includes DNA and RNA. The present invention includes any polynucleotide of the present invention, for example, encoding a VEGF mini-trap polypeptide set forth herein (e.g., any of SEQ ID NOs: 10-13, 26, 27, 28, 30, 32 or 33). Optionally, the polynucleotide is operably linked to a promoter or other expression control sequence. In an embodiment of the invention, a polynucleotide of the present invention is fused to a secretion signal sequence. Polypeptides encoded by such polynucleotides are also within the scope of the present invention.

The present invention includes a polynucleotide comprising the following nucleotide sequence which encodes a precursor VEGF trap which may be cleaved e.g., with an enzyme, to remove the Fc multimerizing component leaving a hinge sequence which may bind to another hinge sequence on a similar molecule, thus creating a homodimeric VEGF mini-trap:

REGN7843-VEGF mini trap-hFc DKTHCPPCPAPELLG
(SEQ ID NO: 14)
agtgataccggtagacctttcgtagagatgtacagtgaaatccccgaaattatacacatgactgaaggaagggagct
cgtcattccctgccgggttacgtcacctaacatcactgttactttaaaaaagtttccacttgacactttgatccctg
atggaaaacgcataatctgggacagtagaaagggcttcatcatatcaaatgcaacgtacaaagaaatagggcttctg
acctgtgaagcaacagtcaatgggcatttgtataagacaaactatctcacacatcgacaaaccaatacaatcataga
tgtggttctgagtccgtctcatggaattgaactatctgttggagaaaagcttgtcttaaattgtacagcaagaactg
aactaaatgtggggattgacttcaactgggaatacccttcttcgaagcatcagcataagaaacttgtaaaccgagac
ctaaaaacccagtctgggagtgagatgaagaaatttttgagcaccttaactatagatggtgtaacccggagtgacca
aggattgtacacctgtgcagcatccagtgggctgatgaccaagaagaacagcacatttgtcagggtccatgaaaagg
acaaaactcacacatgcccaccgtgcccagcacctgaactcctgggg
REGN7850-VEGF mini trap-hFc DKTHCPPCPPC
(SEQ ID NO: 15)
agtgataccggtagacctttcgtagagatgtacagtgaaatccccgaaattatacacatgactgaaggaagggagct
cgtcattccctgccgggttacgtcacctaacatcactgttactttaaaaaagtttccacttgacactttgatccctg
atggaaaacgcataatctgggacagtagaaagggcttcatcatatcaaatgcaacgtacaaagaaatagggcttctg
acctgtgaagcaacagtcaatgggcatttgtataagacaaactatctcacacatcgacaaaccaatacaatcataga
tgtggttctgagtccgtctcatggaattgaactatctgttggagaaaagcttgtcttaaattgtacagcaagaactg
aactaaatgtggggattgacttcaactgggaatacccttcttcgaagcatcagcataagaaacttgtaaaccgagac
ctaaaaacccagtctgggagtgagatgaagaaatttttgagcaccttaactatagatggtgtaacccggagtgacca
aggattgtacacctgtgcagcatccagtgggctgatgaccaagaagaacagcacatttgtcagggtccatgaaaagg
acaaaactcacacatgcccaccgtgcccaccgtgctga
REGN7851-VEGF mini trap-hFc DKTHCPPCPPCPPC
(SEQ ID NO: 16)
agtgataccggtagacctttcgtagagatgtacagtgaaatccccgaaattatacacatgactgaaggaagggagct
cgtcattccctgccgggttacgtcacctaacatcactgttactttaaaaaagtttccacttgacactttgatccctg
atggaaaacgcataatctgggacagtagaaagggcttcatcatatcaaatgcaacgtacaaagaaatagggcttctg
acctgtgaagcaacagtcaatgggcatttgtataagacaaactatctcacacatcgacaaaccaatacaatcataga
tgtggttctgagtccgtctcatggaattgaactatctgttggagaaaagcttgtcttaaattgtacagcaagaactg
aactaaatgtggggattgacttcaactgggaatacccttcttcgaagcatcagcataagaaacttgtaaaccgagac
ctaaaaacccagtctgggagtgagatgaagaaatttttgagcaccttaactatagatggtgtaacccggagtgacca
aggattgtacacctgtgcagcatccagtgggctgatgaccaagaagaacagcacatttgtcagggtccatgaaaagg
acaaaactcacacatgcccaccgtgcccaccgtgcccaccgtgctga

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide of the invention. Promoters which may be used to control gene expression include, but are not limited to, cytomegalovirus (CMV) promoter (U.S. Pat. Nos. 5,385,839 and 5,168,062), the SV40 early promoter region (Benoist et al., (1981) Nature 290:304-310), the promoter contained in the 3′ long terminal repeat of Rous sarcoma virus (Yamamoto et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster et al., (1982) Nature 296:39-42); prokaryotic expression vectors such as the beta-lactamase promoter (VIIIa-Komaroff et al., (1978) Proc. Natl. Acad. Sci. USA 75:3727-3731), or the tac promoter (DeBoer et al., (1983) Proc. Natl. Acad. Sci. USA 80:21-25); see also “Useful proteins from recombinant bacteria” in Scientific American (1980) 242:74-94; and promoter elements from yeast or other fungi such as the Gal4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

The present invention includes polynucleotides encoding VEGF mini-trap polypeptide chains which are variants of those whose nucleotide sequence is specifically set forth herein. A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein (e.g., any of SEQ ID NOs: 14-16); when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1,-2; gap costs: linear). In an embodiment of the invention, a variant of a nucleotide sequence specifically set forth herein comprises one or more (e.g., 1, 2, 3, 4, 5, 6, 7, 8, 9, 10, 11 or 12) point mutations, insertions (e.g., in frame insertions) or deletions (e.g., in frame deletions) of one or more nucleotides relative to any of SEQ ID NOs: 14-16. Such mutations may, in an embodiment of the invention, be missense or nonsense mutations. In an embodiment of the invention, such a variant polynucleotide encodes a VEGF mini-trap polypeptide chain which retains specific binding to VEGF.

Eukaryotic and prokaryotic host cells, including mammalian cells, may be used as hosts for expression of a VEGF mini-trap polypeptide. Such host cells are well known in the art and many are available from the American Type Culture Collection (ATCC). These host cells include, inter alia, Chinese hamster ovary (CHO) cells, CHO K1, EESYR, NICE, NSO, Sp2/0, embryonic kidney cells and BHK cells. The present invention includes an isolated host cell (e.g., a CHO cell or any type of host cell set forth above) comprising one or more VEGF mini-trap polypeptides (or variant thereof) and/or a polynucleotide encoding such a polypeptide(s) (e.g., as discussed herein).

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4399216; 4912040; 4740461 and 4959455. Thus, the present invention includes recombinant methods for making a VEGF mini-trap comprising
(i) introducing, into a host cell, one or more polynucleotides (e.g., including the nucleotide sequence in any one or more of SEQ ID NOs: 14-16; or a variant thereof) encoding a VEGF mini-trap polypeptide, for example, wherein the polynucleotide is in a vector; and/or integrates into the host cell chromosome and/or is operably linked to a promoter;
(ii) culturing the host cell (e.g., CHO or Pichia or Pichia pastoris) under conditions favorable to expression of the polynucleotide and,
(iii) optionally, isolating the VEGF mini-trap or chain thereof from the host cell and/or medium in which the host cell is grown.
   When making a VEGF mini-trap that includes two or more polypeptide chains, co-expression of the chains in a single host cell leads to association of the chains, e.g., in the cell or on the cell surface or outside the cell if such chains are secreted, so as to form homodimeric mini-trap. The present invention also includes VEGF mini-traps which are the product of the production methods set forth herein, and, optionally, the purification methods set forth herein.

There are several methods by which to produce recombinant antibodies which are known in the art. One example of a method for recombinant production of antibodies is disclosed in U.S. Pat. No. 4,816,567. Recombinant VEGF mini-traps (e.g., REGN7483^{R}, REGN7850 or REGN7851) are part of the present invention.

The present invention also provides a method for making a VEGF mini-trap (e.g., a homodimeric VEGF mini-trap) set forth herein, from a VEGF trap (e.g., aflibercept or conbercept), comprising, consisting of or consisting essentially of proteolyzing VEGF Trap with a protease which cleaves the VEGF Trap in the immunoglobulin Fc multimerizing component below (to the C-terminal side of) the Fc hinge domain. For example, the proteolysis can be done with S. pyogenes IdeS (e.g., FabRICATOR protease; Genovis, Inc.; Cambridge, MA; Lund, Sweden) or Streptococcus equi subspecies zooepidemicus IdeZ (New England Biolabs; Ipswich, Mass.). In an embodiment of the invention, such a method lacks any steps that include significant modification of the amino acid residues of such VEGF mini-trap polypeptide (e.g., directed chemical modification such as PEGylation or iodoacetamidation) and/or disulfide bridge reduction. A VEGF mini-trap product of such a method for making is also part of the present invention. For example, in an embodiment of the invention, the Fc domain of a VEGF trap comprises the amino acid sequence: DKTHTCPPCPAPELLG//GPSVFLFPPKPKDTLMISRTPEV TCVVVDVSHEDPEVKFNWYVDGVEVHNAKTKPREEQYNSTYRVVSVLTVLHQ DWLNGKEYKCKVSNKALPAPIEKTISKAKGQPREPQVYTLPPSRDELTKNQVSLTCLVKGFYPSDIAVEWESNGQPE NNYKTTPPVLDSDGSFFLYSKLTVDKSRWQQGNVFSCSVMHEALHNHYTQKSLSLSPGK; wherein the enzyme cleavage site is denoted by “//”.

Such a method for making a VEGF mini-trap may be followed by a method for purifying VEGF mini-trap, e.g., from contaminants such as an Fc fragment (e.g., SEQ ID NO: 19), proteolytic enzyme or other material. See e.g., FIG. 1. In an embodiment of the invention, the method for purifying is done under conditions promoting the formation of homodimeric VEGF mini-trap (e.g., under non-reducing conditions, e.g., in the absence of reducing agents such as dithiothreitol (DTT) or beta-mercaptoethanol). The VEGF mini-trap product of such a method for making and a method for purifying is also part of the present invention. In an embodiment of the invention, purification is performed by a method including chromatographic purification.

In an embodiment of the invention the VEGF trap cleaved with the protease comprises the amino acid sequence:

SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKDKTHTCPPCPAPELLGGPSVFLFPPK
PKDTLMISRTPEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
(SEQ ID NO: 17; optionally wherein K432 is missing)
or
(SEQ ID NO: 18)
GRPFVEMYSEIPEIIHMTEGRELVIPCRVTSPNITVTLKKFPLDTLIPDGKRIIWDSRKGFIISNATYKEIGLLTCE
ATVNGHLYKTNYLTHRQTNTIIDVVLSPSHGIELSVGEKLVLNCTARTELNVGIDFNWEYPSSKHQHKKLVNRDLKT
QSGSEMKKFLSTLTIDGVTRSDQGLYTCAASSGLMTKKNSTFVRVHENLSVAFGSGMESLVEATVGERVRIPAKYLG
YPPPEIKWYKNGIPLESNHTIKAGHVLTIMEVSERDTGNYTVILTNPISKEKQSHVVSLVVYVPPGPGDKTHTCPLC
PAPELLGGPSVFLFPPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCLVKGFYPSDIAVEWES
NGQPENNYKATPPVLDSDGSFFLYSKLTVDKSRWQQGNVFSCSVMHEALHNHYTQKSLSLSPGK

In an embodiment of the invention the VEGF trap is aflibercept (sold commercially as Eylea) or conbercept. See WO2000/75319 or U.S. Pat. No. 9,669,069.

### Combinations and Pharmaceutical Formulations

The present invention provides compositions that include VEGF mini-traps (e.g., REGN7483^{R}, REGN7483^{F}, REGN7850 or REGN7851) in association with one or more ingredients; as well as methods of use thereof and methods of making such compositions. Pharmaceutic formulations comprising a VEGF mini-trap and a pharmaceutically acceptable carrier or excipient are part of the present invention. In an embodiment of the invention, a pharmaceutical formulation of the present invention has a pH of approximately 5.5, 5.6, 5.7, 5.8, 5.9, 6.0, 6.1 or 6.2.

To prepare pharmaceutical formulations of the VEGF mini-traps (e.g., REGN7483^{R}, REGN7483^{F}, REGN7850 or REGN7851), the mini-traps are admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, N.Y.; Weiner and Kotkoskie (2000) Excipient Toxicity and Safety, Marcel Dekker, Inc., New York, N.Y. In an embodiment of the invention, the pharmaceutical formulation is sterile. Such compositions are part of the present invention.

Pharmaceutical formulations of the present invention include a VEGF mini-trap (e.g., REGN7483^{R}, REGN7483^{F}, REGN7850 or REGN7851) and a pharmaceutically acceptable carrier including, for example, water, buffering agents, preservatives and/or detergents.

The present invention provides a pharmaceutical formulation comprising any of the VEGF mini-traps set forth herein (e.g., REGN7483^{R}, REGN7483^{F}, REGN7850 or REGN7851) and a pharmaceutically acceptable carrier; e.g., wherein the concentration of polypeptide is about 40 mg/ml, about 60 mg/ml, about 80 mg/ml; 90 mg/ml; about 100 mg/ml; about 110 mg/ml, about 120 mg/ml, about 133 mg/ml, about 140 mg/ml, about 150 mg/ml, about 200 mg/ml or about 250 mg/ml.

The scope of the present invention includes desiccated, e.g., freeze-dried, compositions comprising a VEGF mini-trap (e.g., REGN7483^{R}, REGN7483^{F}, REGN7850 or REGN7851) or a pharmaceutical formulation thereof that includes a pharmaceutically acceptable carrier but substantially lacks water.

In a further embodiment of the invention, a further therapeutic agent that is administered to a subject in association with a VEGF mini-trap (e.g., REGN7483^{R}, REGN7483^{F}, REGN7850 or REGN7851) disclosed herein is administered to the subject in accordance with the Physicians' Desk Reference 2003 (Thomson Healthcare; 57th edition (Nov. 1, 2002)).

The present invention provides a vessel (e.g., a plastic or glass vial, e.g., with a cap or a chromatography column, hollow bore needle or a syringe cylinder) comprising any of the VEGF mini-traps (e.g., REGN7483^{R}, REGN7483^{F}, REGN7850 or REGN7851) or a pharmaceutical formulation comprising a pharmaceutically acceptable carrier thereof. The present invention also provides an injection device comprising a VEGF mini-trap or formulation set forth herein, e.g., a syringe, a pre-filled syringe or an autoinjector. In an embodiment of the invention, a vessel is tinted (e.g., brown) to block out light.

The present invention includes combinations including a VEGF mini-trap (e.g., REGN7483^{R}, REGN7483^{F}, REGN7850 or REGN7851) in association with one or more further therapeutic agents. The VEGF mini-trap and the further therapeutic agent can be in a single composition or in separate compositions. For example, in an embodiment of the invention, the further therapeutic agent is an Ang-2 inhibitor (e.g., nesvacumab), a Tie-2 receptor activator, an anti-PDGF antibody or antigen-binding fragment thereof, an anti-PDGF receptor or PDGF receptor beta antibody or antigen-binding fragment thereof and/or an additional VEGF antagonist such as aflibercept, conbercept, bevacizumab, ranibizumab, an anti-VEGF aptamer such as pegaptanib (e.g., pegaptanib sodium), a single chain (e.g., V_{L}-V_{H}) anti-VEGF antibody such as brolucizumab, an anti-VEGF DARPin such as the Abicipar Pegol DARPin, a bispecific anti-VEGF antibody, e.g., which also binds to ANG2, such as RG7716, or a soluble form of human vascular endothelial growth factor receptor-3 (VEGFR-3) comprising extracellular domains 1-3, expressed as an Fc-fusion protein.

### Administration and Treatment

The present invention provides methods for treating or preventing a cancer (e.g., whose growth and/or metastasis is mediated, at least in part, by VEGF, e.g., VEGF-mediated angiogenesis) or an angiogenic eye disorder, in a subject, comprising administering a therapeutically effective amount of VEGF mini-trap (e.g., REGN7483^{R}, REGN7483^{F}, REGN7850 or REGN7851) to the subject.

The expression “angiogenic eye disorder,” as used herein, means any disease of the eye which is caused by or associated with the growth or proliferation of blood vessels or by blood vessel leakage.

The term “treat” or “treatment” refers to a therapeutic measure that reverses, stabilizes or eliminates an undesired disease or disorder (e.g., an angiogenic eye disorder or cancer), for example, by causing the regression, stabilization or elimination of one or more symptoms or indicia of such disease or disorder by any clinically measurable degree, e.g., with regard to an angiogenic eye disorder, by causing a reduction in or maintenance of diabetic retinopathy severity score (DRSS), by improving or maintaining vision (e.g., in best corrected visual acuity e.g., as measured by an increase in ETDRS letters), increasing or maintaining visual field and/or reducing or maintaining central retinal thickness and, with respect to cancer, stopping or reversing the growth, survival and/or metastasis of cancer cells in the subject. Typically, the therapeutic measure is administration of one or more doses of a therapeutically effective amount of VEGF mini-trap to the subject with the disease or disorder.

The present invention also provides a method for administering a VEGF mini-trap set forth herein (e.g., REGN7483^{R}, REGN7483^{F}, REGN7850 or REGN7851) to a subject (e.g., a human) comprising introducing the VEGF mini-trap (e.g., about 0.5 mg, 2 mg, 4 mg, 6 mg, 8 mg, 10 mg, 12 mg, 14 mg, 16 mg, 18 mg or 20 mg of the polypeptides, for example, in no more than about 100 μl, e.g., about 50, 70 μl or 100 μl), and optionally a further therapeutic agent, into the body of the subject, e.g., by intraocular injection such as by intravitreal injection.

The present invention provides a method for treating cancer (e.g., whose growth and/or metastasis is mediated, at least in part, by VEGF, e.g., VEGF-mediated angiogenesis) or an angiogenic eye disorder in a subject in need thereof, the method comprising administering a therapeutically effective amount of VEGF mini-trap (e.g., 2 mg, 4 mg, 6 mg, 8 mg or 10 mg, e.g., in no more than about 100 μl) set forth herein, and optionally a further therapeutic agent, to the body of the subject, e.g., into an eye of the subject. In an embodiment of the invention, administration is done by intravitreal injection. Non-limiting examples of angiogenic eye disorders that are treatable or preventable using the methods herein, include:
age-related macular degeneration (e.g., wet or dry),
macular edema,
macular edema following retinal vein occlusion,
retinal vein occlusion (RVO),
central retinal vein occlusion (CRVO),
branch retinal vein occlusion (BRVO),
diabetic macular edema (DME),
choroidal neovascularization (CNV),
iris neovascularization,
neovascular glaucoma,
post-surgical fibrosis in glaucoma,
proliferative vitreoretinopathy (PVR),
optic disc neovascularization,
corneal neovascularization,
retinal neovascularization,
vitreal neovascularization,
pannus,
pterygium,
vascular retinopathy,
diabetic retinopathy in a subject with diabetic macular edema; and
diabetic retinopathies (e.g., non-proliferative diabetic retinopathy (e.g., characterized by a Diabetic Retinopathy Severity Scale (DRSS) level of about 47 or 53) or proliferative diabetic retinopathy; e.g., in an subject that does not suffer from DME).

The mode of administration of a VEGF mini-trap (e.g., REGN7483^{R}, REGN7483^{F}, REGN7850 or REGN7851) or composition thereof can vary. Routes of administration include parenteral, non-parenteral, oral, rectal, transmucosal, intestinal, intramuscular, subcutaneous, intradermal, intramedullary, intrathecal, direct intraventricular, intravenous, intraperitoneal, intranasal, intraocular, inhalation, insufflation, topical, cutaneous, intraocular, intravitreal, transdermal or intra-arterial.

The present invention provides methods for administering a VEGF mini-trap (e.g., REGN7483^{R}, REGN7483^{F}, REGN7850 or REGN7851) to a subject, comprising introducing the mini-trap or a pharmaceutical formulation thereof into the body of the subject. For example, in an embodiment of the invention, the method comprises piercing the body of the subject, e.g., with a needle of a syringe, and injecting the antigen-binding protein or a pharmaceutical formulation thereof into the body of the subject, e.g., into the eye, vein, artery, muscular tissue or subcutis of the subject.

In an embodiment of the invention, intravitreal injection of a pharmaceutical formulation of the present invention (which includes a VEGF mini-trap of the present invention (e.g., REGN7483^{R}, REGN7483^{F}, REGN7850 or REGN7851)) includes the step of piercing the eye with a syringe and needle (e.g., 30-gauge injection needle) containing the formulation and injecting the formulation (e.g., less than or equal to about 100 microliters; about 40, 50, 55, 56, 57, 57.1, 58, 60 or 70 microliters) into the vitreous of the eye (e.g., with a sufficient volume as to deliver a therapeutically effective amount as set forth herein, e.g., of about 2, 4, 6, 8.0, 8.1, 8.2, 8.3, 8.4, 8.5, 8.6, 8.7, 8.8 or 8.9, 10 or 20 mg VEGF mini-trap). Optionally, the method includes the steps of administering a local anesthetic (e.g., proparacaine, lidocaine or tetracaine), an antibiotic (e.g., a fluoroquinolone), antiseptic (e.g., povidone-iodine) and/or a pupil dilating agent to the eye being injected. In an embodiment of the invention, a sterile field around the eye to be injected is established before the injection. In an embodiment of the invention, following intravitreal injection, the subject is monitored for elevations in intraocular pressure, inflammation and/or blood pressure.

The term “in association with” indicates that components, a VEGF mini-trap of the present invention (e.g., REGN7483^{R}, REGN7483^{F}, REGN7850 or REGN7851), along with another agent such as anti-ANG2, can be formulated into a single composition, e.g., for simultaneous delivery, or formulated separately into two or more compositions (e.g., a kit including each component). Components administered in association with each another can be administered to a subject at a different time than when the other component is administered; for example, each administration may be given non-simultaneously (e.g., separately or sequentially) at intervals over a given period of time. Separate components administered in association with each another may also be administered essentially simultaneously (e.g., at precisely the same time or separated by a non-clinically significant time period) during the same administration session. Moreover, the separate components administered in association with each another may be administered to a subject by the same or by a different route.

An effective or therapeutically effective amount of VEGF mini-trap (e.g., REGN7483^{R}, REGN7483^{F}, REGN7850 or REGN7851) for treating or preventing cancer (e.g., which is mediated, at least in part, by angiogenesis) or an angiogenic eye disorder refers to the amount of the VEGF mini-trap sufficient to cause the regression, stabilization or elimination of the cancer or angiogenic eye disorder, e.g., by regressing, stabilizing or eliminating one or more symptoms or indicia of the cancer or angiogenic eye disorder by any clinically measurable degree, e.g., with regard to an angiogenic eye disorder, by causing a reduction in or maintenance of diabetic retinopathy severity score (DRSS), by improving or maintaining vision (e.g., in best corrected visual acuity e.g., as measured by an increase in ETDRS letters), increasing or maintaining visual field and/or reducing or maintaining central retinal thickness and, with respect to cancer, stopping or reversing the growth, survival and/or metastasis of cancer cells in the subject. In an embodiment of the invention, an effective or therapeutically effective amount of VEGF mini-trap for treating or preventing an angiogenic eye disorder is about 0.5 mg, 2 mg, 3 mg, 4 mg, 5 mg, 6 mg, 7 mg, 7.25 mg, 7.7 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, 9 mg, 10 mg, 11 mg, 12 mg, 13 mg, 14 mg, 15 mg, 16 mg, 17 mg, 18 mg, 19 mg or 20 mg, e.g., in no more than about 100 μl. The amount may vary depending upon the age and the size of a subject to be administered, target disease, conditions, route of administration, and the like. In certain embodiments, the initial dose may be followed by administration of a second or a plurality of subsequent doses of VEGF mini-trap in an amount that can be approximately the same or less or more than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

As used herein, the term “subject” refers to a mammal (e.g., rat, mouse, cat, dog, cow, sheep, horse, goat, rabbit), preferably a human, for example, in need of prevention and/or treatment of a cancer or an angiogenic eye disorder. The subject may have cancer or an angiogenic eye disorder or be predisposed to developing cancer or an angiogenic eye disorder.

### Diagnostic Uses

The VEGF mini-traps of the present invention (e.g., REGN7483^{R}, REGN7483^{F}, REGN7850 or REGN7851) may also be used to detect and/or measure VEGF or VEGF-expressing cells in a sample, e.g., for diagnostic purposes. For example, VEGF mini-trap, may be used to diagnose a condition or disease characterized by aberrant expression (e.g., over-expression, under-expression, lack of expression, etc.) of VEGF, e.g., to identify tumor cells and/or tissue expressing VEGF. Exemplary diagnostic assays for VEGF may comprise, e.g., contacting a sample, obtained from a patient, with a VEGF mini-trap of the invention, wherein the VEGF mini-trap is labeled with a detectable label or reporter molecule. The presence of labeled VEGF mini-trap on the sample indicates that VEGF is present on the cells and/or tissue. Alternatively, an unlabeled VEGF mini-trap can be used in diagnostic applications in combination with a secondary antibody (having binding affinity for the VEGF mini-trap) which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. The presence of labeled secondary antibody bound to the VEGF mini-trap on the sample indicates that VEGF is present on the cells and/or tissue. For example, in an embodiment of the invention, such a method includes the steps of contacting a sample containing the cells and/or tissue to be determined for VEGF expression with the VEGF mini-trap and, if binding between the VEGF mini-trap and the cells and/or tissue is observed, determining that the cells and/or tissue express VEGF.

### Conjugates

The invention encompasses VEGF mini-traps (e.g., REGN7483^{R}, REGN7483^{F}, REGN7850 or REGN7851) conjugated to another moiety, e.g., a therapeutic moiety. As used herein, the term “conjugate” refers to a VEGF mini-trap which is chemically or biologically linked to VEGF trap or mini-trap or antibody or antigen-binding fragment thereof, a drug, a radioactive agent, a reporter moiety, an enzyme, a peptide, a protein or a therapeutic agent.

In certain embodiments, the therapeutic moiety may be a cytotoxin, a chemotherapeutic drug, an immunosuppressant or a radioisotope. Cytotoxic agents include any agent that is detrimental to cells. Examples of suitable cytotoxic agents and chemotherapeutic agents for forming immunoconjugates are known in the art, (see for example, WO2005/103081).

Conjugates of VEGF mini-traps linked to a cytotoxin can be used therapeutically to treat cancer. Binding of the conjugated mini-trap to tumor tissue localizes the cytotoxin to the tumor and, thereby, causes the cells of the tumor to die or cease growing and/or metastasizing. Such methods of use of conjugated VEGF mini-traps are part of the present invention.

### EXAMPLES

The following examples are provided for illustrative purposes only and are not intended to limit the scope of the invention. Efforts have been made to ensure accuracy with respect to numbers used, but some experimental errors and deviations should be accounted for. Any formulations set forth in these examples are part of the present invention.

### Example 1: Recombinant Expression of VEGF Mini-Traps

The coding regions of recombinant VEGF mini-traps were linked to a signal sequence and cloned into mammalian expression vectors, transfected into Chinese hamster ovary (CHO-K1) cells and stably transfected pools were isolated after selection with 400 pg/ml hygromycin for 12 days. The stable CHO cell pools, grown in chemically-defined protein-free medium, were used to produce proteins for testing. The recombinant polypeptides were secreted from the cells into the growth medium, cells were depth filtered and then the polypeptides were chromatographically purified from the growth medium and other contaminants.

Sequences of Constituent Domains of the VEGF Mini-Traps
Human Flt1 (accession #NP_001153392.1)
Human Flk1 (accession #NP_002244.1)
Human Fc (IGHG1, accession #P01857-1)

VEGF mini-trap sequences

REGN7483^{F} (Homodimer mini-trap FABricator cleaved from Aflibercept)
hFlt1 Ig Domain 2(S129-D231).hFLK1 Ig Domain 3(V226-K327).hFc(D104-G119)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKDKTHTCPPCPAPELLG
(SEQ ID NO: 12)
REGN7483^{R} (Homodimer mini-trap, recombinant)
hFlt1 Ig Domain 2(S129-D231).hFLK1 Ig Domain 3(V226-K327).hFc(D104-G119)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKDKTHTCPPCPAPELLG
(SEQ ID NO: 12)
REGN7850 (VEGF mini-trap-hFc DKTHCPPCPPC)
hFlt1 Ig Domain 2(S129-D231).hFLK1 Ig Domain 3(V226-K327).hFc(D104-G112).PPC
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKDKTHTCPPCPPC
(SEQ ID NO: 27)
REGN7851 (VEGF mini-trap-hFc DKTHCPPCPPCPPC)
hFlt1 Ig Domain 2(S129-D231).hFLK1 Ig Domain 3(V226-K327).hFc(D104-C112).PPCPPC
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKDKTHTCPPCPPCPPC
(SEQ ID NO: 28)
REGN6824 (hVEGF minitrap-G4Sx3-hVEGF minitrap-mmH)
hFlt1 Ig Domain 2(S129-D231).hFLK1 Ig Domain 3(V226-K327).G4Sx3 Linker Flt1 Ig
Domain 2(S129-D231).hFLK1 Ig Domain 3(V226-K327).mycmyc6His
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKGGGGSGGGGSGGGGSSDTGRPFVEMY
SEIPEIIHMTEGRELVIPCRVTSPNITVTLKKFPLDTLIPDGKRIIWDSRKGFIISNATYKEIGLLTCEATVNGHLY
KTNYLTHRQTNTIIDVVLSPSHGIELSVGEKLVLNCTARTELNVGIDFNWEYPSSKHQHKKLVNRDLKTQSGSEMKK
FLSTLTIDGVTRSDQGLYTCAASSGLMTKKNSTFVRVHEKEQKLISEEDLGGEQKLISEEDLHHHHHH
(SEQ ID NO: 34)
REGN7080 (VEGF minitrap-G4Sx6-VEGF minitrap mmH)
hFlt1 Ig Domain 2(S129-D231).hFLK1 Ig Domain 3(V226-K327).G4SM Linker Flt1 Ig
Domain 2(S129-D231).hFLK1 Ig Domain 3(V226-K327).mycmyc6His
SDTGRPFVEMYSEIPEIIHMTEGRELVIPCRVTSPNITVTLKKFPLDTLIPDGKRIIWDSRKGFIISNATYKEIGLL
TCEATVNGHEYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEK [symbol] [symbol] [symbol]
SDTGRPFVEMYSEIPEIIHMTEGRELVIPCRVTSPNITVTLKKFPLDTLIPDGKRIIWDSRKGFIISNATYKE
IGLLTCEATVNGHLYKTNYLTHRQTNTIIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFVRVHEKEQKEISEEDEGGEQKEISEEDE
HHHHHH
(SEQ ID NO: 35)
REGN7991 (hVEGF minitrap-G4Sx9-hVEGF minitrap)
hFlt1 Ig Domain 2(S129-D231).hFLK1 Ig Domain 3(V226-K327).G4Sx9 Linker.Flt1 Ig
Domain 2(S129-D231).hFLK1 Ig Domain 3(V226-K327)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEK [symbol] [symbol] [symbol] [symbol]
SDTGRPFVEMYSEIPEIIHMTEGRELVIPCRVTSPNITVTLKKFPLDTLIPDGKRIIW
DSRKGFIISNATYKEIGLLTCEATVNGHLYKTNYLTHRQTNTIIDVVLSPSHGIELSVGEKLVLNCTARTELNVGID
FNWEYPSSKHQHKKLVNRDLKTQSGSEMKKFLSTLTIDGVTRSDQGLYTCAASSGLMTKKNSTFVRVHEK
(SEQ ID NO: 32)
REGN7992 (hVEGF minitrap-G4Sx12-hVEGF minitrap)
hFlt1 Ig Domain 2(S129-D231).hFLK1 Ig Domain 3(V226-K327).G4Sx12 Linker.Flt1 Ig
Domain 2(S129-D231).hFLK1 Ig Domain 3(V226-K327)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEK [symbol] [symbol] [symbol] [symbol] [symbol]
SDTGRPFVEMYSEIPEIIHMTEGRELVIPCRVTSPNITVTLKK
FPLDTLIPDGKRIIWDSRKGFIISNATYKEIGLLTCEATVNGHLYKTNYLTHRQTNTIIDVVLSPSHGIELSVGEKL
VLNCTARTELNVGIDFNWEYPSSKHQHKKLVNRDLKTQSGSEMKKFLSTLTIDGVTRSDQGLYTCAASSGLMTKKNS
TFVRVHEK
(SEQ ID NO: 33)

### Example 2: Proteolytic Cleavage of Aflibercept

In order to generate the VEGF mini-trap molecule REGN7483^{F}, immobilised IdeS enzyme (FabRICATOR® obtained from Genovis (Cambridge, Mass.; Lund, Sweden)) was used.

To generate REGN7483^{F}, a column containing the FabRICATOR enzyme was used. Aflibercept (20 mg in 1.0 mL cleavage buffer) was then added to the column and incubated on the column for 30 min at 18° C. After 30 min, the column was washed with cleavage buffer (1.0 mL). The digestion mixture and washing solutions were combined.

The mixture was eluted over an analytical proA column (Applied Biosystems™ POROS™ 20 uM Protein A Cartridge 2.1×30 mm, 0.1 mL (Cat #2-1001-00). The purification can be carried out according to Applied Biosystems'™ protocol for POROS™ 20 uM Protein A Cartridge 2.1×30mm, 0.1mL (Cat #2-1001-00).

### Example 3: Binding Kinetic Analysis of VEGF Mini-Trap And VEGF on Receptor Captured Surface

The ability of various VEGF mini-trap molecules to bind VEGF₁₆₅ was assessed by surface plasmon resonance (SPR).

TABLE 3-1
VEGF Trap Proteins and Ligands Tested

| REGN# | Description | Construct Details |
|---|---|---|
| REGN3 | Full-length VEGF Trap | Flt1 Ig Domain 2(S129-D231).hFLK1 Ig Domain |
| | (Aflibercept) | 3(V226-K327).hIgG1_Fc_v2(D104-K330) |
| REGN7483^{F} | dimer mini-trap from | Same as below |
| | FabRICATOR (IdeS) | |
| | cleavage of REGN3 | |
| REGN7483^{R} | Recombinant dimer | Flt1 Ig Domain 2(S129-D231).hFLK1 Ig Domain |
| | mini-trap | 3(V226-K327).hFc DKTHTCPPCPAPELLG(D104- |
| | | G119) |
| REGN6824 | Single chain mini-trap | Flt1 Ig Domain 2(S129-D231).hFLK1 Ig Domain |
| | with (G₄S)₃ | 3(V226-K327).G4Sx3. |
| | linker and C-terminal | Flt1 Ig Domain 2(S129-D231).hFLK1 Ig Domain |
| | mmH tag | 3(V226-K327).mmH |
| REGN7080 | Single chain mini-trap | Flt1 Ig Domain 2(S129-D231).hFLK1 Ig Domain |
| | with (G₄S)₆ | 3(V226-K327).G4Sx6. |
| | linker and C-terminal | Flt1 Ig Domain 2(S129-D231).hFLK1 Ig Domain |
| | mmH tag | 3(V226-K327).mmH |
| REGN7991 | Single chain mini-trap | Flt1 Ig Domain 2(S129-D231).hFLK1 Ig Domain |
| | with (G₄S)₉. | 3(V226-K327).G4Sx9. |
| | No tag | Flt1 Ig Domain 2(S129-D231).hFLK1 Ig Domain |
| | | 3(V226-K327) |
| REGN7992 | Single chain mini-trap | Flt1 Ig Domain 2(S129-D231).hFLK1 Ig Domain |
| | with (G₄S)₁₂. | 3(V226-K327).G4Sx12. |
| | No tag | Flt1 Ig Domain 2(S129-D231).hFLK1 Ig Domain |
| | | 3(V226-K327) |
| REGN110 | VEGF₁₆₅ | hVEGF₁₆₅(M1-R191) |

VEGF₁₆₅:
(SEQ ID NO: 31)
APMAEGGGQNHHEVVKFMDVYQRSYCHPIETLVDIFQEYP
DEIEYIFKPSCVPLMRCGGCCNDEGLECVPTEESNITMQI
MRIKPHQGQHIGEMSFLQHNKCECRPKKDRARQENPCGPC
SERRKHLFVQDPQTCKCSCKNTDSRCKARQLELNERTCRC
DKPRR

An mmh Tag is myc-myc-His6

Experimental Procedure. Equilibrium dissociation constants (K_{D} values) for human VEGF₁₆₅ binding to various purified VEGF mini-trap constructs were determined using a real-time surface plasmon resonance biosensor using a Biacore 3000 instrument. All binding studies were performed in 10 mM HEPES, 150 mM NaCl, 3 mM EDTA, and 0.05% v/v Surfactant Tween-20, pH 7.4 (HBS-ET) running buffer at 25° C. The Biacore sensor surface was first derivatized by amine coupling with a monoclonal mouse anti-VEGFR1 antibody to capture VEGF mini-trap constructs. Binding studies were performed on the human VEGF reagent, human VEGF₁₆₅ (human VEGF₁₆₅; SEQ ID NO: 31). Different concentrations of VEGF₁₆₅ reagent, prepared in HBS-ET running buffer (2 nM — 62.5 pM; 2-fold serial dilution for human VEGF₁₆₅), were injected over an anti-VEGFR1-captured VEGF mini-trap construct surface for 1.8 minutes at a flow rate of 90 μL/minute, while the dissociation of VEGF mini-trap constructs bound VEGF₁₆₅ reagent was monitored for 60 minutes in HBS-ET running buffer. Kinetic association (kₐ) and dissociation (k_{d}) rate constants were determined by fitting the real-time sensorgrams to a 1:1 binding model using Scrubber 2.0c curve fitting software. Binding dissociation equilibrium constants (K_{D}) and dissociative half-lives (t½) were calculated from the kinetic rate constants as:

$${K}_{D} ( M ) = \frac{k d}{k a} , and t 1 / 2 ( min ) = \frac{ln ( 2 )}{60 * k d}$$

Binding kinetic parameters for human VEGF₁₆₅ binding to different VEGF mini-trap constructs at 25° C. are shown in Tables 1-2 and 1-3.

TABLE 3-2
Binding Kinetic Parameters of Human VEGF165 Binding
to Different VEGF Mini-Trap Constructs at 25° C.

| VEGF mini | | | | | | |
|---|---|---|---|---|---|---|
| trap | Receptor | 100 nM | | | | |
| constructs | Capture | hVEGF₁₆₅ | | | | |
| captured | Level (RU) | Bound | ka(1/Ms) | kd(1/s) | K_{D}(M) | t½(min) |
| REGN3 | 78 ± 0.3 | 21 | 5.60E+06 | ≤1e−5 | 1.79E−12 | ≥1155 |
| REGN7483^{F} | 65 ± 0.8 | 36 | 7.58E+06 | ≤1e−5 | 1.32E−12 | ≥1155 |
| REGN7483^{R} | 68 ± 0.2 | 30 | 6.30E+06 | ≤1e−5 | 1.58E−12 | ≥1155 |
| REGN6824 | 59 ± 1.2 | 33 | 6.44E+06 | 5.54E−04 | 8.61E−11 | 20.9 |
| REGN7080 | 67 ± 0.9 | 36 | 5.41E+06 | 3.87E−04 | 7.15E−11 | 29.8 |

TABLE 3-3
Binding Kinetic Parameters of Human VEGF₁₆₅ Binding to Different VEGF
Mini-Trap Constructs at 25° C.*

| | | BIACORE Binding Kinetics at 25° C. | | | | | |
|---|---|---|---|---|---|---|---|
| | | Trap | | | | | |
| | | Capture | 2nM | | | | |
| VEGF Trap | | Level | hVEGF₁₆₅ | | | | t1/2 |
| format | REGN# | (RU) | Bound | ka(1/Ms) | kd(1/s) | K_{D}(M) | (min) |
| full-length | REGN3 | 70 + 0.7 | 29 | 7.90E+06 | ≥1e-5 | 1.27E-12 | ≥1155 |
| Trap | (aflibercept) | | | | | | |
| Dimer | REGN7483^{F} | 67 + 0.4 | 43 | 7.59E+06 | ≥1e-5 | 1.32E-12 | ≥1155 |
| Mini-Trap | (CPPCPAPELLG) | | | | | | |
| | REGN7483^{R} | 69 + 0.6 | 46 | 6.30E+06 | ≥1e-5 | 1.59E-12 | ≥1155 |
| | (CPPCPAPELLG) | | | | | | |
| | REGN7850 | 73 + 1.0 | 56 | 6.46E+06 | ≥1e-5 | 1.55E-12 | ≥1155 |
| | (CPPCPPC) | | | | | | |
| | REGN7851 | 74 + 1.1 | 51 | 5.51E+06 | ≥1e-5 | 1.81E-12 | ≥1155 |
| | (CPPCPPCPPC) | | | | | | |
| Monomeric | REGN6824 (G₄Sx3 | 59 + 1.2 | 33 | 6.44E+06 | 5.54E-04 | 8.61E-11 | 20.9 |
| single | linker) | | | | | | |
| chain | REGN7080 (G₄Sx6 | 67 + 0.9 | 36 | 5.41E+06 | 3.87E-04 | 7.15E-11 | 29.8 |
| Mini-Trap | linker) | | | | | | |
| | REGN7991 (G₄Sx9 | 55 + 0.4 | 40 | 7.10E+06 | 9.79E-05 | 1.38E-11 | 118 |
| | linker) | | | | | | |
| | REGN7992 (G₄Sx12 | 38 + 0.3 | 22 | 6.30E+06 | 9.70E-05 | 1.53E-11 | 119 |
| | linker) | | | | | | |

Measurements were repeated resulting some variation in reported values

As shown in this Example, certain VEGF mini-traps of the present invention exhibited binding affinities for VEGF molecules that were comparable to full length aflibercept.

### Example 4: Evaluation of the Ability of VEGF Mini-Traps to Block the Activation of VEGFR1 by VEGF₁₁₀, VEGF₁₂₁ and VEGF₁₆₅ in A Luciferase Bioassay

The ability of various VEGF mini-traps to inhibit VEGF₁₁₀, VEGF₁₂₁, and VEGF₁₆₅ mediated activation of VEGFR1 in vitro was assessed.

TABLE 4-1
VEGF Trap Proteins and Ligands Tested

| REGN# | Description | Construct Details |
|---|---|---|
| REGN3 | Full-length VEGF Trap | Flt1 Ig Domain 2(S129-D231).hFLK1 Ig |
| | (aflibercept) | Domain 3(V226-K327).hIgG1_Fc_v2(D104- |
| | | K330) |
| REGN7483^{F} | dimer mini-trap from | Same as below |
| | FabRICATOR (IdeS) cleavage | |
| | of REGN3 | |
| REGN7483^{R} | Recombinant dimer mini-trap | Flt1 Ig Domain 2(S129-D231).hFLK1 Ig |
| | | Domain 3(V226-K327).hFc |
| | | DKTHTCPPCPAPELLG(D104-G119) |
| REGN6824 | Single chain mini-trap with | Flt1 Ig Domain 2(S129-D231).hFLK1 Ig |
| | (G₄S)₃ linker and Cter mmH tag | Domain 3(V226-K327).G4Sx3. |
| | | Flt1 Ig Domain 2(S129-D231).hFLK1 Ig |
| | | Domain 3(V226-K327).mmH |
| REGN7080 | Single chain mini-trap with | Flt1 Ig Domain 2(S129-D231).hFLK1 Ig |
| | (G₄S)₆ linker and Cter mmH tag | Domain 3(V226-K327).G4Sx6. |
| | | Flt1 Ig Domain 2(S129-D231).hFLK1 Ig |
| | | Domain 3(V226-K327).mmH |
| REGN7991 | Single chain mini-trap with | Flt1 Ig Domain 2(S129-D231).hFLK1 Ig |
| | (G₄S)₉ linker | Domain 3(V226-K327).G4SX9 linker.FIt1 Ig |
| | | Domain 2(S129-D231).hFLK1 Ig Domain 3- |
| | | (V226-K327) |
| REGN7992 | Single chain mini-trap with | Flt1 Ig Domain 2(S129-D231).hFLK1 Ig |
| | (G₄S)₁₂ linker | Domain 3(V226-K327).G4SX12 linker.Flt1 |
| | | Ig Domain 2(S129-D231).hFLK1 Ig Domain |
| | | 3-(V226-K327) |
| REGN7850 | homodimer mini Trap | |
| | recombinantly expressed from | |
| | CHO. Cter DKTHTCPPCPPC | |
| | (extra Cys) | |
| REGN7851 | homodimer mini Trap | |
| | recombinantly expressed from | |
| | CHO. Cter | |
| | DKTHTCPPCPPCPPC (2 extra | |
| | Cys) | |
| REGN110 | VEGF₁₆₅ | hVEGF₁₆₅ (M1-R191) |
| | VEGF₁₂₁ | hVEGF₁₂₁ (aa 207-327) |
| | VEGF₁₁₀ | hVEGF₁₁₀ (aa 207-318) |

Experimental Procedure.

Cell Line

The cell line, HEK293/D9/Flt-IL18Rα/Flt-IL18Rβ clone V3H9 was constructed with two chimeric receptors incorporating the VEGFR1 extracellular domain fused to the cytoplasmic domain of either IL18Rα or IL18Rβ. The chimeric receptors were transfected into a cell line with an integrated NFκB-luciferase-IRES-eGFP reporter gene. The extracellular VEGFR1 is dimerized upon binding VEGF, resulting in interaction of the IL18Rα and IL18Rβ intracellular domains, NFκB signaling, and subsequent luciferase production.

Assay Procedure

HEK293/D9/Flt-IL18Rα/Flt-IL18Rβ clone V3H9 cells were plated in 96-well white opaque plates (Nunc, Cat #136101) at 10,000 cells/well in OptiMEM (Invitrogen, Cat #31985) with 0.5% FBS (Seradigm, Cat #1500-500) and incubated at 37° C., 5% CO₂ overnight. The next day, cells were differentially treated with a 1:3 serial dilution of VEGF-Trap or mini-trap protein ranging in concentration from 5000 pM to 0.085 pM, followed by the addition of a fixed concentration of 20 pM VEGF₁₁₀ (R&D Systems Cat #298-VS), VEGF₁₂₁ (R&D Systems Cat #4644-VS) or VEGF₁₆₅ (R&D Systems Cat #293-VE) ligand protein and incubated for 6 hours at 37° C., 5% CO₂. One-Glo luciferase substrate (Promega, Cat #E6130) was then added to the cells and luminescence was measured using a VICTOR™ X5 Multilabel plate reader (PerkinElmer, Model 2030-0050). Data were analyzed using a 4-parameter logistic equation over an 11-point response curve with GraphPad Prism software to determine EC₅₀ and IC₅₀ values.

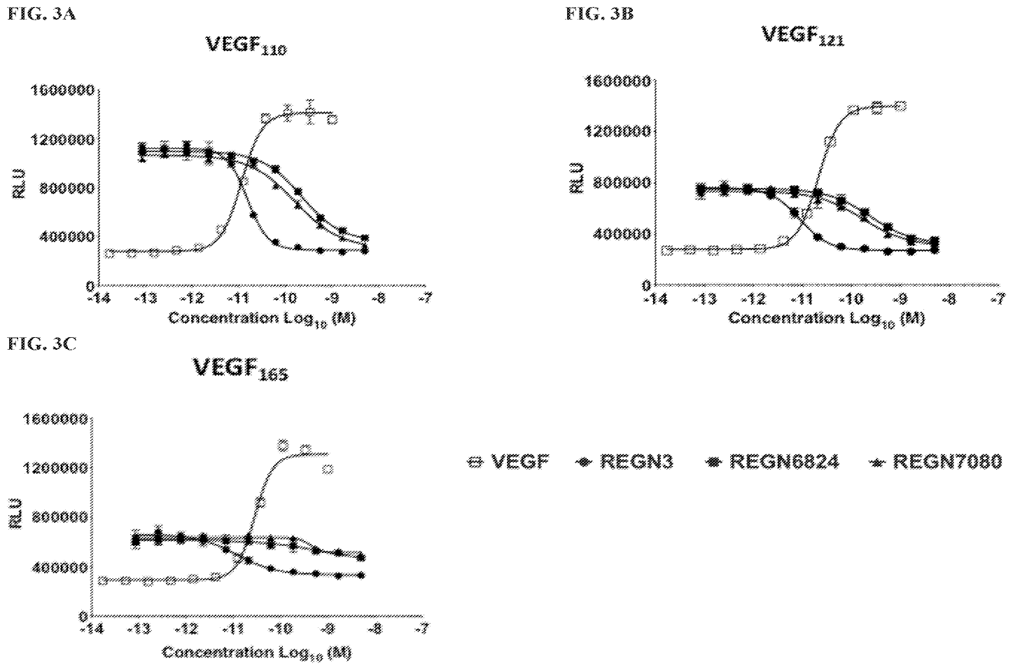

Results summary and conclusions. VEGF₁₁₀, VEGF_{121,} and VEGF₁₆₅ activated HEK293/D9/Flt-IL18Rα/Flt-IL18Rβ clone V3H9 cells with EC50 values of ˜11-24 pM, ˜21-44 pM, and ˜28-43 pM, respectively (FIGS. 3-5, Tables 4-2, 4-4 and 4-6) across experiments.

Single chain mini-traps with a (G₄5)₃ linker (REGN6824) or (G₄5)₆ linker (REGN7080) inhibited VEGFR1 signaling in the presence of 20 pM VEGF₁₁₀ or 20 pM VEGF₁₂₁ at IC50 values of ˜0.2 nM, and partially blocked in the presence of VEGF₁₆₅. (FIG. 3 and Table 4-2 and 4-3)

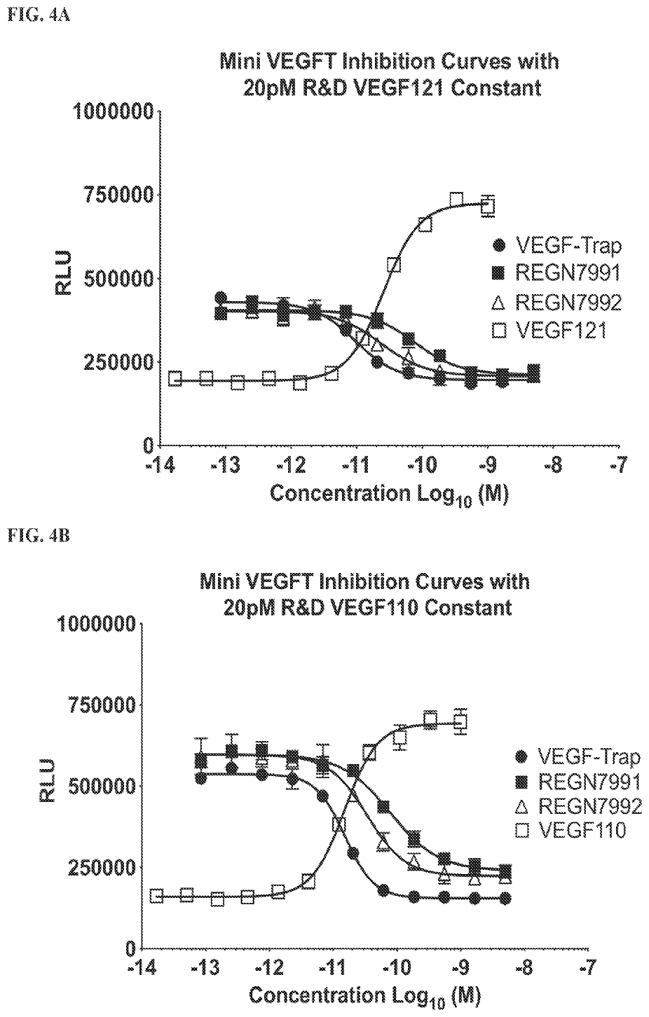

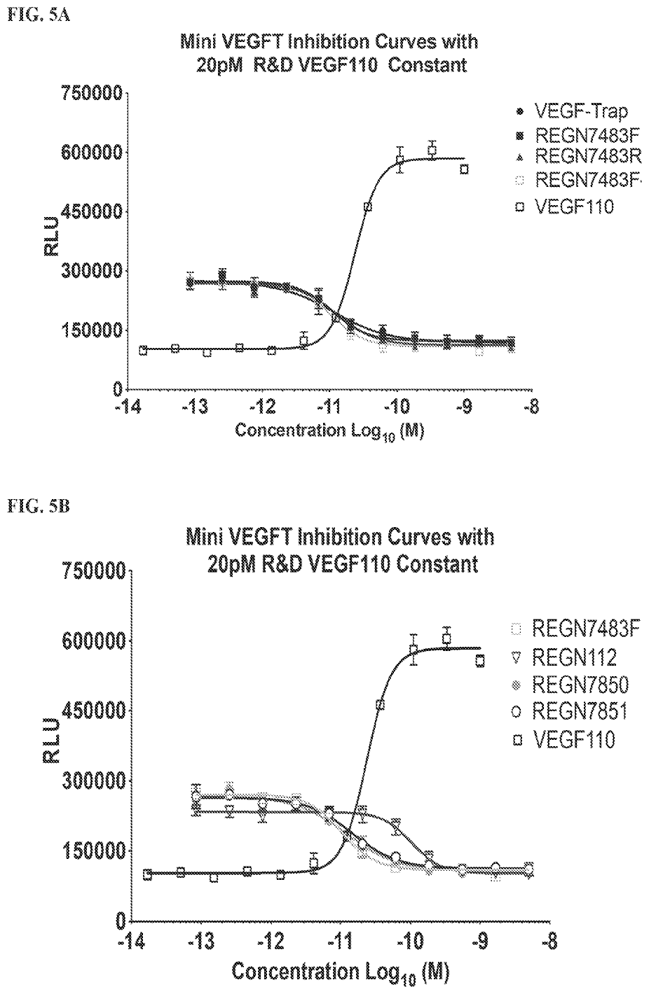

Single chain min-traps with longer G₄5 linkers ((G₄S)₉ or (G₄S)₁₂) (REGN7991 and REGN7992) inhibited VEGFR1 signaling with improved IC₅₀ values ranging from ˜24 to ˜79 pM (FIG. 4(A-B) and Table 4-4 and 4-5).

VEGF Trap (REGN3; aflibercept) inhibited signaling of the VEGF isoforms at IC50 values ranging from ˜9-21 pM across experiments.

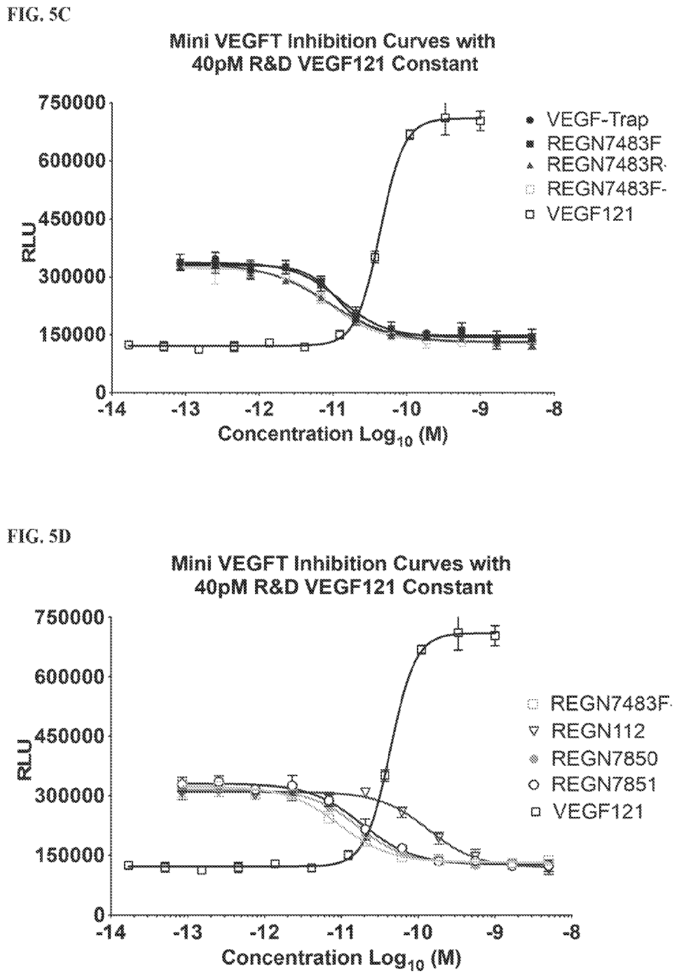

The inhibitory activity of the FabRICATOR cleaved mini-trap, REGN7483^{F}, the recombinant dimer mini-trap, REGN7483^{R}, and REGN7850 and REGN7851, were compared to that of VEGF Trap (aflibercept). REGN7843F, REGN7483^{R}, REGN7850 and REGN7851 inhibited VEGF₁₁₀, VEGF_{121,} and VEGF₁₆₅ mediated activation of VEGFR1 with similar IC₅₀ values to that observed with full length VEGF Trap (FIG. 5 and Table 4-6 and 4-7). Inhibition of VEGF₁₁₀, VEGF₁₂₁, and VEGF₁₆₅ mediated activation of VEGFR1 was observed with IC₅₀ values of ˜9-12 pM for REGN7483^{F}, ˜8-19 pM for REGN7483^{R}; ˜12-30 pM for REGN7850 and ˜15-27 pM for REGN7851.

Data were gathered in three separate experiments which are set forth below.

Bioassay Experiment 1

TABLE 4-2
Activation of HEK293/D9/Flt-IL18Ra/Flt-
IL18Rb with Various VEGF Variants

| VEGF Dose Response | VEGF₁₁₀ | VEGF₁₂₁ | VEGF₁₆₅ |
|---|---|---|---|
| EC₅₀ (M) | 1.14E−11 | 2.12E−11 | 2.80E−11 |

TABLE 4-3
Inhibition VEGFR1 Signaling by Aflibercept or a Single-chain
Mini-trap in the Presence of VEGF110, VEGF121 or VEGF165

| | IC₅₀ (M) | IC₅₀ (M) | IC₅₀ (M) |
|---|---|---|---|
| | 20 pM | 20 pM | 20 pM |
| REGN# | VEGF₁₁₀ | VEGF₁₂₁ | VEGF₁₆₅ |
| REGN3 | 1.53E−11 | 8.94E−12 | 1.18E−11 |
| REGN7080 | 1.61E−10 | 1.66E−10 | IC₅₀ not determined |
| | | | Partial Blocker |
| REGN6824 | 2.22E−10 | 2.44E−10 | IC₅₀ not determined |
| | | | Partial Blocker |

Bioassay Experiment 2

TABLE 4-4
Activation of HEK293/D9/Flt-IL18Ra/Flt-
IL18Rb with Various VEGF Variants

| VEGF Dose Response | VEGF₁₁₀ | VEGF₁₂₁ | VEGF₁₆₅ |
|---|---|---|---|
| EC₅₀ (M) | 1.509E−11 | 2.559E−11 | Not tested |

TABLE 4-5
Inhibition VEGFR1 Signaling by Aflibercept or A Single-
Chain Mini-Trap in the Presence of VEGF110 or VEGF121

| | IC₅₀ (M) | IC₅₀ (M) | IC₅₀ (M) |
|---|---|---|---|
| | 20 pM | 20 pM | 20 pM |
| REGN# | VEGF₁₁₀ | VEGF₁₂₁ | VEGF₁₆₅ |
| REGN3 | 1.528E−11 | 9.639E−12 | Not tested |
| REGN7991 | 7.880E−11 | 7.714E−11 | Not tested |
| REGN7992 | 3.423E−11 | 2.374E−11 | Not tested |

Bioassay Experiment 3

TABLE 4-6
Activation of HEK293/D9/Flt-IL18Ra/Flt-
IL18Rb with Various VEGF Variants

| VEGF Dose Response | VEGF₁₁₀ | VEGF₁₂₁ | VEGF₁₆₅ |
|---|---|---|---|
| EC₅₀ (M) | 2.4E−11 | 4.4E−11 | 4.3E−11 |

TABLE 4-7
Inhibition VEGFR1 Signaling by Aflibercept or A Mutated Dimeric Mini-Trap
in the Presence of VEGF110, VEGF121 or VEGF165 (The different C-terminal amino
acids are underlined)

| | | IC50 (M) | IC50 (M) | IC50 (M) |
|---|---|---|---|---|
| VEGF Trap | | 20 pM | 20 pM | 20 pM |
| format | REGN# | VEGF₁₁₀ | VEGF₁₂₁ | VEGF₁₆₅ |
| Full-length trap | REGN3 | 1.146E-11 | 1.265E-11 | 2.089E-11 |
| | (aflibercept) | | | |
| Dimer | REGN7483^{F} (FabRICATOR) | 1.040E-11 | 9.274E-12 | 1.231E-11 |
| mini-trap | (CPPCPAPELLG) | | | |
| | REGN7483^{R} (recombinant) | 1.298E-11 | 8.793E-12 | 1.922E-11 |
| | (CPPCPAPELLG) | | | |
| | REGN7850 | 1.217E-11 | 1.665E-11 | 2.988E-11 |
| | (CPPCPPC) | | | |
| | REGN7851 | 1.454E-11 | 1.833E-11 | 2.676E-11 |
| | (CPPCPPCPPC) | | | |

VEGF Mini-Trap Key:
REGN7483F = homodimer mini Trap fabricator cleaved from aflibercept [symbol] DKTHTCPPCPAPELLG
REGN7483R = homodimer mini Trap recombinantly expressed from CHO. [symbol] DKTHTCPPCPAPELLG
REGN7850 = homodimer min Trap recombinantly expressed from CHO. [symbol] DKTHTCPPCPPC (extra Cys)
REGN7851 = homodimer min Trap recombinantly expressed from CHO. [symbol] DKTHTCPPCPPCFPC (2 extra Cys)
REGN6824 = monomer single chain mini trap with (G4S)3 linker between two VEGFR1(d2)-R2(d3)
REGN7080 = monomer single chain mini trap with (G4S)6 linker between two VEGFR1(d2)-R2(d3)
REGN7991 = monomer single chain mini trap with (G4S)9 linker between two VEGFR1(d2)-R2(d3)
REGN7992 = monomer single chain mini trap with (G4S)12 linker between two VEGFR1(d2)-R2(d3)

This Example demonstrates that certain VEGF mini-traps of the present invention exhibited equivalent or better potency in regards to blocking VEGF-mediated VEGFR1 activity.

### Example 5: Size Analysis of In Vitro Complexes Formed between VEGF Mini-Trap and VEGF by Size Exclusion Chromatography Coupled to Multi-Angle Light Scattering (SEC-MALS)

The stoichiometry of various VEGF mini-trap molecules with VEGF was determined.

TABLE 5-1
VEGF Trap Proteins and Ligands Tested

| REGN# | Description | Construct Details |
|---|---|---|
| REGN7483^{F} | Disulfide-linked homodimer mini- | Flt1 Ig Domain 2(S129-D231).hFLK1 |
| | trap from FabRICATOR (IdeS) | Ig Domain 3(V226-K327).hFc |
| | cleavage of full-length VEGF Trap | DKTHTCPPCPAPELLG(D104- |
| | REGN3 | G119) |
| REGN6824 | Single chain monomer mini-trap | Flt1 Ig Domain 2(S129-D231).hFLK1 |
| | with (G₄S)₃ | Ig Domain 3(V226-K327).G4Sx3. |
| | linker and Cter mmH | Flt1 Ig Domain 2(S129-D231).hFLK1 |
| | tag | Ig Domain 3(V226-K327).mmH |
| REGN7080 | Single chain monomer mini-trap | Flt1 Ig Domain 2(S129-D231).hFLK1 |
| | with (G₄S)₆ | Ig Domain 3(V226-K327).G4Sx6. |
| | linker and Cter mmH tag | Flt1 Ig Domain 2(S129-D231).hFLK1 |
| | | Ig Domain 3(V226-K327).mmH |
| REGN110 | VEGF₁₆₅ | hVEGF₁₆₅(M1-R191) |

Experimental Procedure.

Size Exclusion Chromatography with Multi Angle Light Scattering (SEC-MALS) Titrations

To understand the stoichiometry of the different mini-trap-VEGF complexes, a series of solutions containing mini-trap and VEGF proteins at different molar ratios was prepared as indicated in Table 5-3 and incubated them overnight at 4° C. The complexes that were studied were as follows: REGN110-REGN7483^{F}, REGN110-REGN6824 and REGN110-REGN7080. Control samples containing REGN110, REGN7483^{F}, REGN6824 and REGN7080 alone were prepared in the same manner. The incubated samples were injected into a SEC-MALS system composed of a miniDAWN Treos MALS device and Optilab T-rEX (refractive index measurement) (Wyatt Technology Corporation) coupled to a Superose 12 Inc 10/300 GI column operated with an ÄKTA micro system (GE Healthcare Life Sciences). The column running buffer for all samples was 10 mM Phosphate pH 7.0, 500 mM NaCl. 100 ug BSA (Bovine Serum Albumin, Thermo Scientific) was injected separately as a standard of known molecular weight to calibrate the MALS measurement. Size exclusion chromatography data were evaluated using Unicorn (Version 5.20 GE Healthcare Life Sciences) by plotting mAU (Absorbance at 280 nm) vs. retention volume (ml). The MALS data was evaluated using ASTRA (Version 7.0.0.69 Wyatt Technology) by plotting the molar mass vs. volume (ml) and Rayleigh ratio vs. volume (ml).

Results summary and conclusions. SEC-MALS was used to assess the molar mass and elution profile of complexes formed between the different versions of mini-trap (REGN7483^{F}, REGN6824, REGN7080) and VEGF (REGN110). Table 5-2 provides the theoretical expected molar mass (calculated from the peptide sequence, not including glycosylation), the observed molar mass for the VEGF mini-trap proteins and REGN110 as well as the oligomeric states of the reagents. Table 5-3 shows the observed weight-averaged molar mass for each peak in the chromatograms for the complexes analyzed.

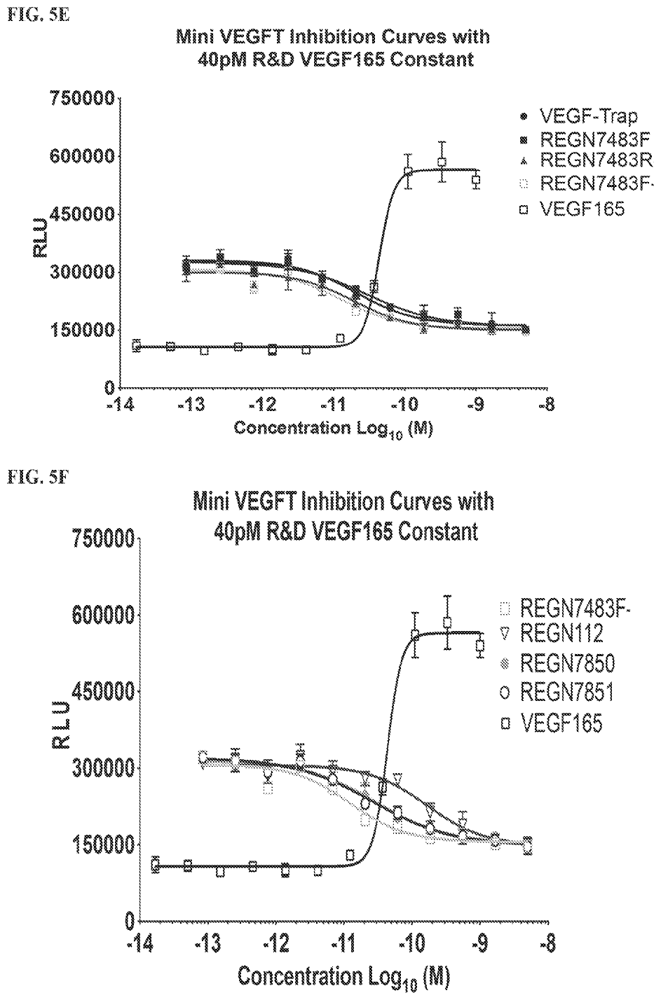

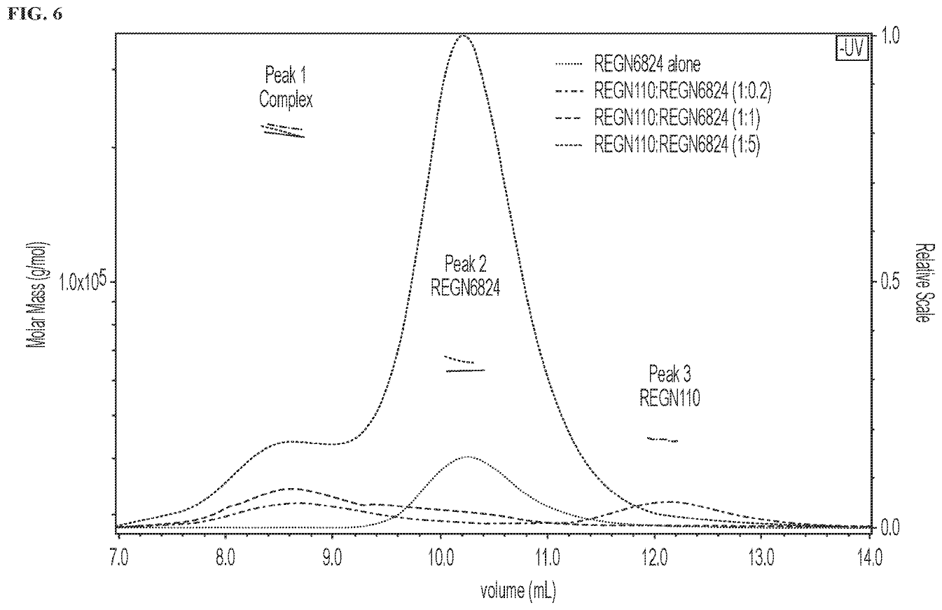

REGN110 eluted as a single peak of ˜42 kDa, consistent with the disulfide-linked homodimer that the literature has shown to be the primary species of VEGF (FIGS. 6-8, Peak 3). The mini-trap proteins ran as monomers with a molar mass of ˜63 kDa, which is consistent with their theoretical peptide molar mass of 50-51kDa and an additional ˜12kDa that is contributed by 8 N-linked glycosylations (FIGS. 6-8, Peak 2). REGN7483^{F}is expected to be a disulfide-linked homodimer, since FabRICATOR cleavage does not break apart the hinge disulfides in the Fc domain of intact REGN3.

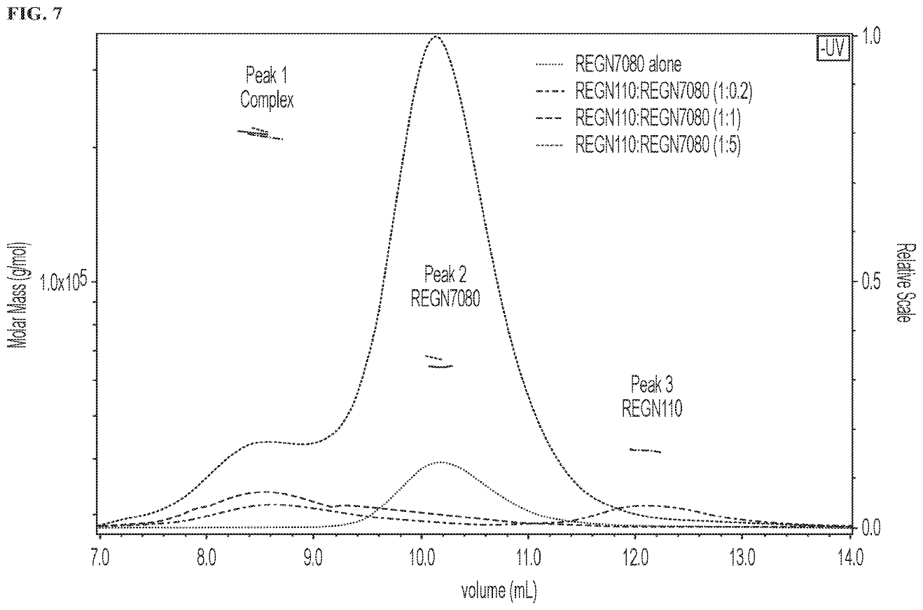

REGN6824 and REGN7080 formed similar complexes with REGN110 under all conditions tested (Table 5-3; FIGS. 6 and 7). When the single chain monomer (REGN6824) was combined with a molar equivalent of the VEGF homodimer (REGN110), a peak (FIG. 6, Peak 1) with a molar mass of -215 KDa was observed, which suggested a complex of 2 REGN6824 molecules bound to 2 REGN110 homodimers. Similar results were observed for REGN7080. At different molar ratios, where REGN110 or REGN6824/REGN7080 are present in excess, the only complex species observed is the 2:2 complex of ˜215 kDa, along with peaks representing excess VEGF or excess mini-trap.

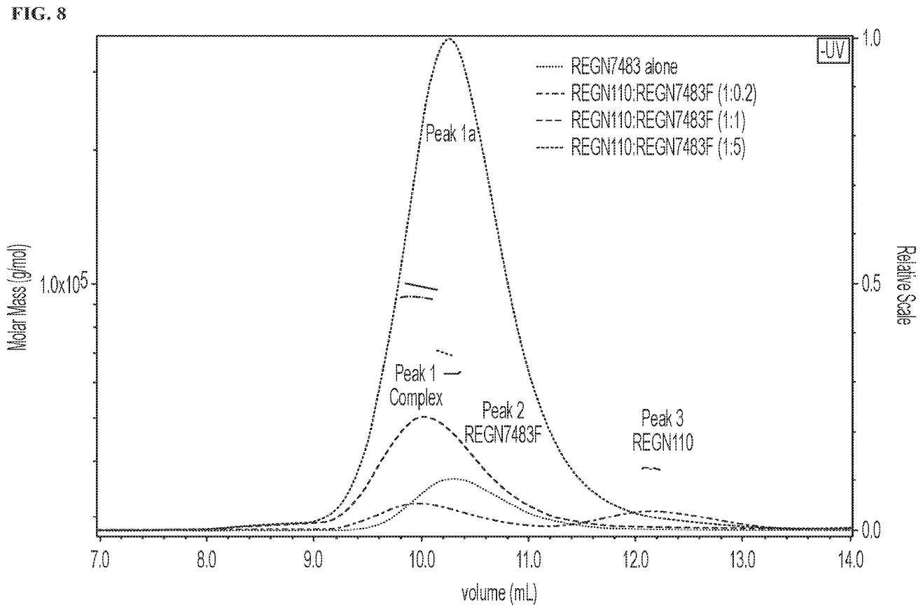

On the other hand, REGN7483^{F} showed a ˜99 KDa complex peak when it was combined with either an equimolar or excess amount of REGN110 (FIG. 8, Peak 1). This peak is consistent with a complex of one REGN7483^{F} disulfide-linked homodimer bound to one REGN110 homodimer. In the presence of excess REGN7483^{F} (FIG. 8, Peak 1a) the MALS peak has a molar mass around ˜70 kDa. In this case, the Superose 12 column could not fully separate the REGN7483^{F}+REGN110 complex from excess REGN7483^{F}; therefore, the observed molar mass represented an average between the complex (99 kDa) and REGN7483^{F} alone (63 kDa).

TABLE 5-2
Summary Table of Approximate Molar Mass
of Mini-Trap Proteins and Ligand Tested

| | Theoretical | Observed | Oligomeric |
|---|---|---|---|
| Sample | M_{w}, kDa | M_{w}, kDa | state |
| REGN110 | 38.6 (Dimer) | 42.5 | Homodimer |
| REGN6824 | 50.3 (8 N-linked Glycos) | 63 | Monomer |
| REGN7080 | 51.3 (8 N-linked Glycos) | 64.2 | Monomer |
| REGN7483^{F} | 49.9 (Dimer, 8 N-linked | 62.9 | Homodimer |
| | Glycos) | | |

kDa: kilodalton; Mw: Weight Average Molar Mass.

TABLE 5-3
Summary Table of Approximate Molar Mass
of Mini-Trap Complexes with REGN110

| | | Observed M_{w}, kDa | | |
|---|---|---|---|---|
| Sample composition (molar ratio)* | | Peak 1 | Peak 2 | Peak 3 |
| REGN110:REGN6824 | (1 dimer:0.2 monomer) | 221 | NA | 44 |
| REGN110:REGN6824 | (1 dimer:1 monomer) | 213 | NA | NA |
| REGN110:REGN6824 | (1 dimer:5 monomer) | 218 | 66 | NA |
| REGN110:REGN7080 | (1 dimer:0.2 monomer) | 211 | NA | 41 |
| REGN110:REGN7080 | (1 dimer:1 monomer) | 216 | NA | NA |
| REGN110:REGN7080 | (1 dimer:5 monomer) | 220 | 67 | NA |
| REGN110:REGN7483^{F} | (1 dimer:0.2 dimer) | 93 | NA | 38 |
| REGN110:REGN7483^{F} | (1 dimer:1 dimer) | 99 | NA | NA |
| REGN110:REGN7483^{F} | (1 dimer:5 dimer) | 70 | NA | NA |

kDa: kilodalton; Mw: Weight Average Molar Mass; NA: not applicable.
*REGN6824 and REGN7080 are single polypeptide chains; REGN110 and REGN7483^{F} are covalent (disulfide-linked) homodimers.

### Example 6: Intravitreal and Systemic Administration of VEGF Trap and Dimer Mini-Trap in a Mouse OIR Model

Mouse pups were placed in a hyperoxic environment (75% O₂) at postnatal day 6 (P6; post-natal day #6) and returned to room air (21% O₂) at P11. This leads to pathological neovascularization over the next several days. Pups were injected intravitreally at P13 with equimolar doses of:
VEGF Trap (aflibercept) (.25μg/eye, n=3),
single-chain mini-trap (REGN7080) (.125μg/eye, n=3),
dimer-mini-trap (REGN7483^{F}) (.125μg/eye, n=3), or
a control protein, hFc (.125μg/eye, n=3);
or
systemically (intra-peritoneally), on P12, with:
3 mg/kg control protein, hFc,
3 mg/kg dimer mini-trap (REGN7483^{F}),
30 mg/kg dimer mini-trap (REGN7483^{F}); or
100 mg/kg dimer mini-trap (REGN7483^{F}).

At P16, eyes were harvested. Retinas were dissected, stained with FITC-labeled Griffornia simplicifolia lectin I (Vector Laboratories) and flat-mounted with Prolong Gold (Invitrogen). To measure abnormal area, flat-mounts were imaged with Nikon 80i with a 4x objective and the area of retinal neovascularization was quantified with image analysis software (Adobe Photoshop CC 2015 extended).

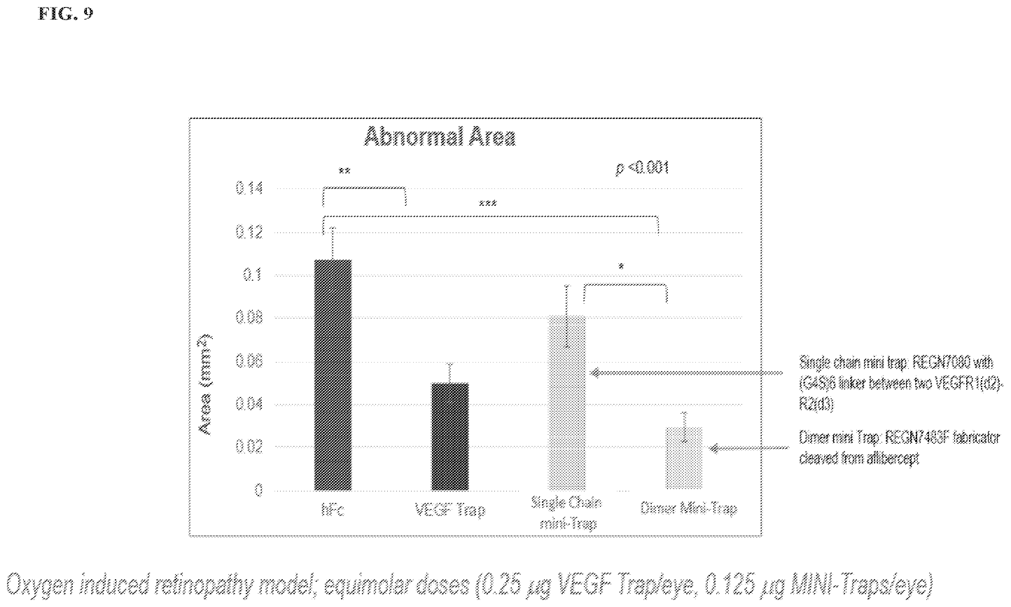

The area of abnormal vascularization (mm²) in mice administered human Fc control, aflibercept (VEGF Trap), single chain mini-trap having a (G₄S)₆ linker between two VEGFR1(d2)-VEGFR2(d3) fusion proteins or dimeric mini-trap which is the product of FabRICATOR protease cleavage of aflibercept (Dimer mini-trap) was evaluated. The dimer mini-trap performed significantly better than the single chain mini-trap and aflibercept in reducing the area of abnormal vascularization in the mouse retina. See FIG. 9.

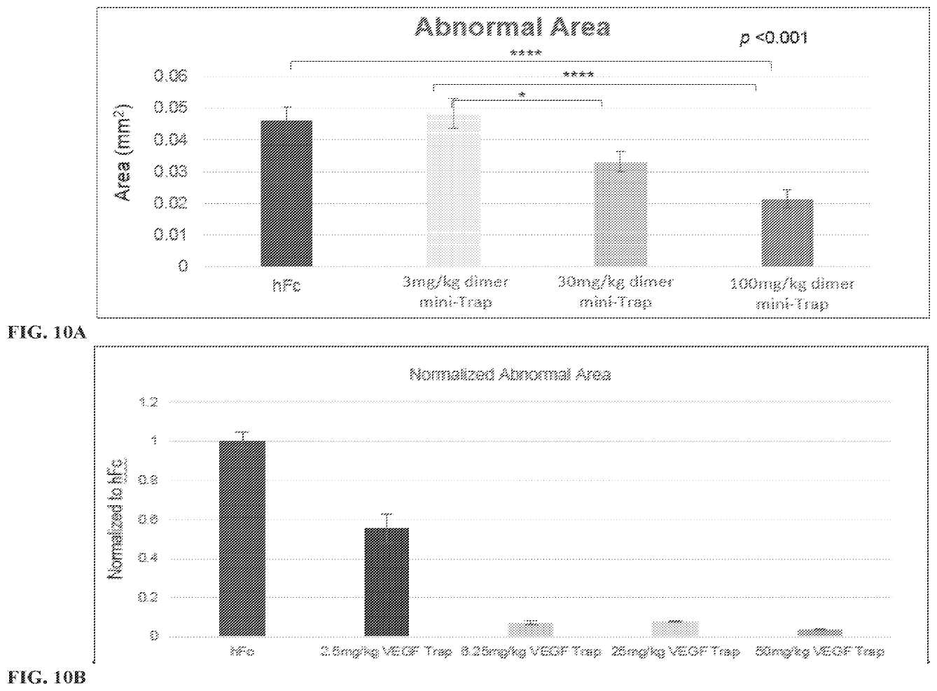

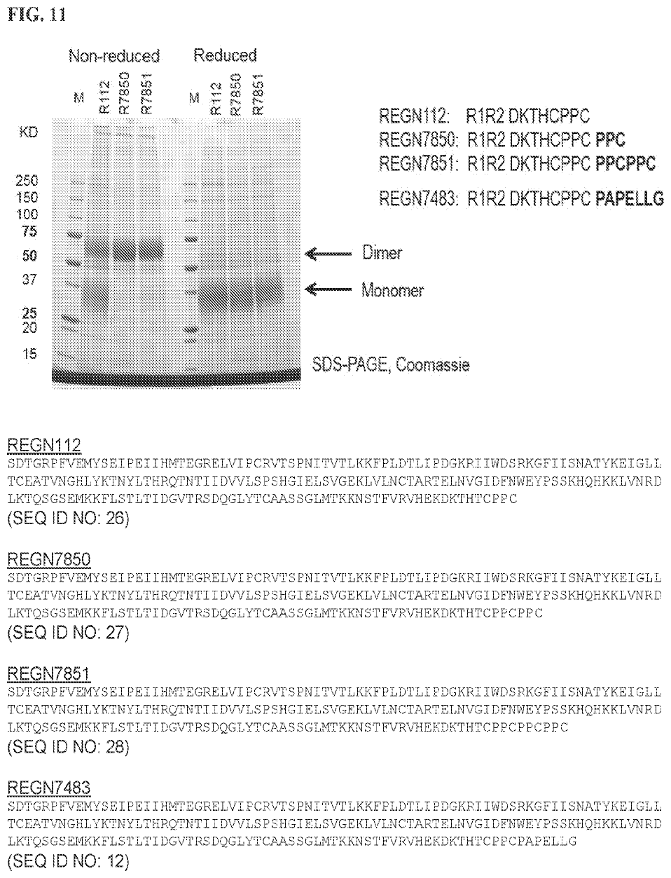

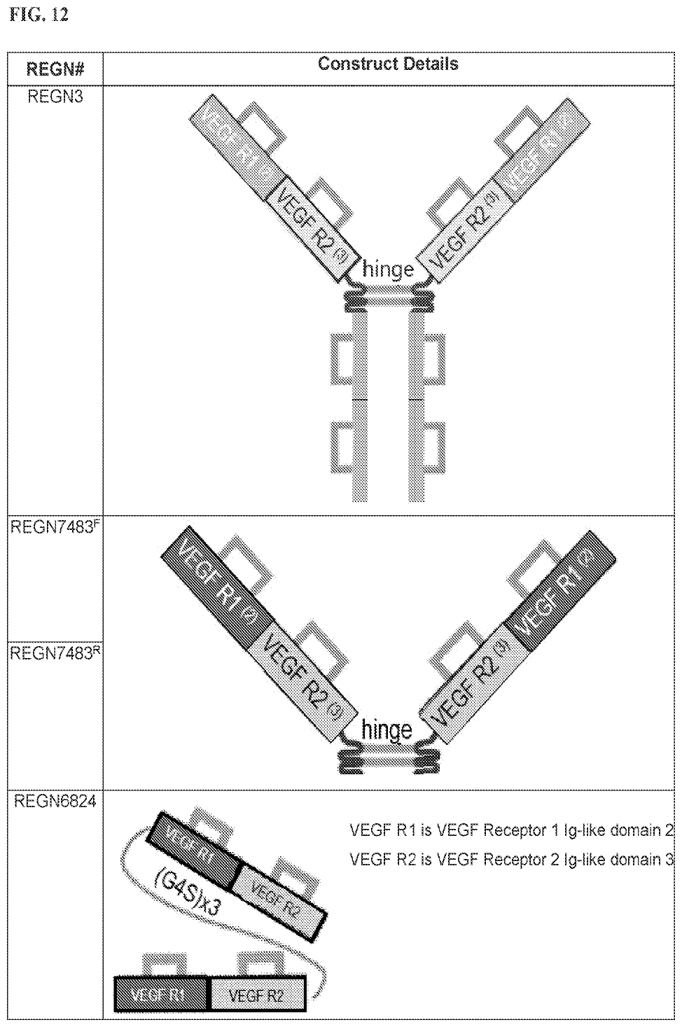

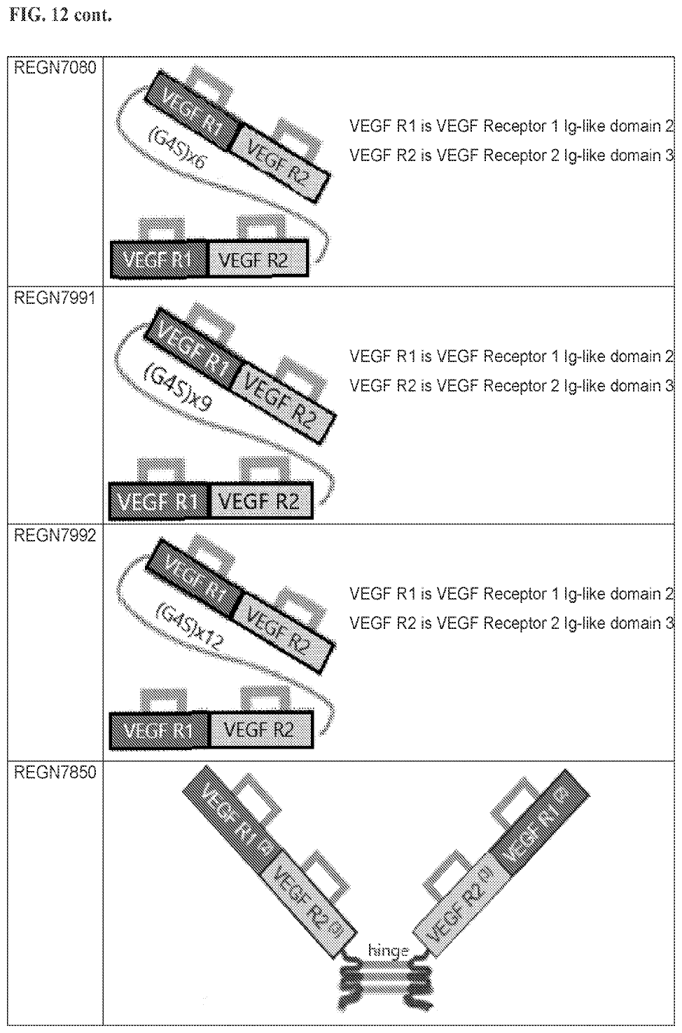

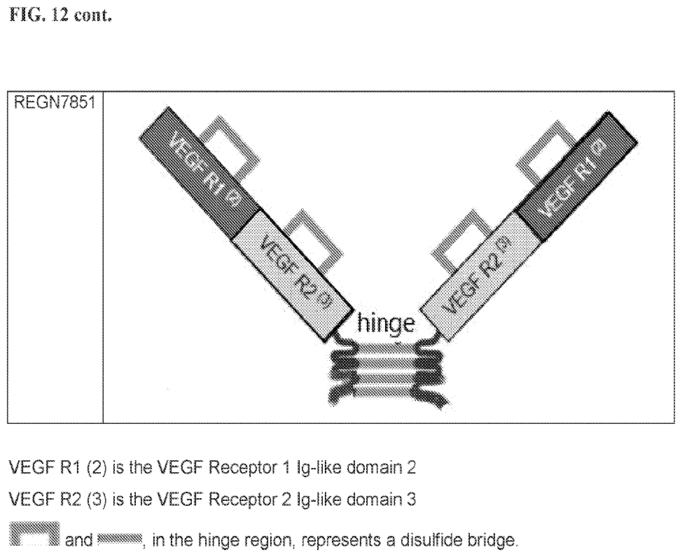

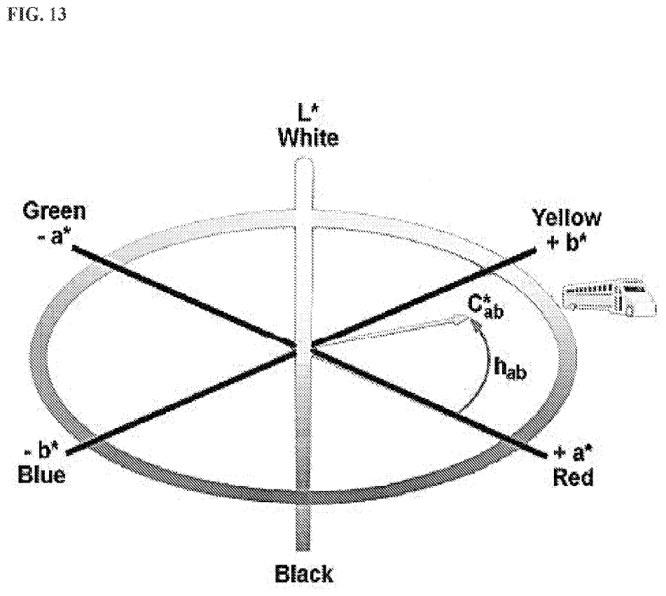

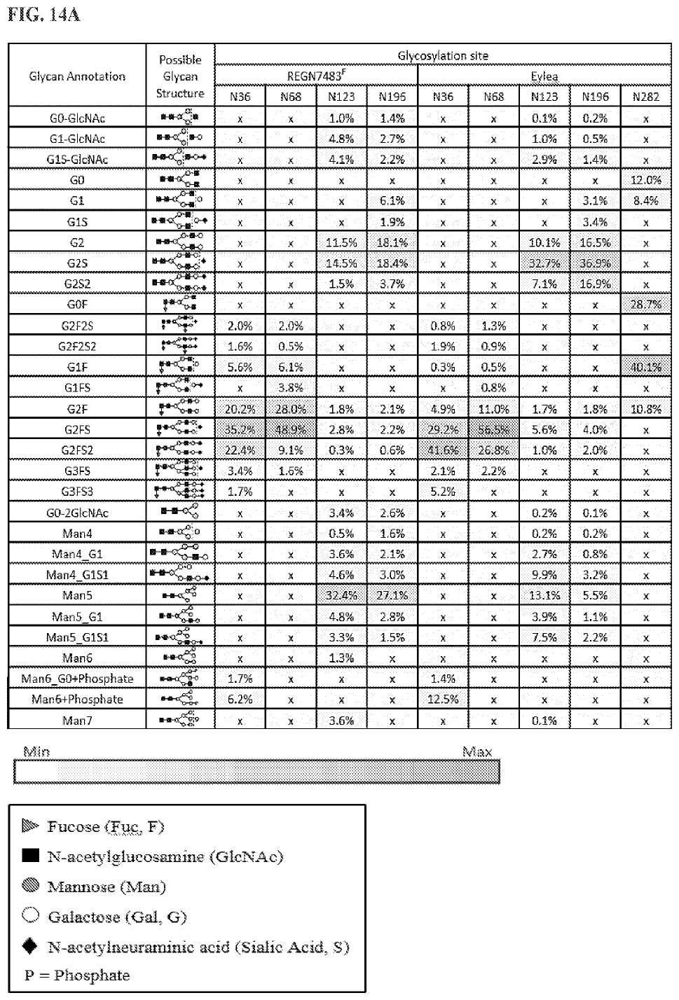

The dimeric mini-trap, not reaching complete inhibition of neovascularization even at 100 mg/kg, was much less potent than VEGF Trap (aflibercept) when delivered systemically (ip). See FIG. 10A. Historical data with VEGF Trap (aflibercept) showed near complete inhibition when delivered systemically (ip) at 6.25 mg/kg in an OIR mouse model. See FIG. 10B. This suggests that dimeric mini-trap has a shorter half-life than aflibercept when administered systemically. Such a short half-life can lead to a better safety profile since dimer mini-trap that leaks from the intravitreal space into the blood would be eliminated relatively quickly.

### Example 7: Addition of PPC to C-Terminus of REGN112 Expressed in EESYR CHO Cells

In this example, the ability of various mini-traps for form dimer or monomer were assessed.

Recombinant mini-traps encoding either REGN112, REGN7850 or REGN7851, were cloned into expression plasmids, transfected into CHO cells and stably transfected pools were isolated after selection with 400 μg/ml hygromycin for 12 days. The stable CHO cell pools, grown in chemically-defined protein-free medium, were used to produce proteins for testing. Prior to purification, an aliquot (10 μl) of the mini-trap containing medium was loaded on a 4-20% Novex Trys-Glycine (10 well, 1.0 mm minigel) SDS-PAGE gel in 1× Tris-Glycine SDS Running Buffer under reducing or non-reducing conditions. Proteins were visualized by staining with Coomassie Blue reagent. The monomer and dimer species were marked by arrow.

Visual examination of the SDS-PAGE gels (FIG. 11) indicated that the cells expressing REGN112 secreted about half of the protein as a preformed dimer with the remaining half as a monomer. Addition of one (REGN7850) or two (REGN7851) PPC motifs at the carboxy terminus of REGN112 improved production of the preformed dimer to nearly 100%.

### Example 8: Anion-Exchange Chromatography (AEX) for Mini-Trap Color Reduction

The AEX setpoint optimized during a prior multivariate characterization study (negative mode, pH 8.0, 7.0 mS/cm) did not provide adequate clearance of more brown REGN7483 species. A new AEX setpoint (pH 8.4, 2.0 mS/cm) was evaluated in bind-and-elute mode for three chromatography resins to determine whether a new setpoint could provide additional reduction of brown colored REGN7483 species. This setpoint had shown separation of more brown REGN3 species from less brown REGN3 species during previous REGN3 AEX development using Capto Q resin. Three AEX separations evaluated this setpoint on Q Sepharose FF, POROS 50 HQ, and Capto Q for REGN7483. A fourth AEX separation evaluated this setpoint on Capto Q for REGN3. A fifth AEX separation evaluated the original setpoint (pH 8.0, 7.0 mS/cm) for REGN7483 as a control to determine if the first 4 AEX separations could provide additional color reduction.

Design. Five AEX separations were performed for this study as detailed in Table 8-1. AEX separations 1 through 4 were performed using the method detailed in Table 8-3, while AEX separation 5 was performed using the method detailed in Table 8-2. All AEX loads were sourced from a similar bioreactor. A 15.7 mL Capto Q column (20.0 cm bed height, 1.0 cm I.D.), a 14.1 mL POROS 50 HQ column (18.0 cm bed height, 1.0 cm I.D.), and a 16.5 mL Q Sepharose FF column (21.0 cm bed height, 1.0 cm I.D.) were integrated into an AKTA Avant benchtop liquid chromatography controller for this experiment.

AEX load pH was adjusted to target ±0.05 pH units using 2 M tris base or 2 M acetic acid. AEX load conductivity was adjusted to target ±0.1 mS/cm using 5 M sodium chloride or RODI (reverse osmosis deionized water). All pool samples were analyzed for HMW, color, and yield.

TABLE 8-1
Summary of the Study Design for AEX Color Reduction Study

| AEX Separation | Load Source | Resin |
|---|---|---|
| 1 | REGN7483 Filtered Pool | Capto Q |
| 2 | REGN7483 Filtered Pool | POROS |
| | | 50 HQ |
| 3 | REGN7483 Filtered Pool | Q Sepharose |
| | | FF |
| 4 | REGN3 Filtered Pool | Capto Q |
| 5 | REGN7483 Filtered Pool | POROS |
| | | 50 HQ |

TABLE 8-2
Flow-Through AEX Protocol Used for Color Reduction Study (Separation 5)

| | | | Column | | Linear |
|---|---|---|---|---|---|
| | | | Volumes | Flow | Velocity |
| Step | Description | Mobile Phase | (CVs) | Direction | (cm/h) |
| 1 | Pre- | 2M Sodium Chloride (NaCl) | 2 | ↓ | 200 |
| | Equilibration | | | | |
| 2 | Equilibration | 50 mM Tris, 40 mM NaCl | 2 | ↓ | 200 |
| | | pH 7.90-8.10, 6.50-7.50 mS/cm | | | |
| 3 | Load | REGN7483 Load in Tris-Acetate | 30 g/L- | ↓ | 200 |
| | | buffer | resin | | |
| | | pH 7.90-8.10, 6.50-7.50 mS/cm | | | |
| 4 | Wash | 50 mM Tris, 40 mM NaCl | 2 | ↓ | 200 |
| | | pH 7.90-8.10, 6.50-7.50 mS/cm | | | |
| 5 | Strip 1 | 2M Sodium Chloride (NaCl) | 2 | ↑ | 200 |
| 6 | Strip 2 | 1N Sodium Hydroxide (NaOH) | 2 | ↑ | 200 |

AEX, anion exchange chromatography;
CV, column volume

TABLE 8-3
Bind-And-Elute AEX Protocol Used for Color Reduction Study (Separations 1-4)

| | | | Column | | Linear |
|---|---|---|---|---|---|
| | | | Volumes | Flow | Velocity |
| Step | Description | Mobile Phase | (CVs) | Direction | (cm/h) |
| 1 | Pre-Equilibration | 2M Sodium Chloride (NaCl) | 2 | ↓ | 200 |
| 2 | Equilibration | 50 mM Tris | 2 | ↓ | 200 |
| | | pH 8.30-8.50, 1.90-2.10 mS/cm | | | |
| 3 | Load | REGN3 or REGN7483 Load in Tris- | 30 g/L- | ↓ | 200 |
| | | Acetate buffer | resin | | |
| | | pH 8.30-8.50, 1.90-2.10 mS/cm | | | |
| 4 | Wash | 50 mM Tris | 2 | ↓ | 200 |
| | | pH 8.30-8.50, 1.90-2.10 mS/cm | | | |
| 5 | Elution | 50 mM Tris, 70 mM NaCl | 2 | ↓ | 200 |
| | | pH 8.30-8.50, 8.50-9.50 mS/cm | | | |
| 6 | Strip 1 | 2M Sodium Chloride (NaCl) | 2 | ↑ | 200 |
| 7 | Strip 2 | 1N Sodium Hydroxide (NaOH) | 2 | ↑ | 200 |

AEX, anion exchange chromatography;
CV, column volume

Results. Five AEX separations were performed to determine the optimal resin and setpoint capable of reducing color in AEX pool to acceptable levels. All pools were concentrated to 11 g/L before color was analyzed using the CI ELAB color space (L*, a* and b* variables). See CIEL*C*h* Color Scale, Application Notes, 8(11): 1-4 (Hunter Lab; Reston, Va.) (2008) and Objective Colour Assessment and Quality Control in the Chemical, Pharmaceutical and Cosmetic Industries”, Application Report No. 3.9 e from Hach Lange GmbH, pp. 1-28, Feb. 2013. While the first four AEX separations (1-4) were intended to be evaluated in a bind-and-elute mode, the majority of the product was present in the load and wash blocks (62-94%), i.e., the column operated in negative, or flow-through, modality.

The first 3 separations (1-3) evaluated the pH 8.4 and 2.0 mS/cm setpoint for Capto Q, POROS 50 HQ, and Q Sepharose FF resins with REGN7483 as the load material. All 3 separations showed a yield of >80% and a pool HMW (high molecular weight species content) of <3.4%. The POROS 50 HQ AEX pool showed the lowest yellow color in AEX pool (b*=2.09) followed by the Q Sepharose FF AEX pool (b*=2.22) and the Capto Q AEX pool (b*=2.55).

The fourth AEX separation (4) evaluated the pH 8.4 and 2.0 mS/cm setpoint for Capto Q with REGN3 as the load material. This setpoint showed a yield of 61.9% collected during load and wash and 34.0% collected during the elution. This AEX pool was the least yellow (b*=1.44). While this AEX condition resulted in the least yellow AEX pool, it had been observed that yellow color increases after cleavage with the FabRICATOR enzyme and subsequent removal of the cleaved Fc portion (b*=3.52 in pre-cleavage pool and b*=4.17 in post-cleavage and Fc removal pool). This is presumably as the brown color is more present in the REGN7483 portion of the REGN3 molecule rather than the Fc portion, so removing the Fc and resultantly doubling the molarity of REGN7483 at constant concentration in g/L terms caused by the enzymatic cleavage intensifies the color Adding this expected increase in yellow color (Δb*=+0.65) to the color of the REGN3 AEX pool (b*=1.44+0.65=2.09) would indicate that after the FabRICATOR unit operation it would have a similar color as the least yellow REGN7483 AEX pool (b*=2.09). In addition, the load and wash yield of 62% was below the development goal (>80%), making this a less desirable setpoint for the AEX separation.

The fifth AEX separation (5) evaluated the previously optimized setpoint (pH 8.0 and 7.0 mS/cm) on POROS 50 HQ resin with REGN7483 as the load material. While this AEX separation showed a yield of >80% and pool HMW of <3.4%, it was the most yellow pool (b*=3.40).

TABLE 8-5
Summary of Experimental Results of AEX Color Reduction Study*

| AEX | | Yield | HMW | Color | Color | Color |
|---|---|---|---|---|---|---|
| Separation | Fraction | (%) | (%) | (L*) | (a*) | (b*) |
| 1 | Load Flow-through | 90.7 | 0.49 | 99.11 | −0.27 | 2.55 |
| | and Wash | | | | | |
| 2 | Load Flow-through | 93.8 | 0.33 | 99.20 | −0.28 | 2.09 |
| | and Wash | | | | | |
| 3 | Load Flow-through | 86.7 | 0.23 | 98.88 | −0.23 | 2.22 |
| | and Wash | | | | | |
| 4 | Load Flow-through | 61.9 | 1.66 | 98.21 | −0.17 | 1.44 |
| | and Wash | | | | | |
| 4 | Elution | 34.0 | 4.20 | 97.55 | −0.42 | 4.49 |
| 5 | Load Flow-through | 99.5 | 1.13 | 98.90 | −0.39 | 3.40 |
| | and Wash | | | | | |
| N/A | REGN7483 Filtered | N/A | 0.65 | 98.18 | −0.37 | 4.17 |
| | Pool (AEX Load) | | | | | |
| N/A | REGN3 | N/A | 4.14 | 98.42 | −0.33 | 3.53 |
| | Filtered Pool | | | | | |
| | (AEX Load) | | | | | |
| N/A | REGN3 | N/A | N/A | 99.07 | −0.12 | 1.96 |

*Color was determined in samples at a protein concentration of 11 g/l
AEX, anion exchange chromatography;
HMW, high molecular weight species;
N/A, not applicable

Conclusion. Five AEX separations were performed to evaluate resins (Capto Q, Q Sepharose FF, and POROS 50 HQ) and setpoints (pH 8.0 and 7.0 mS/cm, pH 8.4 and 2.0 mS/cm). Performing an AEX separation with REGN7483 on POROS 50 HQ with a setpoint of pH 8.4 and 2.0 mS/cm resulted in a less yellow AEX pool compared to Q Sepharose FF AEX pool and Capto Q AEX pool with the same process and load source. The fourth AEX separation (REGN3 load source, Capto Q resin, pH 8.4 and 2.0 mS/cm setpoint) is predicted to have a comparable color to the REGN7483 POROS 50 HQ AEX pool after the enzymatic cleavage unit operation.

Finally, the fifth AEX separation (REGN7483 load source, POROS 50 HQ resin, pH 8.0 and 7.0 mS/cm setpoint) resulted in the most yellow pool. The excess yellow color is believed to be due to relative low pH (7.9-8.1) and high conductivity (6.5-7.5 mS/cm). These two factors have been shown to cause higher levels of yellow color in CDM expressed REGN7483^{F}.

Purification of aflibercept expressed in CDM (and having a brown-yellow color) by protein-A chromatography and then activated charcoal filtration did not result in significant brown-yellow color reduction (Data not shown).

### Example 9: Analysis of Color and 2-Oxo-Histidine in Mini-Trap AEX Purified at Higher pH and Lower Conductivity

The brown-yellow color and the quantity of mini-trap (REGN7483^{F}) histidines which have been oxidized to 2-oxo-histidine in various mini-trap production lots were evaluated in this Example.

Sample preparation. Tryptic mapping of reduced and alkylated mini-trap (REGN7483^{F}) sample lots (10, 23 and 14) were performed to identify and quantify the 2-oxo-histidine posttranslational modification. A 200 μg aliquot of each drug substance lot was denatured in 8.0 M Urea in 0.1 M Tris-HCl, pH 7.5, reduced with DTT and then alkylated with iodoacetamide. The denatured, reduced, and alkylated drug substance was first digested with recombinant Lys-C (rLys-C) at an enzyme to substrate ratio of 1:100 (w/w) at 37° C. for 30 minutes, diluted with 0.1 M Tris-HCl, pH 7.5 such that the final urea concentration was 1.8 M, subsequently digested with trypsin at an enzyme to substance ratio of 1:20 (w/w) at 37° C. for 2 hours, and then deglycosylated with PNGase F at an enzyme substrate ratio of 1:5 (w/w) for 37° C. for 1 hour. The digestion was stopped by bringing the pH below 2.0 using formic acid (FA).

Mini-trap productions. A bioreactor working volume of 500 liters was used to express aflibercept. Cell culture containing aflibercept was subjected to three filtration steps (depth, polish and guard), followed by protein-A affinity capture chromatography (bind-and-elute) and, a further filtration. This material was then enzymatically cleaved with Streptococcus pyogenes IdeS protease (FabRICATOR, Genovis; Cambridge, MA; Lund, Sweden) which has been immobilized on a resin to generate mini-trap and a cleaved Fc fragment by-product. The Fc fragment was removed from the reaction by protein-A affinity capture chromatography (mini-trap product in flow-through fraction) which was followed by a filtration step (only for mini-trap productions 162, 29 and 30). Following a viral low pH hold inactivation and filtration step, the mini-trap was purified by anion-exchange (AEX) chromatography (flow-through mode) using the parameters set forth in Table 9-1.

TABLE 9-1
AEX Chromatography Conditions

| Mini-trap | Mini-trap | Mini-trap | Mini-trap | Mini-trap | Mini-trap | Mini-trap |
|---|---|---|---|---|---|---|
| production | production | production | production | production | production | production |
| 10 | 14 | 162 | 22 | 23 | 29 | 30 |
| Resin: Q | Resin: | Resin: | Resin: | Resin: | Resin: | Resin: |
| Sepharose | POROS 50 | POROS 50 | POROS 50 | POROS 50 | POROS 50 | POROS 50 |
| Fast Flow | HQ (Life | HQ (Life | HQ (Life | HQ (Life | HQ (Life | HQ (Life |
| (GE Healthcare) | Technologies) | Technologies) | Technologies) | Technologies) | Technologies) | Technologies) |
| Column height: | Column | Column | Column | Column | Column | Column |
| 20 ± 1.0 cm | height: Same | height: Same | height: Same | height: Same | height: Same | height: Same |
| Loading: ≤100 | Loading: ≤40 | Loading: ≤40 | Loading: ≤40 | Loading: ≤40 | Loading: ≤40 | Loading: ≤40 |
| g/L resin | g/L resin | g/L resin | g/L resin | g/L resin | g/L resin | g/L resin |
| Pre- | Pre- | Pre- | Pre- | Pre- | Pre- | Pre- |
| equilibration: | equilibration: | equilibration: | eguilibration: | equilibration: | equilibration: | equilibration: |
| 2.0M NaCl | Same | Same | Same | Same | Same | Same |
| Equilibration/ | Equilibration/ | Equilibration/ | Equilibration/ | Equilibration/ | Equilibration/ | Equilibration/ |
| wash: 50 mM | wash: 50 mM | wash: 50 mM | wash: 50 mM | wash: 50 mM | wash: 50 mM | wash: 50 mM |
| Tris, 60 mM | Tris pH 8.4 ± | Tris pH | Tris pH | Tris pH | Tris pH | Tris pH |
| NaCl, pH 7.7 ± | 0.1 | 8.4 ± 0.1 | 8.4 ± 0.1 | 8.4 ± 0.1 | 8.4 ± 0.1 | 8.4 ± 0.1 |
| 0.1 | Strip 1: Same | Strip 1: Same | Strip 1: Same | Strip 1: Same | Strip 1: Same | Strip 1: Same |
| Strip 1: 2.0M | Strip 2: Same | Strip 2: Same | Strip 2: Same | Strip 2: Same | Strip 2: Same | Strip 2: Same |
| NaCl | Load | Load | Load | Load | Load | Load |
| Strip 2: 1.0N | adjustment: | adjustment: | adjustment: | adjustment: | adjustment: | adjustment: |
| NaOH | 2.0M Tris | 2.0M Tris | 2.0M Tris | 2.0M Tris | 2.0M Tris | 2.0M Tris |
| Load | Base and | Base and | Base and | Base and | Base and | Base and |
| adjustment: 2.0M | RODI, | RODI, | RODI, | RODI, | RODI, | RODI, |
| Tris Base | adjusted to | adjusted to | adjusted to | adjusted to | adjusted to | adjusted to |
| and 2.0M | pH 8.4 ± 0.1 | pH 8.4 ± 0.1 | pH 8.4 ± 0.1 | pH 8.4 ± 0.1 | pH 8.4 ± 0.1 | pH 8.4 ± 0.1 |
| NaCl, adjusted | and 2.0 ± 0.1 | and 2.0 ± 0.1 | and 2.0 ± | and 2.0 ± 0.1 | and 2.0 ± 0.1 | and 2.0 ± 0.1 |
| to pH 7.7 ± 0.1 | mS/cm | mS/cm | 0.1 mS/cm | mS/cm | mS/cm | mS/cm |
| and 9.0 ± 0.1 | | | | | | |
| mS/cm | | | | | | |

RODI = reverse osmosis deionized water

The purified material was then further purified by hydrophobic interaction chromatography (resin with a phenyl ligand), followed by concentration and diafiltration.

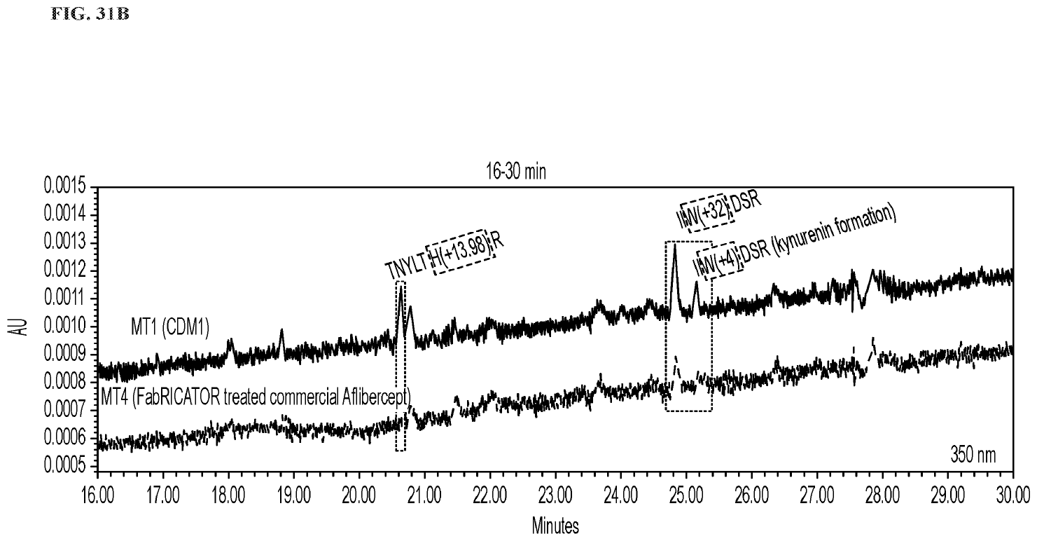

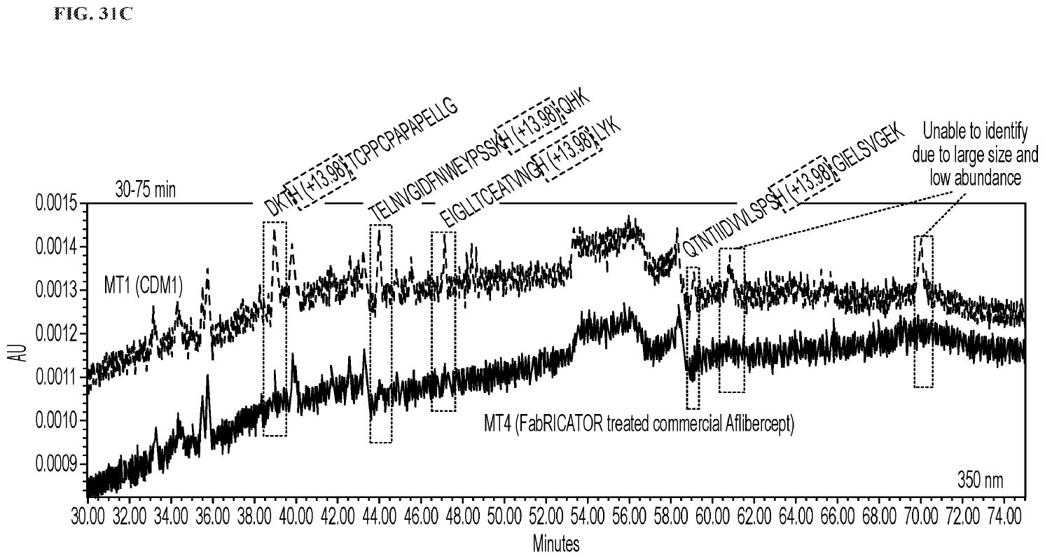

Localization of peptide fragments responsible for increased absorbance at 350 nm. The PTMs on mini-trap production 10, possibly responsible for the intense color of the mini-trap production 10 sample, were observed on comparing the tryptic peptide maps for mini-trap production 10 and VEGF Mini-Trap obtained by cleavage of aflibercept produced using the commercial process (non-CDM) (FIG. 31(A) which shows the absorbance of peptides eluted from 20.0 to 75 minutes). The peptides with varying UV peaks are highlighted. The expanded view of the chromatogram is shown in FIG. 31(B) which shows the absorbance of peptides eluted from 16 to 30 minutes. The peptides with sharp contrast in UV absorbance between mini-trap production 10 and VEGF Mini-Trap obtained by cleavage of aflibercept produced using the commercial process (non-CDM) were TNYLTH*R, IIW*DSR and IIIW*DSR (* represents oxidation of the residue). Further, the expanded view of the chromatogram is shown in FIG. 31(C) which shows the absorbance of peptides eluted from 30 to 75 minutes. The peptides with sharp contrast in UV absorbance between mini-trap production 10 and VEGF Mini-Trap obtained by cleavage of aflibercept produced using the commercial process (non-CDM) were DKTH*TCPPCPAPELLG, TELNVGIDFNWEYPSSKH*QHK, EIGLLTCEATVNGH*LYK and QTNTIIDWLSPSH*GIELSVGEK (* represents oxidation of the residue). The peptide mapping revealed identity of peptides that are significantly different in abundance between the VEGF Mini-Traps. The relative abundance of the peptides identified form the peptide mapping analysis is shown in Table 9-2. The amount of 2-oxo-histidines in mini-trap production 10 were higher than VEGF Mini-Trap obtained by cleavage of aflibercept produced using the commercial process (non-CDM) suggesting that presence of 2-oxo-histidines could be responsible for the intense yellow-brown color.

TABLE 9-2
Relative Abundance of Peptides identified from Peptide Mapping Analysis

| | | | | Fold change |
|---|---|---|---|---|
| | | | | Mini-trap |
| | | | | production |
| | | | Mini- | 10/Mini-Trap |
| | | Mini-Trap | trap | from |
| | | from | prodn. | Commercial |
| Peptide | Peptide Modified Sequence | Aflibercept | 10 | Aflibercept |
| EIGLLTCEATVNGHLYK | EIGLLTC[+57]EATVNGH[+14] | 0.004% | 0.011% | 2.75 |
| | LYK | | | |
| QTNTIIDVVLSPSHGIELSVG | QTNTIiDVVLSPSH[+14]GIELS | 0.001% | 0.015% | 15.00 |
| EK | VGEK | | | |
| TELNVGIDFNWEYPSSKHQ | TELNVGIDFNWEYPSSKH[+14] | 0.026% | 0.204% | 7.85 |
| HK | QHK | | | |
| DKTHTCPPCPAPELLG | DKTH[+14]TC[+57]PPC[+57] | 0.018% | 0.115% | 6.39 |
| | PAPELLG | | | |
| TNYLTHR | TNYLTH[+14]R | 0.020% | 0.130% | 6.50 |

LC-MS analysis. A 20 μg aliquot of resulting rLys-C/tryptic peptides from lots 10, 14 and 23 was separated and analyzed by reverse-phase ultra-performance liquid chromatography (UPLC) using Waters ACQUITY UPLC CSH C18 column (130 Å, 1.7 μm, 2.1×150 mm) followed by on-line PDA detection (at wavelengths of 280 nm, 320 nm and 350 nm) and mass spectrometry analysis. Mobile phase A was 0.1% FA in water, and mobile phase B was 0.1% FA in acetonitrile. After sample injection, the gradient started with 5 minutes hold at 0.1% B followed by a linear increase to 35% B over 75 minutes for optimum peptide separation. MS and MS/MS experiments were conducted on a Thermo Scientific Q Exactive Hybrid Quadrupole-Orbitrap mass spectrometer with higher-energy collisional dissociation (HCD) employed for peptide fragmentation for MS/MS experiments. Peptide identity assignments were based on the experimentally determined accurate mass of a given peptide in the full MS spectrum as well as the b and y fragment ions in the corresponding HCD MS/MS spectrum. Extracted ion chromatograms of the 2-oxo-histidine-containing peptide and corresponding native peptide were generated with the peak areas integrated to calculate the site-specific percentage of 2-oxo-his within REGN7483^{F} samples.

2-oxo-histidine Quantitation of Mini-Trap Productions 10, 23 and 14. The color of various mini-trap preparations (in the AEX column flow-through fractions) or in the material stripped from the AEX column, relative to the European Brown-Yellow Color Standards (BY), are set forth below in Table 9-3. The percentage of 2-oxo-histidines in the peptides that were generated by protease digestion, as measured by mass spectrometry, are also shown.

TABLE 9-3
Correlation between Brown-Yellow Color and Percentage of 2-Oxo-
Histidine in REGN7483^{F}

| | | AEX Flow-Through | |
|---|---|---|---|
| | AEX Strip | Mini-trap production | Mini-trap production |
| Modified Peptides | Intense yellow | 10 BY1, 110 mg/mL | 23 <BY3,110 mg/mL |
| EIGLLTC[+57]EATVNGH[+14]LYK | 0.080% | 0.013% | 0.008% |
| QTNTIIDVVLSPSH[+14]GIELSVGEK | 0.054% | 0.028% | 0.023% |
| TELNVGIDFNWEYPSSKH[+14]QHK | 0.235% | 0.085% | 0.049% |
| DKTH[+14]TC[+57]PPC[+57]PAPELLG | 0.544% | 0.092% | 0.077% |
| TNYLTH[+14]R | 0.089% | 0.022% | 0.011% |
| IIW[+32]DSR | 0.738% | 0.252% | 0.198% |

AEX Flow-Through

| | Acidic | Acidic | |
|---|---|---|---|
| | fraction 1 | fraction 2 | Main fraction from |
| | from Mini-trap | from Mini-trap | Mini-trap |
| Mini-trap production 14 | production 14 | production 14 | production 14 |
| ≤BY3, 110 mg/mL | yellow | yellow | No Color |
| 0.006% | 0.009% | 0.008% | 0.004% |
| 0.019% | 0.013% | 0.015% | 0.006% |
| 0.049% | 0.131% | 0.151% | 0.049% |
| 0.057% | 0.117% | 0.132% | 0.068% |
| 0.010% | 0.014% | 0.008% | 0.008% |
| 0.298% | 0.458% | 0.269% | 0.185% |

[+57]: Alkylation of cysteine by iodoactamide adds a carboxymethyl amine moiety on the cysteine which results in a net mass increase of about +57 over unmodified cysteine:

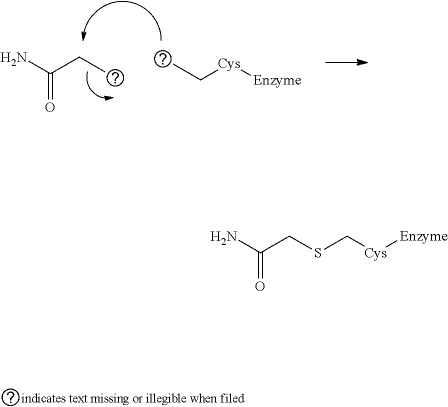

[+14]: From His to 2-oxo-His one oxygen atom is added on carbon 2, but two hydrogen atoms are lost (one from Carbon 2, the other from Nitrogen 3), which results in a net mass increase of about +14 over unmodified histidine.

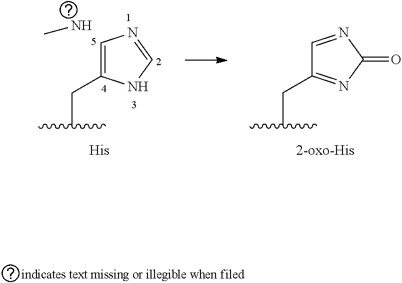

[+32]: Tryptophan dioxidation results in the formation of N-formylkynurenine which is a net mass increase of about +32 over unmodified tryptophan.

Color Analysis of Mini-Trap Productions 10, 14, 22, 23, 162, 29, 30 and REGN3. A CIEL*a*b* color analysis of the mini-trap productions is set forth below in Table 9-4.

TABLE 9-4
Color Analysis of Mini-Trap Productions

| | Conc. | | | | |
|---|---|---|---|---|---|
| Sample | (g/L) | L* | a* | b* | Color |
| REGN7483^{F} (10) FCP | 169 | 88.61 | 0.53 | 31.17 | >BY1 |
| REGN7483^{F} (14) FCP | 161 | 95.01 | −1.68 | 18.16 | <BY2 |
| REGN7483^{F} (22) | 158 | 96.10 | −1.05 | 14.34 | <BY3 |
| DS-Formulated version of REGN7483^{F} | 106 | 97.18 | −0.93 | 10.31 | <BY3 |
| (22), incl. sucrose | | | | | |
| REGN7483^{F} (23) | 154 | 96.06 | −1.02 | 14.48 | <BY3 |
| REGN7483^{F} (162) | 159 | 96.96 | −0.85 | 14.89 | <BY3 |
| DS-Formulated version of REGN7483^{F} | 128 | 97.76 | −1.02 | 12.16 | <BY3 |
| (162), incl. sucrose | | | | | |
| REGN7483^{F} (29) FCP | 205 | 95.06 | −1.07 | 20.87 | <BY2 |
| REGN7483^{F} (30) FCP | 158 | 96.93 | −1.55 | 14.02 | <BY3 |
| REGN3 (343) FCP (Produced in CDM) | 150 | 97.36 | −0.39 | 10.64 | <BY3 |
| REGN3 (310) FCP (Produced in non- | 144 | 99.16 | −0.35 | 3.41 | <BY5 |
| CDM containing Soy hydrolysate) | | | | | |
| REGN3 (309) FCP (Produced in non- | 79.3 | 99.33 | −0.19 | 2.39 | <BY5 |
| CDM containing Soy hydrolysate) | | | | | |
| BY2 Standard | N/A | >94.25 | N/A | <26.28 | N/A |
| BY1 Standard | N/A | >92.84 | N/A | <31.15 | N/A |

The parenthetical number following REGN7483^{F} indicated the mini-trap production number whose conditions are specified herein. The parenthetical number following REGN3 indicates the aflibercept (REGN3) production number.
FCP = final concentrated pool. DS = drug substance.
L*, a* and b* are values in the CIEL*a*b* color space.
< and > indicates whether color was less than or greater than the BY reference solution; for example, “<BY2” means that the color was in between BY3 and BY2.

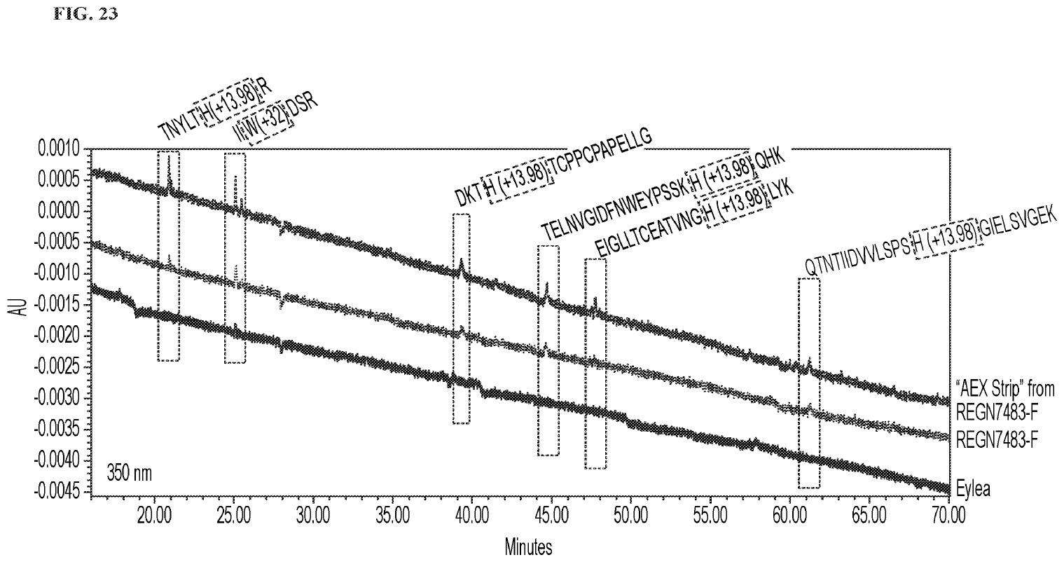

A set of experiments were performed to evaluate the percentage of 2-oxo-histidines (and tryptophan dioxidation) in aflibercept (produced in a non-chemically defined medium) and REGN7483^{F}. Aflibercept, REGN7483^{F} material flowing through the AEX column in mini-trap production 10, as well as material stripped from the AEX column were protease digested with trypsin and LysC; as well as with PNGase F. The peptides were then applied to a Waters BEH200, 4.6 cm×150 mm size-exclusion (SEC) column. Material corresponding to absorbent peaks were retained and analyzed by mass spectrometry to determine their content. It was determined that the material stripped from the AEX column was enriched for the presence of 2-oxo-his and tryptophan dioxidation species. Moreover, the level of 2-oxo-histidines and dioxidated tryptophan was very low. See FIG. 23 and Table 9-5.

These data indicated that the 2-oxo-his and tryptophan dioxidation species have an affinity for the AEX resin and that AEX chromatography in flow-through mode is an effective means by which to eliminate these species from REGN7483.

TABLE 9-5
Quantitation of 2-oxo-his or Tryptophan Dioxidation in Aflibercept,
REGN7483^{F} or AEX Strip

| | | | AEX Strip | Fold Change |
|---|---|---|---|---|
| | | REGN7483F | from | AEX |
| Peptide | Eylea | (10) | REGN7483^{F} | Strip/REGN7483^{F} |
| IIW[+32]DSR | 0.22% | 0.34% | 0.81% | 2.4 |
| EIGLLTC[+57]EATVNGH[+14]LYK | 0.00% | 0.02% | 0.08% | 4.0 |
| QTNTIIDVVLSPSH[+14]GIELSVGEK | 0.01% | 0.04% | 0.07% | 1.8 |
| TELNVGIDFNWEYPSSKH[+14]QHK | 0.01% | 0.19% | 0.42% | 2.2 |
| DKTH[+14]TC[+57]PPC[+57]PAPELLG | 0.01%^{a} | 0.11% | 0.63% | 5.7 |
| TNYLTH[+14]R | 0.00% | 0.03% | 0.10% | 3.3 |

^{a}value calculated using a different peptide for REGN3, as the C-terminal peptide is different from mini-Trap.

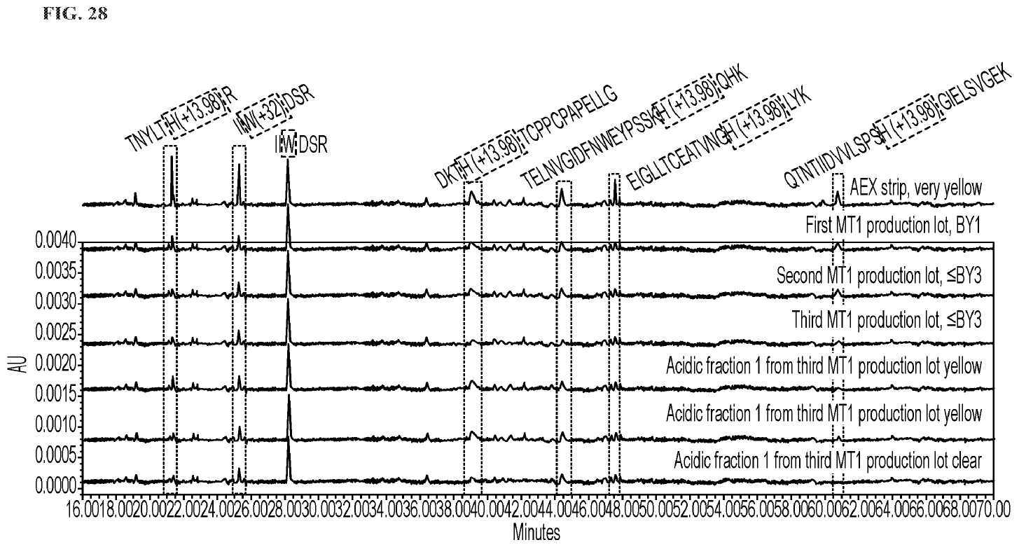

The comparison of acidic species (Table 9-5) present in AEX strip for mini-trap production 10 (prior to any purification procedure, BY1), mini-trap production 23 (prior to any purification procedure, ≤BY3), mini-trap production 14 (prior to any purification procedure, ≤BY3), acidic fraction 1 from mini-trap production 10 (obtained after AEX procedure, yellow colored), acidic fraction 2 from mini-trap production 10 (obtained after AEX procedure, yellow colored) and main fraction from mini-trap production 10 (obtained after AEX procedure, clear) is shown in FIG. 28.

Strong Cation Exchange chromatogram (CEX). This method was employed towards the identification of the acidic species and other variants present in cell culture harvest samples.

Strong cation exchange chromatography was performed on a [Dionex ProPac WCX-10, Analytical column (Dionex, CA)]. For the samples, the mobile phases used were [10 mM Sodium Phosphate dibasic pH 7.5 (Mobile phase A) and 10 mM Sodium Phosphate dibasic, 500 mM Sodium Chloride pH 5.5 (Mobile phase B). A binary gradient (94% A, 6% B: 0-20 min; 84% A, 16% B: 20-22 min; 0% A, 100%B: 22-28 min; 94% A, 6% B: 28-34 min) was used with detection at 280 nm]. The peaks that elute at relative residence time earlier than the main peak corresponding to the acidic peaks.

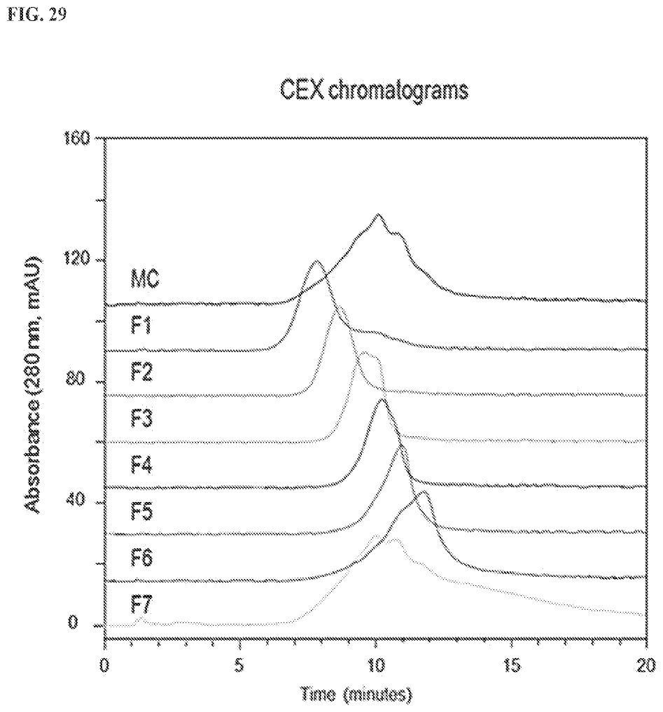

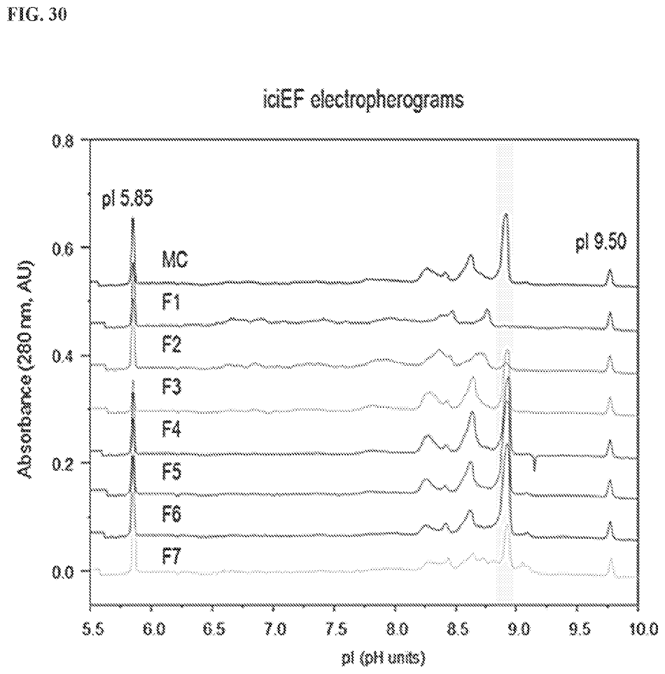

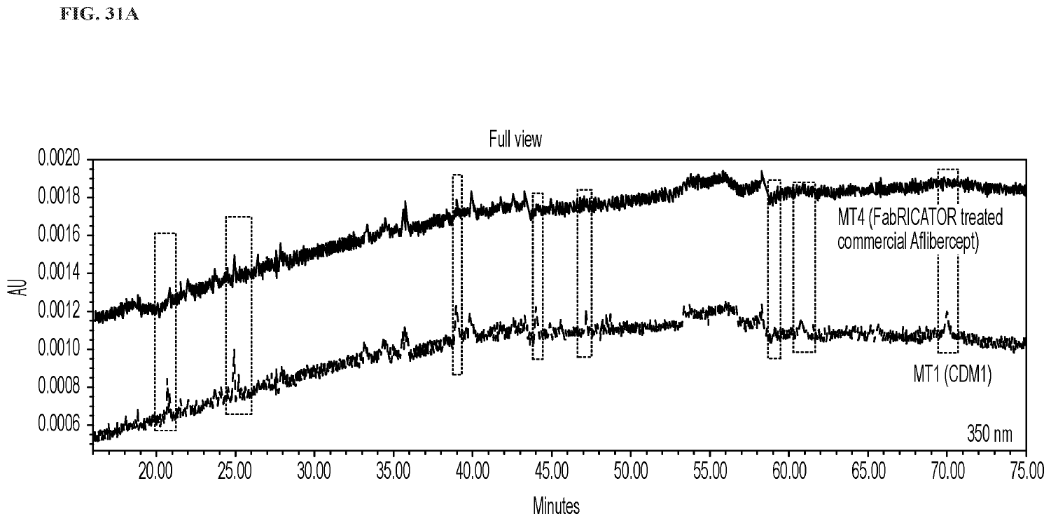

A sample from the mini-trap production 23 (prior to any purification procedure, ≤BY3) was subjected to CEX. Desialylation was applied to the sample to reduce the complexity of variants of the mini-trap productions. Subsequently, strong cation exchange (CEX) chromatography was applied to enrich for variants of desialylated minitrap (dsMT1) using a dual salt-pH gradient. The procedure resulted in a total of 7 fractions (F1-F7, MC is the method control). Yellow-brown variants were observed only in the two most acidic protein variant fractions 1 and 2. This result was supported by the AEX strip sample produced, which was used to remove the majority of yellow-brown variants of MT1 and contains acidic variants of MT1 (FIG. 29).

Imaged capillary isoelectric focusing (iciEF) electropherograms. The distribution of variants in fractions F1-7 and MC (from mini-trap production 23 after CEX) were further assessed by iCIEF using an [iCE280 analyzer (ProteinSimple) with a fluorocarbon coated capillary cartridge (100 μm×5 cm). The ampholyte solution consisted of a mixture of 0.35% methyl cellulose (MC), 0.75% Pharmalyte 3-10 carrier ampholytes, 4.2% Pharmalyte 8-10.5 carrier ampholytes, and 0.2% pi marker 7.40 and 0.15% pi marker 9.77 in purified water. The anolyte was 80 mM phosphoric acid, and the catholyte was 100 mM sodium hydroxide, both in 0.10% methylcellulose. Samples were diluted in purified water and CpB was added to each diluted sample at an enzyme to substrate ratio of 1:100 followed by incubation at 37° C. for 20 minutes. The CpB treated samples were mixed with the ampholyte solution and then focused by introducing a potential of 1500 V for one minute, followed by a potential of 3000 V for 10 minutes. An image of the focused ot-PDLI variants was obtained by passing 280 nm ultraviolet light through the capillary and into the lens of a charge coupled device digital camera. This image was then analyzed to determine the distribution of the various charge variants (FIG. 30).

### Example 10: Photostability Study of REGN7483^{F}

In this example, the photostability of REGN7483^{F} from mini-trap production 14 (discussed above) was determined after exposure to varying amounts of cool white light or ultra-violet A light. The color and 2-oxo-histidine content of the light exposed samples were determined.

TABLE 10-1
REGN7483^{F} Photostability Study Design

| | Cumulative Exposure (x ICH) | | | | |
|---|---|---|---|---|---|
| | 0.2 | 0.5 | 0.8 | 1.0 | 2.0 |
| CW fluorescent | 0.24 million | 0.6 million | 0.96 million | 1.2 Million | 2.4 million |
| exposure | lux*hr | lux*hr | lux*hr | lux*hr | lux*hr |
| (lux*hr) | | | | | |
| Incubation time | 30 hours | 75 hours | 100 hours | 150 hours | 300 hours |
| with CW | | | | | |
| fluorescent | | | | | |
| light (at 8 klux) | | | | | |
| UVA exposure | 40 | 100 | 160 | 200 | 400 |
| (W*hr/m²) | | | | | |
| Incubation time | 4 hours | 10 hours | 16 hours | 20 hours | 40 hours |
| with UVA (at 10 | | | | | |
| W/m²) | | | | | |

ICH refers to ICH Harmonised Tripartite Guideline: Stability Testing: Photostability Testing of New Drug Substances And Products Q1B which specifies photostability studies to be conducted with net less than 1.2 Million lux*hours light.

TABLE 10-2
Color of Samples Exposed to Cool White Light
and Ultra-Violet A Light ~♦

| | | | | |
|---|---|---|---|---|
| Photo exposure | L* | a* | b* | EP BY Value |

Cool White Light

| | | | | |
|---|---|---|---|---|
| T = 0 | 97.37 | −1.12 | 9.58 | 4.0 |
| 0.2× (0.24 million lux*hr) | 96.46 | −0.72 | 11.75 | 3.7 |
| 0.5× (0.6 million lux*hr) | 95.47 | −0.4 | 11.3 | 3.7 |
| 0.8× (0.96 million lux*hr) | 95.33 | −0.38 | 11.96 | 3.6 |
| 1.0× (1.2 million lux*hr) | 94.42 | −0.2 | 13.72 | 3.3 |
| 2.0× (2.4 million lux*hr) | 92.70 | 0.41 | 22.14 | 2.0 |

UVA

| | | | | |
|---|---|---|---|---|
| 0.2× (40 W*h/m²) | 97.26 | −0.92 | 12.66 | 3.5 |
| 0.5× (100 W*h/m²) | 100.39 | −1.01 | 11.83 | 3.7 |
| *0.8× (160 W*h/m²) | 79.69 | −0.18 | 10.1 | 3.6 |
| 1.0× (200 W*h/m²) | 97.48 | −0.95 | 11.36 | 3.7 |
| 2.0× (400 W*h/m²) | 97.76 | −0.98 | 10.72 | 3.8 |

^{~} Sample colors are indicated using the CIELAB color space (L*, a* and b* variables) and relative to the EP BY color standard.
^{♦} Samples measured in 80 mg/mL REGN7483^{F} (mini-trap production 19) in 10 mM histidine, 7% sucrose, 0.03% PS20-SR, pH 5.8
*Values of outliers

TABLE 10-3
2-oxo-His Levels in Peptides from Ultra-violet light and Cool White Light
Stressed Mini-Trap

| Peptides | t0 | UV_4h | UV_10h | UV_16h |
|---|---|---|---|---|
| DKTH[+14]TC[+57]PPC[+57]PAPELLG | 0.056% | 0.067% | 0.081% | 0.088% |
| EIGLLTC[+57]EATVNGH[+14]LYK | 0.010% | 0.020% | 0.034% | 0.037% |
| QTNTIIDWLSPSH[+14]GIELSVGEK | 0.024% | 0.031% | 0.028% | 0.028% |
| TELNVGIDFNWEYPSSKH[+14]QHK | 0.096% | 0.147% | 0.163% | 0.173% |
| TNYLTH[+14]R | 0.014% | 0.032% | 0.044% | 0.056% |

| UV_20h | UV_40h | CW_30h | CW_75h | CW_100h | CW_150h | CW_300h |
|---|---|---|---|---|---|---|
| 0.077% | 0.091% | 0.152% | 0.220% | 0.243% | 0.258% | 0.399% |
| 0.033% | 0.035% | 0.063% | 0.110% | 0.132% | 0.170% | 0.308% |
| 0.027% | 0.027% | 0.085% | 0.120% | 0.128% | 0.148% | 0.180% |
| 0.147% | 0.125% | 0.423% | 0.585% | 0.634% | 0.697% | 0.748% |
| 0.058% | 0.078% | 0.103% | 0.175% | 0.198% | 0.267% | 0.437% |

Exposure of REGN7483^{F} cool white light or UVA light was correlated with the appearance of oxidized histidines (2-oxo-his). Two species of 2-oxo-histidine were observed, a 13.98 Da species

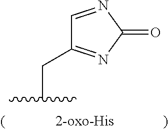 [SMILES: *CC1=NC(=O)N=C1]

and a 15.99 Da species

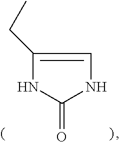

with the 13.98 Da species being predominant in light stressed Mini-trap samples. Evidence suggests that the brown-yellow color observed is dependent on the 13.98 Da species, but not the 15.99 Da species. The 15.99 Da species is known to be a product of a copper metal-catalyzed process. Schöneich, J. Pharm. Biomed Anal. 21:1093-1097 (2000). Spiking mini-trap with copper did not result in appreciable color change (Data not shown). The 13.98 Da species, however, is a product of a light-driven process. Liu et al., Anal. Chem. 86(10: 4940-4948 (2014)).

### Example 11: Analysis of Post-Translational Modifications (PTMs) by Reduced Peptide Mapping

The glycosylation profile and presence of other post-translational modifications of mini-trap (including REGN7483^{F}) and aflibercept was evaluated in this Example.

Sample preparation. Tryptic mapping of reduced and alkylated mini-trap (REGN7483^{F}; mini-trap production 22) and Eylea drug substance lots was performed to identify and quantify post-translational modifications (e.g., site-specific glycosylation, deamidation and oxidation, etc.). A 1 mg aliquot of each drug substance was denatured in 6.0 M guanidine hydrochloride, reduced with DTT, and alkylated with iodoacetamide at pH 7.5. The denatured, reduced and alkylated drug substance was then desalted and buffer exchanged into 0.1 M Tris HCl using a NAP-5 column, and subsequently digested with trypsin at an enzyme to substance ratio of 1:20 (w/w) at 37° C. for 2 hours. The digestion was stopped by bringing the pH below pH 2.0 using TFA.

LC-MS analysis. An aliquot of 7.6 μg resulting tryptic peptides and glycopeptides from each drug substance lot was separated and analyzed by reverse-phase ultra-performance liquid chromatography (UPLC) using Waters ACQUITY UPLC BEH130 C18 column (1.7 μm, 2.1×150 mm) followed by on-line mass spectrometry analysis to determine the peptide and glycopeptide masses and confirm peptide sequences. Mobile phase A was 0.05% TFA in water, and mobile phase B was 0.045% TFA in acetonitrile. After sample injection, the gradient started with 5 minutes hold at 0.1% B followed by a linear increase to 35% B over 75 minutes for optimum peptide separation. MS and MS/MS experiments were conducted on a Thermo Scientific Q Exactive Plus Hybrid Quadrupole-Orbitrap mass spectrometer with higher-energy collisional dissociation (HCD) employed for peptide fragmentation for MS/MS experiments. Peptide and glyopeptide identity assignments were based on the experimentally determined accurate mass of a given peptide or glycopeptide in the full MS spectrum as well as the b and y fragment ions in the corresponding HCD MS/MS spectrum. For PTMs analysis, the extracted ion chromatograms of the PTM-containing peptide and corresponding native peptide were generated with the peak areas integrated to calculate the site-specific percentage of PTMs within REGN7483^{F} and Eylea samples.

FIG. 14(A) sets forth the glycoforms identified at each asparagine glycosylation site on REGN7483^{F} and aflibercept (Eylea commercial lot). The structures of the glycan residues in FIG. 14(A) (G0-GlcNAc; G1-GlcNAc; G1S-GlcNAc; G0; G1, G1S; G2; G2S; G2S2; G0F; G2F2S; G2F2S2; G1F; G1FS; G2F; G2FS; G2FS2; G3FS; G3FS3; G0-2GlcNAc; Man4; Man4_A1G1; Man4_A1G1S1; Man5; Man5_A1G1; Man5_A1G1S1; Man6; Man6_G0+Phosphate; Man6+Phosphate and Man7) are shown. The nomenclature applied to the various glycan structures is standardized-see Varki et al., Symbol nomenclature for glycan Representation, Proteomics 9: 5398-5399 (2009); Harvey et al., Proposal for a standard system for drawing structural diagrams of N- and O-linked carbohydrates and related compounds. Proteomics 2009, 9, 3796-3801; Kornfeld et al., The synthesis of complex-type oligosaccharides II characterization of the processing intermediates in the synthesis of the complex oligosaccharide units of the vesicular stomatitis virus G protein. J Biol Chem. 1978, 253, 7771-7778; Varki et al. (Eds.), Essentials of Glycobiology, 1st Edn., Cold Spring Harbor Laboratory Press, Plainview, N.Y. 1999; Varki et al. (Eds.), Essentials of Glycobiology, 2nd Edn., Cold Spring Harbor Laboratory Press, Plainview, N.Y. 2009; and Dwek, Glycobiology: Moving into the mainstream. Cell 2009, 137, 1175-1176.

FIG. 14(B) sets forth post-translational modifications, other than glycosylation, observed in REGN7483^{F} and aflibercept.

FIG. 14(C) sets forth the glycosylation profile of a separate lot of REGN7483^{F} (mini-trap production 10) as well as REGN7483^{R}, aflibercept and REGN7711.

### Example 12: Short-term Mini-trap Vascular Permeability

Young New Zealand White rabbits were injected in the eye with 80 mcl of an 80 mM solution of DL-α-aminoadipic acid (DL-AAA). Four months later, vascular permeability was assessed by performing fluorescein angiography. Eyes were distributed into 6 groups with similar baseline vascular permeability area (FIG. 15). Then, for each group, eyes were treated with a single intravitreal injection of one of the following:
Group 1: Aflibercept, 500 mcg in 50 mcl, n=6;
Group 2: Aflibercept, 2 mg in 50 mcl, n=6;
Group 3: Recombinant (R) mini-trap (REGN7483^{R}), 250.5 mcg (equimolar dose to group 1) in 50 mcl, n=6;
Group 4: FabRICATOR (F)-cleaved mini-trap (REGN7483^{F}), 254.4 mcg (equimolar dose to group 1) in 50 mcl, n=6;
Group 5: FabRICATOR (F)-cleaved mini-trap (REGN7483^{F}) 1.4 mg in 50 mcl, n=6;
Group 6: 50 mcl of placebo buffer, n=6

Ophthalmic examination was performed at baseline and at weeks 1, 2, 3, 4, 5 and 6. Each ophthalmic examination comprised measurements of intra-ocular pressure (IOP), as well as red-free (RF) imaging (to determine blood vessel morphology), fluorescein angiography (FA; to determine vascular leak), and optical coherence tomography (OCT; to identify vitreal inflammation). Serum (ADA) and plasma (drug levels) were collected at baseline and at weeks 1, 2 and 4.

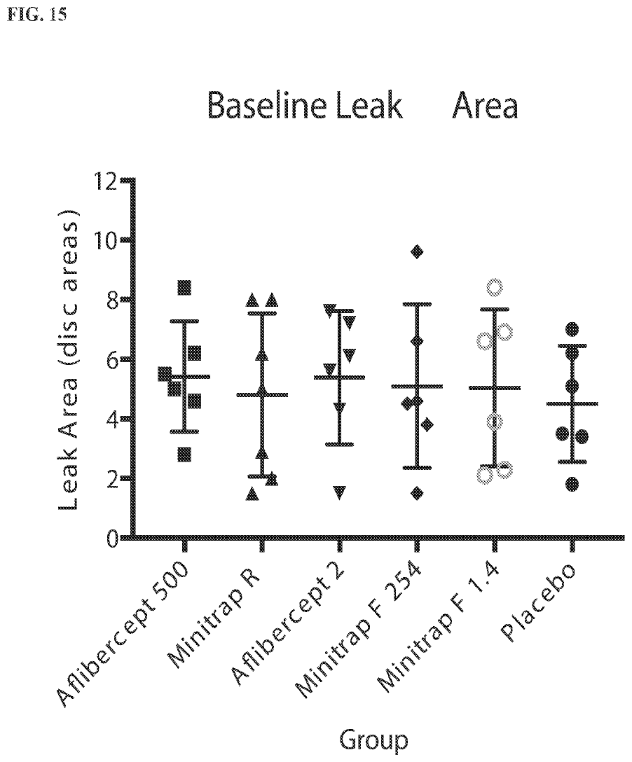

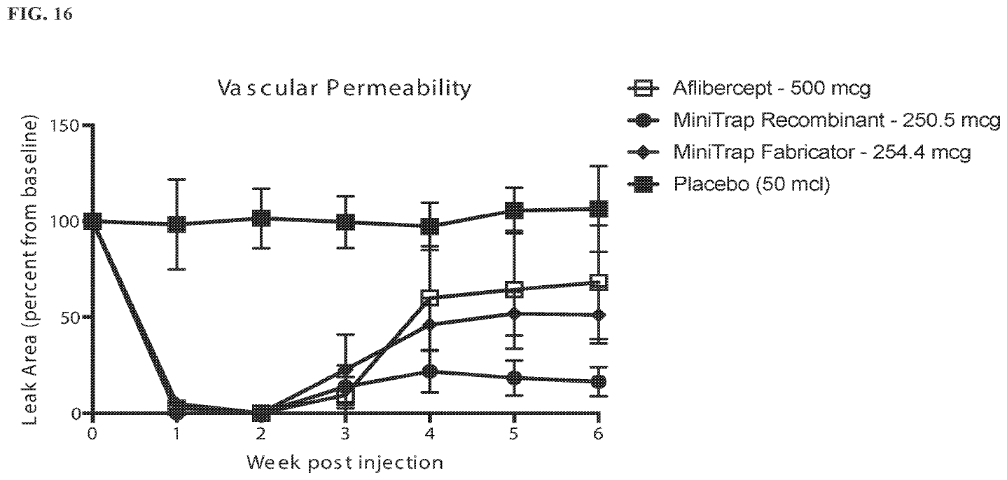

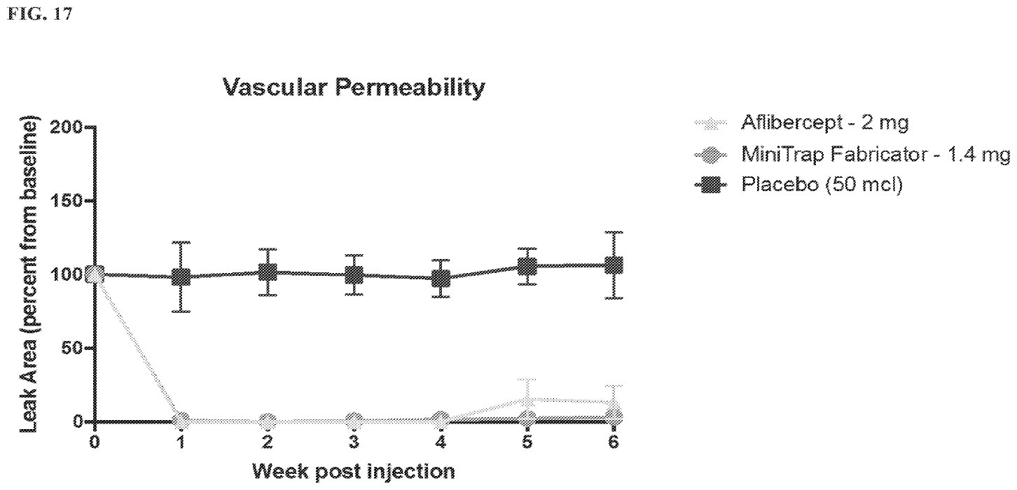

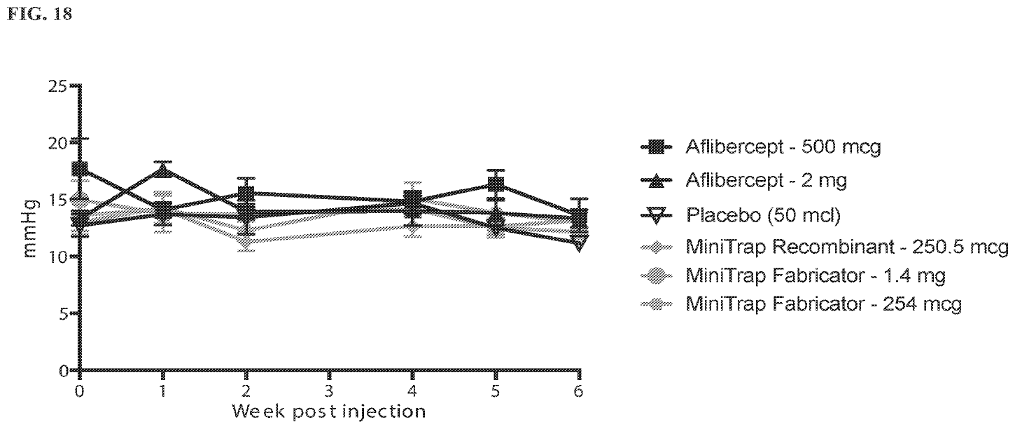

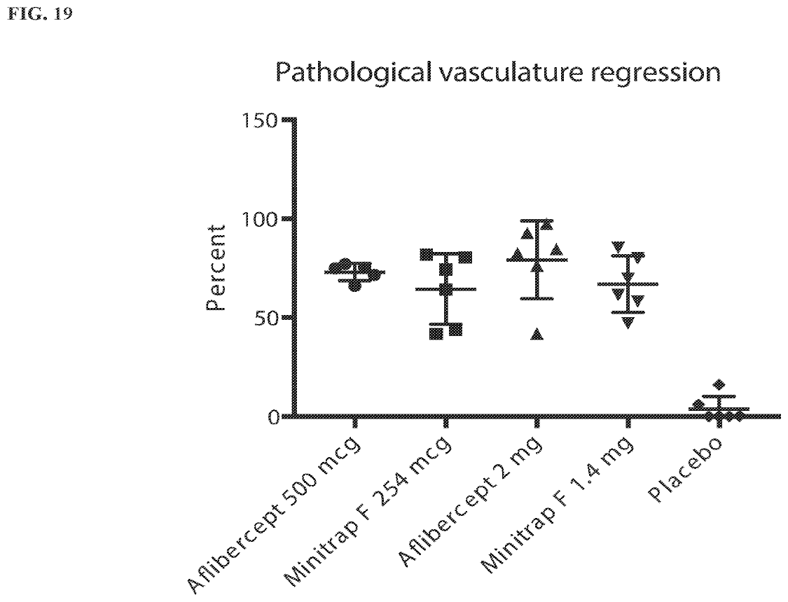

Equimolar doses of aflibercept (500 mcg) and mini-trap (250.5 or 254.4 mcg) blocked vascular permeability for a similar amount of time (FIG. 16). A higher dose of either aflibercept or FabRICATOR-cleaved mini-trap (REGN7483^{F}) blocked vascular permeability for a longer period of time (FIG. 17). Neither FabRICATOR-cleaved mini-trap nor recombinant mini-trap (REGN7483^{R}), at the tested doses, caused significant changes in intraocular pressure (FIG. 18). All treatments caused a similar level of pathological vascular regression (FIG. 19).

### Example 13: Long-Term Mini-Trap Vascular Permeability

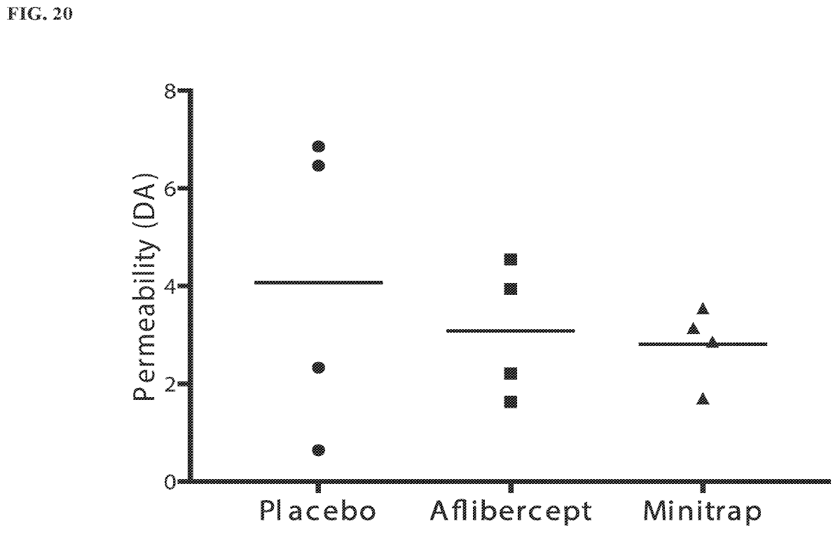

Young New Zealand White rabbits were injected in the eye with 80 mcl of an 80 mM solution of DL-a-aminoadipic acid (DL-AAA). Twenty-two months later, vascular permeability was assessed by performing a fluorescein angiography. Eyes were distributed into 3 groups with similar baseline vascular permeability area (FIG. 20). Following the eye distribution, eyes were treated with a single intravitreal injection of one of the following:
Group 1: Aflibercept, 500 mcg in 50 mcl, n=4;
Group 2: FabRICATOR-cleaved mini-trap (REGN7483^{F}) 213 mcg/eye in 50 mcl, n=4;
Group 3: 50 mcl of placebo buffer, n=4

Ophthalmic examination was performed at baseline and at weeks 1, 2, 4, 5, 6, 8, 10 and 14. Each ophthalmic examination comprised measurements of intra-ocular pressure (IOP), as well as red-free (RF) imaging (to determine blood vessel morphology), fluorescein angiography (FA; to determine vascular leak), and optical coherence tomography (OCT; to identify vitreal inflammation).

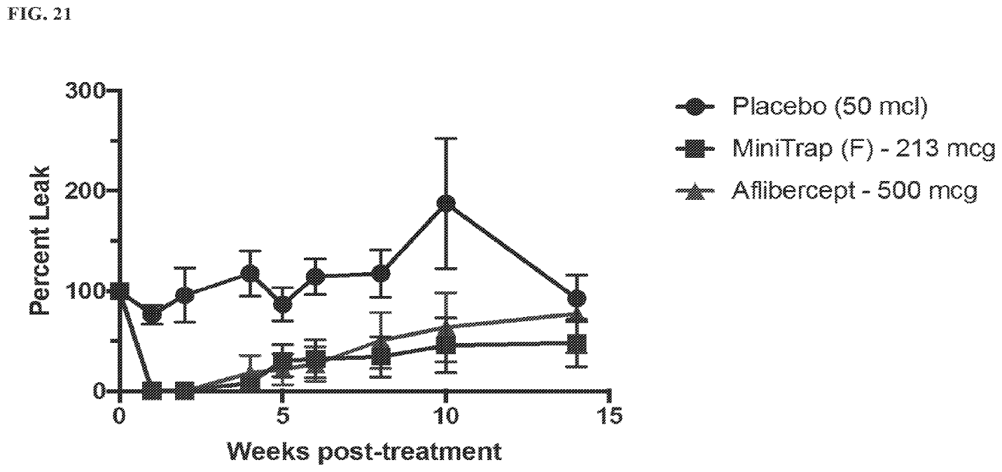

Both aflibercept and FabRICATOR-cleaved mini-trap (REGN7483^{F}) blocked vascular permeability. There were no statistically significant differences in the length of the blockade between aflibercept and mini-trap treatments (FIG. 21).

### Example 14: Fresh Chemically-Defined Medium Incubation Study

The effect of various constituents spiked into fresh chemically-defined media (CDM) containing aflibercept (REGN3) on color was investigated.

The operating parameters for the incubation study were:
50 mL vent-capped shaker tubes with 10mL working volume
Incubated for 7 days, taking samples on Day 0 and 7
Temperature=35.5° C.
pH adjusted to 7.35 with 5N HCl or 5N NaOH
CO₂=6.9%
Humidity=75%
Agitation=150prm
Component Additions (Run as a DOE)
Aflibercept drug substance spiked into shaker tubes at 6 g/L concentration
Matrix=Fresh CDM

Components added to reach a final concentration listed below:
Cysteine: 16.6 mM
Riboflavin: 0.014 mM
Folic Acid: 0.17 mM
Vitamin B12: 0.014mM
Thiamine : 0.18 mM
Niacinamide: 0.84 mM
D-pantothenic acid: 0.62 mM
D-biotin: 0.002 mM
Pyridoxine: 0.49 mM
Iron: 0.22 mM
Copper: 0.0071 mM
Zinc: 0.54 mM

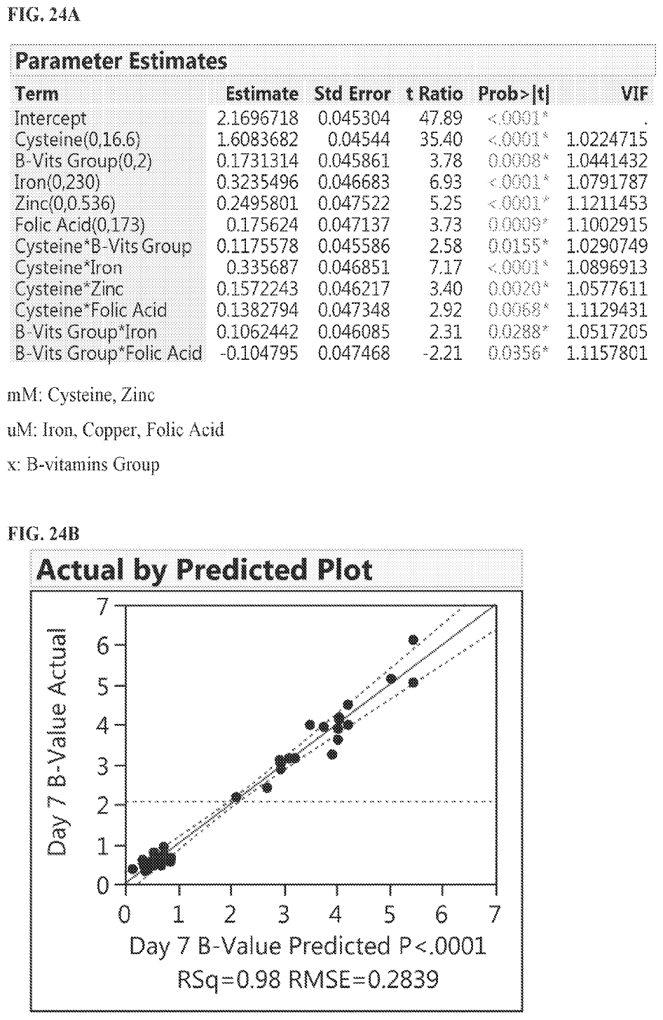

The effect of each constituent addition on the b* value (CIEL*a*b* color space) is set forth in FIG. 24(A-B). Cysteine resulted in the largest color increase. Iron and Zinc generated color when incubated with cysteine. Riboflavin and Vitamin B12 did not statistically impact color.

### Example 15: Evaluation of the Effect of Decreasing Cysteine and Metals on b*-value.

The effect of lowering the concentration of cysteine and of metals on color when REGN3 is expressed was evaluated. The operating parameters for the cell culture study were:
2L Bioreactors
Temperature: about 35° C.
pH about 7
Medium=CDM+Fe, Zn, Cu, Ni, EDTA and citrate as set forth below including cysteine
Nutrient Feeds (Control):
   Day 2 =20× Base CDF
   Day 4 =20× Base CDF
   Day 6 =13× Base CDF
   Day 8 =13× Base CDF
*CDF=chemically-defined nutrient feed
   The following constituents are added to the culture as part of the Base CDF (whether 20× or 13×) at days 2, 4, 6 and 8: about 1-3 micromoles Fe, about 6-19 micromoles Zn, about 0.1-0.3 micromoles Cu, about 8-24 micromoles EDTA, and about 1-3 micromoles citrate per liter of culture.

The bioreactor experimental conditions were as follows:
Dissolved Oxygen Setpoint=20.0%, 40.4% (Control), or 60.0%
Cysteine addition per feed*=about 1.2-1.3 millimoles per L of culture, 1.6-1.7 millimoles per L of culture (Control), or 2.0-2.1 millimoles per L of culture
Metals in the starting CDM*=0.5×, 1×, or 1.5× CDM levels-1× levels are listed below:
   Fe=68-83 micromoles per liter of culture
   Zn=6-7 micromoles per liter of culture
   Cu=0.1-0.2 micromoles per liter of culture
   EDTA=76-95 micromoles per liter of culture
   Citrate=45-55 micromoles per liter of culture
   Ni=0.5-1 micromoles per liter of culture
*Cysteine is fed to the culture every other day.

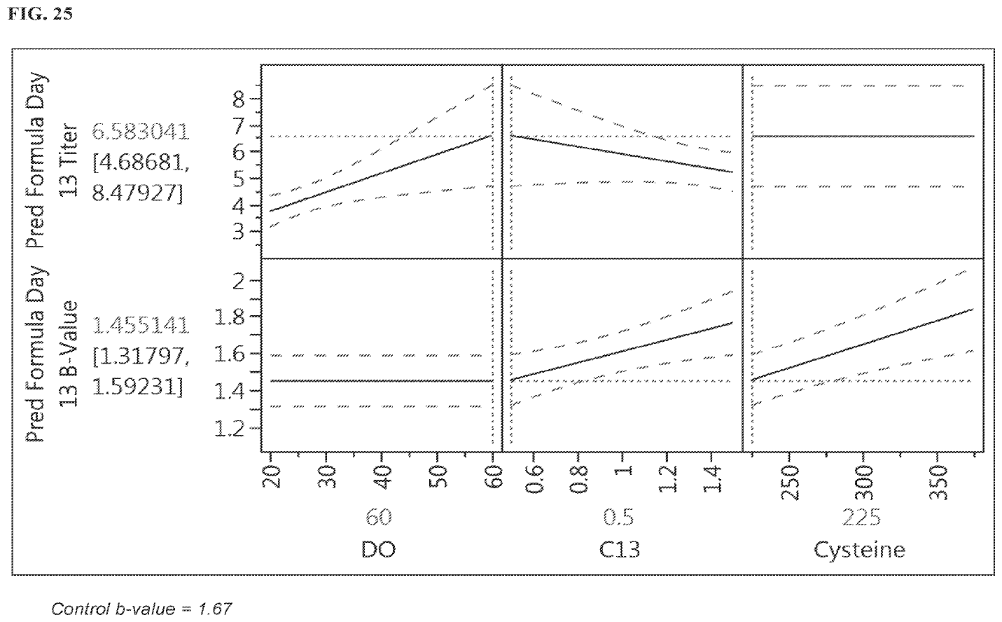

Decreasing cysteine level to 1.2-1.3 millimoles per L per feed reduced color with no significant impact to titer. Decreasing metal concentrations to 0.5× in the medium reduced color with significant increase in titer. There was a minimal impact to VCC (viable cell concentration), viability, ammonia or osmolality. The predicted effect of metal content and cysteine on b*-value is set forth in FIG. 25.

### Example 16: Evaluation of the Effect of Antioxidants on b*-Value

The effect of the antioxidants, taurine, hypotaurine, thioctic acid, glutathione, glycine and vitamin C, on color, spiked into spent CDM containing aflibercept (REGN3), was evaluated. The operating parameters for the incubation study were:
50 mL vent-capped shaker tubes with 10 mL working volume
Incubated for 7 days, taking samples on Day 0 and 7
Temperature=35.5° C.
pH adjusted to 7.35 with 5N HCl or 5N NaOH
CO₂=6.9%
Humidity=75%
Agitation=150 prm

The conditions for component additions to spent CDM were as follows:
Aflibercept drug substance (purified aflibercept recombinant protein in an aqueous buffered solution, pH 6.2, containing 5 mM sodium phosphate, 5 mM sodium citrate and 100 mM sodium chloride) spiked into shaker tubes at 6 g/L concentration Matrix=Spent Medium from mini-trap 2L Control Bioreactor
Antioxidants added to reach the following final concentrations:
   Taurine=10 mM of culture
   Hypotaurine=10 mM of culture
   Glycine=10 mM of culture
   Thioctic Acid=0.0024 mM of culture
   Glutathione, reduced=2 mM of culture
   Choline=1.43 mM of culture
   Hydrocortisone=0.0014 mM of culture
   Vitamin C (ascorbic acid)=0.028 mM of culture
   Vitamin E (a-tocopherol)=0.009 mM of culture

Multiple antioxidants decreased color formation in spent medium: a combination of hypotaurine, taurine and glycine; thioctic acid; and vitamin C. Glutathione increased b*-value.

TABLE 16-1
Summary of Anti-oxidant Effect on Color
Formation of Mini-trap in Spent CDM

| Condition | b*-Value |
|---|---|
| Spent Medium Day 0 | 0.37 |
| Spent Medium Day 7 Control | 1.47 |
| Spent Medium Day 7 + Antioxidants* | 1.02 |

*Antioxidants that significantly decreased b*-value: Hypotaurine/Taurine/Glycine, Thioctic Acid, Vitamin C.

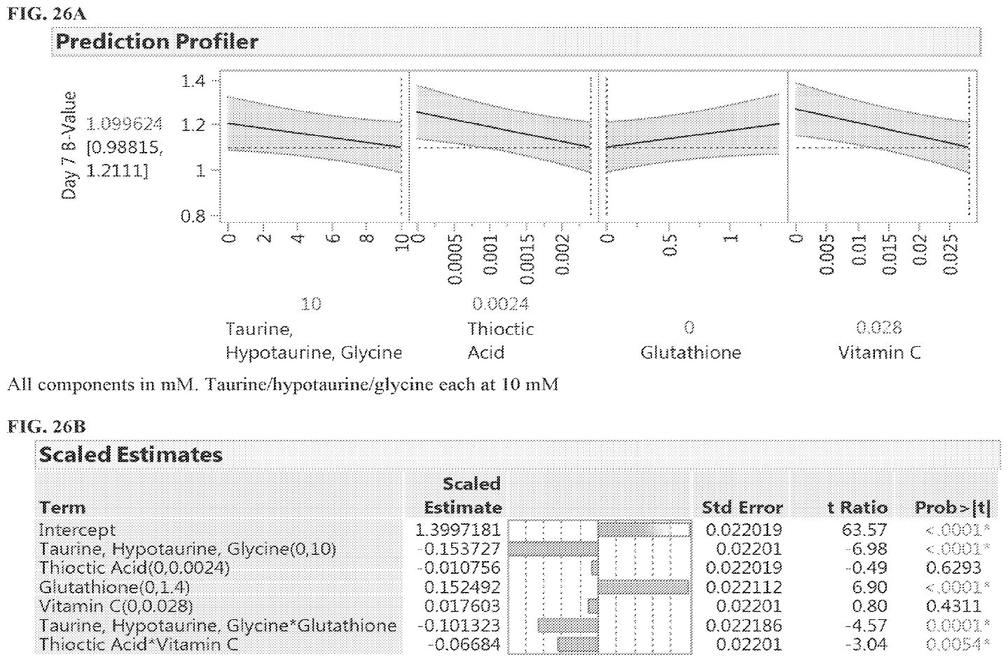

A summary of the predicted effect of various anti-oxidants on b*-value (CIEL*a*b* color space) is set forth in FIG. 26(A-B).

### Example 17: Color Assay Linearity

Mini-trap taken from mini-trap production 23 was diluted from 154 mg/ml to 3.5 mg/ml and the color of each dilution, in the CIEL*a*b* color space, was determined. Table 17-1 sets for the colors that were observed.

TABLE 17-1
Protein Concentration vs b*-value

| Diluted Conc g/L | L* | a* | b* |
|---|---|---|---|
| 154 | 97.1 | −0.85 | 9.97 |
| 100 | 98.04 | −0.67 | 6.75 |
| 75 | 98.5 | −0.51 | 5.03 |
| 50 | 98.94 | −0.36 | 3.58 |
| 25 | 99.47 | −0.13 | 1.65 |
| 10 | 99.77 | −0.02 | 0.66 |
| 5 | 99.9 | 0.01 | 0.36 |
| 3 | 99.95 | 0.06 | 0.08 |

Colors in the various dilutions were plotted on a graph and linear regression analysis of the points was also performed and the relationship between concentration and b* was determined to be expressed by the equation:

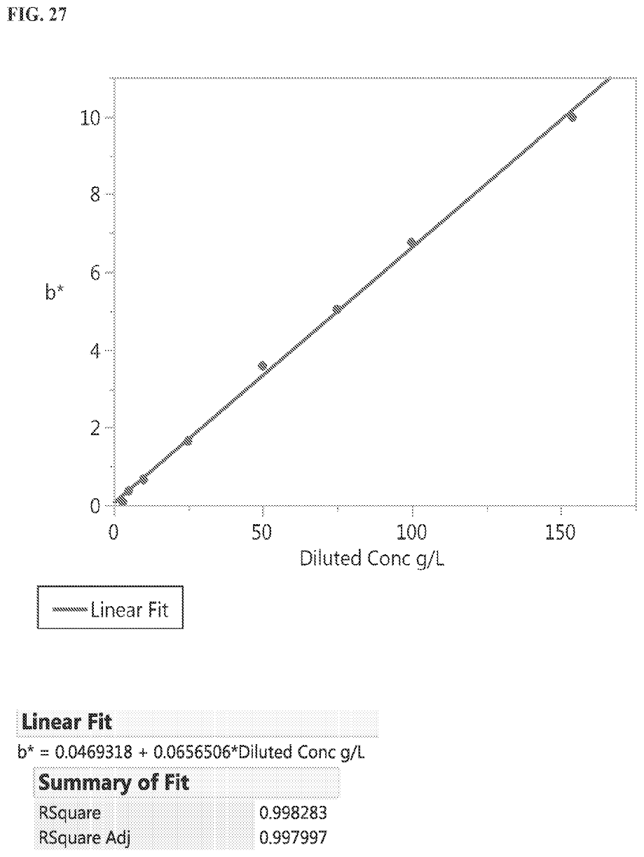

b*=0.046+(0.066×concentration (mg/ml));

wherein L* is about 97-99 and a* is about 0.06-0.85. See FIG. 27.

### Example 18: Evaluation of Color Reduction over Anion Exchange Chromatography (AEX) for REGN3

Color reduction was evaluated for REGN3 on two AEX resins (POROS 50 HQ and Q Sepharose Fast Flow) and three setpoints (pH 8.40 and 2.00 mS/cm, pH 8.00 and 2.50 mS/cm, and pH 7.80 and 4.00 mS/cm).

Five AEX separations were performed for this study as detailed in Table 8-1 with the AEX protocol as detailed in Table 18-2. All AEX loads were sourced from pilot bioreactor S504-190828 (REGN3 X0SP filtered pool, CCF38105-L8). A 15.7 mL Q Sepharose Fast Flow column (19.5 cm bed height, 1.0 cm I.D.) and a 14.1 mL POROS 50 HQ column (18.0 cm bed height, 1.0 cm I.D.) were integrated into an AKTA Avant benchtop liquid chromatography controller for this experiment.

AEX load pH was adjusted to target ±0.05 pH units using 2 M tris base or 2 M acetic acid. AEX load conductivity was adjusted to target ±0.1 mS/cm using 5 M sodium chloride or RODI. All pool samples were analyzed for HMW, color, and yield.

TABLE 18-1
Summary of the Study Design for the
REGN3 AEX Color Reduction Evaluation.

| AEX Separation | Condition Evaluated | Resin |
|---|---|---|
| 1 | pH 8.30-8.50, 1.90-2.10 mS/cm | POROS 50 HQ |
| 2 | pH 7.90-8.10, 2.40-2.60 mS/cm | Q Sepharose |
| | | FF |
| 3 | pH 7.90-8.10, 2.40-2.60 mS/cm | POROS 50 HQ |
| 4 | pH 7.70-7.90, 3.90-4.10 mS/cm | Q Sepharose |
| | | FF |
| 5 | pH 7.70-7.90, 3.90-4.10 mS/cm | POROS 50 HQ |

TABLE 18-2
Flow-through AEX Protocol Used for the REGN3 Color Reduction Evaluation.

| | | | Column | | Linear |
|---|---|---|---|---|---|
| | | | Volumes | Flow | Velocity |
| Step | Description | Mobile Phase | (CVs) | Direction | (cm/h) |
| 1 | Pre-Equilibration | 2M Sodium Chloride (NaCl) | 2 | ↓ | 200 |
| 2 | Equilibration | 50 mM Tris, Variable mM NaCl | 2 | ↓ | 200 |
| | | Variable pH and Conductivity | | | |
| 3 | Load | AEX Load | 40 g/L- | ↓ | 200 |
| | | Variable pH and Conductivity | resin | | |
| 4 | Wash | 50 mM Tris, Variable mM NaCl | 2 | ↓ | 200 |
| | | Variable pH and Conductivity | | | |
| 5 | Strip 1 | 2M Sodium Chloride (NaCl) | 2 | ↑ | 200 |
| 6 | Strip 2 | 1N Sodium Hydroxide (NaOH) | 2 | ↑ | 200 |

AEX, anion exchange chromatography;
CV, column volume

Five AEX separations were performed to evaluate the impact of resin (Q Sepharose FF or POROS 50 HQ) and pH and conductivity setpoint (pH 8.40 and 2.00 mS/cm, pH 8.00 and 2.50 mS/cm, or pH 7.80 and 4.00 mS/cm) on color reduction for REGN3. For POROS 50 HQ, yields (64.4, 81.9, and 91.4%) and pool HMW levels (1.02, 1.29, and 1.83%) increased as the setpoint was changed to a lower pH and higher conductivity. Color (b* values) also increased (1.05, 1.33, and 1.55) as the setpoint was changed to a lower pH and higher conductivity. This indicated that higher pH levels and lower conductivities provided the most reduction in color over the AEX separation for POROS 50 HQ.

The column equilibration buffers and the buffers in which REG3 was formulated when applied to the columns were as follows:
50 mM Tris pH 8.4 and 2.0 mS/cm,
50 mM Tris, 10 mM Acetate pH 8.0 and 2.5 mS/cm, or
50 mM Tris, 10 mM Acetate, 10 mM NaCl pH 7.8 and 4.0 mS/cm

For Q Sepharose Fast Flow, yields (49.5 and 77.7%) and pool HMW levels (0.59 and 1.25%) also increased as the setpoint was changed to a lower pH and higher conductivity. Color (b* values) also increased (0.96 and 1.35) as the setpoint was changed to a lower pH and higher conductivity. This indicated that higher pH levels and lower conductivities provided the most reduction in color over the AEX separation for Q Sepharose Fast Flow.

In addition, Q Sepharose Fast Flow reduced color more than POROS 50 HQ for the two setpoints evaluated on both resins. At the pH 8.00 and 2.50 mS/cm setpoint, POROS 50 HQ pool had a b* value of 1.33 while Q Sepharose Fast Flow pool had a b* value of 0.96. Similarly, at the pH 7.80 and 4.00 mS/cm setpoint, POROS 50 HQ pool had a b* value of 1.55 while Q Sepharose Fast Flow pool had a b* value of 1.35.

TABLE 18-3
Summary of Experimental Results
of the AEX Color Reduction Study*

| AEX | | Yield | HMW | Color | Color | Color |
|---|---|---|---|---|---|---|
| Separation | Fraction | (%) | (%) | (L*) | (a*) | (b*) |
| 1 | Load Flow-through | 64.4 | 1.02 | 98.89 | 0.01 | 1.05 |
| | and Wash | | | | | |
| 2 | Load Flow-through | 49.5 | 0.59 | 98.30 | −0.03 | 0.96 |
| | and Wash | | | | | |
| 3 | Load Flow-through | 81.9 | 1.29 | 99.07 | −0.07 | 1.33 |
| | and Wash | | | | | |
| 4 | Load Flow-through | 77.7 | 1.25 | 99.42 | −0.04 | 1.35 |
| | and Wash | | | | | |
| 5 | Load Flow-through | 91.4 | 1.83 | 99.19 | −0.09 | 1.55 |
| | and Wash | | | | | |
| N/A | REGN3 .011 | N/A | 3.66-3.98 | 98.73 | −0.21 | 3.06 |
| | Filtered Pool | | | | | |

AEX, anion exchange chromatography;
HMW, high molecular weight species;
N/A, not applicable
*All color readings were done at a concentration of 10 g/liter

Color reduction was evaluated for REGN3 on two AEX resins (POROS 50 HQ and Q Sepharose Fast Flow) and three setpoints (pH 8.40 and 2.00 mS/cm, pH 8.00 and 2.50 mS/cm, and pH 7.80 and 4.00 mS/cm). For both resins, color reduction was most optimal for the higher pH and lower conductivity setpoints. In addition, Q Sepharose Fast Flow provided more color reduction than POROS 50 HQ at the two setpoints evaluated on both resins (pH 8.00 and 2.50 mS/cm and pH 7.80 and 4.00 mS/cm).

### Example 19: Glycosylation and Viability Studies for Aflibercept Production Using CDM

In this Example, production of a host cell line expressing the aflibercept fusion protein was carried out using CDM 1, CDM 2 (commercially obtained), and CDM 3 (commercially obtained). A set of experiments was carried out using CDM 1, 2, and 3 with no additional media components. Another set of experiments was performed using CDMs 1-3 to which manganese (manganese chloride trihydrate, Sigma, 3.2 mg/L), galactose (Sigma, 8 g/L), and uridine (Sigma, 6 g/L) were added to the feeds to modify the galactosylation profile. Lastly, a set of experiments was performed using CDMs 1-3 to which manganese (manganese chloride trihydrate, Sigma, 3.2 mg/L), galactose (Sigma, 8 g/L), and uridine (Sigma, 6 g/L) were added to the feeds to modify the galactosylation profile and dexamethasone (Sigma, 12 mg/L) was added to the feeds to modify the sialyation profile of the composition. The harvest using each of the CDMs was prepared by centrifugation followed by 0.45 μm filtration.

Samples were purified by ProA prior to N-glycan analysis.

### Titer Measurements

Aflibercept titers were measured daily using an Agilent (Santa Clara, Calif.) 1200 Series HPLC, or equivalent, operating with a low pH, and step elution gradient with detection at 280 nm. Absolute concentrations were assigned with respect to reference standard calibration curves.

Viable cell density (VCD) and cell viability values

Viable cell density (VCD) and cell viability values were measured through trypan blue exclusion via Nova BioProfile Flex automated cell counters (Nova Biomedical, Waltham, Mass.). Glucose, lactate, offline pH, dissolved oxygen (DO), pCO₂ measurements, and osmolality were measured with the Nova BioProfile Flex (Nova Biomedical, Waltham, Mass.).

### N-Glycan Oligosaccharide Profiling

Approximately 15 μg of Protein A purified samples from harvest of CDM1-3 were prepared for N-glycan analysis in accordance with the Waters GlycoWorks protocol, using the GlycoWorks Rapid Deglycosylation and GlycoWorks RapiFluor-MS Label kits (Waters part numbers 186008939 and 186008091, respectively). N-glycans were removed from the protein by treating the samples with PNGase-F at 50.5° C. for 5 minutes, followed by a cool down at 25° C. for 5 minutes. The released glycans were labeled with RapiFluor-MS fluorescent dye through reaction at room temperature for 5 minutes. The protein was precipitated by adding acetonitrile to the reaction mixture and pelletized to the bottom of the well through centrifugation at 2,204×g for 10 minutes. The supernatant containing the labeled glycans was collected and analyzed on an UPLC using hydrophilic interaction liquid chromatography (Waters BEH Amide column) with post-column fluorescence detection. After binding to the column, the labeled glycans were separated and eluted using a binary mobile phase gradient comprised of acetonitrile and aqueous 50 mM ammonium formate (pH 4.4). The labeled glycans were detected using a fluorescence detector with an excitation wavelength of 265 nm and an emission wavelength of 425 nm. Using the relative area percentages of the N-glycan peaks in the resultant chromatograms, the N-glycan distribution was reported as the total percentage of N-glycans (1) containing a core fucose residue (Total Fucosylation, Table 19-1), (2) containing at least one sialic acid residue (Total Sialylation, Table 19-2), (3) identified as Mannose-5 (Mannose-5, Table 19-3), (4) containing at least one galactose residue (Total Galactosylation, Table 19-4), and (5) of known identity (Total Identified Peaks, Table 19-5).

### Results

Amongst the nine cultures, the culture with CDM1 comprising uridine, manganese, and galactose showed the highest titer at 12 days (5.5 g/L). The culture CDM1 comprising without additional components also showed the high titer at 12 days (about 4.25 g/L); compared to the other seven cultures.

Cell viability results were similar across the various conditions up to process day 6. After process day 7, the CDM2 and CDM3 cultures with or without additional media components showed more than about 90% viability.

CDM1 culture with uridine, manganese and galactose showed the highest VCC around day 6.

The impact of cultures and supplements had a significant impact on the overall N-glycan distribution (Tables 19-1 to 19-5). The glycan levels compared were made using the commercial upstream process to the protein A purified aflibercept (two samples were evaluated) of making aflibercept, which does not employ CDM. The total identified peaks are listed in Table 19-5.

TABLE 19-1
Total Fucosylation (%)

| Condition | Day 6 | Day 10 | Day 12 |
|---|---|---|---|
| CDM1 | 48.75 | — | 46.26 |
| CDM1 + UMG | 49.21 | — | 44.38 |
| CDM1 + UMG + Dex | 48.88 | — | 46.23 |
| CDM2 | — | 45.68 | 45.14 |
| CDM2 + UMG | — | 46.36 | 45.27 |
| CDM2 + UMG + Dex | — | 46.92 | — |
| CDM3 | 49.24 | — | 45.59 |
| CDM3 + UMG | 48.71 | — | 42.61 |
| CDM3 + UMG + Dex | 49.36 | — | 44.56 |

| | |
|---|---|
| Commercial process | 51.37 |
| Commercial process | 52.43 |

U is uridine,
M is manganese,
G is galactose,
Dex is dexamethasone

TABLE 19-2
Total Sialylation (%)

| Condition | Day 6 | Day 10 | Day 12 |
|---|---|---|---|
| CDM1 | 44.06 | — | 39.14 |
| CDM1 + UMG | 43.72 | — | 35.8 |
| CDM1 + UMG + Dex | 43.2 | — | 36.72 |
| CDM2 | — | 37.62 | 36.67 |
| CDM2 + UMG | — | 37.57 | 36.29 |
| CDM2 + UMG + Dex | — | 38.06 | — |
| CDM3 | 44 | — | 31.21 |
| CDM3 + UMG | 42.48 | — | 30.84 |
| CDM3 + UMG + Dex | 43.82 | — | 32.74 |

| | |
|---|---|
| Commercial process | 58.24 |
| Commercial process | 59.23 |

U is uridine,
M is manganese,
G is galactose,
Dex is dexamethasone

TABLE 19-3
Mannose-5 (%)

| Condition | Day 6 | Day 10 | Day 12 |
|---|---|---|---|
| CDM1 | 6.76 | — | 10.1 |
| CDM1 + UMG | 6.9 | — | 13.17 |
| CDM1 + UMG + Dex | 6.23 | — | 8.86 |
| CDM2 | — | 9.71 | 11.96 |
| CDM2 + UMG | — | 9.44 | 10.93 |
| CDM2 + UMG + Dex | — | 8.21 | — |
| CDM3 | 2.31 | — | 12.63 |
| CDM3 + UMG | 2.71 | — | 13.38 |
| CDM3 + UMG + Dex | 2.05 | — | 11.98 |

| | |
|---|---|
| Commercial process | 5.19 |
| Commercial process | 5.24 |

U is uridine,
M is manganese,
G is galactose,
Dex is dexamethasone

TABLE 19-4
Total Galactosylation (%)

| Condition | Day 6 | Day 10 | Day 12 |
|---|---|---|---|
| CDM1 | 68.44 | — | 62.9 |
| CDM1 + UMG | 69.25 | — | 59.02 |
| CDM1 + UMG + Dex | 69.05 | — | 63.26 |
| CDM2 | — | 65.33 | 63.68 |
| CDM2 + UMG | — | 68.13 | 66 |
| CDM2 + UMG + Dex | — | 69.35 | — |
| CDM3 | 74.57 | — | 62.28 |
| CDM3 + UMG | 74.82 | — | 62.2 |
| CDM3 + UMG + Dex | 76.48 | — | 65.18 |

| | |
|---|---|
| Commercial process | 79.64 |
| Commercial process | 80.55 |

U is uridine,
M is manganese,
G is galactose,
Dex is dexamethasone

TABLE 19-5
Total Identified Peaks (%)

| Condition | Day 6 | Day 10 | Day 12 |
|---|---|---|---|
| CDM1 | 87.28 | — | 84.67 |
| CDM1 + UMG | 88.43 | — | 83.82 |
| CDM1 + UMG + Dex | 87.36 | — | 83.44 |
| CDM2 | — | 86.23 | 86.67 |
| CDM2 + UMG | — | 87.81 | 86.87 |
| CDM2 + UMG + Dex | — | 87.53 | — |
| CDM3 | 86.38 | — | 86.31 |
| CDM3 + UMG | 87.07 | — | 86.13 |
| CDM3 + UMG + Dex | 87.18 | — | 87.43 |

| | |
|---|---|
| Commercial process | 93.93 |
| Commercial process | 94.74 |

U is uridine,
M is manganese,
G is galactose,
Dex is dexamethasone

The total fucosylation, total sialylation, total galactosylation and mannose-5 observed on day 12 of the cultures for CDMs was 42.61% to 46.26%, 30.84% to 39.14%, 59.02 to 66% and 8.86% to 13.38%, respectively. These values of glycosylation differ significantly from the glycosylation values obtained using the upstream process to the protein A purified Aflibercept.

### Example 20: VEGF Mini-Trap Intravitreal Photofluorimetry Pharmacokinetics (PK) in Rabbits

The pharmacokinetics of various VEGF traps and mini-traps were analyzed in the eyes of New Zealand White Rabbits.

TABLE 20-1
VEGF Trap and Mini-Trap Proteins Used

| REGN# | Description | Construct Details |
|---|---|---|
| REGN3 | VEFGR1d2- | Flt1 Ig Domain 2(S129-D231).hFLK1 Ig |
| | VEGFR2d3 VEGF | Domain 3(V226- |
| | Trap | K327).higG1_Fc_v2(D104-K330) |
| REGN7483^{F} | Disulfide-linked | Flt1 Ig Domain 2(S129-D231).hFLK1 Ig |
| | homodimer mini Trap | Domain 3(V226-K327).hFc |
| | from Fabricator | DKTHTCPPCPAPELLG(D104-G119) |
| | cleavage of full- | |
| | length VEGF Trap | |
| | REGN3 | |
| REGN7850 | Recombinant | Flt1 Ig Domain 2(S129-D231).hFLK1 Ig |
| | homodimer mini trap | Domain 3(V226- |
| | -hFc | K327).higG1_Fc_v3(D104-C112).PPC |
| | DKTHCPPCPPC | |
| REGN7851 | Recombinant | Flt1 Ig Domain 2(S129-D231).hFLK1 Ig |
| | homodimer mini trap | Domain 3(V226- |
| | -hFc | K327).hIgG1_Fc_v3(D104- |
| | DKTHCPPCPPCPPC | C112).PPCPPC |

### REGN3 and REGN7483 (Experiment 1)

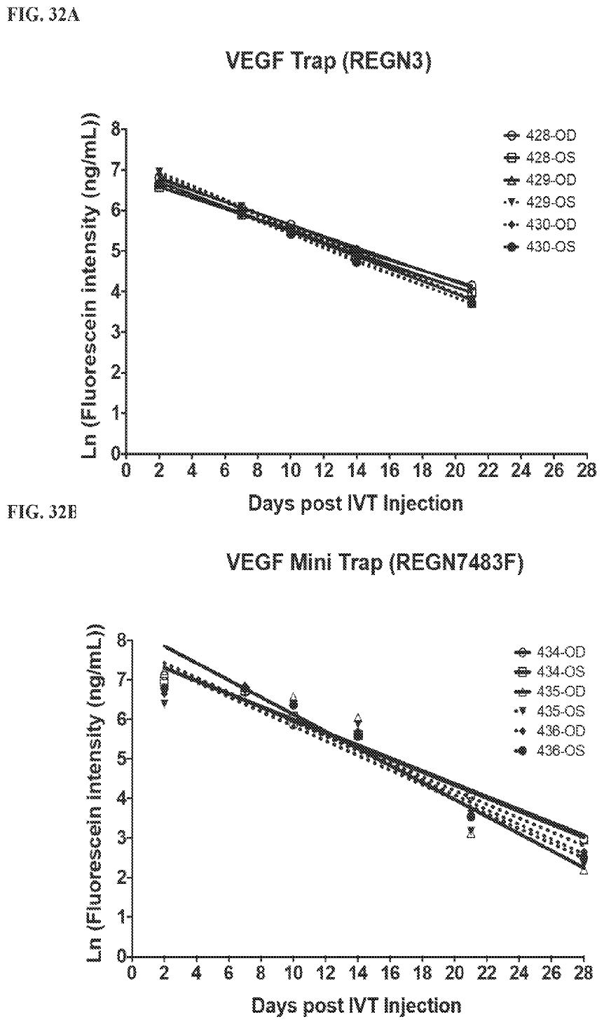

VEGF Trap (REGN3) and VEGF mini-trap (REGN7483^{F}) were molecules tagged with Alexa Fluor 488 (AF488) through amine conjugation. Protein concentrations, endotoxin levels, and Degree of Labeling (DOL) are provided in Table 20-2. The natural log plots of the decay curves for the traps and mini-traps are set forth in FIG. 32. Bilateral intravitreal (IVT) injections were made to 6 male New Zealand White (NZVV) rabbits (6 eyes/3 rabbits/molecule). All eyes were examined for vitreous baseline fluorescence with OcuMetrics Fluorotron fluorophotometer (Mountain View, CA) before injection, and followed up for vitreous fluorescence intensity post injection at Day 2, 7, 10, 14 and 28. General ocular examination included intraocular pressure (IOP), inflammation signs, corneal and conjunctival edema, hemorrhages, floaters in anterior chamber, pupil size and shape, cataract, and retinal detachment before and 10 minutes after IVT injection, and at each follow-up time point. Fluorescence intensity and position information were extracted and imported in GraphPad Prism for graphical display and analysis. The data were fitted to a first order, single compartment model.

TABLE 20-2
Half-life (t_{1/2}) of VEGF Trap and VEGF Mini-Trap
in Intravitreal Photofluorimetry PK in Rabbits

| | | | | DOL (Degree | | |
|---|---|---|---|---|---|---|
| | | Conc. | Endotoxin | of AF488 | Volume | t_{1/2} (Std) |
| Test article | Conjugation | mg/ml | (EU/mL) | Labeling) | (uL)/eye | Days |
| REGN3 | AF488 | 4.17 | <0.5 | 2.38 | 50 | 4.6 (0.3) |
| REGN7483^{F} | AF488 | 4.18 | 1.07 | 2.24 | 50 | 3.9 (0.4) |

### Results

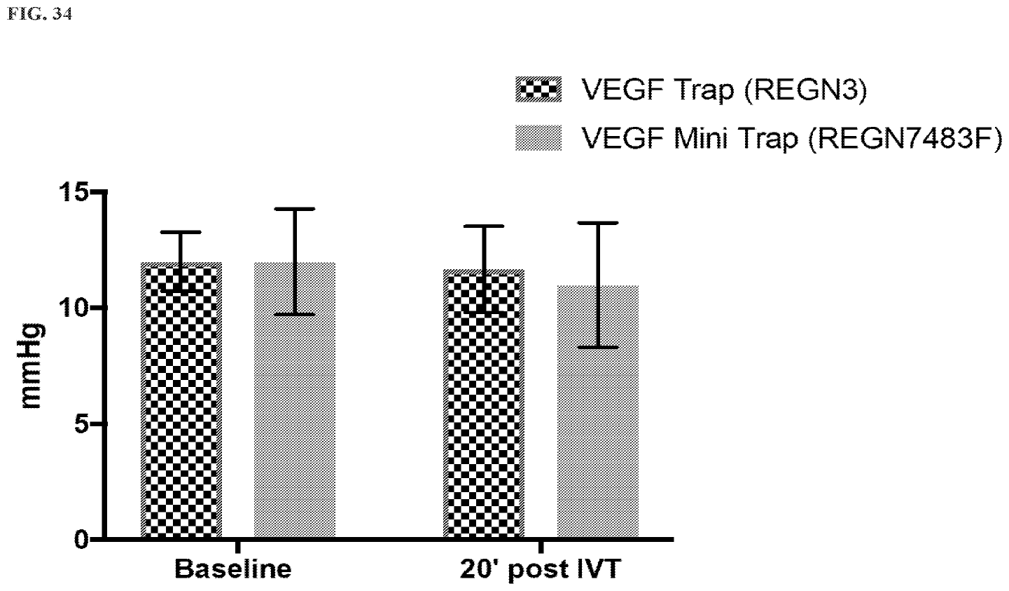

The PK study of VEGF Trap (REGN3) and VEGF mini-trap (REGN7483^{F}) in NZW rabbit vitreous showed the half-lives were 4.6 (±0.3) and 3.9 (±0.4) days, respectively. There was no significant IOP change before and after IVT injection of either molecule. See FIG. 34. There were no clinically notable signs observed in general ocular examination.

### Conclusions

The rabbit vitreal half-life of VEGF Trap measured conventionally in previous studies was 4.8 days, comparable to that measured by in vivo fluorophotometry. The current study showed the half-life of VEGF mini-trap (REGN7483^{F}) is shorter than that of VEGF Trap (REGN3) in NZW rabbit vitreous, VEGF mini-trap persisting about 15% shorter (3.9 days vs 4.6 days).

### REGN3, REGN7850 and REGN7851 (Experiment 2)

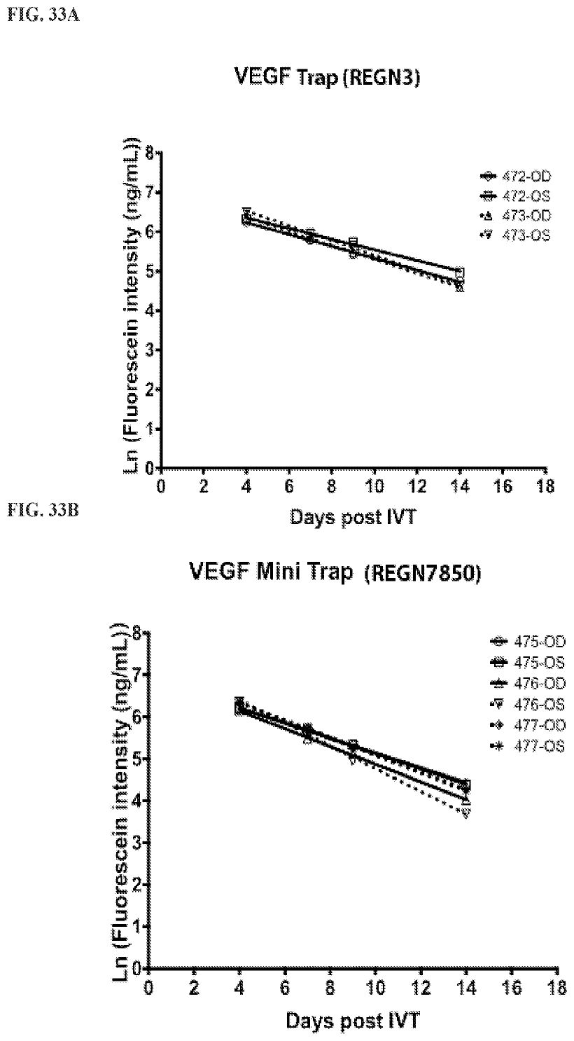

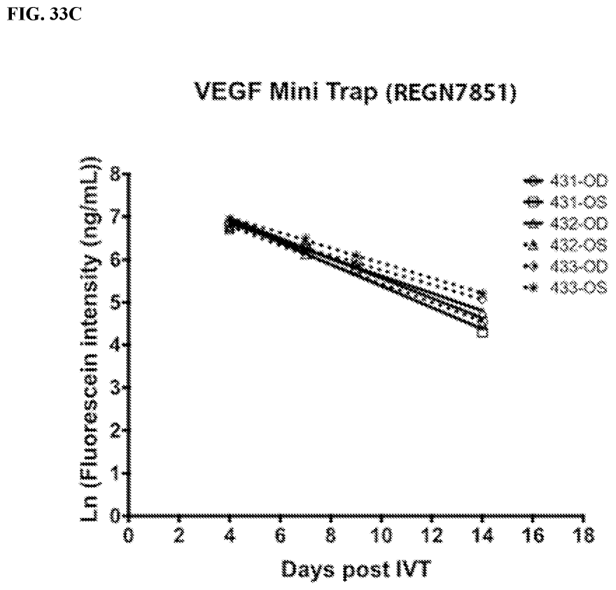

VEGF Trap (REGN3), and two VEGF mini-trap (REGN7850 and REGN7851) were molecules tagged with Alexa Fluor 488 (AF488) through amine conjugation. Protein concentrations, endotoxin levels, and Degree of Labeling (DOL) are provided in Table 20-3. The natural log plots of the decay curves for the traps and mini-traps are set forth in FIG. 33. Bilateral intravitreal (IVT) injections were made to 6 male New Zealand White (NZW) rabbits (6 eyes/3 rabbits/molecule). All eyes were examined for vitreous baseline fluorescence with OcuMetrics Fluorotron fluorophotometer (Mountain View, Calif.) before injection, and followed up for vitreous fluorescence intensity post injection at Day 4, 7, 9, and 14. General ocular examination included intraocular pressure (IOP), inflammation signs, corneal and conjunctival edema, hemorrhages, floaters in anterior chamber, pupil size and shape, cataract, and retinal detachment before and 10 minutes after IVT injection, and at each follow-up time point. Fluorescence intensity and position information were extracted and imported in GraphPad Prism for graphical display and analysis. The data were fitted to a first order, single compartment model.

TABLE 20-3
Half-Life of VEGF Trap and Different Variants of Mini-
Traps in Intravitreal Photofluorimetry PK in Rabbits

| | | | | DOL (Degree | | |
|---|---|---|---|---|---|---|
| | | Conc. | Endotoxin | of AF488 | Volume | t_{1/2} (Std) |
| Test article | Conjugation | mg/ml | (EU/mL) | Labeling) | (uL)/eye | Days |
| REGN3 | AF488 | 3 | <0.5 | 3.94 | 50 | 4.3 (0.7) |
| REGN7850 | AF488 | 2.09 | <0.5 | 2.18 | 50 | 3.4 (0.5) |
| REGN7851 | AF488 | 4.14 | <0.5 | 2.13 | 50 | 3.4 (0.5) |

### Results

The PK study of VEGF Trap (REGN3) and VEGF mini-traps (REGN7850 and REGN7851) in NZW rabbit vitreous showed the half-lives were 4.3 (±0.3), 3.4 (±0.5), and 3.4 (±0.5) days, respectively. There was no significant IOP change before and after IVT injection of either molecule. There were no clinically notable signs observed in general ocular examination.

### Conclusions

PK study measured by in vivo fluorophotometry shows the half-lives of another two variants of VEGF mini-traps (REGN7850 and REGN7851) are shorter than that of VEGF Trap (REGN3) in NZW rabbit vitreous, both VEGF mini-traps persisting about 21% shorter (3.4 days vs. 4.3 days).

TABLE 20-4
Summary of Half-Life of Individual Eyes

| Experiment# | REGN # | Animal ID | OD | OS | t_{1/2} | Std |
|---|---|---|---|---|---|---|
| 1 | REGN3 | 428 | 5.0 | 4.5 | 4.6 | 0.3 |
| | | 429 | 4.2 | 5.1 | | |
| | | 430 | 4.3 | 4.4 | | |
| | REGN7483^{F} | 434 | 4.2 | 4.3 | 3.9 | 0.4 |
| | | 436 | 3.2 | 3.7 | | |
| | | 437 | 4.0 | 3.7 | | |
| 2 | REGN3 | 472 | 4.6 | 5.1 | 4.3 | 0.7 |
| | | 473 | 3.9 | 3.7 | | |
| | REGN7850 | 475 | 3.9 | 3.8 | 3.4 | 0.5 |
| | | 476 | 3.3 | 2.6 | | |
| | | 477 | 3.4 | 3.3 | | |
| | REGN7851 | 478 | 3.0 | 2.7 | 3.4 | 0.5 |
| | | 479 | 3.4 | 3.1 | | |
| | | 480 | 4.0 | 4.0 | | |

All references cited herein are incorporated by reference to the same extent as if each individual publication, database entry (e.g., Genbank sequences or GeneID entries), patent application, or patent, was specifically and individually indicated to be incorporated by reference.

1. An isolated VEGF mini-trap comprising the following domain structure:
((R1D2)-(R2D3))ₐ-(MC)_{c};
wherein one or more histidines of said VEGF mini-trap are oxidized to 2-oxo-histidine,
and/or one or more tryptophans are dioxidated,
and/or one or more asparagines thereof are glycosylated,
or,
((R1D2)-(R2D3)-(R2D4))ₐ-(MC)_{b},
((R1D2)-(R2D3))_{c}-linker-((R1D2)-(R2D3))_{d}; or
((R1D2)-(R2D3)-(R2D4))ₑ-linker-((R1D2)-(R2D3)-(R2D4))_{f};
wherein,
R1D2 is the VEGFRI Ig domain 2;
R2D3 is the VEGFR2 Ig domain 3;
R2D4 is the VEGFR2 Ig domain 4;
MC is a multimerizing component which is a fragment of an immunoglobulin hinge region or a polypeptide consisting of the amino acid sequence:
(SEQ ID NO: 22)
DKTHTCPPC,
(SEQ ID NO: 23)
DKTHTCPPCPPC,
(SEQ ID NO: 24)
DKTHTCPPCPPCPPC,
(SEQ ID NO: 25)
DKTHTC(PPC)ₕ, wherein h is 1, 2, 3, 4, or 5,
(SEQ ID NO: 6)
DKTHTCPPCPAPELLG,
(SEQ ID NO: 7)
DKTHTCPLCPAPELLG,
(SEQ ID NO: 8)
DKTHTC
or
(SEQ ID NO: 9)
DKTHTCPLCPAP
and
linker is a peptide comprising about 5, 6, 7, 8, 9, 10, 11, 12, 13, 14, 15 or 16 amino acids;
and, independently, a=1, 2, 3, 4, 5, 6, 7, 8, 9, 10, 11, 12, 13, 14 or 15; b=1, 2, 3, 4, 5, 6, 7, 8, 9, 10, 11, 12, 13, 14 or 15; c=1, 2, 3, 4, 5, 6, 7, 8, 9, 10, 11, 12, 13, 14 or 15; d=1, 2, 3, 4, 5, 6, 7, 8, 9, 10, 11, 12, 13, 14 or 15; e=1, 2, 3, 4, 5, 6, 7, 8, 9, 10, 11, 12, 13, 14 or 15; and
f=1, 2, 3, 4, 5, 6, 7, 8, 9, 10, 11, 12, 13, 14 or 15;
or a composition thereof.

2. The VEGF mini-trap or composition thereof of claim 1 selected from
(i) (R1D2)₁-(R2D3)₁-(MC)₁;
and
(ii) (R1D2)₁-(R2D3)₁-(R2D4)₁-(MC)₁.

3. A VEGF mini-trap or composition thereof comprising an amino acid sequence set forth in a member selected from the group consisting of:
(SEQ ID NO: 12)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKDKTHTCPPCPAPELLG;
(SEQ ID NO: 13)
GRPFVEMYSEIPEIIHMTEGRELVIPCRVTSPNITVTLKKFPLDTLIPDGKRIIWDSRKGFIISNATYKEIGLLTCE
ATVNGHLYKTNYLTHRQTNTIIDVVLSPSHGIELSVGEKLVLNCTARTELNVGIDFNWEYPSSKHQHKKLVNRDLKT
QSGSEMKKFLSTLTIDGVTRSDQGLYTCAASSGLMTKKNSTFVRVHENLSVAFGSGMESLVEATVGERVRIPAKYLG
YPPPEIKWYKNGIPLESNHTIKAGHVLTIMEVSERDTGNYTVILTNPISKEKQSHVVSLVVYVPPGPGDKTHTCPLC
PAPELLG;
(SEQ ID NO: 26)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKDKTHTCPPC;
(SEQ ID NO: 27)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKDKTHTCPPCPPC;
(SEQ ID NO: 28)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKDKTHTCPPCPPCPPC;
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKDKTHTC-(PPG)x
(SEQ ID NO: 29; wherein x is 1, 2, 3, 4 or 5);
(SEQ ID NO: 10)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKGGGGSGGGGSGGGGSGGGGSGGGGSG
GGGSSDTGRPFVEMYSEIPEIIHMTEGRELVIPCRVTSPNITVTLKKFPLDTLIPDGKRIIWDSRKGFIISNATYKE
IGLLTCEATVNGHLYKTNYLTHRQTNTIIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFVRVHEK;
(SEQ ID NO: 11)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKGGGGSGGGGSGGGGSSDTGRPFVEMY
SEIPEIIHMTEGRELVIPCRVTSPNITVTLKKFPLDTLIPDGKRIIWDSRKGFIISNATYKEIGLLTCEATVNGHLY
KTNYLTHRQTNTIIDVVLSPSHGIELSVGEKLVLNCTARTELNVGIDFNWEYPSSKHQHKKLVNRDLKTQSGSEMKK
FLSTLTIDGVTRSDQGLYTCAASSGLMTKKNSTFVRVHEK;
(SEQ ID NO: 32)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKGGGGSGGGGSGGGGSGGGGSGGGGSG
GGGSGGGGSGGGGSGGGGSSDTGRPFVEMYSEIPEIIHMTEGRELVIPCRVTSPNITVTLKKFPLDTLIPDGKRIIW
DSRKGFIISNATYKEIGLLTCEATVNGHLYKTNYLTHRQTNTIIDVVLSPSHGIELSVGEKLVLNCTARTELNVGID
FNWEYPSSKHQHKKLVNRDLKTQSGSEMKKFLSTLTIDGVTRSDQGLYTCAASSGLMTKKNSTFVRVHEK;
(SEQ ID NO: 33)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEKGGGGSGGGGSGGGGSGGGGSGGGGSG
GGGSGGGGSGGGGSGGGGSGGGGSGGGGSGGGGSSDTGRPFVEMYSEIPEIIHMTEGRELVIPCRVTSPNITVTLKK
FPLDTLIPDGKRIIWDSRKGFIISNATYKEIGLLTCEATVNGHLYKTNYLTHRQTNTIIDVVLSPSHGIELSVGEKL
VLNCTARTELNVGIDFNWEYPSSKHQHKKLVNRDLKTQSGSEMKKFLSTLTIDGVTRSDQGLYTCAASSGLMTKKNS
TFVRVHEK;
and
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEK-(GGGGS)y-
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDFNWEYPSSKHQHKKLVNRD
LKTQSGSEMKKFLSTLTIDGVTRSDQGLYTCAASSGLMTKKNSTFVRVHEK
(SEQ ID NO: 30; wherein y is 1, 2, 3, 4, 5, 6, 7, 8, 9, 10, 11, 12, 13, 14 or 15).

4. The VEGF mini-trap or composition thereof of claim 1 wherein said mini-trap comprises the domain structure:
(i) (R1D2)ₐ-(R2D3)_{b}-linker-(R1D2)_{c}-(R2D3)_{d}; or
(ii) (R1D2)ₐ-(R2D3)_{b}-(R2D4)_{c}-linker-(R1D2)_{d}-(R2D3)ₑ-(R2D4)_{f}; and
having a secondary structure wherein:
(i) said R1D2 domains coordinate;
(ii) said R2D3 domains coordinate; and/or
(iii) said R2D4 domains coordinate,
to form a VEGF binding domain.

5. The VEGF mini-trap or composition thereof of claim 1, wherein linker is (Gly₄Ser)ₙ wherein n is 1, 2, 3, 4, 5, 6, 7, 8, 9, 10, 11, 12, 13, 14 or 15; or
wherein the MC is a fragment of an immunoglobulin hinge region that forms 2, 3 or 4 cysteine bridges with another MC.

6. The VEGF mini-trap or composition thereof of claim 1 wherein said polypeptides are homodimerized.

7. The VEGF mini-trap or composition thereof of claim 1, wherein one or more histidines of said VEGF mini-trap are oxidized to 2-oxo-histidine, and/or one or more tryptophans of said VEGF mini-trap are dioxidated, and/or one or more asparagines of said VEGF mini-trap thereof are glycosylated.

8. The VEGF mini-trap or composition thereof of claim 1 which is a composition comprising said VEGF mini-trap wherein between 0.1% and 2% of histidines in the VEGF mini-trap are 2-oxo-histidine.

9. The VEGF mini-trap or composition thereof of claim 1 which is a composition comprising said VEGF mini-trap polypeptide wherein oligopeptide products of digestion of said VEGF mini-trap, which comprises one or more carboxymethylated cysteines and 2-oxo-histidines, with Lys-C and trypsin proteases are:
EIGLLTC*EATVNGH*LYK (amino acids 73-89 of SEQ ID NO: 12) which comprises about 0.006-0.013% 2-oxo-histidines,
QTNTIIDVVLSPSH*GIELSVGEK (amino acids 97-119 of SEQ ID NO: 12) which comprises about 0.019-0.028% 2-oxo-histidines,
TELNVGIDFNWEYPSSKH*QHK (amino acids 128-148 of SEQ ID NO: 12) which comprises about 0.049-0.085% 2-oxo-histidines,
DKTH*TC*PPC*PAPELLG (amino acids 206-221 of SEQ ID NO: 12) which comprises about 0.057-0.092% 2-oxo-histidines, and/or
TNYLTH*R (amino acids 90-96 of SEQ ID NO: 12) which comprises about 0.010-0.022% 2-oxo-histidines, and
optionally, IIW*DSR (amino acids 56-61 of SEQ ID NO: 12) which comprises about 0.198-0.298% 2-oxo-histidines,
wherein H* is a histidine that may be oxidized to 2-oxo-histidine, W* is tryptophan that may be dioxidated and wherein C* is a cysteine that may be carboxymethylated.

10. The VEGF mini-trap or composition thereof of claim 1 which is a composition comprising said VEGF mini-trap wherein oligopeptide products of digestion of said VEGF mini-trap, which comprises one or more carboxymethylated cysteines and 2-oxo-histidines, with Lys-C and trypsin proteases are:
EIGLLTC*EATVNGH*LYK (amino acids 73-89 of SEQ ID NO: 12) which comprises about 0.0095% 2-oxo-histidines,
QTNTIIDVVLSPSH*GIELSVGEK (amino acids 97-119 of SEQ ID NO: 12) which comprises about 0.0235% 2-oxo-histidines,
TELNVGIDFNWEYPSSKH*QHK (amino acids 128-148 of SEQ ID NO: 12) which comprises about 0.067% 2-oxo-histidines,
DKTH*TC*PPC*PAPELLG (amino acids 206-221 of SEQ ID NO: 12) which comprises about 0.0745% 2-oxo-histidines, and/or
TNYLTH*R (amino acids 90-96 of SEQ ID NO: 12) which comprises about 0.016% 2-oxo-histidines, and
optionally, IIW*DSR (amino acids 56-61 of SEQ ID NO: 12) which comprises about 0.248% 2-oxo-histidines,
wherein H* is a histidine that may be oxidized to 2-oxo-histidine, W is tryptophan that may be dioxidated and wherein C* is a cysteine that may be carboxymethylated.

11. The VEGF mini-trap or composition thereof of claim 1 which is a composition wherein one or more tryptophans are dioxidized.

12. The VEGF mini-trap or composition thereof of claim 1 which is a composition wherein the 2-oxo-histidine is characterized by the chemical formula:

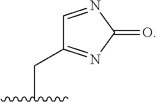 [SMILES: *CC1=NC(=O)N=C1]

13. The VEGF mini-trap or composition thereof of claim 1 which is a composition characterized by a color:
(i) which is no more brown-yellow than European Color Standard BY2;
(ii) which is no more brown-yellow than European Color Standard BY3;
(iii) which is no more brown-yellow than European Color Standard BY4;
(iv) which is no more brown-yellow than European Color Standard BY5;
(v) which is no more brown-yellow than European Color Standard BY6;
(vi) which is no more brown-yellow than European Color Standard BY7;
(vi) which is between European Color Standard BY2 and BY3;
(vii) which is between European Color Standard BY2 and BY4;
(vii) wherein, in the CIEL*a*b* color space, L* is about 70-99, a* is about −2-0 and b* is about 20 or less; and/or
(viii) wherein, in the CIEL*a*b* color space, L* is about 98-99, a* is about −1-0 and b* is about 5-10 and the mini-trap concentration is between 75 and 100 mg/ml; optionally, wherein the concentration of VEGF mini-trap is about 70-200 mg/ml or optionally, wherein the concentration of VEGF mini-trap is about 70-200 mg/ml, but is characterized by said color when diluted to about 10, 11, 10-11, 80 or 90 mg/ml.

14. The VEGF mini-trap or composition thereof of claim 1 which is a composition wherein the color of the composition is characterized by the following formula:
0.046+(0.066×concentration of mini-trap (mg/ml))=b* or b*=(0.11×concentration of mini-trap (mg/ml)−0.56), wherein L*=about 97-99 and a=about −0.085-0.06.

15. The VEGF mini-trap or composition thereof of claim 1 which is a composition that is the product of a process comprising:
(i) expressing aflibercept or said VEGF mini-trap in a host cell in a chemically-defined liquid medium wherein said aflibercept or VEGF mini-trap is secreted from the host cell into the medium; and
(ii) if aflibercept is expressed, further comprising proteolytic cleavage of the aflibercept to produce peptides comprising the Fc domain, or a fragment thereof, and said VEGF mini-trap, and removal of the Fc domain or fragment thereof from the VEGF mini-trap;
(iii) applying the VEGF mini-trap to an anion-exchange chromatography resin; and
(iv) retaining said VEGF mini-trap polypeptide in the chromatographic flow-through thereof.

16. The VEGF mini-trap or composition thereof of claim 15, which is a composition, wherein if said aflibercept is expressed, the process further comprises, prior to said proteolytic cleavage, protein-A purification of the aflibercept.

17. The VEGF mini-trap or composition of claim 15, which is a composition, wherein the anion exchange resin comprises:
a strong anion exchange resin;
a quaternary amine functional group;
a —O—CH₂CHOHCH₂OCH₂CHOHCH₂N⁺(CH₃)₃ functional group; or
a quaternized polyethyleneimine functional group.

18. The VEGF mini-trap or composition of claim 15, which is a composition, wherein the proteolytic cleavage is performed by incubating the aflibercept with Streptococcus pyogenes IdeS protease or a variant thereof comprising one or more point mutations.

19. The VEGF mini-trap or composition of claim 15, which is a composition, wherein said VEGF mini-trap, is applied to the anion-exchange (AEX) chromatography resin under a condition selected from the group consisting of:
(1) the AEX resin comprises a quaternized polyethyleneimine functional group and is equilibrated with a buffer at pH 8.30-8.50 having a conductivity of 1.90-2.10 mS/cm;
(2) the AEX resin comprises a —O—CH₂CHOHCH₂OCH₂CHOHCH₂N⁺(CH₃)₃ or —N⁺(CH₃)₃ or a quaternary amine functional group and is equilibrated with a buffer at pH 7.90-8.10 having a conductivity of 2.40-2.60 mS/cm;
(3) the AEX resin comprises a quaternized polyethyleneimine functional group and is equilibrated with a buffer at pH 7.90-8.10 having a conductivity of 2.40-2.60 mS/cm;
(4) the AEX resin comprises a —O—CH₂CHOHCH₂OCH₂CHOHCH₂N⁺(CH₃)₃ or —N⁺(CH₃)₃ or a quaternary amine functional group and is equilibrated with a buffer at pH 7.70-7.90 having a conductivity of 3.90-4.10 mS/cm;
(5) the AEX resin comprises a quaternized polyethyleneimine functional group and is equilibrated with a buffer at pH 7.70-7.90 having a conductivity of 3.90-4.10 mS/cm;
(6) the AEX resin comprises a —O—CH₂CHOHCH₂OCH₂CHOHCH₂N⁺(CH₃)₃ or —N⁺(CH₃)₃ or a quaternary amine functional group and is equilibrated with a buffer at pH 7.70±0.1 having a conductivity of 9.0±0.1 mS/cm; and
(7) the AEX resin comprises a quaternized polyethyleneimine functional group and is equilibrated with a buffer at pH 8.4±0.1 having a conductivity of 2.0±0.1 mS/cm;
optionally wherein:
the buffer under condition (1) comprises: 50 mM Tris, pH 8.4 and 2.0 mS/cm;
the buffer under conditions (2)-(3) comprises: 50 mM Tris, 10 mM acetate, pH 8.0 and 2.5 mS/cm;
the buffer under conditions (4)-(5) comprises: 50 mM Tris, 10 mM Acetate, 10 mM NaCl, pH 7.8 and 4.0 mS/cm;
the buffer under condition (6) comprises: 50 mM Tris, 60 mM NaCl, pH7.7±0.1; and/or
the buffer under condition (7) comprises: 50 mM Tris, pH 8.4±0.1;
the VEGF mini-trap is in a loading buffer before application to the resin which is the equilibration buffer;
and/or
following application of the VEGF mini-trap to the resin, said resin is washed with said aqueous buffer.

20. The VEGF mini-trap or composition of claim 15, which is a composition, wherein the Fc domain or fragment thereof is chromatographically removed from the VEGF mini-trap, following proteolytic cleavage, by applying the composition comprising Fc domain or fragment and VEGF mini-trap to a protein-A chromatography resin and retaining the VEGF mini-trap in the flow-through fraction.

21. The VEGF mini-trap or composition of claim 15, which is a composition, wherein the process further comprises adjustment of the pH to about 5.5, 5.6, 5.7, 5.8, 5.9, 6.0, 6.1 or 6.2, filtration, diafiltration, viral inactivation, protein-A chromatographic purification and/or hydrophobic interaction chromatographic purification.

22. The VEGF mini-trap or composition of claim 15, which is a composition, wherein the process further comprises hydrophobic interaction chromatographic purification on a resin the comprises a phenyl functional group.

23. The VEGF mini-trap or composition of claim 15, which is a composition, wherein the hydrophobic interaction chromatographic purification is done in bind-and-elute mode or flow-through mode.

24. The VEGF mini-trap or composition of claim 15, which is a composition, wherein:
said mini-trap was expressed in a host cell which is in a chemically defined medium (CDM) or wherein said mini-trap is the product of a process comprising proteolytic cleavage of aflibercept, with IdeS enzyme, wherein said aflibercept was expressed in a host cell which is in a liquid chemically defined medium wherein said host cell was cultured in a process comprising the steps:
(i) introducing the host cell to a CDM comprising:
about 68 micromoles Fe per liter of culture,
about 6 micromoles Zn per liter of culture,
about 0.1 micromoles Cu per liter of culture,
about 76 micromoles EDTA per liter of culture,
about 45 micromoles citrate per liter of culture, and
about 0.5 micromoles Ni per liter of culture and, optionally
about 1.2 millimoles cysteine per liter of culture;
adding, to the culture, every two days:
about 1.2 millimoles cysteine per liter of culture,
about 1 micromole Fe per liter of culture,
about 6 micromoles Zn per liter of culture,
about 0.1 micromoles Cu per liter of culture,
about 8 micromoles EDTA per liter of culture, and
about 1 micromoles citrate per liter of culture;
optionally, wherein the CDM includes thioctic acid, vitamin C and/or a mixture of hypotaurine, taurine and glycine
optionally wherein the CDM includes uridine, manganese, galactose and/or dexamethasone.

25. The VEGF mini-trap or composition of claim 1, wherein:
one or more asparagines of the VEGF mini-trap are N-glycosylated;
one more serines or threonines of the VEGF mini-trap are Oglycosylated;
one or more asparagines of the VEGF mini-trap are deamidated;
one or more Aspartate-Glycine motifs of the VEGF mini-trap are converted to iso-aspartate-glycine and/or Asn-Gly;
one or more methionines of the VEGF mini-trap are oxidized;
one or more tryptophans of the VEGF mini-trap are converted to N-formylkynurenin;
one or more arginines of the VEGF mini-trap are converted to Arg 3-deoxyglucosone; the C-terminal Glycine of the VEGF mini-trap is not present;
there are one or more non-glycosylated glycosites in the VEGF mini-trap;
the VEGF mini-trap comprises about 40% to about 50% total fucosylated glycans;
the VEGF mini-trap comprises about 30% to about 55% total sialylated glycans;
the VEGF mini-trap comprises about 6% to about 15% mannose-5;
the VEGF mini-trap comprises about 60% to about 79% galactosylated glycans;
the VEGF mini-trap is xylosylated;
the VEGF mini-trap is glycated at a lysine;
the VEGF mini-trap comprises a cystine with a free-thiol group;
the VEGF mini-trap comprises a trisulfide bridge;
the VEGF mini-trap comprises an intrachain disulfide bridge;
the VEGF mini-trap comprises disulfide bridges in parallel orientation; and/or the VEGF mini-trap comprises a lysine or arginine which is carboxymethylated.

26. The VEGF mini-trap or composition of claim 1, wherein one or more asparagines of the VEGF mini-trap comprise:
G0-GlcNAc glycosylation;
G1-GlcNAc glycosylation;
G1S-GlcNAc glycosylation;
G0 glycosylation;
G1 glycosylation;
G1S glycosylation;
G2 glycosylation;
G2S glycosylation;
G2S2 glycosylation;
G0F glycosylation;
G2F2S glycosylation;
G2F2S2 glycosylation;
G1F glycosylation;
G1FS glycosylation;
G2F glycosylation;
G2FS glycosylation;
G2FS2 glycosylation;
G3FS glycosylation;
G3FS3 glycosylation;
G0-2GlcNAc glycosylation;
Man4 glycosylation;
Man4_A1G1 glycosylation;
Man4_A1G1S1 glycosylation;
Man5 glycosylation;
Man5_A1G1 glycosylation;
Man5_A1G1S1 glycosylation;
Man6 glycosylation;
Man6_G0+Phosphate glycosylation;
Man6+Phosphate glycosylation; and/or
Man7 glycosylation.

27. The VEGF mini-trap or composition of claim 1, wherein the VEGF mini-trap comprises:
Man5 glycosylation at about 30-35% of asparagine 123 residues;
Man5 glycosylation at about 25-30% of asparagine 196 residues;
Man6-phosphate glycosylation at about 6-8% of asparagine 36 residues;
Man7 glycosylation at about 3-4% of asparagine 123 residues;
High mannose glycosylation at about 38% of asparagine 123 residues; and/or
High mannose glycosylation at about 29% of asparagine 196 residues.

28. The VEGF mini-trap or composition of claim 1, which is a composition that comprises the VEGF mini-trap at a concentration of about 80, 85, 90, 80-90, 100, 105, 110, 115, 120, 125, 130 or 90-120 mg/ml.

29. The VEGF mini-trap or composition of claim 1, wherein
the composition is aqueous;
the mini-trap was expressed in a Chinese hamster ovary cell;
the pH of the composition is about 5.5, 5.6, 5.7, 5.8, 5.9, 6.0, 6.1 or 6.2; and/or
the mini-trap has not been exposed to any greater than about 0.24, 0.6, 0.96, 1.2 or 2.4 million lux*hr white light; and/or any greater than about 40, 100, 160, 200 or 400 W*h/m² ultra-violet A (UVA) light.

30. The VEGF mini-trap or composition of claim 1, wherein the VEGF mini-trap is a monomer, homodimer or multimer.

31. A pharmaceutical formulation comprising the VEGF mini-trap or composition of claim 1 and a pharmaceutically acceptable carrier.

32. An injection device comprising the VEGF mini-trap polypeptide or composition or formulation of claim 1.

33. The injection device of claim 32 which is a sterile pre-filled syringe.

34. The VEGF mini-trap polypeptide or composition of claim 1 in association with a further therapeutic agent.

35. An isolated polynucleotide encoding the VEGF mini-trap of of claim 1.

36. A vector comprising the polynucleotide of claim 35.

37. A host cell comprising the VEGF mini-trap of claim 1.

38. The host cell of claim 37 which is a Chinese hamster ovary cell.

39. A method for making the VEGF mini-trap of claim 1 comprising introducing a polynucleotide encoding said polypeptide into a host cell, culturing the host cell in a medium under conditions wherein the polypeptide is expressed and, optionally, isolating the polypeptide from the host cell and/or medium.

40. The method of claim 39 wherein the host cell is a Chinese hamster ovary cell.

41. A VEGF mini-trap which is the product of claim 39.

42. A method for making the VEGF mini-trap of claim 1 consisting essentially of proteolyzing a VEGF Trap with an enzyme that cleaves an immunoglobulin Fc polypeptide after the following sequence: DKTHTCPPCPAPELLG (SEQ ID NO: 20).

43. The method of claim 42 wherein the VEGF Trap is aflibercept or conbercept.

44. The method of claim 42 wherein the enzyme is S. pyogenes IdeS or Streptococcus equi subspecies zooepidemicus IdeZ.

45. A method for administering said VEGF mini-trap or composition of claim 1 to a subject comprising introducing the VEGF mini-trap or composition or formulation, and optionally a further therapeutic agent, into the body of the subject.

46. The method of claim 45 wherein said VEGF mini-trap is administered to the body of the subject by intraocular injection.

47. The method of claim 45 wherein said VEGF mini-trap is administered intraocularly to the body of the subject by intravitreal injection.

48. A method for treating an angiogenic eye disorder in a subject in need thereof, the method comprising intraocularly injecting a therapeutically effective amount of the VEGF mini-trap or composition of claim 1, and optionally a further therapeutic agent, into an eye of the subject.

49. The method of claim 45, wherein about 0.5 mg, 2 mg, 4 mg, 6 mg, 8 mg or 10 mg of the VEGF mini-trap is intravitreally injected into the eye of the subject.

50. The method of claim 48, wherein the angiogenic eye disorder is
age-related macular degeneration (wet),
age-related macular degeneration (dry),
macular edema,
macular edema following retinal vein occlusion,
retinal vein occlusion (RVO),
central retinal vein occlusion (CRVO),
branch retinal vein occlusion (BRVO),
diabetic macular edema (DME),
choroidal neovascularization (CNV),
iris neovascularization,
neovascular glaucoma,
post-surgical fibrosis in glaucoma,
proliferative vitreoretinopathy (PVR),
optic disc neovascularization,
corneal neovascularization,
retinal neovascularization,
vitreal neovascularization,
pannus,
pterygium,
vascular retinopathy,
diabetic retinopathy, wherein the subject also has diabetic macular edema; and
diabetic retinopathy.

51. The method of claim 45 wherein the VEGF mini-trap is administered in about 100 microliters or less.


SEQUENCE LISTING (52 sequences)

SEQ ID NO: 1 - protein, 103 aa, Homo sapiens
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIID

SEQ ID NO: 2 - protein, 93 aa, Homo sapiens
PFVEMYSEIPEIIHMTEGRELVIPCRVTSPNITVTLKKFPLDTLIPDGKRIIWDSRKGFIISNATYKEIGLLTCEATVNGHLYKTNYLTHRQT

SEQ ID NO: 3 - protein, 102 aa, Homo sapiens
VVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 4 - protein, 97 aa, Homo sapiens
PFVAFGSGMESLVEATVGERVRIPAKYLGYPPPEIKWYKNGIPLESNHTIKAGHVLTIMEVSERDTGNYTVILTNPISKEKQSHVVSLVVYVPPGPG

SEQ ID NO: 5 - protein, 92 aa, Homo sapiens
FVAFGSGMESLVEATVGERVRIPAKYLGYPPPEIKWYKNGIPLESNHTIKAGHVLTIMEVSERDTGNYTVILTNPIKSEKQSHVVSLVVYVP

SEQ ID NO: 6 - protein, 16 aa, Artificial Sequence
  multimerizing component
DKTHTCPPCPAPELLG

SEQ ID NO: 7 - protein, 16 aa, Artificial Sequence
  multimerizing component
DKTHTCPLCPAPELLG

SEQ ID NO: 8 - protein, 6 aa, Artificial Sequence
  multimerizing component
DKTHTC

SEQ ID NO: 9 - protein, 12 aa, Artificial Sequence
  multimerizing component
DKTHTCPLCPAP

SEQ ID NO: 10 - protein, 440 aa, Artificial Sequence
  mini-trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 11 - protein, 425 aa, Artificial Sequence
  mini-trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 12 - protein, 221 aa, Artificial Sequence
  mini-trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLG

SEQ ID NO: 13 - protein, 315 aa, Artificial Sequence
  mini-trap
GRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHENLSVAFGSGMESLVEATVGERVRIPAKYLGYPPPEIKWYKNGIPLESNHTIKAGHVLTIMEVSERDTGNYTVILTNPISKEKQSHVVSLVVYVPPGPGDKTHTCPLCPAPELLG

SEQ ID NO: 14 - DNA, 663 bp, Artificial Sequence
  mini-trap
agtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctgggg

SEQ ID NO: 15 - DNA, 654 bp, Artificial Sequence
  mini-trap
agtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccaccgtgctga

SEQ ID NO: 16 - DNA, 663 bp, Artificial Sequence
  mini-trap
agtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccaccgtgcccaccgtgctga

SEQ ID NO: 17 - protein, 432 aa, Artificial Sequence
  trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 526 aa, Artificial Sequence
  trap
GRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHENLSVAFGSGMESLVEATVGERVRIPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 19 - protein, 211 aa, Artificial Sequence
  Fc fragment
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 16 aa, Artificial Sequence
  cleavage site sequence
DKTHTCPPCPAPELLG

SEQ ID NO: 21 - protein, 227 aa, Artificial Sequence
  Fc domain
  MISC_FEATURE (8)..(8): Leu or Pro
  MISC_FEATURE (173)..(173): Ala or Thr
DKTHTCPXCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKXTPPVLDSDGSFFLYSKLTVDKSRWQQGNVFSCSVMHEALHNHYTQKSLSLSPGK

SEQ ID NO: 22 - protein, 9 aa, Artificial Sequence
  multimerizing component
DKTHTCPPC

SEQ ID NO: 23 - protein, 12 aa, Artificial Sequence
  multimerizing component
DKTHTCPPCPPC

SEQ ID NO: 24 - protein, 15 aa, Artificial Sequence
  multimerizing component
DKTHTCPPCPPCPPC

SEQ ID NO: 25 - protein, 9 aa, Artificial Sequence
  multimerizing component
  MISC_FEATURE (7)..(9): optionally repeated 1, 2, 3, 4 or 5 times
DKTHTCPPC

SEQ ID NO: 26 - protein, 214 aa, Artificial Sequence
  mini-trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPC

SEQ ID NO: 27 - protein, 217 aa, Artificial Sequence
  mini-trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPPC

SEQ ID NO: 28 - protein, 220 aa, Artificial Sequence
  mini-trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPPCPPC

SEQ ID NO: 29 - protein, 214 aa, Artificial Sequence; identical to SEQ ID NO: 26
  mini-trap
  MISC_FEATURE (212)..(214): can be repeated 1, 2, 3, 4 or 5 times

SEQ ID NO: 30 - protein, 415 aa, Artificial Sequence
  mini-trap
  MISC_FEATURE (206)..(210): can be repeated 1, 2, 3, 4, 5, 6, 7, 8, 9, 10, 11, 12, 13, 14 or 15 times
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 31 - protein, 165 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 32 - protein, 455 aa, Artificial Sequence
  mini-trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSGGGGSGGGGSGGGGSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 33 - protein, 470 aa, Artificial Sequence
  mini-trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSGGGGSGGGGSGGGGSGGGGSGGGGSGGGGSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 34 - protein, 453 aa, Artificial Sequence
  mini-trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKEQKLISEEDLGGEQKLISEEDLHHHHHH

SEQ ID NO: 35 - protein, 468 aa, Artificial Sequence
  mini-trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKEQKLISEEDLGGEQKLISEEDLHHHHHH

SEQ ID NO: 36 - protein, 440 aa, Artificial Sequence; identical to SEQ ID NO: 10
  mini-trap

SEQ ID NO: 37 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 38 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 39 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 40 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 41 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 42 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 43 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 44 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 45 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 46 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 47 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 48 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 49 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 50 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 51 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 52 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN